(19) United States
(12) Patent Application Publication
(10) Pub. No.: US 2022/0162163 A1
(43) Pub. Date: May 26, 2022

(54) ALANINE-BASED MODULATORS OF PROTEOLYSIS AND ASSOCIATED METHODS OF USE

(71) Applicant: Arvinas Operations, Inc., New Haven, CT (US)

(72) Inventors: Michael Berlin, Flemington, NJ (US); Andrew P. Crew, Chester, CT (US); Hanqing Dong, Madison, CT (US); Yimin Qian, Plainsboro, NJ (US)

(21) Appl. No.: 17/569,145

(22) Filed: Jan. 5, 2022

Related U.S. Application Data
(63) Continuation of application No. 15/209,648, filed on Jul. 13, 2016.
(60) Provisional application No. 62/192,056, filed on Jul. 13, 2015.

(51) Int. Cl. C07D 207/16; C07D 417/14; C07D 403/04; C07D 417/04; A61K 47/55; C07D 401/04; C07D 495/14; C07D 405/14; A61K 31/40; A61K 31/4178; A61K 31/4439; A61K 31/444; A61K 31/496; A61K 31/506; A61K 31/551; A61K 45/06; C07D 403/12; C07D 471/04; C07D 487/14; C07D 519/00

(52) U.S. Cl. CPC C07D 207/16; A61K 38/00; C07D 403/04; C07D 417/04; A61K 47/55; C07D 401/04; C07D 495/14; C07D 405/14; A61K 31/40; A61K 31/4178; A61K 31/4439; A61K 31/444; A61K 31/496; A61K 31/506; A61K 31/551; A61K 45/06; C07D 403/12; C07D 471/04; C07D 487/14; C07D 519/00; C07D 417/14

(57) ABSTRACT

The description relates to Inhibitors of Apoptosis Proteins (IAPs) binding compounds, including bifunctional compounds comprising the same, which find utility as modulators of targeted ubiquitination, especially inhibitors of a variety of polypeptides and other proteins which are degraded and/or otherwise inhibited by bifunctional compounds according to the present invention. In particular, the description provides compounds, which contain on one end a ligand which binds to the IAP E3 ubiquitin ligase and on the other end a moiety which binds a target protein such that the target protein is placed in proximity to the ubiquitin ligase to effect degradation (and inhibition) of that protein. Compounds can be synthesized that exhibit a broad range of pharmacological activities consistent with the degradation/inhibition of targeted polypeptides of nearly any type.

### CROSS-REFERENCE TO RELATED APPLICATIONS

This application is a continuation of U.S. patent application Ser. No. 15/209,648, filed 13 Jul. 2016, published as U.S. Patent Application Publication No. 2017-0037004 A1 on 9 Feb. 2017, which claims the benefit of and priority to U.S. Provisional Application No. 62/192,056, filed 13 Jul. 2015, both of which are incorporated herein by reference in their entirety for all purposes.

### INCORPORATION BY REFERENCE

U.S. Patent Application Publications US 2015-0291562 entitled “Imide-Based Modulators of Proteolysis and Associated Methods of Use,” and US 2014-0356322 entitled “Compounds and Methods for the Enhanced Degradation of Targeted Proteins and Other Polypeptides by an E3 ubiquitin ligase,” as well as U.S. patent application Ser. No. 15/206,497 filed 11 Jul. 2016 entitled “MDM2-Based Modulators of Proteolysis and Associated Methods of Use,” are incorporated herein by reference in their entirety. Furthermore, all references cited herein are incorporated by reference herein in their entirety.

### FIELD OF THE INVENTION

The description provides imide-based compounds, including bifunctional compounds comprising the same, and associated methods of use. The bifunctional compounds are useful as modulators of targeted ubiquitination, especially with respect to a variety of polypeptides and other proteins, which are degraded and/or otherwise inhibited by bifunctional compounds according to the present invention.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped.

Inhibitors of Apotosis Proteins (IAPs) are a protein family involved in suppressing apoptosis, i.e. cell death. The human IAP family includes 8 members, and numerous other organisms contain IAP homologs. IAPs contain an E3 ligase specific domain and baculoviral IAP repeat (BIR) domains that recognize substrates, and promote their ubiquitination. IAPs promote ubiquitination and can directly bind and inhibit caspases. Caspases are proteases (e.g. caspase-3, caspase-7 and caspace-9) that implement apoptosis. As such, through the binding of caspases, IAPs inhibit cell death. However, pro-apoptotic stimuli can result in the release of mitochondrial proteins DIABLO (also known as second mitrochondria-derived activator of caspases or SMAC) and HTRA2 (also known as Omi). Binding of DIABLO and HTRA2 appears to block IAP activity.

SMAC interacts with essentially all known IAPs including XIAP, c-IAP1, c-IAP2, NIL-IAP. Bruce, and survivin. The first four amino acids (AVPI) of mature SMAC bind to a portion of IAPs, which is believed to be essential for blocking the anti-apoptotic effects of IAPs.

Bifunctional compounds such as those that are described in U.S. Patent Application Publications US 2015-0291562, and US 2014-0356322 (incorporated herein by reference), function to recruit endogenous proteins to an E3 ubiquiuin ligase for degradation. In particular, the publications describe bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds.

An ongoing need exists in the art for effective treatments for disease, especially hyperplasias and cancers, such as multiple myeloma. However, non-specific effects, and the inability to target and modulate certain classes of proteins altogether, such as transcription factors, remain as obstacles to the development of effective anti-cancer agents. As such, small-molecule therapeutic agents that leverage or potentiate IAPs' substrate specificity and, at the same time, are “tunable” such that a wide range of protein classes can be targeted and modulated would be very useful.

### SUMMARY

The present disclosure describes bifunctional compounds which function to recruit endogenous proteins to an E3 ubiquitin ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer, e.g., multiple myeloma.

As such, in one aspect the disclosure provides bifunctional or PROTAC compounds, which comprise an E3 ubiquitin ligase binding moiety (i.e., a ligand for an E3 ubquitin ligase or “ULM” group), and a moiety that binds a target protein (i.e., a protein/polypeptide targeting ligand or “PTM” group) such that the target protein/polypeptide is placed in proximity to the ubiquitin ligase to effect degradation (and inhibition) of that protein. In a preferred embodiment, the ULM is an IAP E3 ubiquitin ligase binding moiety (i.e., a “ILM”). For example, the structure of the bifunctional compound can be depicted as:

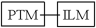

The respective positions of the PTM and ILM moieties as well as their number as illustrated herein is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

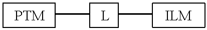

where PTM is a protein/polypeptide targeting moiety, L is a linker, e.g., a bond or a chemical group coupling PTM to ILM, and ILM is a IAP E3 ubiquitin ligase binding moiety.

In certain preferred embodiments, the ILM is an AVPI tetrapeptide fragment. As such, in certain additional embodiments, the ILM of the bifunctional compound comprises the amino acids alanine (A), valine (V), proline (P), and isoleucine (I) or their unnatural mimetics, respectively. In additional embodiments, the amino acids of the AVPI tetrapeptide fragment are connected to each other through amide bonds (i.e., —C(O)NH— or —NHC(O)—).

In certain embodiments, the compounds as described herein comprise multiple ILMs, multiple PTMs, multiple chemical linkers or a combination thereof.

In another aspect, this invention provides bifunctional molecules where PTM can be an IAP binding moiety (ILM), and ULM (ubiquitination ligase modulator) can be Von Hippel-Lindau E3 ubiquitin ligase (VHL) binding moiety (VLM), or a cereblon E3 ubiquitin ligase binding moiety (CLM), or a mouse double minute 2 homolog (MDM2) E3 ubiquitin ligase binding moiety (MLM), and the two functional moieties are connected by linker “L” as shown below:

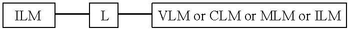

wherein, ILM is IAP binding moiety which binds to IAP; “L” is a bond or a chemical linker group; VLM is Von Hippel-Lindau E3 ubiquitin ligase binding moiety that binds to VHL E3 ligase; CLM is cereblon E3 ubiquitin ligase binding moiety that binds to cereblon, and MLM is an MDM2 E3 ubiquitin ligase binding moiety.

In certain embodiments, IBM comprises chemical moieties such as those described herein.

In additional embodiments, VLM can be hydroxyproline or a derivative thereof. Furthermore, other contemplated VLMs are included in U.S. Patent Application Pub. No. 2014-03022523, which as discussed above, is incorporated herein in its entirety.

In an embodiment, the CLM comprises a chemical group derived from an imide, a thioimide, an amide, or a thioamide. In a particular embodiment, the chemical group is a phthalimido group, or an analog or derivative thereof. In a certain embodiment, the CLM is thalidomide, lenalidomide, pomalidomide, analogs thereof, isosteres thereof, or derivatives thereof. Other contemplated CLMs are described in U.S. Patent Application Publication US 2015-0291562, which is incorporated herein in its entirety.

In certain embodiments, MLM can be nutlin or a derivative thereof. Furthermore, other contemplated MLMs are included in U.S. patent application Ser. No. 15/206,497 filed 11 Jul. 2016, which as discussed above, is incorporated herein in its entirety

In certain embodiments, “L” is a bond. In additional embodiments, the linker “L” is a connector with a linear non-hydrogen atom number in the range of 1 to 20. The connector “L” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bycyclic and tricyclic moieties. Substitution with halogen, such as Cl, F, Br and I can be included in the linker. In the case of fluorine substitution, single or multiple fluorines can be included.

In certain embodiments, VLM is a derivative of trans-3-hydroxyproline, where both nitrogen and carboxylic acid in trans-3-hydroxyproline are functionalized as amides.

In certain embodiments, CLM is a derivative of piperidine-2,6-dione, where piperidine-2,6-dione can be substituted at the 3-position, and the 3-substitution can be bicyclic hetero-aromatics with the linkage as C—N bond or C—C bond. Examples of CLM can be, but not limited to, pomalidomide, lenalidomide and thalidomide and their derivatives.

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer. In yet another aspect, the present invention provides a method of ubiquitinating/degrading a target protein in a cell. In certain embodiments, the method comprises administering a bifunctional compound as described herein comprising an ILM and a PTM, a IBM and a VLM, or a IBM and a CLM, or an ILM and a MLM preferably linked through a linker moiety, as otherwise described herein, wherein the ILM is coupled to the PTM through a linker to target protein that binds to PTM for degradation. Similarly, wherein IBM is coupled to VLM or CLM or MLM through a linker to target IAP for degradation. Degradation of the target protein will occur when the target protein is placed in proximity to the E3 ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

In still another aspect, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of proteins of interest in a biological system using compounds according to the present invention.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional aspects and embodiments are expressly included within the scope of the present invention. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIGS. 1A and 1B. Illustration of general principle for PROTAC function. (FIG. 1A) Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (FIG. 1B) Illustrates the functional use of the PROTACs as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degration by the proteosomal machinery of the cell.

### DETAILED DESCRIPTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

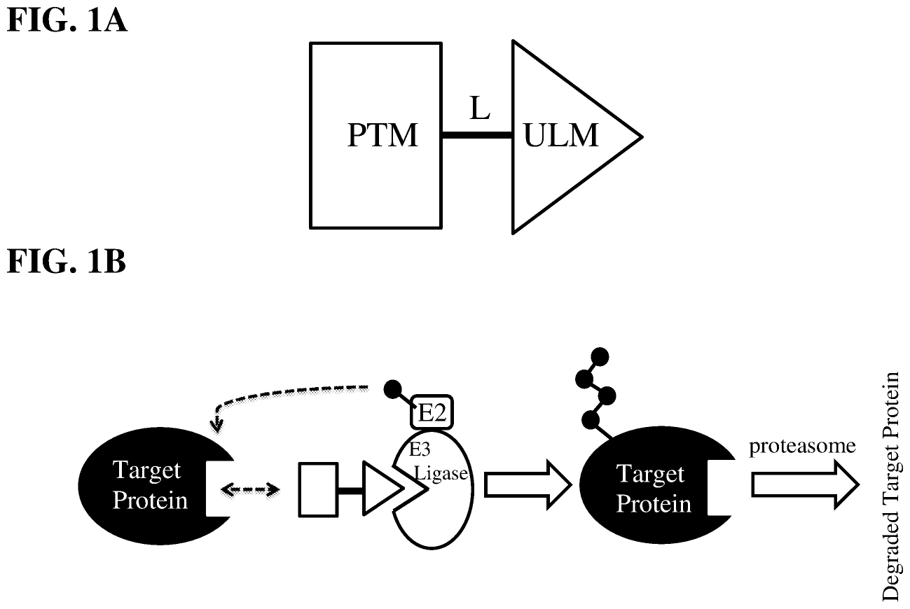

Presently described are compositions and methods that relate to the surprising and unexpected discovery that an E3 ubiquitin ligase protein, e.g., inhibitors of apoptosis proteins (IAP), ubiquitinates a target protein once it and the target protein are placed in proximity by a bifunctional or chimeric construct that binds the E3 ubiquitin ligase protein and the target protein. Accordingly the present invention provides such compounds and compositions comprising an E3 ubiquintin ligase binding moiety (“ULM”) coupled to a protein target binding moiety (“PTM”), which result in the ubiquitination of a chosen target protein, which leads to degradation of the target protein by the proteasome (see FIGS. 1A and 1B). The present invention also provides a library of compositions and the use thereof.

In certain aspects, the disclosure provides compounds which contain a ligand, e.g., a small molecule ligand (i.e., having a molecular weight of below 2,000, 1,000, 500, or 200 Daltons), which is capable of binding to an E3 ubiquitin ligase, such as IAP, and a moiety that is capable of binding to a target protein, in such a way that the target protein is placed in proximity to the ubiquitin ligase to effect degradation (and/or inhibition) of that protein.

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

The term “ubiquitin ligase” refers to a family of proteins that facilitate the transfer of ubiquitin to a specific substrate protein, targeting the substrate protein for degradation. For example, IAP an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

The term “patient” or “subject” is used throughout the specification to describe an animal, preferably a human or a domesticated animal, to whom treatment, including prophylactic treatment, with the compositions according to the present invention is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

Comounds and Compositions

In one aspect, the description provides compounds comprising an E3 ubiquitin ligase binding moiety (“ULM”) that is a IAP E3 ubiquitin ligase binding moiety (“an ILM”). In an exemplary embodiment, the ILM is coupled to a chemical linker (L) according to the structure:

L-ILM (I)

wherein L is a bond or a chemical linker group and ILM is a IAP E3 ubiquitin ligase binding moiety. The number and/or relative positions of the moieties in the compounds illustrated herein is provided by way of example only. As would be understood by the skilled artisan, compounds as described herein can be synthesized with any desired number and/or relative position of the respective functional moieties.

The terms ULM and ILM are used in their inclusive sense unless the context indicates otherwise. For example, the term ULM is inclusive of all ULMs, including those that bind IAP (i.e., ILMs). Further, the term ILM is inclusive of all possible IAP E3 ubiquitin ligase binding moieties.

In another aspect, the present invention provides bifunctional or multifunctional compounds (e.g., PROTACs) useful for regulating protein activity by inducing the degradation of a target protein. In certain embodiments, the compound comprises an ILM coupled, e.g., linked covalently, directly or indirectly, to a moiety that binds a target protein (i.e., protein targeting moiety or “PTM”). In certain embodiments, the ILM and PTM are joined or coupled via a chemical linker (L). The ILM binds the IAP E3 ubiquitin ligase and the PTM recognizes a target protein and the interaction of the respective moieties with their targets facilitates the degradation of the target protein by placing the target protein in proximity to the ubiquitin ligase protein. An exemplary bifunctional compound can be depicted as:

PTM-ILM (II)

In certain embodiments, the bifunctional compound further comprises a chemical linker (“L”). For example, the bifunctional compound can be depicted as:

PTM-L-ILM (III)

wherein PTM is a protein/polypeptide targeting moiety, L is a chemical linker, and ILM is a IAP E3 ubiquitin ligase binding moiety.

In certain embodiments, the ILM shows activity or binds to IAP with an IC₅₀ of less than about 200 μM. The IC₅₀ can be determined according to any method known in the art, e.g., a fluorescent polarization assay.

In certain additional embodiments, the bifunctional compounds described herein demonstrate an activity with an IC₅₀ of less than about 100, 50, 10, 1, 0.5, 0.1, 0.05, 0.01, 0.005, 0.001 mM, or less than about 100, 50, 10, 1, 0.5, 0.1, 0.05, 0.01, 0.005, 0.001 μM, or less than about 100, 50, 10, 1, 0.5, 0.1, 0.05, 0.01, 0.005, 0.001 nM, or less than about 100, 50, 10, 1, 0.5, 0.1, 0.05, 0.01, 0.005, 0.001 pM.

In certain embodiments, the compounds as described herein comprise multiple PTMs (targeting the same or different protein targets), multiple ILMs, one or more ULMs (i.e., moieties that bind specifically to another E3 ubiquitin ligase, e.g., VHL) or a combination thereof. In any of the aspects of embodiments described herein, the PTMs, ILMs, and ULMs can be coupled directly or via one or more chemical linkers or a combination thereof. In additional embodiments, where a compound has multiple ULMs, the ULMs can be for the same E3 ubiquintin ligase or each respective ULM can bind specifically to a different E3 ubiquitin ligase. In still further embodiments, where a compound has multiple PTMs, the PTMs can bind the same target protein or each respective PTM can bind specifically to a different target protein.

In another embodiment, the description provides a compound which comprises a plurality of ILMs coupled directly or via a chemical linker moiety (L). For example, a compound having two ILMs can be depicted as:

ILM-ILM or (IV)

ILM-L-ILM (V)

In certain embodiments, where the compound comprises multiple ILMs, the ILMs are identical. In additional embodiments, the compound comprising a plurality of ILMs further comprises at least one PTM coupled to a ILM directly or via a chemical linker (L) or both. In certain additional embodiments, the compound comprising a plurality of ILMs further comprises multiple PTMs. In still additional embodiments, the PTMs are the same or, optionally, different. In still further embodiments, wherein the PTMs are different the respective PTMs may bind the same protein target or bind specifically to a different protein target.

In additional embodiments, the description provides a compound comprising at least two different ILMs coupled directly or via a chemical linker (L) or both. For example, such a compound having two different ILMs can be depicted as:

ILM-ILM′ or (VI)

ILM-L-ILM′ (VII)

wherein ILM′ indicates a IAP E3 ubiquitin ligase binding moiety that is structurally different from ILM. In certain embodiments, the compound may comprise a plurality of ILMs and/or a plurality of ILM's. In further embodiments, the compound comprising at least two different ILMs, a plurality of ILMs, and/or a plurality of ILM's further comprises at least one PTM coupled to a ILM or a ILM′ directly or via a chemical linker or both. In any of the embodiments described herein, a compound comprising at least two different ILMs can further comprise multiple PTMs. In still additional embodiments, the PTMs are the same or, optionally, different. In still further embodiments, wherein the PTMs are different the respective PTMs may bind the same protein target or bind specifically to a different protein target. In still further embodiments, the PTM itself is a ULM or ILM (or ULM′ or ILM′).

In a preferred embodiment, the ILM comprises a moiety that is a ligand of the IAP E3 ubiquitin ligase.

In additional embodiments, the description provides the compounds as described herein including their enantiomers, diastereomers, solvates and polymorphs, including pharmaceutically acceptable salt forms thereof, e.g., acid and base salt forms.

Exemplary ILMs

AVPI Tetrapeptide Fragments

In any of the compounds described herein, the ILM can comprise an alanine-valine-proline-isoleucine (AVPI) tetrapeptide fragment or an unnatural mimetic thereof. In certain embodiments, the ILM is selected from the group consisting of chemical structures represented by Formulas (I), (II), (III), (IV), and (V):

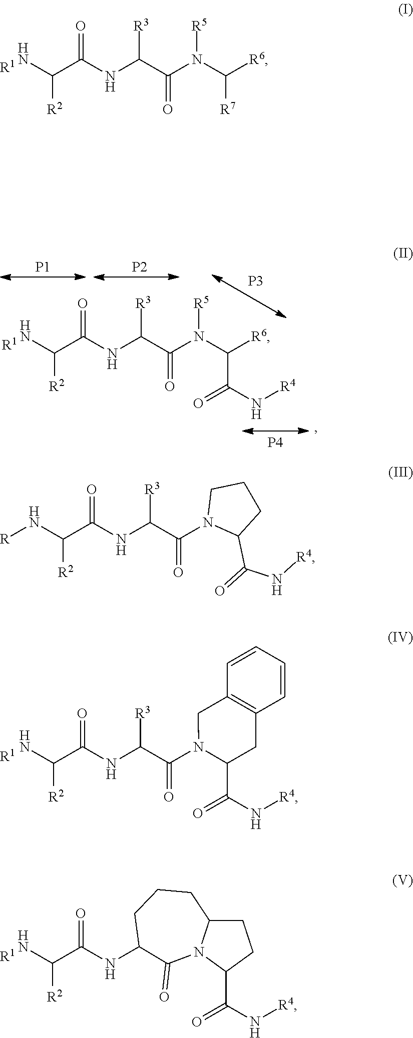

wherein:
R¹ for Formulas (I), (II), (III), (IV), and (V) is selected from H or alkyl;

R² for Formulas (I), (II), (III), (IV), and (V) is selected from H or alkyl;
R³ for Formulas (I), (II), (III), (IV), and (V) is selected from H, alkyl, cycloalkyl and heterocycloalkyl;
R⁵ and R⁶ for Formulas (I), (II), (III), (IV), and (V) are independently selected from H, alkyl, cycloalkyl, heterocycloalkyl, or more preferably, R⁵ and R⁶ taken together for Formulas (I), (II), (III), (IV), and (V) form a pyrrolidine or a piperidine ring further optionally fused to 1-2 cycloalkyl, heterocycloalkyl, aryl or heteroaryl rings, each of which can then be further fused to another cycloalkyl, heterocycloalkyl, aryl or heteroaryl ring;
R³ and R⁵ for Formulas (I), (II), (III), (IV), and (V) taken together can form a 5-8-membered ring further optionally fused to 1-2 cycloalkyl, heterocycloalkyl, aryl or heteroaryl rings;
R⁷ for Formulas (I), (II), (III), (IV), and (V) is selected from cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or R⁷ is —C(O)NH—R⁴; and
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above.

As shown above, P1, P2, P3, and P4 of Formular (II) correlate with A, V, P, and I, respectively, of the AVPI tetrapeptide fragment or an unnatural mimetic thereof. Similarly, each of Formulas (I) and (III) through (V) have portions correlating with A, V, P, and I of the AVPI tetrapeptide fragment or an unnatural mimetic thereof.

In any of the compounds described herein, the ILM can have the structure of Formula (VI), which is a derivative of IAP antagonists described in WO Pub. No. 2008/014236, or an unnatural mimetic thereof:

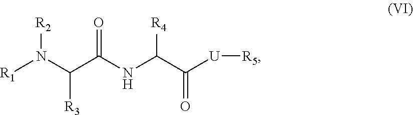

wherein:
R₁ of Formula (VI) is, independently selected from H, C₁-C₄-alky, C₁-C₄-alkenyl, C₁-C₄-alkynyl or C₃-C₁₀-cycloalkyl which are unsubstituted or substituted;
R² of Formula (VI) is, independently selected from H. C₁-C₄-alkyl, C₁-C₄-alkenyl, C₁-C₄-alkynyI or C₃-C₁₀-cycloalkyl which are unsubstituted or substituted;
R³ of Formula (VI) is, independently selected from H, —CF₃, —C₂H₅, C₁-C₄-alkyl, C₁-C₄-alkenyl, C₁-C₄-alkynyl, —CH₂—Z or any R₂ and R³ together form a heterocyclic ring;
each Z of Formula (VI) is, independently selected from H, —OH, F, Cl, —CH₃, —CF₃, —CH₂Cl, —CH₂F or —CH₂OH;
R₄ of Formula (VI) is, independently selected from C₁-C₁₆ straight or branched alkyl, C₁-C₁₆-alkenyl. C₁-C₁₆-alkynyl, C₃-C₁₀-cycloalkyl, —(CH₂)₀₋₆—Z₁, —(CH₂)₀₋₆-aryl, and —(CH₂)₀₋₆-bet, wherein alkyl, cycloalkyl, and phenyl are unsubstituted or substituted;
R₅ of Formula (VI) is, independently selected from H, C₁₋₁₀-alkyl, aryl, phenyl, C₃₋₇-cycloalkyl, —(CH₂)₁₋₆—C₃₋₇-cycloalkyl, —C₁₋₁₀-alkyl-aryl, —(CH₂)₀₋₆—C₃₋₇-cycloalkyl-(CH₂)₀₋₆-phenyl, —(CH₂)₀₋₄—CH[(CH₂)₁₋₄-phenyl]₂, indanyl, —C(O)—C₁₋₁₀-alkyl, —C(O)—(CH₂)₁₋₆—C₃₋₇-cycloalkyl, —C(O)—(CH₂)₀₋₆-phenyl, —(CH₂)₀₋₆—C(O)-phenyl, —(CH₂)₀₋₆-het, —C(O)—(CH₂)₁₋₆-het, or R₅ is selected from a residue of an amino acid, wherein the alkyl, cycloalkyl, phenyl, and aryl substituents are unsubstituted or substituted;
Z₁ of Formula (VI) is, independently selected from —N(R₁₀)—C(O)—C₁₋₁₀-alkyl, —N(R₁₀)—C(O)—(CH₂)₀₋₆—C₃₋₇-cycloalkyl, —N(R₁₀)—C(O)—(CH₂)₀₋₆-phenyl, —N(R₁₀)—C(O)(CH₂)₁₋₆-het, —C(O)—N(R₁₁)(R₁₂), —C(O)—O—C₁₋₁₀-alkyl, —C(O)—O—(CH₂)₁₋₆—C₃₋₇-cycloalkyl, —C(O)—O—(CH₂)₀₋₆-phenyl, —C(O)—O—(CH₂)₁₋₆-het, —O—C(O)—C₁₋₁₀-alkyl, —O—C(O)—(CH₂)₁₋₆—C₃₋₇-cycloalkyl, —O—C(O)—(CH₂)₀₋₆-phenyl, —O—C(O)—(CH₂)₁₋₆-het, wherein alkyl, cycloalkyl, and phenyl are unsubstituted or substituted;
het of Formula (VI) is, independently selected from a 5-7 member heterocyclic ring containing 1-4 heteroatoms selected from N, O, and S, or an 8-12 member fused ring system including at least one 5-7 member heterocyclic ring containing 1, 2, or 3 heteroatoms selected from N, O, and S, which heterocyclic ring or fused ring system is unsubstituted or substituted on a carbon or nitrogen atom;
R₁₀ of Formula (VI) is selected from H, —CH₃, —CF₃, —CH₂OH, or —CH₂Cl;
R₁₁ and R₁₂ of Formula (VI) are independently selected from H, C₁₋₄-alkyl. C₃₋₇-cycloalkyl. —(CH₂)₁₋₆—C₃₋₇-cycloakyl, (CH₂)₀₋₆-phenyl, wherein alkyl, cycloalkyl, and phenyl are unsubstituted or substituted; or R₁₁ and R₁₂ together with the nitrogen form het, and
U of Formula (VI) is, independently, as shown in Formula (VII):

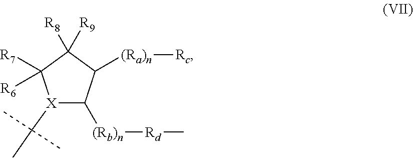

wherein:
each n of Formula (VII) is, independently selected from 0 to 5;
X of Formula (VII) is selected from the group —CH and N;
Rₐ and R_{b}, of Formula (VII) are independently selected from the group O. S, or N atom or C₀₋₈-alkyl wherein one or more of the carbon atoms in the alkyl chain are optionally replaced by a heteroatom selected from O, S, or N, and where each alkyl is, independently, either unsubstituted or substituted;
R_{d} of Formula (VII) is selected from the group Re-Q-(R_{f})ₚ(R_{g})_{q}, and Ar₁-D-Ar₂;
Rₑ of Formula (VII) is selected from the group H or any R_{c} and R_{d} together form a cycloalkyl or het; where if R_{c} and R_{d} form a cycloalkyl or het, R₅ is attached to the formed ring at a C or N atom;
p and q of Formula (VII) are independently selected from 0 or 1;
Rₑ of Formula (VII) is selected from the group C₁₋₈-alkyl and alkylidene, and each Re is either unsubstituted or substituted;
Q is selected from the group N, O, S, S(O), and S(O)₂;
Ar₁ and Ar₂ of Formula (VII) are independently selected from the group of substituted or unsubstituted aryl and het;
R_{f} and R_{g} of Formula (VII) are independently selected from H, —C1-10-alkyl, C₁₋₁₀-alkylaryl. —OH, —O—C₁₋₁₀-alkyl, —(CH₂)₀₋₆—C₃₋₇-cycloalkyl, —O—(CH₂)₀₋₆-aryl, phenyl, aryl, phenyl-phenyl, —(CH₂)₁₋₆-het. —O—(CH₂)₁₋₆-het, —OR₁₃, —C(O)—R₁₃, —C(O)—N(R₁₃)(R₁₄), —N(R₁₃)(R₁₄), —S—R₁₃, —S(O)—R₃, —S(O)₂—R₁₃, —S(O)₂—NR₁₃R₁₄, —NR₁₃—S(O)₂—R₁₄, —S—Cₜ₋₁₀-aIkyl, aryl-C₁₋₄-alkyl, or het-C₁₋₄-alkyl, wherein alkyl, cycloalkyl, het, and aryl are unsubstituted or substituted, —SO₂—C₁₋₂-alkyl, —SO₂—C₁₋₂-alkylphenyl, —O—C₁₋₄-alkyl, or any R_{g} and R_{f} together form a ring selected from het or aryl;
D of Formula (VII) is selected from the group —CO—, —C(O)—C₁₋₇-alkylene or arylene, —CF₂—, —O—, —S(O)ᵣ where r is 0-2, 1,3-dioxalane, or C₁₋₇-alkyl-OH; where alkyl, alkylene, or arylene are unsubstituted or substituted with one or more halogens, OH, —O—C₁₋₆-alkyl, —S—C₁₋₆-alkyl, or —CF₃; or each D is, independently selected from N(Rₕ);
Rh is selected from the group H, unsubstituted or substituted C₁₋₇-alkyl, aryl, unsubstituted or substituted —O—(C₁₋₇-cycloalkyl), —C(O)—C₁₋₁₀-alkyl, —C(O)—C₀₋₁₀-alkyl-aryl, —C—O—C₀₁₋₁₀-alkyl, —C—O—C₀₋₁₀-alkyl-aryl, —SO₂—C₁₋₁₀-alkyI, or —SO₂—(C₀₋₁₀-alkylaryl);
R₆, R⁷, R₈, and R₉ of Formula (VII) are, independently, selected from the group H, —C₁₋₁₀-alkyl, —C₁₋₁₀-alkoxy, aryl-C₁₋₁₀-alkoxy, —OH, —O—C₁₋₁₀-alkyl, —(CH₂)₀₋₆—C₃₋₇-cycloalkyI, —O—(CH₂)₀₋₆-aryl, phenyl, —(CH₂)₁₋₆-het, —O—(CH₂)₁₋₆-het, —OR₁₃, —C(O)—R₁₃, —C(O)—N(R₁₃)(R₁₄), —N(R¹³)(R₁₄), —S—R₁₃, —S(O)—R₁₃, —S(O)₂—R₁₃, —S(O)₂—NR₁₃R₁₄, or —NR₁₃—S(O)₂—R₁₄; wherein each alkyl, cycloalkyl, and aryl is unsubstituted or substituted; and any R₆, R₇, R₈, and R₉ optionally together form a ring system;
R₁₃ and R₁₄ of Formula (VII) are independently selected from the group H, C₁₋₁₀-alkyl, —(CH₂)₀₋₆—C₃₋₇-cycloalkyl, —(CH₂)₀₋₆—(CH)₀₋₁-(aryl)₁₋₂, —C(O)—C₁₋₁₀-alkyl, —C(O)—(CH₂)₁₋₆—C₃₋₇-cycloalkyl, —C(O)—O—(CH₂)₀₋₆-aryl, —C(O)—(CH₂)₀₋₆—O-fluorenyl, —C(O)—NH—(CH₂)₀₋₆-aryl, —C(O)—(CH₂)₀₋₆-aryl, —C(O)—(CH₂)₀₋₆-het, —C(S)—C₁₋₁₀-alkyl, —C(S)—(CH₂)₁₋₆—C₃₋₇-cycloalkyl, —C(S)—O—(CH₂)₀₋₆-aryl, —C(S)—(CH₂)₀₋₆—O-fluorenyl, —C(S)—NH—(CH₂)₀₋₆-aryl, —C(S)—(CH₂)₀₋₆-aryl, or —C(S)—(CH₂)₁₋₆-het, wherein each alkyl, cycloalkyl, and aryl is unsubstituted or substituted: or any R₁₃ and R₁₄ together with a nitrogen atom form het;
wherein alkyl substituents of R₁₃ and R₁₄ of Formula (VII) are unsubstituted or substituted and when substituted, are substituted by one or more substituents selected from C₁₋₁₀-alkyl, halogen, OH, —O—C₁₋₆-alkyl, —S—C₁₋₆-alkyl, and —CF₃; and substituted phenyl or aryl of R₁₃ and R₁₄ are substituted by one or more substituents selected from halogen, hydroxyl, C₁₋₄-alkyl, C₁₋₄-alkoxy, nitro, —CN, —O—C(O)—C₁₋₄-alkyl, and —C(O)—O—C₁₋₄-aryl; or a pharmaceutically acceptable salt or hydrate thereof.

In certain embodiments, the compound further comprises an independently selected second ILM attached to the ILM of Formula (VI), or an unnatural mimetic thereof, by way of at least one additional independently selected linker group. In an embodiment, the second ILM is a derivative of Formula (VI), or an unnatural mimetic thereof. In a certain embodiment, the at least one additional independently selected linker group comprises two additional independently selected linker groups chemically linking the ILM and the second ILM. In an embodiment, the at least one additional linker group for an ILM of the Formula (VI), or an unnatural mimetic thereof, chemically links groups selected from R₄ and R₅. For example, an ILM of Formula (VI) and a second ILM of Formula (VI), or an unnatural mimetic thereof, can be linked as shown below:

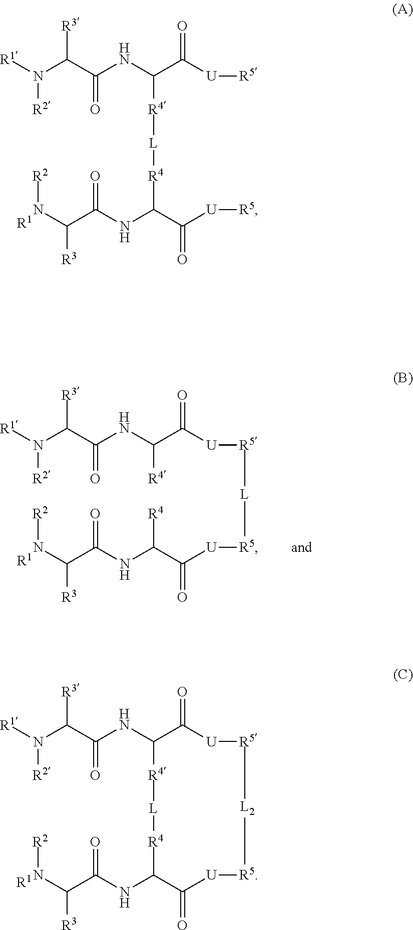

In certain embodiments, the ILM, the at least one additional independently selected linker group L, and the second ILM has a structure selected from the group consisting of:

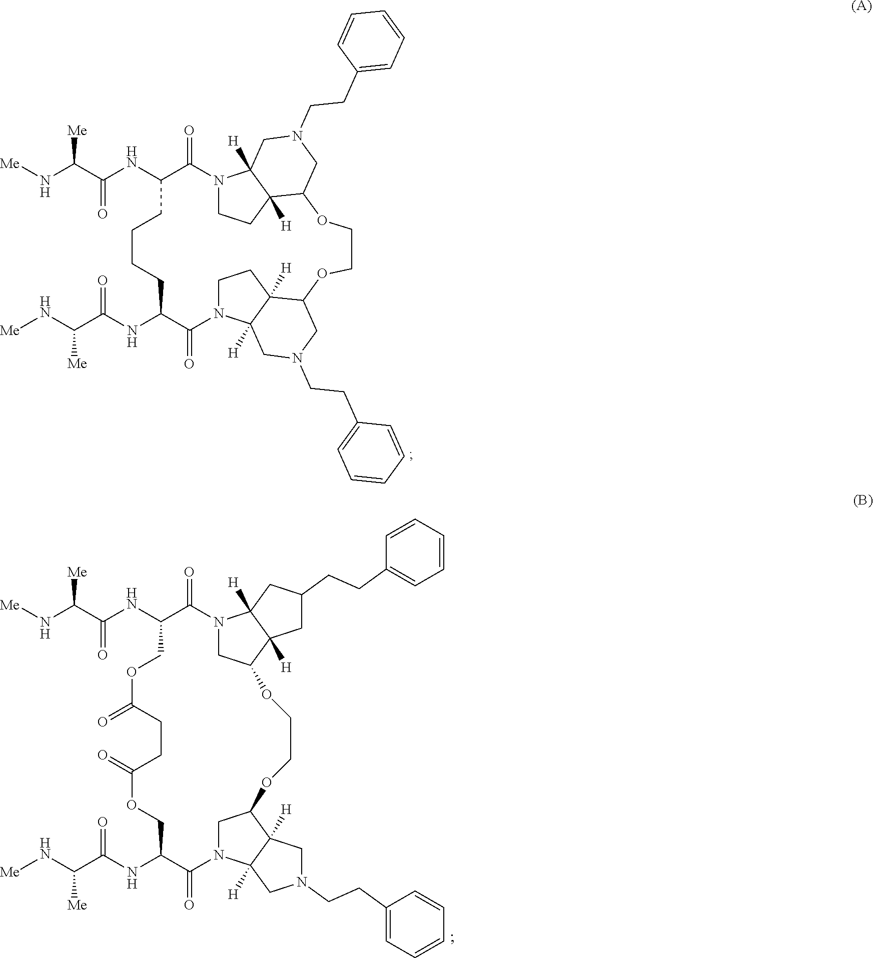 [SMILES: "(A)" ; CN[C@@H](C)C(=O)N[C@H]1CCCC[C@H](NC(=O)[C@H](C)NC)C(=O)N2CC[C@H]3C(CN(CCc4ccccc4)C[C@H]32)OCCOC2CN(CCc3ccccc3)C[C@@H]3[C@H]2CCN3C1=O ; "(B)" ; CN[C@@H](C)C(=O)N[C@H]1COC(=O)CCC(=O)OC[C@H](NC(=O)[C@H](C)NC)C(=O)N2C[C@@H](OCCO[C@@H]3CN(C1=O)[C@@H]1CC(CCc4ccccc4)C[C@@H]13)[C@H]1CN(CCc3ccccc3)C[C@H]12]

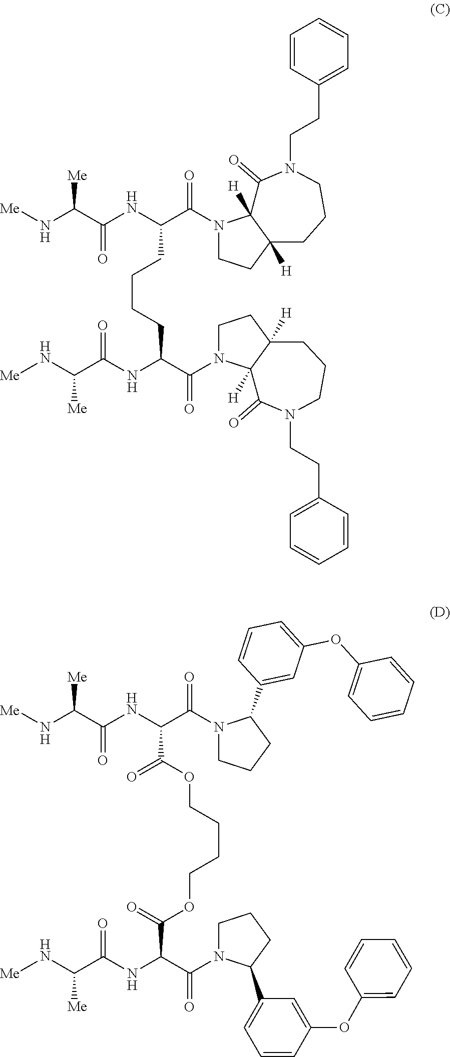 [SMILES: CN[C@@H](C)C(=O)N[C@@H](CCCC[C@H](NC(=O)[C@H](C)NC)C(=O)N1CC[C@H]2CCCN(CCc3ccccc3)C(=O)[C@H]21)C(=O)N1CC[C@H]2CCCN(CCc3ccccc3)C(=O)[C@H]21 ; CN[C@@H](C)C(=O)N[C@@H](C(=O)OCCCCOC(=O)[C@H](NC(=O)[C@H](C)NC)C(=O)N1CCC[C@H]1c1cccc(Oc2ccccc2)c1)C(=O)N1CCC[C@H]1c1cccc(Oc2ccccc2)c1]

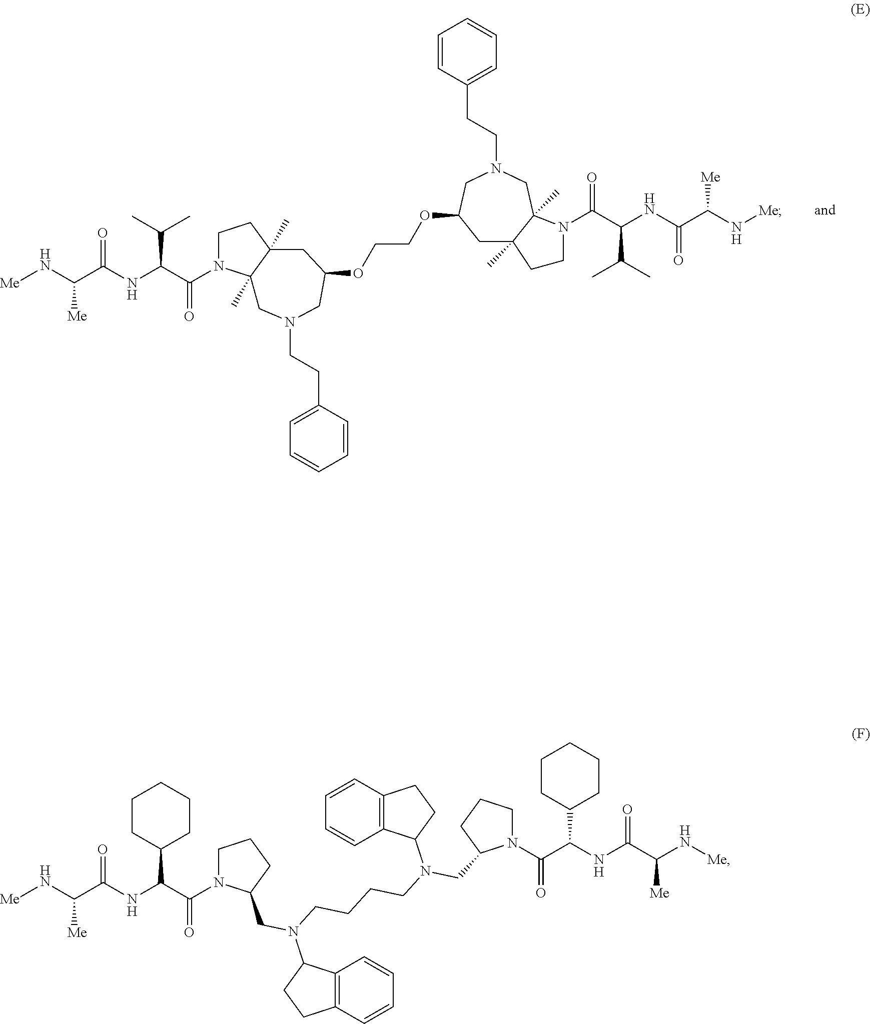

which are derivatives of IAP antagonists described in WO Pub. No. 2008/014236.

In any of the compounds described herein, the ILM can have the structure of Formula (VIII), which is based on the IAP ligrands described in Ndubaku, C., et al. Antagonism of c-IAP and XIAP proteins is required for efficient induction of cell death by small-molecule IAP antagonists, ACS Chem. Biol., 557-566, 4 (7) (2009), or an unnatural mimetic thereof:

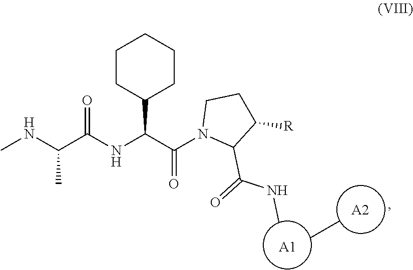

wherein:
each of A1 and A2 of Formula (VIII) is independently selected from optionally substituted monocyclic, fused rings, aryls and hetoroaryls; and
R of Formula (VIII) is selected from H or Me.

In a particular embodiment, the linker group L is attached to A1 of Formula (VIII). In another embodiment, the linker group L is attached to A2 of Formula (VIII).

In a particular embodiment, the ILM is selected from the group consisting of

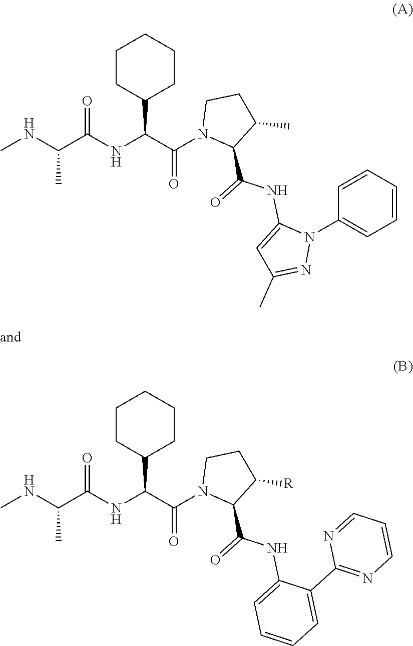

In any of the compounds described herein, the ILM can have the structure of Formula (IX), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

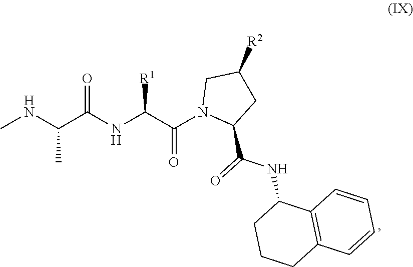

wherein:
R¹ is selected from alkyl, cycloalkyl and heterocycloalkyl and, most preferably, from isopropyl, tert-butyl, cyclohexyl and tetrahydropyranyl; and
R² of Formula (IX) is selected from —OPh or H.

In any of the compounds described herein, the ILM can have the structure of Formula (X), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

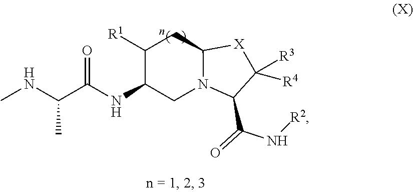

wherein:
R¹ of Formula (X) is selected from H, —CH₂OH, —CH₂CH₂OH, —CH₂NH₂, —CH₂CH₂NH₂; X of Formula (X) is selected from S or CH₂;
R² of Formula (X) is selected from:

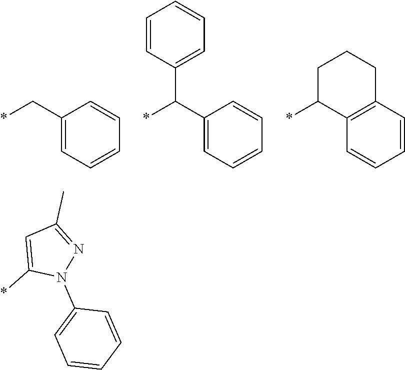

R³ and R⁴ of Formula (X) are independently selected from H or Me

In any of the compounds described herein, the ILM can have the structure of Formula (XI), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

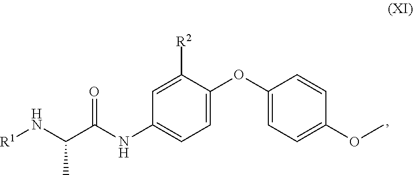

wherein:
R¹ of Formula (XI) is selected from H or Me, and
R² of Formula (XI) is selected from H or

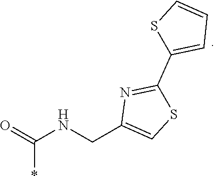 [SMILES: *C(=O)NCc1csc(-c2cccs2)n1]

In any of the compounds described herein, the ILM can have the structure of Formula (XII), which is derived from the chemotypes cross-referenced in Mannhold, R., et al. IAP antagonists: promising candidates for cancer therapy, Drug Discov. Today, 15 (5-6), 210-9 (2010), or an unnatural mimetic thereof:

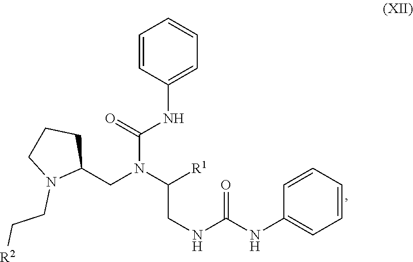

wherein:
R¹ of Formula (XII) is selected from:

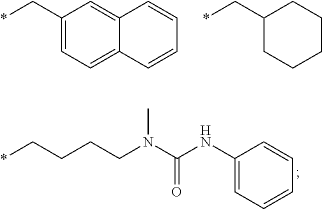

and
R² of Formula (XII) is selected from:

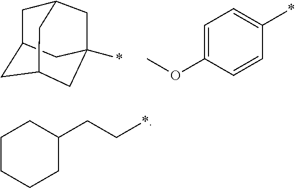

In any of the compounds described herein, the IAP E3 ubiquitin ligase binding moiety is selected from the group consisting of:

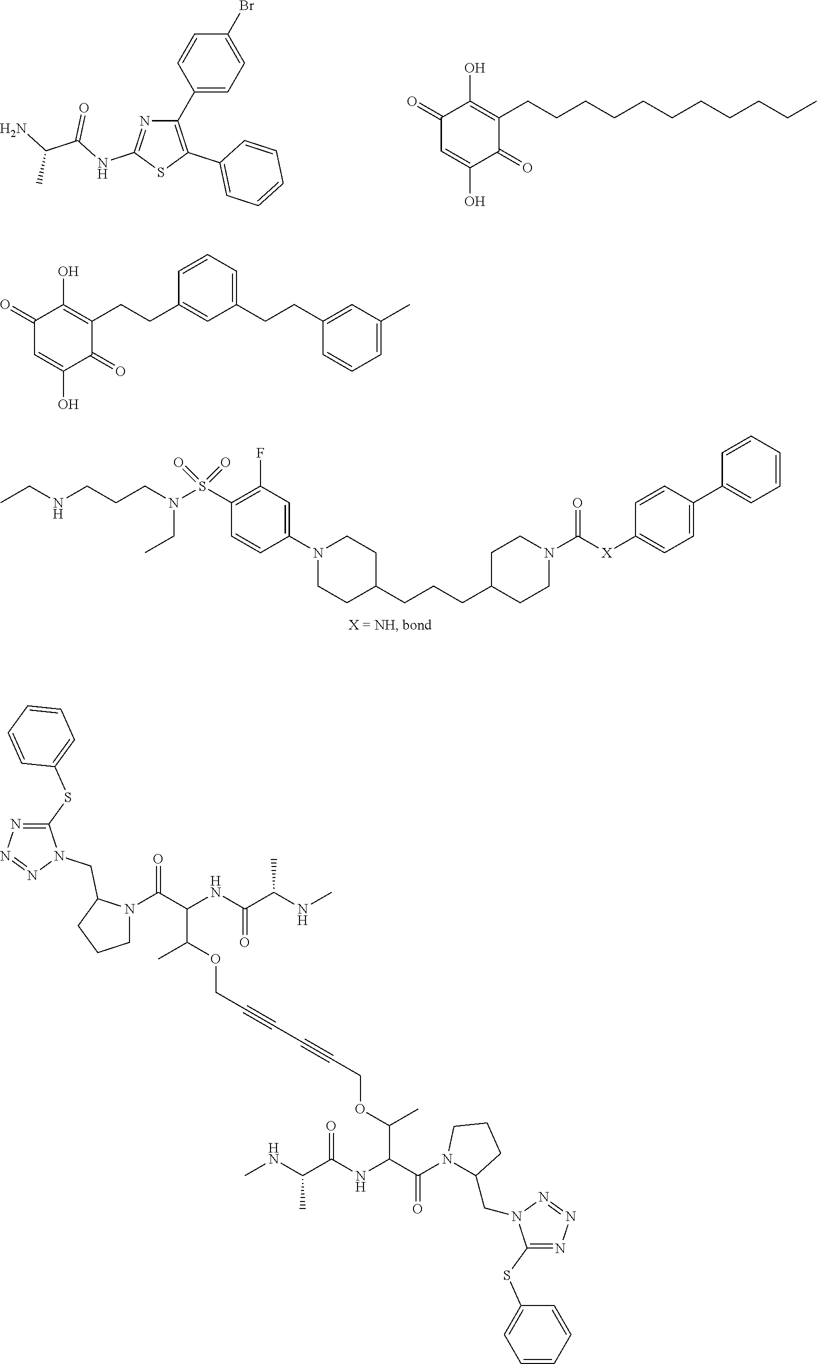

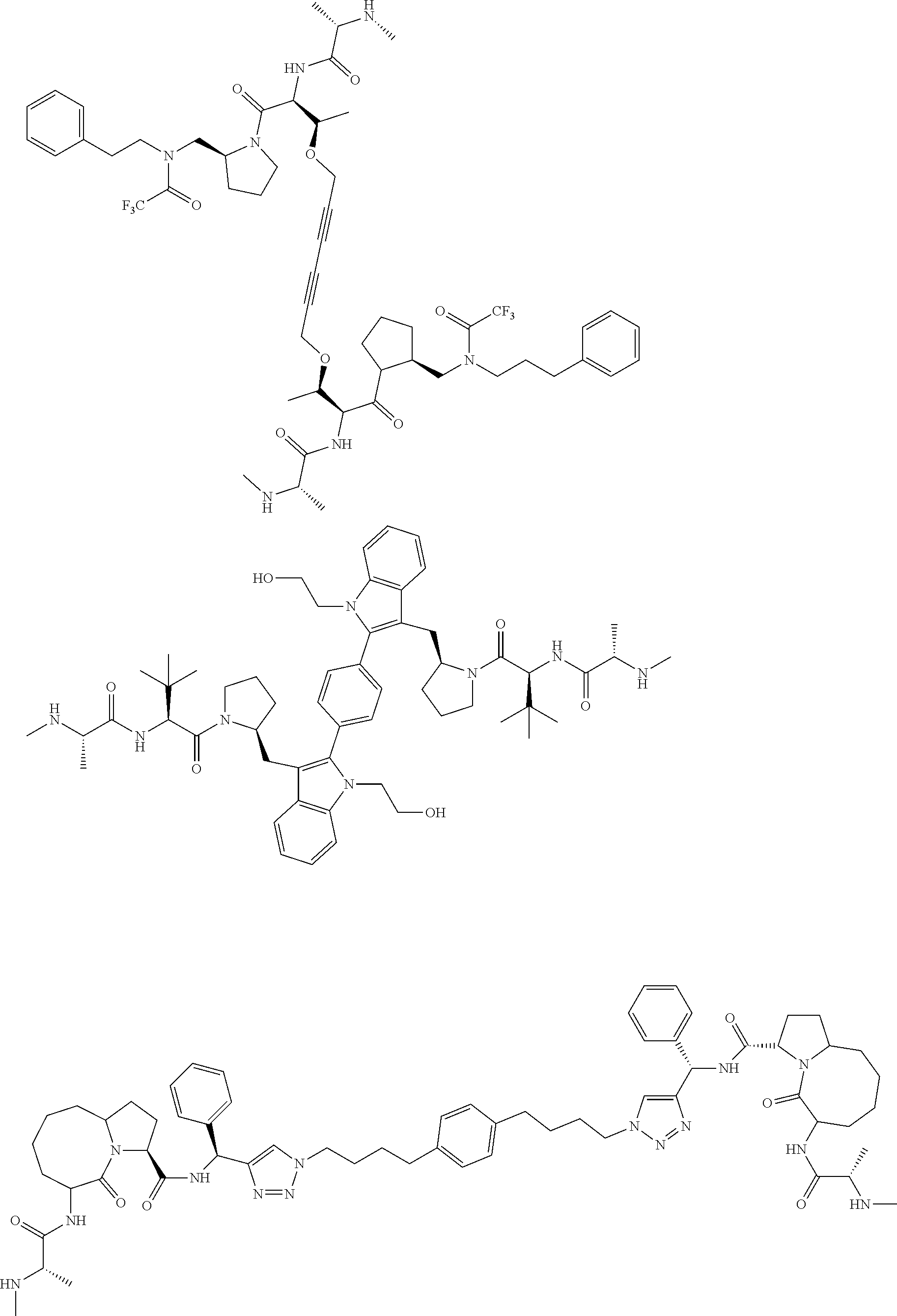

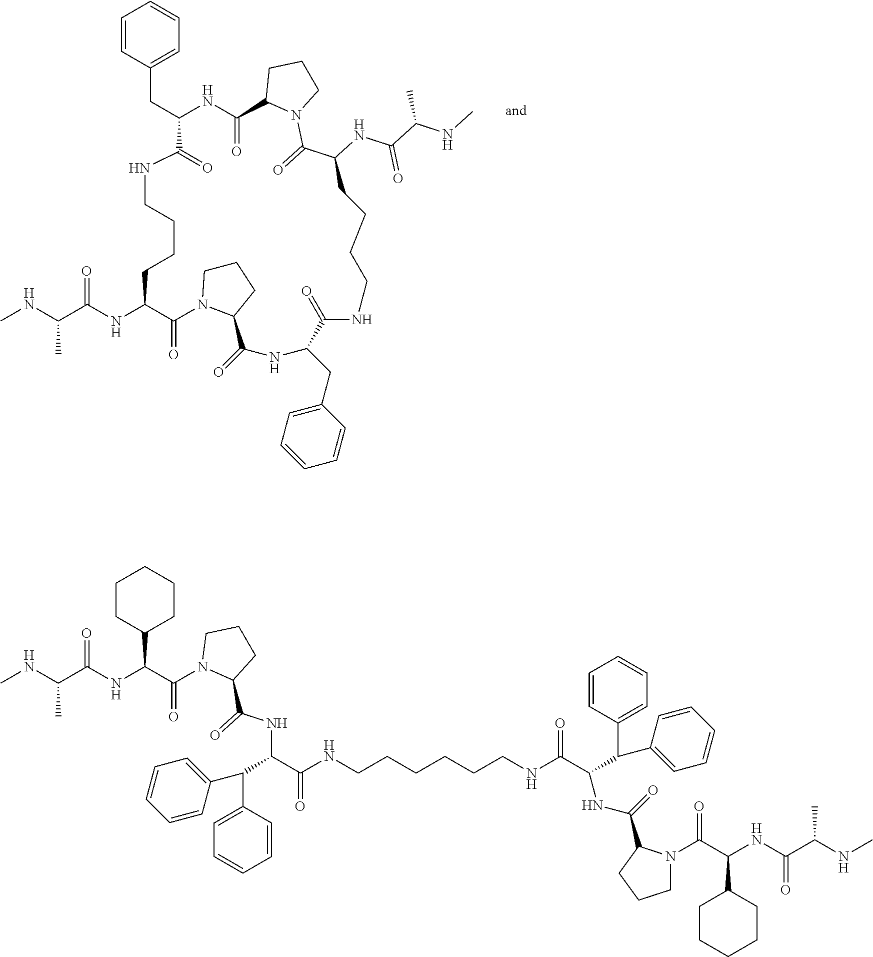

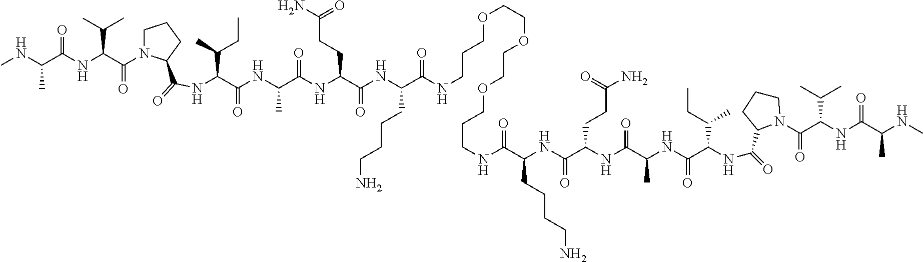

In any of the compounds described herein, the ILM can have the structure of Formula (XIII), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

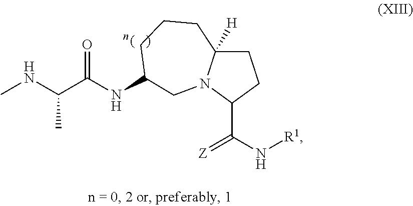

wherein:
Z of Formula (XIII) is absent or O;
R¹ of Formula (XIII) is selected from:

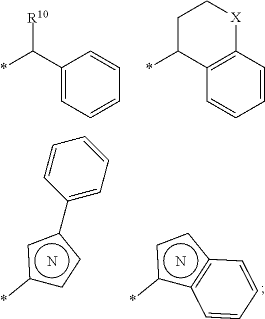

R¹⁰ of

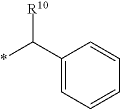

is selected from H, alkyl, or aryl;
X is selected from CH2 and O; and

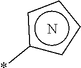

is a nitrogen-containing heteroaryl.

In any of the compounds described herein, the ILM can have the structure of Formula (XIV), which is based on the IAP ligands summarized in Flygare, J. A., et al. Small-molecule pan-IAP antagonists: a patent review, Expert Opin. Ther. Pat., 20 (2), 251-67 (2010), or an unnatural mimetic thereof:

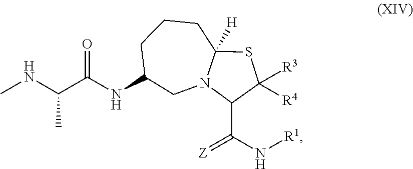

wherein:
Z of Formula (XIV) is absent or 0;
R³ and R⁴ of Formula (XIV) are independently selected from H or Me;
R¹ of Formula XIV is selected from:

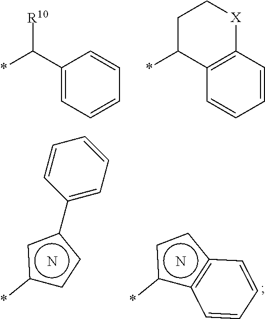

R¹⁰ of

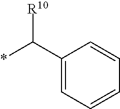

is selected from H, alkyl, or aryl;
X of

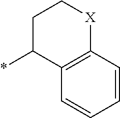

is selected from CH2 and O; and

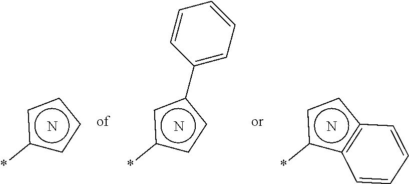

is a nitrogen-containing heteroaryl.

In any of the compounds described herein, the ILM is selected from the group consisting of:

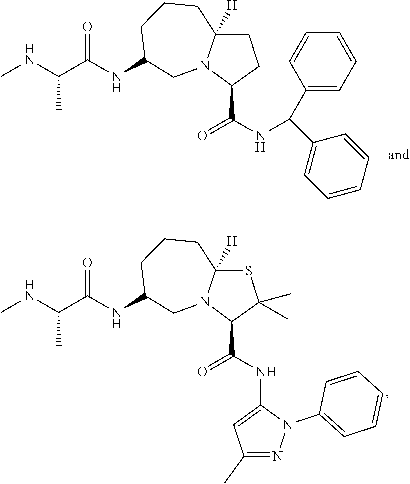

which are derivatives of ligands disclose in US Patent Pub. No. 2008/0269140 and U.S. Pat. No. 7,244,851.

In any of the compounds described herein, the ILM can have the structure of Formula (XV), which was a derivative of the IAP ligand described in WO Pub. No. 2008/128171, or an unnatural mimetic thereof:

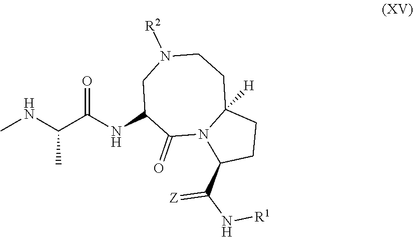

wherein:
Z of Formula (XV) is absent or O;
R¹ of Formula (XV) is selected from:

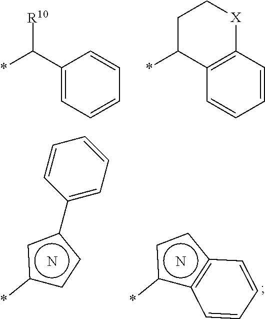

R¹⁰ of

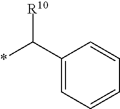

is selected from H, alkyl, or aryl;
X of

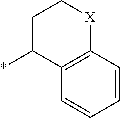

is selected from CH₂ and O; and

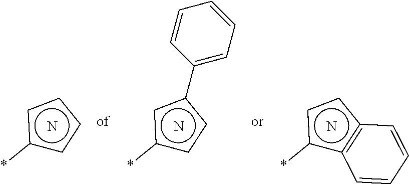

is a nitrogen-containing heteroaryl; and
R² of Formula (XV) selected from H, alkyl, or acyl;

In a particular embodiment, the ILM has the following structure:

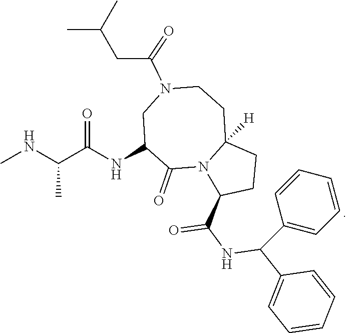

In any of the compounds described herein, the ILM can have the structure of Formula (XVI), which is based on the IAP ligand described in WO Pub. No. 2006/069063, or an unnatural mimetic thereof:

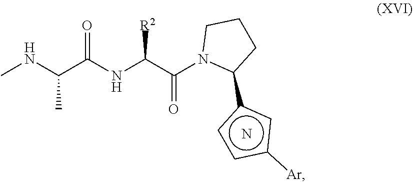

wherein:
R² of Formula (XVI) is selected from alkyl, cycloalkyl and heterocycloalkyl; more preferably, from isopropyl, tert-butyl, cyclohexyl and tetrahydropyranyl, most preferably from cyclohexyl;

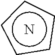

of Formula (XVI) is a 5- or 6-membered nitrogen-containing heteroaryl; more preferably, 5-membered nitrogen-containing heteroaryl, and most preferably thiazole; and Ar of Formula (XVI) is an aryl or a heteroaryl.

In any of the compounds described herein, the ILM can have the structure of Formula (XVII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

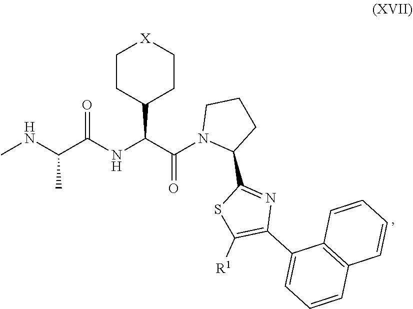

wherein:
R¹ of Formula (XVII is selected from the group halogen (e.g. fluorine), cyano,

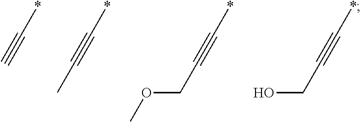

X of Formula (XVII) is selected from the group O or CH2.

In any of the compounds described herein, the ILM can have the structure of Formula (XVIII), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

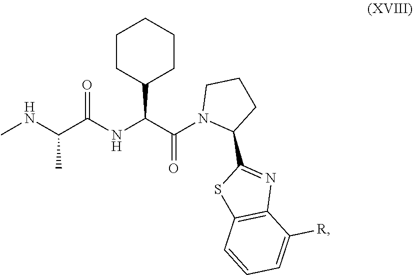

wherein R of Formula (XVIII) is selected from alkyl, aryl, heteroaryl, arylalkyl, heteroarylalkyl or halogen (in variable substitution position).

In any of the compounds described herein, the ILM can have the structure of Formula (XIX), which is based on the IAP ligands described in Cohen, F. et al., Antogonists of inhibitors of apoptosis proteins based on thiazole amide isosteres, Bioorg. Med. Chem. Lett., 20(7), 2229-33 (2010), or an unnatural mimetic thereof:

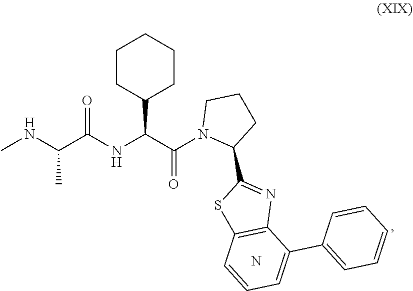

wherein

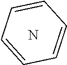

is a 6-member nitrogen heteroaryl.

In a certain embodiment, the ILM of the composition is selected from the group consisting of:

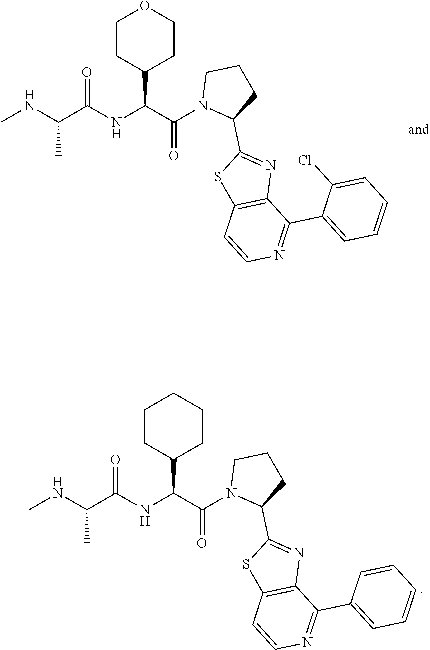 [SMILES: CN[C@@H](C)C(=O)N[C@H](C(=O)N1CCC[C@H]1c1nc2c(-c3ccccc3Cl)nccc2s1)C1CCOCC1 ; CN[C@@H](C)C(=O)N[C@H](C(=O)N1CCC[C@H]1c1nc2c(-c3ccccc3)nccc2s1)C1CCCCC1]

In certain embodiments, the ILM of the composition is selected from the group consisting of:

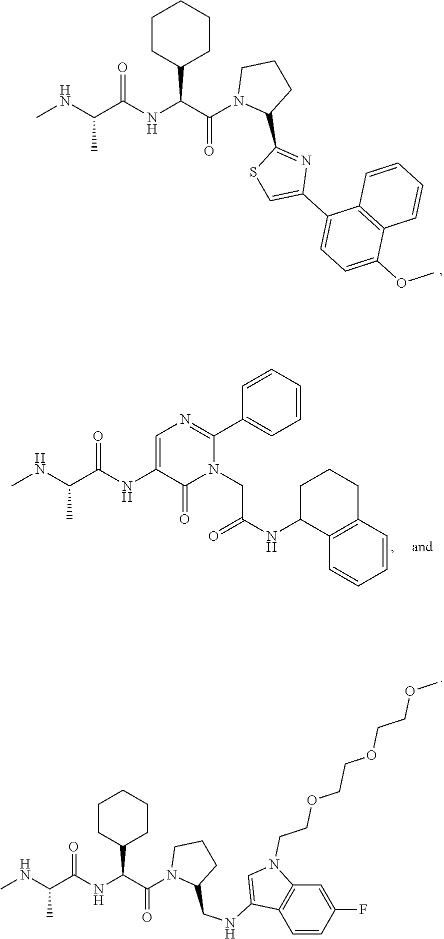

In any of the compounds described herein, the ILM can have the structure of Formula (XX), which is based on the IAP ligands described in WO Pub. No. 2007/101347, or an unnatural mimetic thereof:

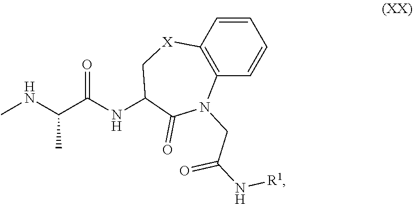

wherein X of Formula (XX) is selected from CH₂, O, NH, or S.

In any of the compounds described herein, the ILM can have the structure of Formula (XXI), which is based on the IAP ligands described in U.S. Pat. Nos. 7,345,081 and 7,419,975, or an unnatural mimetic thereof:

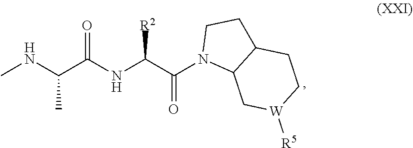

wherein:
R² of Formula (XXI) is selected from:

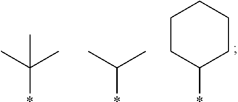

R⁵ of Formula (XXI) is selected from:

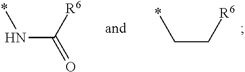

and
W of Formula (XXI) is selected from CH or N; and
R⁶ of

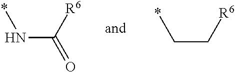

and are independently a mono- or bicyclic fused aryl or heteroaryl.

In certain embodiments, the ILM of the compound is selected from the group consisting of:

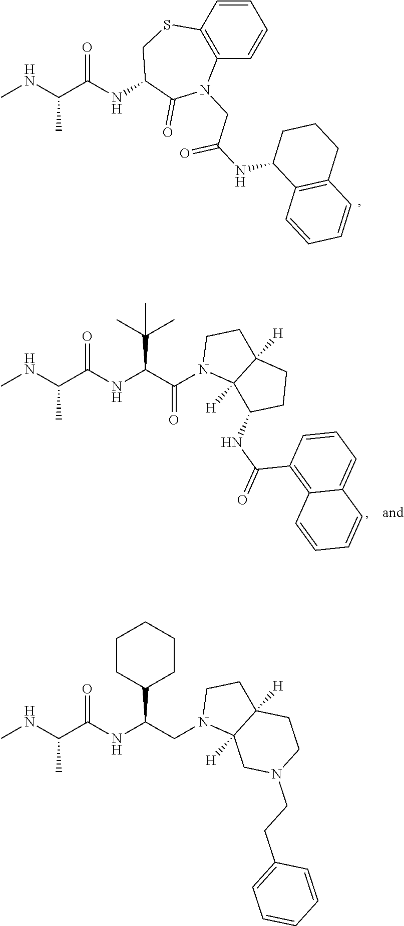 [SMILES: CN[C@@H](C)C(=O)N[C@@H]1CSc2ccccc2N(CC(=O)N[C@@H]2CCCc3ccccc32)C1=O ; CN[C@@H](C)C(=O)N[C@H](C(=O)N1CC[C@H]2CC[C@H](NC(=O)c3cccc4ccccc34)[C@H]21)C(C)(C)C ; CN[C@@H](C)C(=O)N[C@H](CN1CC[C@H]2CCN(CCc3ccccc3)C[C@H]21)C1CCCCC1]

In certain embodiments, the ILM of the compound is selected from the group consisting of:

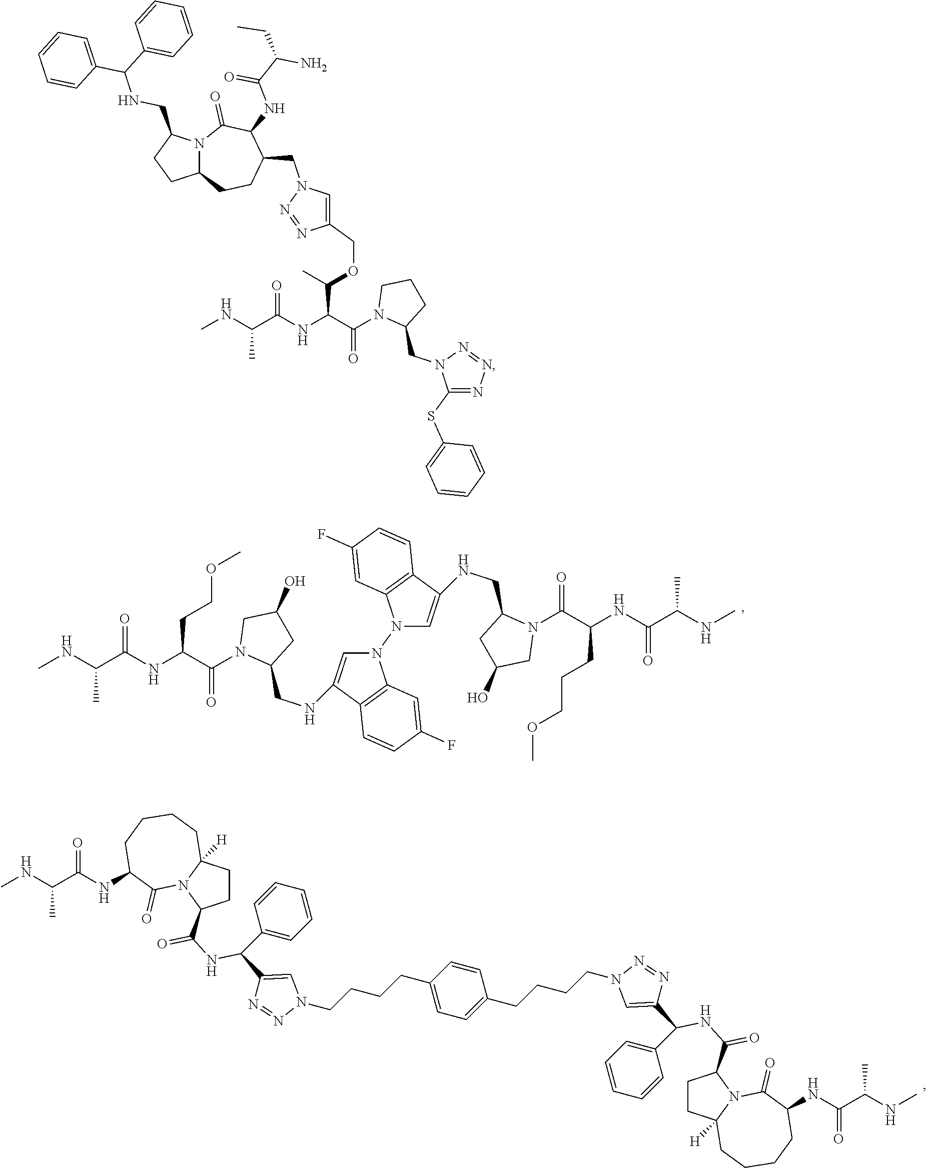

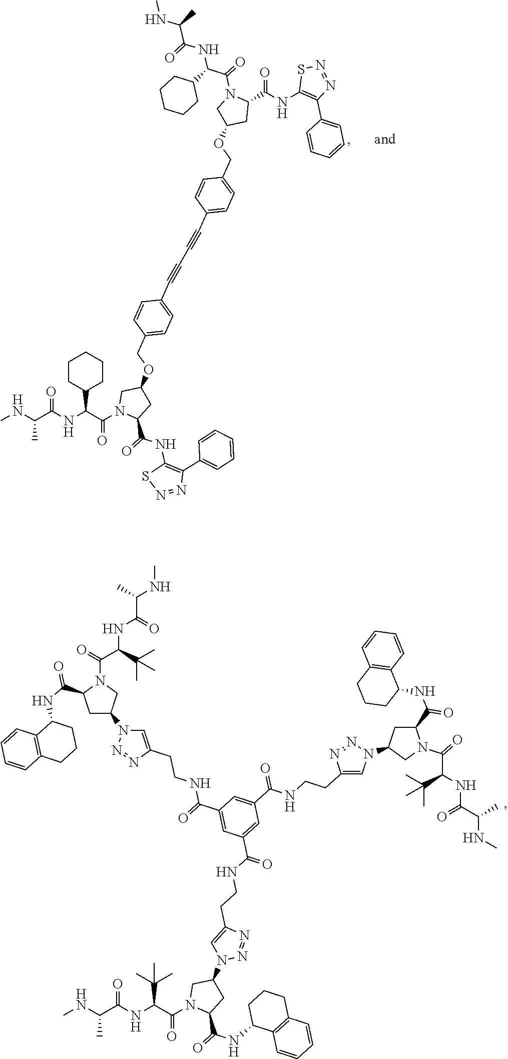 [SMILES: CN[C@@H](C)C(=O)N[C@H](C(=O)N1C[C@@H](OCc2ccc(C#CC#Cc3ccc(CO[C@H]4C[C@@H](C(=O)Nc5snnc5-c5ccccc5)N(C(=O)[C@@H](NC(=O)[C@H](C)NC)C5CCCCC5)C4)cc3)cc2)C[C@H]1C(=O)Nc1snnc1-c1ccccc1)C1CCCCC1 ; CN[C@@H](C)C(=O)N[C@H](C(=O)N1C[C@@H](n2cc(CCNC(=O)c3cc(C(=O)NCCc4cn([C@H]5C[C@@H](C(=O)N[C@@H]6CCCc7ccccc76)N(C(=O)[C@@H](NC(=O)[C@H](C)NC)C(C)(C)C)C5)nn4)cc(C(=O)NCCc4cn([C@H]5C[C@@H](C(=O)N[C@@H]6CCCc7ccccc76)N(C(=O)[C@@H](NC(=O)[C@H](C)NC)C(C)(C)C)C5)nn4)c3)nn2)C[C@H]1C(=O)N[C@@H]1CCCc2ccccc21)C(C)(C)C]

which are described in WO Pub. No. 2009/060292, U.S. Pat. No. 7,517,906, WO Pub. No. 2008/134679, WO Pub. No. 2007/130626, and WO Pub. No. 2008/128121.

In any of the compounds described herein, the ILM can have the structure of Formula (XXII) or (XXIII), which are derived from the IAP ligands described in WO Pub. No. 2015/006524 and Perez H L, Discovery of potent heterodimeric antagonists of inhibitor of apoptosis proteins (IAPs) with sustained antitumor activity. J. Med. Chem. 58(3), 1556-62 (2015), or an unnatural mimetic thereof:

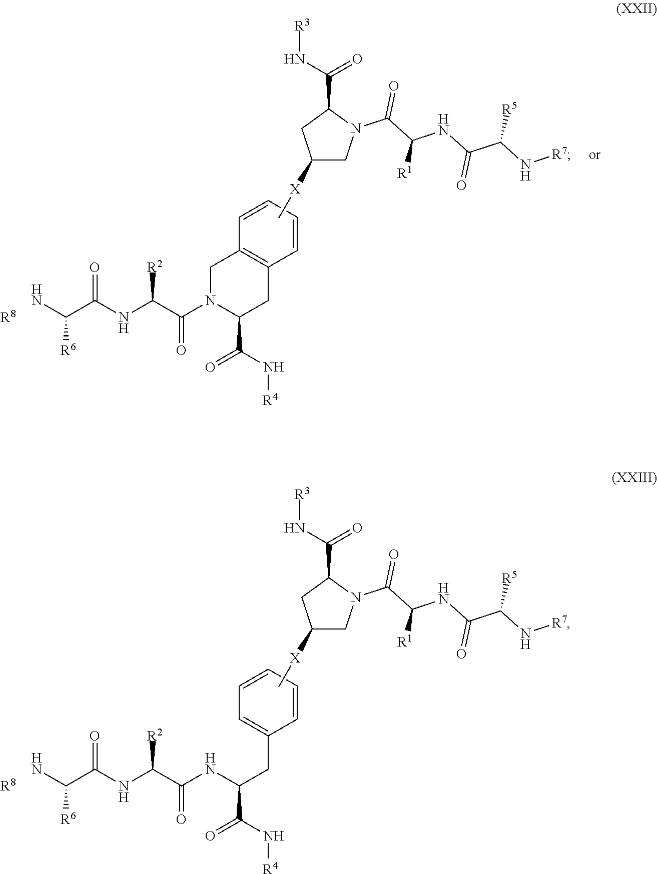

wherein:
R¹ of Formula (XXII) or (XXIII) is optionally substituted alkyl, optionally substituted cycloalkyl, optionally substituted cycloalkylalkyl, optionally substituted heterocyclyl, optionally substituted arylalkyl or optionally substituted aryl;
R² of Formula (XXII) or (XXIII) is optionally substituted alkyl, optionally substituted cycloalkyl, optionally substituted cycloalkylalkyl, optionally substituted heterocyclyl, optionally substituted arylalkyl or optionally substituted aryl;
or alternatively, R¹ and R² of Formula (XXII) or (XXIII) are independently optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²;
wherein:
v is an integer from 1-3;
R²⁰ and R²² of —(CH₂)ᵥCOR²⁰ and —CH₂R²³ are independently selected from OH, NR²⁴R²⁵ or OR²⁶;
R²¹ of —CH₂CHR²¹COR² is selected from the group NR²⁴R²⁵.
R²³ of —CH₂R²³ is selected from optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH₂O)ₘCH₃, or a polyamine chain, such as spermine or spermidine;
R²⁶ of OR²⁶ is selected from optionally substituted alkyl, wherein the optional substituents are OH, halogen or NH₂; and
m is an integer from 1-8;
R³ and R⁴ of Formula (XXII) or (XXIII) are independently selected from optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
R⁵, R⁶, R⁷ and R⁸ of Formula (XXII) or (XXIII) are independently selected from hydrogen, optionally substituted alkyl or optionally substituted cycloalkyl; and
X is selected from a bond or a chemical linker group, and/or a pharmaceutically acceptable salt, tautomer or stereoisomer thereof.

In certain embodiments, X is a bond or is selected from the group consisting of:

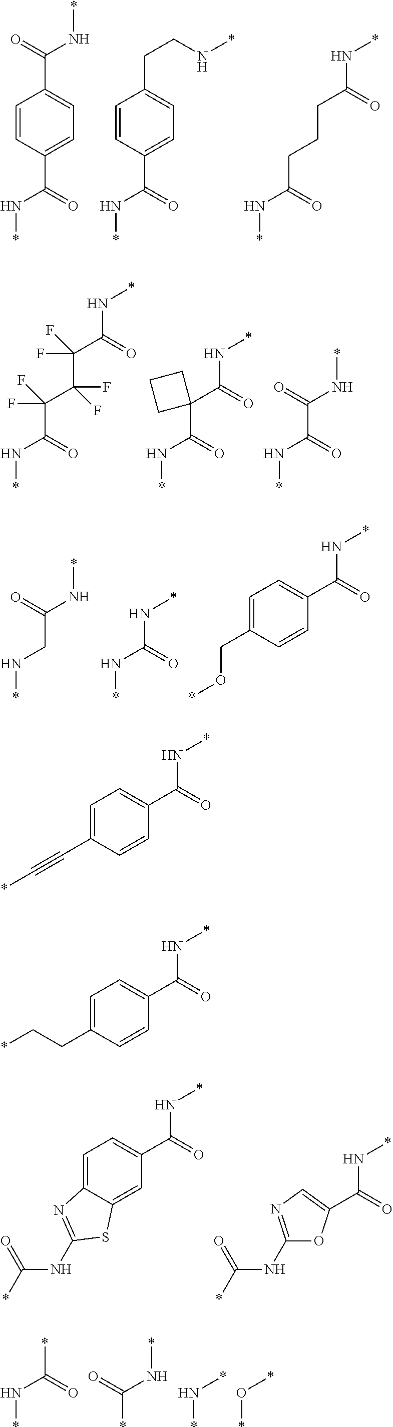

wherein “*” is the point of attachment of a PTM, L or ULM, e.g., an ILM.

In any of the compounds described herein, the ILM can have the structure of Formula (XXIV) or (XXVI), which are derived from the IAP ligands described in WO Pub. No. 2015/006524 and Perez H L, Discovery of potent heterodimeric antagonists of inhibitor of apoptosis proteins (IAPs) with sustained antitumor activity. J. Med. Chem. 58(3), 1556-62 (2015), or an unnatural mimetic thereof, and the chemical linker to linker group L as shown:

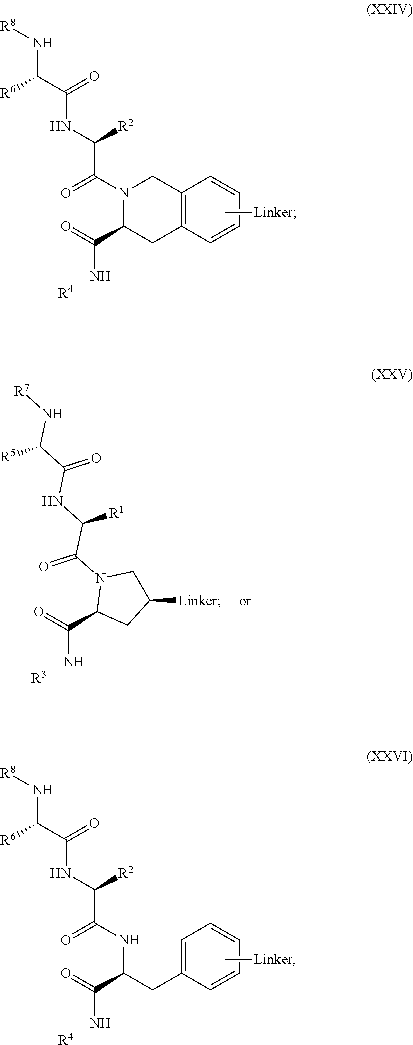

wherein:
R¹ of Formula (XXIV), (XXV) or (XXVI) is selected from optionally substituted alkyl, optionally substituted cycloalkyl, optionally substituted cycloalkylalkyl, optionally substituted heterocyclyl, optionally substituted arylalkyl or optionally substituted aryl;
R² of Formula (XXIV), (XXV) or (XXVI) is selected from optionally substituted alkyl, optionally substituted cycloalkyl, optionally substituted cycloalkylalkyl, optionally substituted heterocyclyl, optionally substituted arylalkyl or optionally substituted aryl; or alternatively,
R¹ and R² of Formula (XXIV), (XXV) or (XXVI) are independently selected from optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²,
wherein:
   v is an integer from 1-3;
   R²⁰ and R²² of —(CH₂)ᵥCOR²⁰ and —CH₂R² are independently selected from OH, NR²⁴R²⁵ or OR²⁶;
   R²¹ of —CH₂CHR²¹COR² is selected from NR²⁴R²⁵;
   R²³ of —CH₂R²³ is selected from optionally substituted aryl or optionally substituted heterocyclyl, wherein the optional substituents include alkyl and halogen;
   R²⁴ of NR²⁴R²⁵ is selected from hydrogen or optionally substituted alkyl;
   R² of NR²⁴R²⁵ is selected from hydrogen, optionally substituted alkyl, optionally substituted branched alkyl, optionally substituted arylalkyl, optionally substituted heterocyclyl, —CH₂(OCH₂CH₂O)ₘCH₃, or a polyamine chain, such as spermine or spermidine;
   R⁶ of OR⁶ is selected from optionally substituted alkyl, wherein the optional substituents are OH, halogen or NH₂; and
   m is an integer from 1-8;
   R³ and R⁴ of Formula (XXIV), (XXV) or (XXVI) are independently optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
   R⁵, R⁶, R⁷ and R⁸ of Formula (XXIV), (XXV) or (XXVI) are independently hydrogen, optionally substituted alkyl or optionally substituted cycloalkyl; and/or a pharmaceutically acceptable salt, tautomer or stereoisomer thereof.

In a particular embodiment, the ILM according to Formulas (XXII) through (XXVI):

R⁷ and R⁸ are selected from the H or Me;
R⁵ and R⁶ are selected from the group comprising:

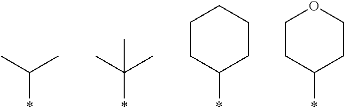

R³ and R⁴ are selected from the group comprising:

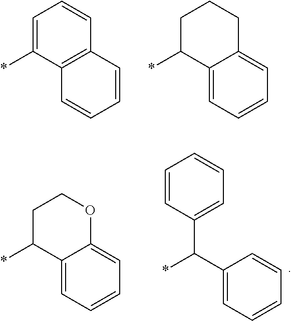

In any of the compounds described herein, the ILM can have the structure of Formula (XXVII) or (XXVII), which are derived from the IAP ligands described in WO Pub. No. 2014/055461 and Kim, K S, Discovery of tetrahydroisoquinoline-based bivalent heterodimeric IAP antagonists. Bioorg. Med. Chem. Lett. 24(21), 5022-9 (2014), or an unnatural mimetic thereof:

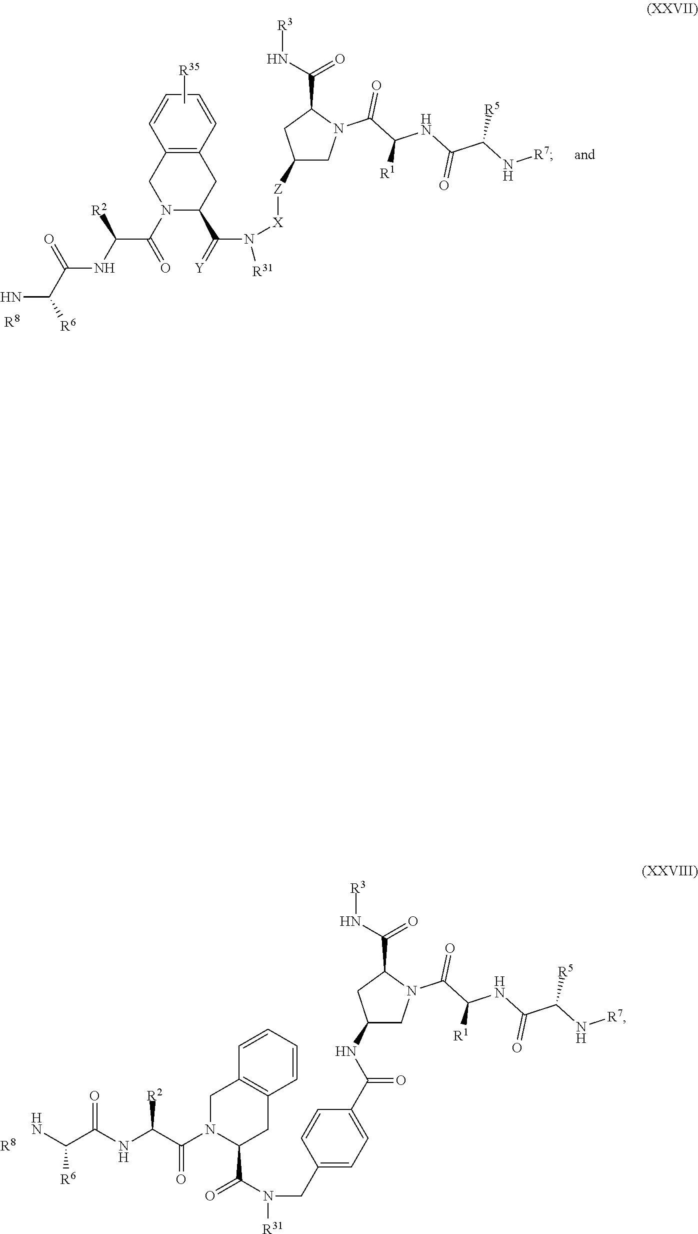

wherein:
R³⁵ is 1-2 substituents selected from alkyl, halogen, alkoxy, cyano and haloalkoxy;
R¹ of Formula (XXVII) and (XXVIII) is selected from H or an optionally substituted alkyl, optionally substituted cycloalkyl, optionally substituted cycloalkylalkyl, optionally substituted heterocyclyl, optionally substituted arylalkyl or optionally substituted aryl;
R² of Formula (XXVII) and (XXVIII) is selected from H or an optionally substituted alkyl, optionally substituted cycloalkyl, optionally substituted cycloalkylalkyl, optionally substituted heterocyclyl, optionally substituted arylalkyl or optionally substituted aryl; or alternatively,
R¹ and R² of Formula (XXVII) and (XXVIII) are independently selected from an optionally substituted thioalkyl —CR⁶⁰R⁶¹SR⁷⁰, wherein R⁶⁰ and R⁶¹ are selected from H or methyl, and R⁷⁰ is selected from an optionally substituted alkyl, optionally substituted branched alkyl, optionally substituted heterocyclyl, —(CH₂)ᵥCOR²⁰, —CH₂CHR²¹COR²² or —CH₂R²³,
wherein:
   v is an integer from 1-3;
   R²⁰ and R²² of —(CH₂)ᵥCOR²⁰ and —CH₂CHR²¹COR²² are independently selected from OH, NR²⁴R²⁵ or OR²⁶;
   R²¹ of —CH₂CHR²¹COR²² is selected from NR²⁴R²⁵;
   R²³ of —CH₂R²³ is selected from an optionally substituted aryl or optionally substituted heterocyclyl, where the optional substituents include alkyl and halogen;
   R²⁴ of NR²⁴R²⁵ is selected from hydrogen or optionally substituted alkyl;
   R²⁵ of NR²⁴R²⁵ is selected from hydrogen, optionally substituted alkyl, optionally substituted branched alkyl, optionally substituted arylalkyl, optionally substituted heterocyclyl, —CH₂CH₂(OCH₂CH₂)ₘCH₃, or a polyamine chain —[CH₂CH₂(CH₂)_{δ}NH]_{ψ}CH₂CH₂(CH₂)ω̅NH₂, such as spermine or spermidine;
   wherein δ=0-2, ψ=1-3, ω̅=0-2;
   R²⁶ of OR²⁶ is an optionally substituted alkyl, wherein the optional substituents are OH, halogen or NH₂; and
   m is an integer from 1-8,
   R³ and R⁴ of Formula (XXVII) and (XXVIII) are independently selected from an optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
   R⁵, R⁶, R⁷ and R⁸ of Formula (XXVII) and (XXVIII) are independently selected from hydrogen, optionally substituted alkyl or optionally substituted cycloalkyl;
   R³¹ of Formulas (XXVII) and (XXVIII) is selected from alkyl, aryl, arylalkyl, heteroaryl or heteroarylalkyl optionally further substituted, preferably selected form the group consisting of:

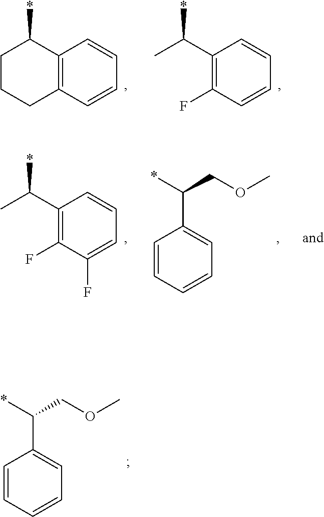

X of Formulas (XXVII) and (XXVIII) is selected from —(CR⁸¹R⁸²)ₘ, optionally substituted heteroaryl or heterocyclyl,

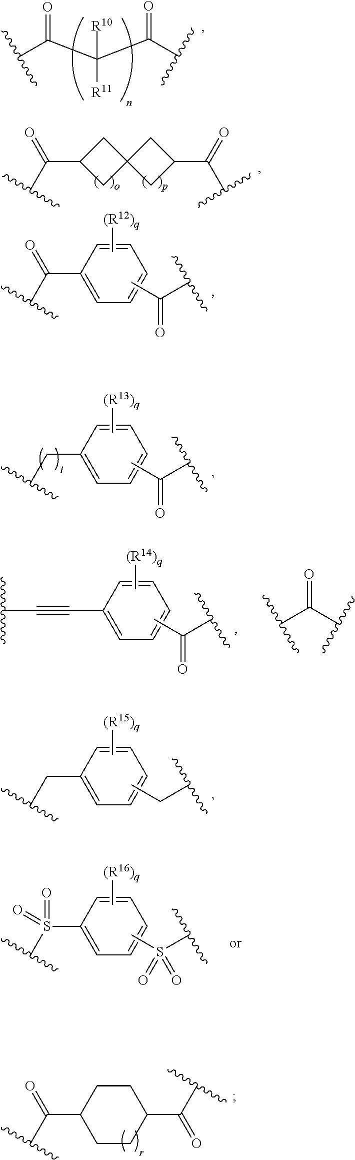

Z of Formulas (XXVII) is selected from C═O, —O—, —NR, —CONH—, —NHCO—, or may be absent;
R⁸¹ and R⁸² of —(CR⁸¹R⁸²)ₘ— are independently selected from hydrogen, halogen, alkyl or cycloalkyl, or R⁸¹ and R⁸² can be taken together to form a carbocyclic ring;
R¹⁰ and R¹¹ of

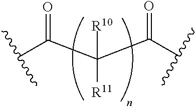

are independently selected from hydrogen, halogen or alkyl;
R¹², R¹³, R¹⁴, R¹⁵ and R¹⁶ of

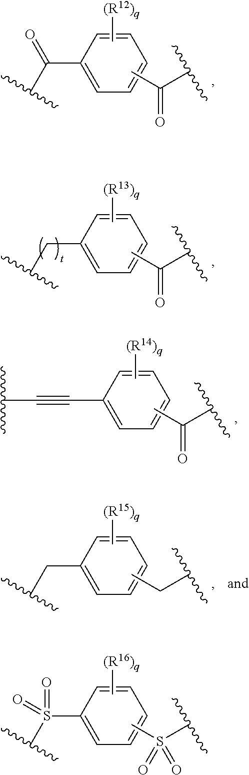

are independently selected from hydrogen, halogen or optionally substituted alkyl or OR¹⁷;
R¹⁷ is selected from hydrogen, optionally substituted alkyl or optionally substituted cycloalkyl;
m and n of —(CR²¹R²²)ₘ— and

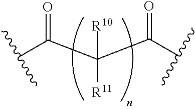

are independently 0, 1, 2, 3, or 4;
o and p of

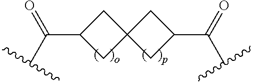

are independently 0, 1, 2 or 3;
q and t of

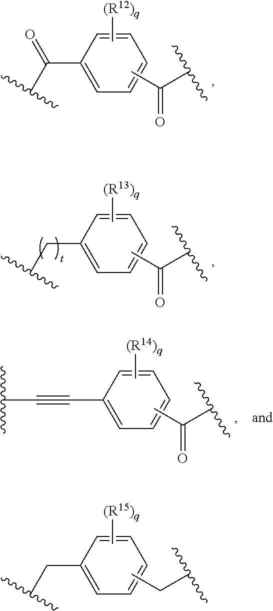

are independently 0, 1, 2, 3, or 4;
r of

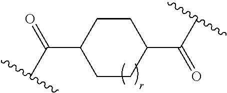

is 0 or 1;
and/or a pharmaceutically acceptable salt, tautomer or stereoisomer thereof.

In any of the compounds described herein, the ILM can have the structure of Formula (XXIX), (XXX), (XXXI), or (XXXII), which are derived from the IAP ligands described in WO Pub. No. 2014/055461 and Kim, K S, Discovery of tetrahydroisoquinoline-based bivalent heterodimeric IAP antagonists. Bioorg. Med. Chem. Lett. 24(21), 5022-9 (2014), or an unnatural mimetic thereof, and the chemical linker to linker group L as shown:

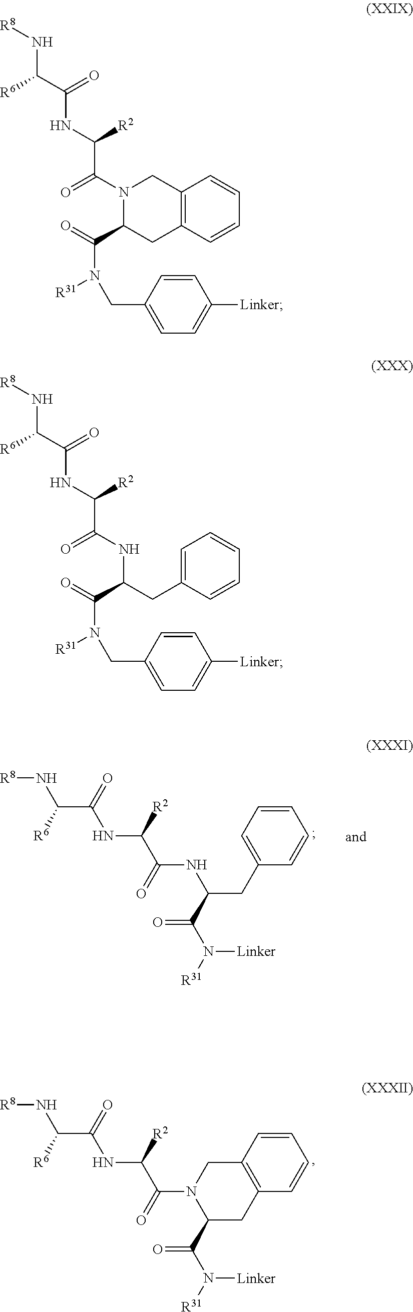

wherein:
R² of Formula (XXIX) through (XXXII) is selected from H, an optionally substituted alkyl, optionally substituted cycloalkyl, optionally substituted cycloalkylalkyl, optionally substituted heterocyclyl, optionally substituted arylalkyl or optionally substituted aryl;
or alternatively;
R¹ and R² of Formula (XXVII) and (XXVIII) are independently selected from H, an optionally substituted thioalkyl —CR⁶⁰R⁶¹SR⁷⁰ wherein R⁶⁰ and R⁶¹ are selected from H or methyl, and R⁷⁰ is an optionally substituted alkyl, optionally substituted branched alkyl, optionally substituted heterocyclyl, —(CH₂)ᵥCOR²⁰, —CH₂CHR²¹COR²² or —CH₂R²³.
wherein:
v is an integer from 1-3;
R²⁰ and R²² of —(CH₂)ᵥCOR²⁰ and —CH₂CHR²¹COR²² are independently selected from OH, NR²⁴R²⁵ or OR²⁶;
R²¹ of —CH₂CHR²¹COR²² is selected from NR²⁴R²⁵;
R²³ of —CH₂R²³ is selected from an optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂CH₂(OCH₂CH₂)ₘCH₃, or a polyamine chain —[CH₂CH₂(CH₂)_{δ}NH]_{ψ}CH₂CH₂(CH₂)ω̅ᵣNH₂, such as spermine or spermidine,
wherein δ=0-2, ψ=1-3, ω̅=0-2;
R²⁶ of OR²⁶ is an optionally substituted alkyl, wherein the optional substituents are OH, halogen or NH₂;
m is an integer from 1-8;
R⁶ and R⁸ of Formula (XXIX) through (XXXII) are independently selected from hydrogen, optionally substituted alkyl or optionally substituted cycloalkyl; and
R³¹ of Formulas (XXIX) through (XXXII) is selected from alkyl, aryl, arylalkyl, heteroaryl or heteroarylalkyl optionally further substituted, preferably selected form the group consisting of:

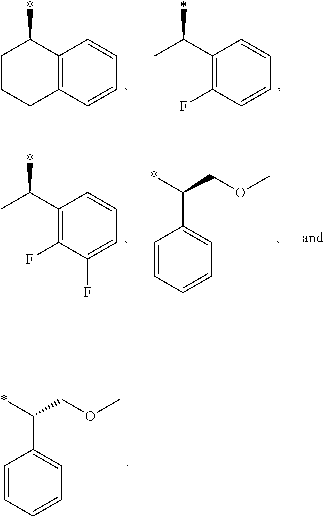

In certain embodiments, the ILM of the compound is:

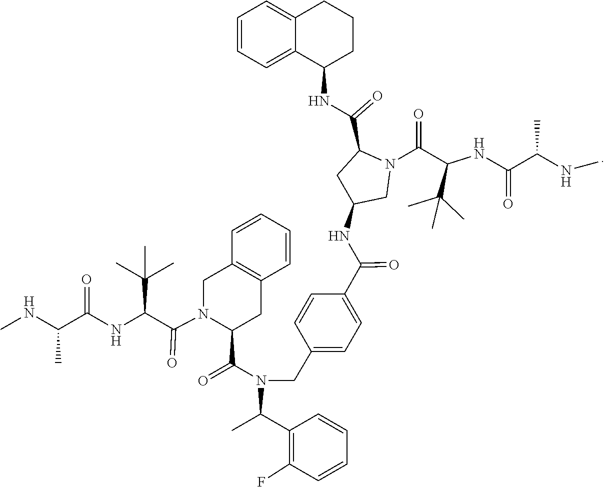

In any of the compounds described herein, the ILM can have the structure of Formula (XXXIII), which are derived from the IAP ligands described in WO Pub. No. 2014/074658 and WO Pub. No. 2013/071035, or an unnatural mimetic thereof:

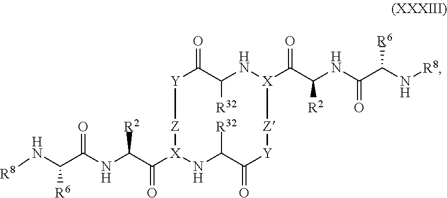

wherein:
R² of Formula (XXXIII) is selected from H, an optionally substituted alkyl, optionally substituted cycloalkyl, optionally substituted cycloalkylalkyl, optionally substituted heterocyclyl, optionally substituted arylalkyl or optionally substituted aryl;
R⁶ and R⁸ of Formula (XXXIII) are independently selected from hydrogen, optionally substituted alkyl or optionally substituted cycloalkyl;
R³² of Formula (XXXIII) is selected from (C₁-C₄ alkylene)-R³³ wherein R³³ is selected from hydrogen, aryl, heteroaryl or cycloalkyl optionally further substituted;
X of Formula (XXXIII) is selected from:

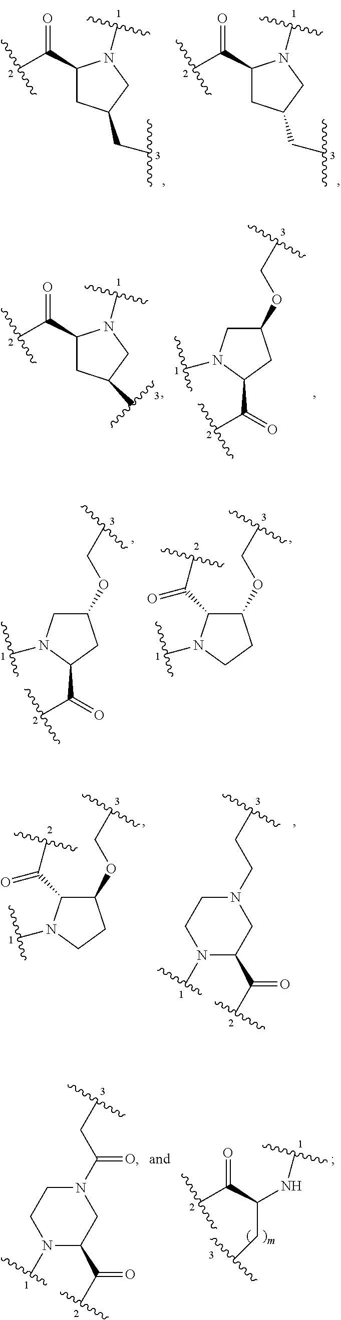

Z and Z′ of Formula (XXXIII) are independently selected from:

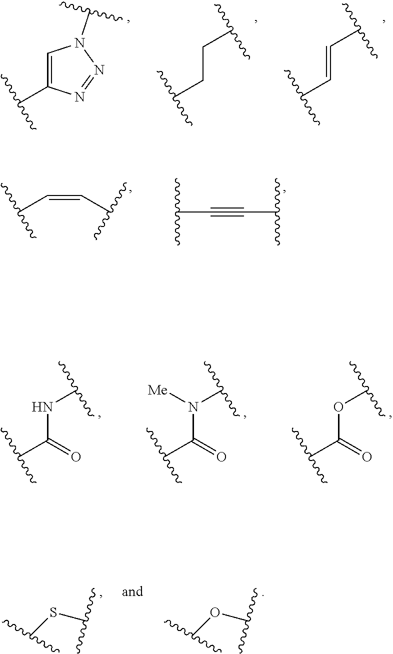

wherein each

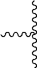

represents a point of attachment to the compound, and Z and Z′ cannot both be

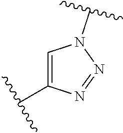 [SMILES: *c1cn(*)nn1]

in any given compound;
Y of Formula (XXXIII) is selected from:

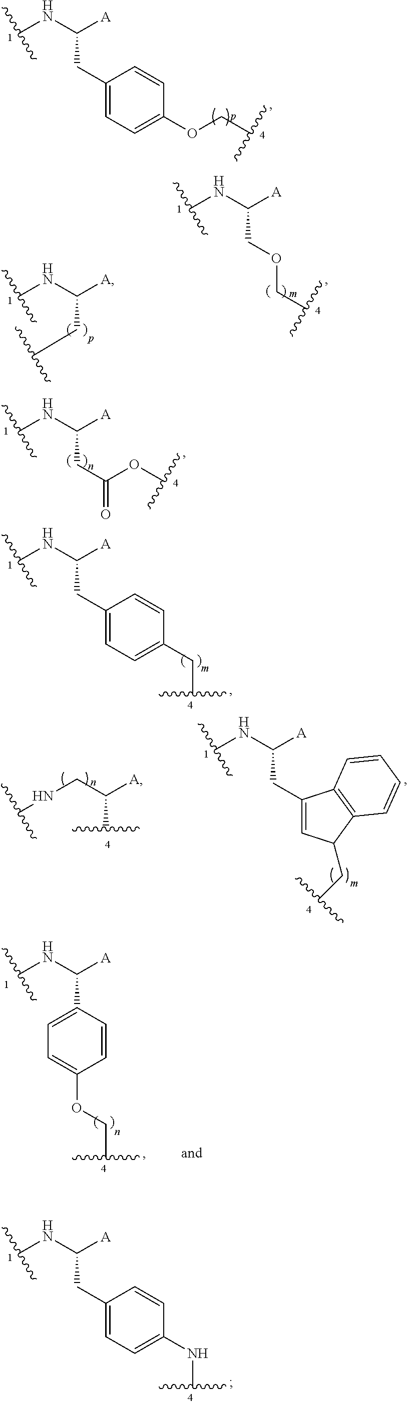

wherein Z and Z′ of Formula (XXXIII) are the same and Z is

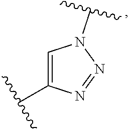 [SMILES: *c1cn(*)nn1]

wherein each

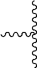

represents a point of attachment to the compound,
X is selected from:

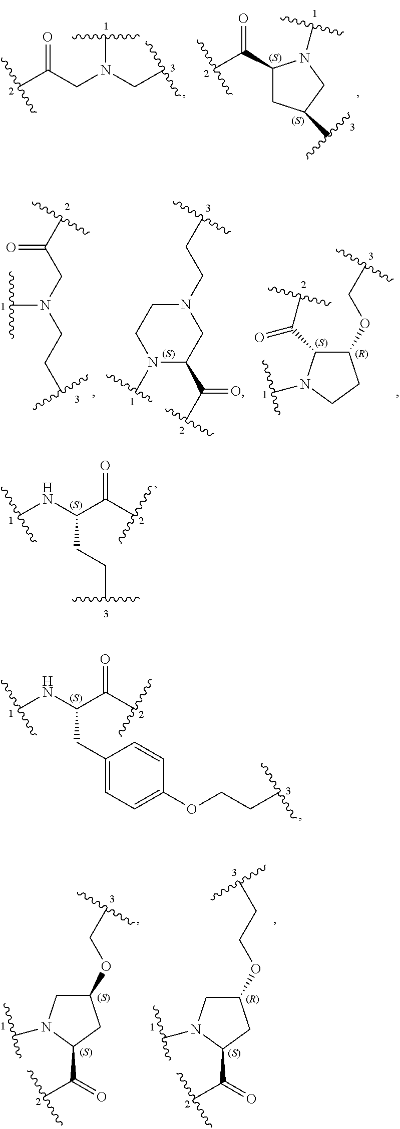

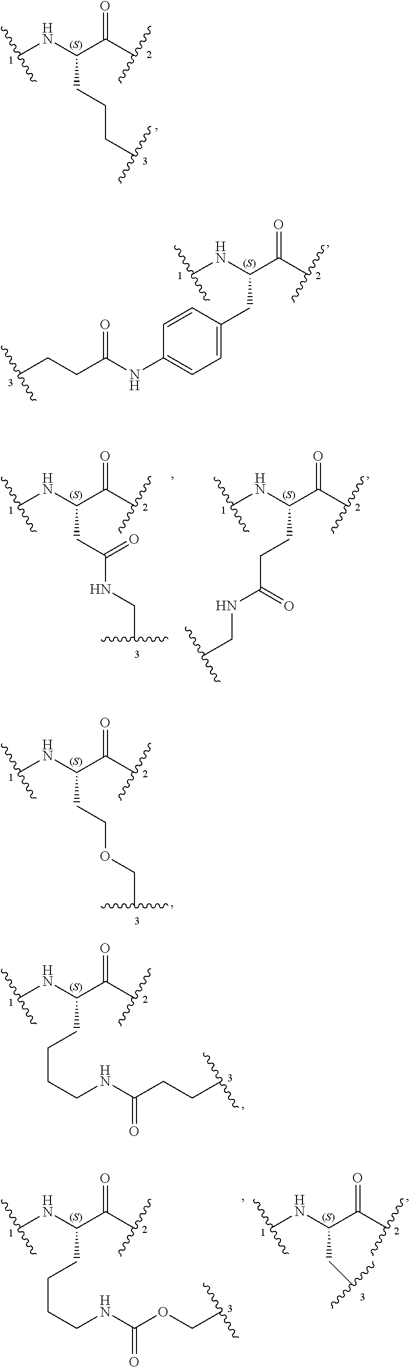

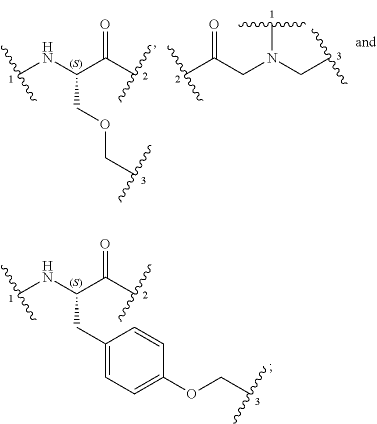

and
Y of Formula (XXXIII) is independently selected from:

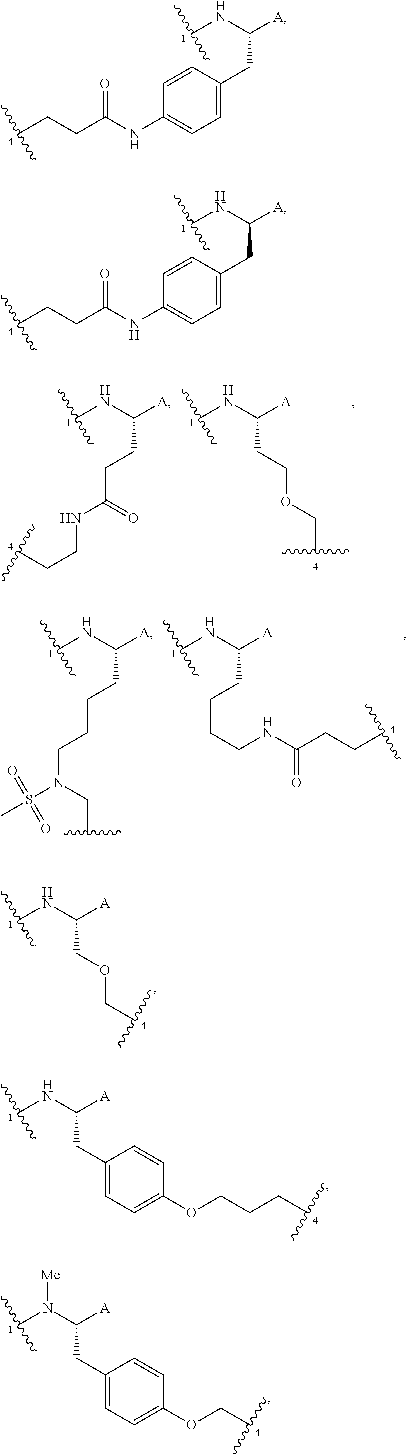

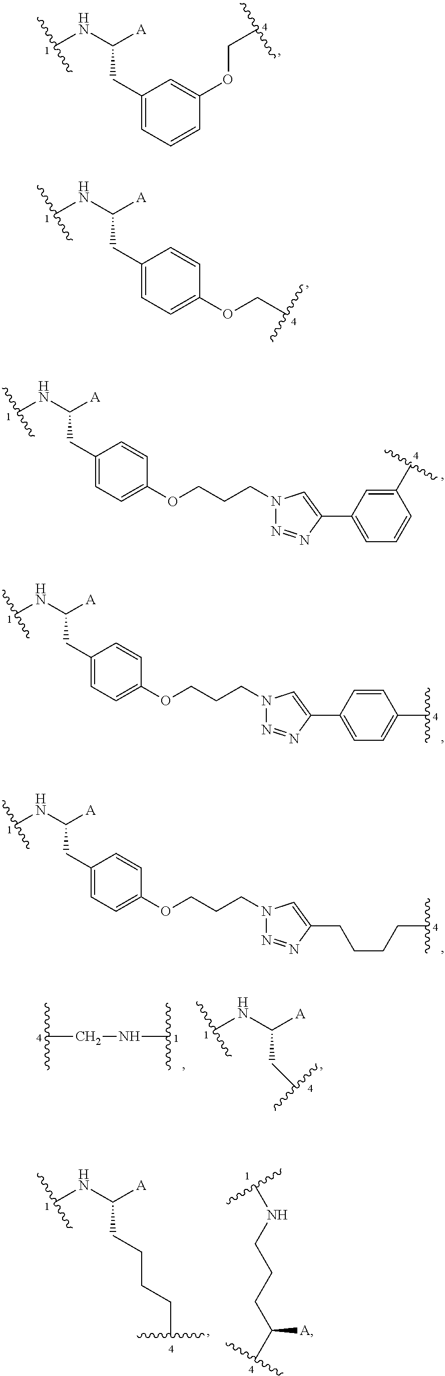

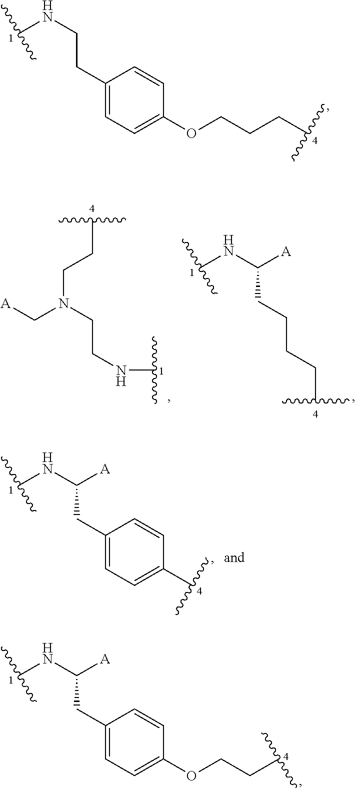

wherein:
[symbol] represents a point of attachment to a —C═O portion of the compound;
[symbol] represents a point of attachment to a —NH portion of the compound;
[symbol] represents a first point of attachment to Z;
[symbol] represents a second point of attachment to Z;
m is an integer from 0-3;
n is an integer from 1-3;
p is an integer from 0-4; and
A is —C(O)R³;
R³ is selected from —C(O)R³ is OH, NHCN, NHSO₂R¹⁰, NHOR¹¹ or N(R¹²)(R¹³);
R¹⁰ and F¹¹ of NHSO₂R¹⁰ and NHOR¹¹ are independently selected from hydrogen, optionally substituted —C₁-C₄ alkyl, cycloalkyl, aryl, heteroaryl, heterocyclyl or heterocycloalkyl;
R¹² and R¹³ of N(R¹²)(R¹³) are independently selected from hydrogen, —C₁-C₄ alkyl, —(C₁-C₄) alkylene)-NH—(C₁-C₄ alkyl), and —(C₁-C₄ alkylene)-O—(C₁-C₄ hydroxyalkyl), or R¹² and R¹³ taken together with the nitrogen atom to which they are commonly bound to form a saturated heterocyclyl optionally comprising one additional heteroatom selected from N, O and S, and wherein the saturated heterocycle is optionally substituted with methyl.

In any of the compounds described herein, the ILM can have the structure of Formula (XXXIV) or (XXXV), which are derived from the IAP ligands described in WO Pub. No. 2014/047024, or an unnatural mimetic thereof:

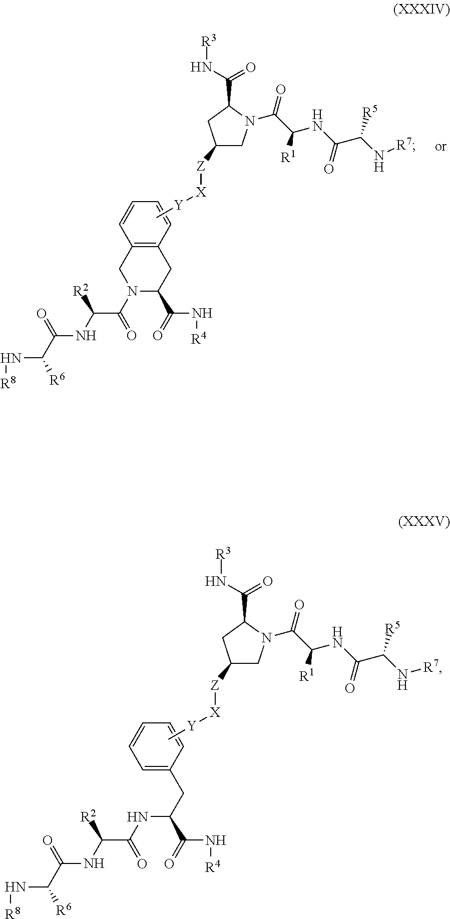

wherein:
X of Formula (XXXIV) or (XXXV) is absent or a group selected from —(CR¹⁰R¹¹)ₘ—, optionally substituted heteroaryl or optionally substituted heterocyclyl,

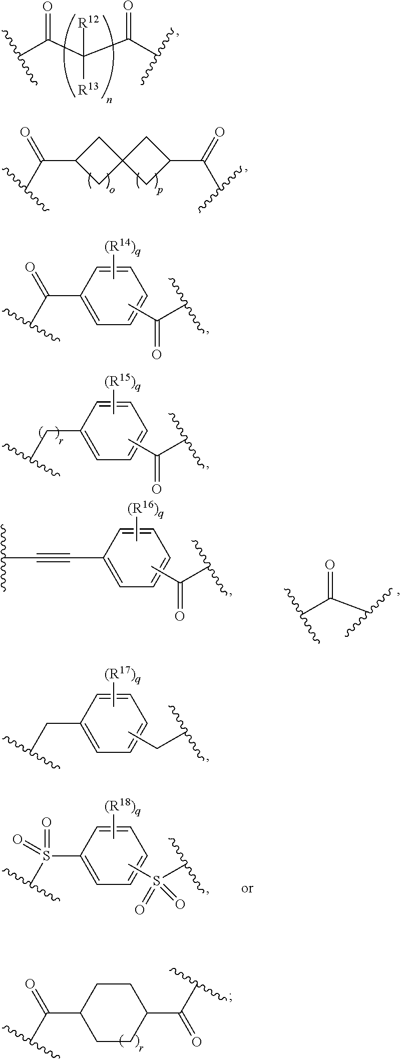

Y and Z of Formula (XXXIV) or (XXXV) are independently selected from C═O, —O—, —NR⁹—, —CONH—, —NHCO— or may be absent;
R¹ and R² of Formula (XXXIV) or (XXXV) are independently selected from an optionally substituted alkyl, optionally substituted cycloalkyl, optionally substituted cycloalkylalkyl, optionally substituted arylalkyl, optionally substituted aryl, or
R¹ and R² of Formula (XXXIV) or (XXXV) are independently selected from optionally substituted thioalkyl wherein the substituents attached to the S atom of the thioalkyl are optionally substituted alkyl, optionally substituted branched alkyl, optionally substituted heterocyclyl, —(CH₂)ᵥCOR²⁰, —CH₂CHR²¹COR²² or —CH₂R²; wherein
v is an integer from 1-3;
R²⁰ and R²² of —(CH₂)ᵥCOR²⁰ and —CH₂CHR²¹COR² are independently selected from OH, NR²⁴R²⁵ or OR²⁶;
R²¹ of —CH₂CHR²¹COR²² is selected from NR²⁴R²⁵;
R²³ of —CH₂R²³ are selected from an optionally substituted aryl or optionally substituted heterocyclyl, where the optional substituents include alkyl and halogen;
R²⁴ of NR²⁴R²⁵ is selected from hydrogen or optionally substituted alkyl;
R²⁵ of NR²⁴R²⁵ is selected from hydrogen, optionally substituted alkyl, optionally substituted branched alkyl, optionally substituted arylalkyl, optionally substituted heterocyclyl, —CH₂(OCH₂CH²⁰)mCH₃, or a polyamine chain;
R²⁶ is an optionally substituted alkyl, wherein the optional substituents are OH, halogen or NH₂;
m of —(CR¹⁰R¹¹)ₘ— is an integer from 1-8;
R³ and R⁴ of Formula (XXXIV) or (XXXV) are independently selected from optionally substituted alkyl, optionally substituted cycloalkyl, optionally substituted aryl, optionally substituted arylalkyl, optionally substituted arylalkoxy, optionally substituted heteroaryl, optionally substituted heterocyclyl, optionally substituted heteroarylalkyl or optionally substituted heterocycloalkyl, wherein the substituents are alkyl, halogen or OH;
R⁵, R⁶, R⁷ and R⁸ of Formula (XXXIV) or (XXXV) are independently selected from hydrogen, optionally substituted alkyl or optionally substituted cycloalkyl;
R¹⁰ and R¹¹ of —(CR¹⁰R¹¹)ₘ— are independently selected from hydrogen, halogen or optionally substituted alkyl;
R¹² and R¹³ of

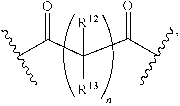

are independently selected from hydrogen, halogen or optionally substituted alkyl, or R¹² and R¹³ can be taken together to form a carbocyclic ring;
R¹⁴, R¹⁵, R¹⁶, R¹⁷ and R¹⁸ of

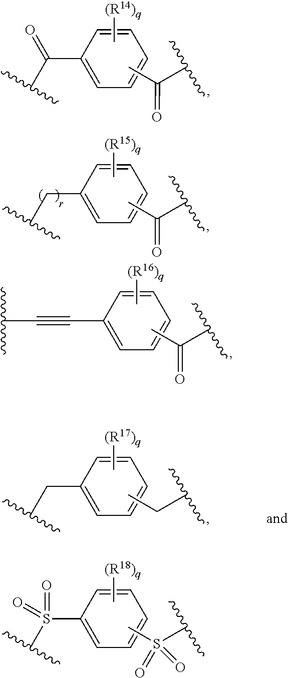

are independently selected from hydrogen, halogen, optionally substituted alkyl or OR¹⁹;
R¹⁹ of OR¹⁹ is selected from hydrogen, optionally substituted alkyl or optionally substituted cycloalkyl;
m and n of —(CR¹⁰R¹¹)ₘ— are independently 0, 1, 2, 3, or 4;
and p of —(CR¹⁰R¹¹)ₘ— are independently 0, 1, 2 or 3;
q of —(CR¹⁰R¹¹)ₘ— is 0, 1, 2, 3, or 4; r is 0 or 1;
t of —(CR¹⁰R¹¹)ₘ— is 1, 2, or 3; and/or a pharmaceutically acceptable salt, tautomer or stereoisomer thereof.

In any of the compounds described herein, the ILM can have the structure of Formula (XXXVI), which are derived from the IAP ligands described in WO Pub. No. 2014/025759, or an unnatural mimetic thereof:

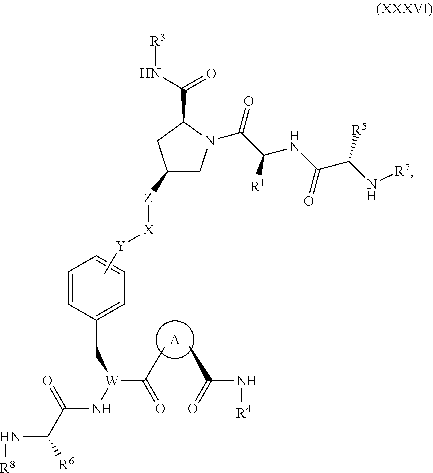

where:
A of Formula (XXXVI) is selected from:

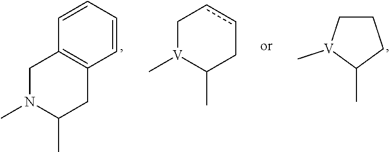

where the dotted line represents an optional double bond;
X of Formula (XXXVI) is selected from: —(CR²¹R²²)ₘ—,

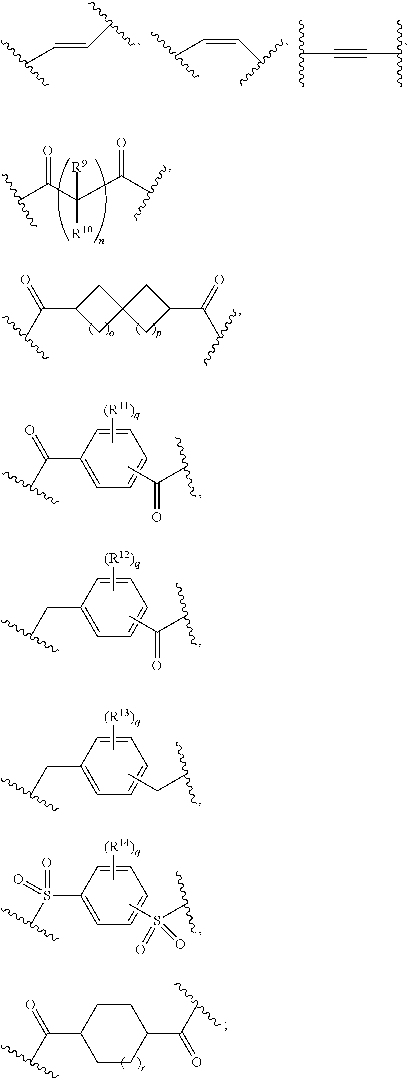

Y and Z of Formula (XXXVI) are independently selected from —O—, —NR⁶— or are absent;
V of Formula (XXXVI) is selected from —N— or —CH—;
W of Formula (XXXVI) is selected from —CH— or —N—;
R¹ of Formula (XXXVI) is selected from an optionally substituted alkyl, optionally substituted cycloalkyl, optionally substituted cycloalkylalkyl, optionally substituted arylalkyl or optionally substituted aryl;
R³ and R⁴ of Formula (XXXVI) are independently selected from optionally substituted alkyl, optionally substituted cycloalkyl, optionally substituted aryl, optionally substituted heteroaryl, optionally substituted heterocyclyl, optionally substituted arylalkyl, optionally substituted heteroarylalkyl or optionally substituted heterocycloalkyl;
R⁵, R⁶, R⁷ and R⁸ of Formula (XXIV), (XXV) or (XXVI) are independently selected from hydrogen, optionally substituted alkyl or optionally substituted cycloalkyl, or preferably methyl;
R⁹ and R¹⁰ of

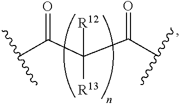

are independently selected from hydrogen, halogen or optionally substituted alkyl, or R⁹ and R¹⁰ can be taken together to form a ring;
R¹¹, R¹², R¹³ and R¹⁴ of

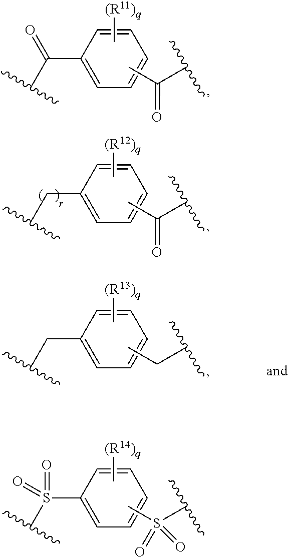

are independently selected from hydrogen, halogen, optionally substituted alkyl or OR¹⁵;
R¹⁵ of OR¹⁵ is selected from hydrogen, optionally substituted alkyl or optionally substituted cycloalkyl;
m and n of —(CR²¹R²²)ₘ— and

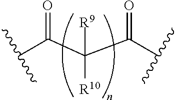

are independently selected from 0, 1, 2, 3, or 4;
o and p of

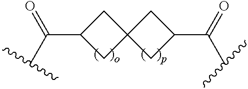

and are independently selected from 0, 1, 2 or 3;
q of

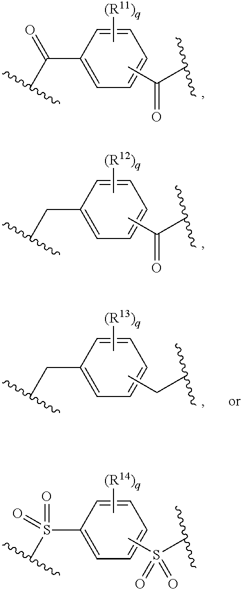

is selected from 0, 1, 2, 3, or 4;
r of

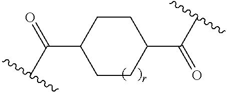

is selected from 0 or 1, and/or or a pharmaceutically acceptable salt, tautomer or stereoisomer thereof.

In any of the compounds described herein, the ILM can have the structure of Formula (XXXVII) or (XXXVIII), which are derived from the IAP ligands described in WO Pub. No. 2014/011712, or an unnatural mimetic thereof:

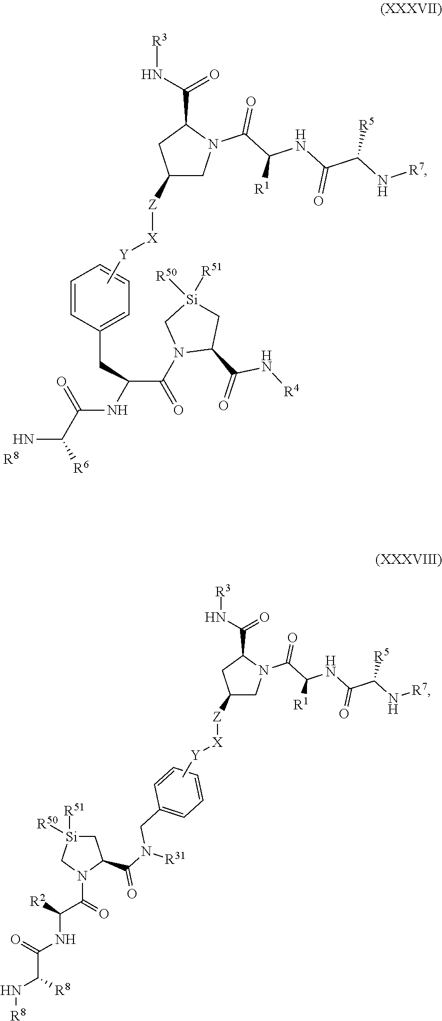

where:
X of Formulas (XXXVII) and (XXXVIII) is —(CR¹⁶R¹⁷)ₘ—,

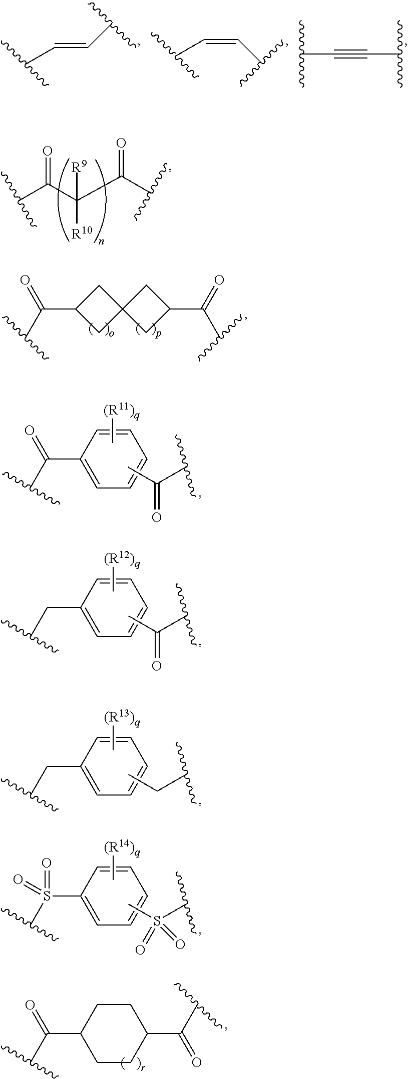

or absent;
Y and Z of Formula (XXXVII) and (XXXVIII) are independently selected from —O—, C═O, NR⁶ or are absent;
R¹ and R² of Formula (XXXVII) and (XXXVIII) are selected from optionally substituted alkyl, optionally substituted cycloalkyl, optionally substituted alkylaryl or optionally substituted aryl;
R³ and R⁴ of Formula (XXXVII) and (XXXVIII) are independently selected from optionally substituted alkyl, optionally substituted cycloalkyl, optionally substituted cycloalkylalkyl, optionally substituted arylalkyl or optionally substituted aryl;
R⁵ and R⁶ of Formula (XXXVII) and (XXXVIII) are independently selected from optionally substituted alkyl or optionally substituted cycloalkyl;
R⁷ and R⁸ of Formula (XXXVII) and (XXXVIII) are independently selected from hydrogen, optionally substituted alkyl or optionally substituted cycloalkyl, or prefereably methyl;
R⁹ and R¹⁰ of

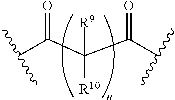

are independently selected from hydrogen, optionally substituted alkyl, or R⁹ and R¹⁰ may be taken together to form a ring;
R¹¹ to R¹⁴ of

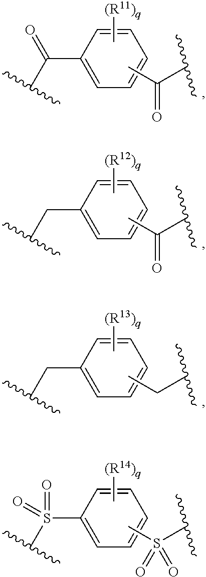

are independently selected from hydrogen, halogen, optionally substituted alkyl or OR¹⁵;
R¹⁵ of OR¹⁵ is selected from hydrogen, optionally substituted alkyl or optionally substituted cycloalkyl;
R¹⁶ and R¹⁷ of —(CR¹⁶R¹⁷)ₘ are independently selected from hydrogen, halogen or optionally substituted alkyl;
R⁵⁰ and R⁵¹ of Formula (XXXVII) and (XXXVIII) are independently selected from optionally substituted alkyl, or R⁵⁰ and R⁵¹ are taken together to form a ring;
m and n of —(CR¹⁶R¹⁷)ₘ and

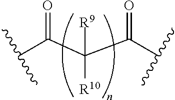

are independently an integer from 0-4;
and p of

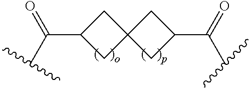

are independently an integer from 0-3;
q of

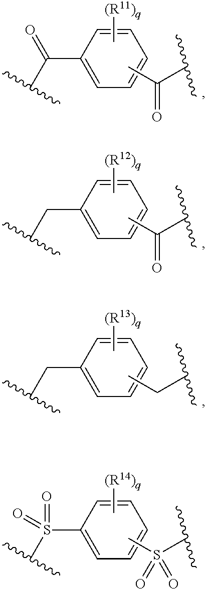

is an integer from 0-4; and
r of

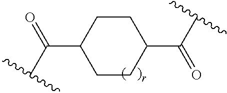

is an integer from 0-1;
or a pharmaceutically acceptable salt, tautomer or stereoisomer thereof.

In an embodiment, R¹ and R² of the ILM of Formula (XXXVII) or (XXXVIII) are t-butyl and R³ and R⁴ of the ILM of Formula (XXXVII) or (XXXVIII) are tetrahydronaphtalene.

In any of the compounds described herein, the ILM can have the structure of Formula (XXXIX) or (XL), which are derived from the IAP ligands described in WO Pub. No. 2013/071039, or an unnatural mimetic thereof:

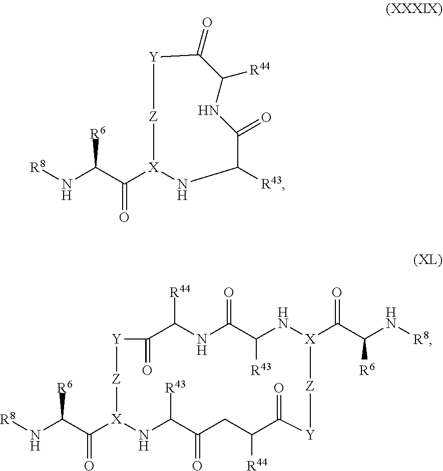

wherein:
R⁴³ and R⁴⁴ of Formulas (XXXIX) and (XL) are independently selected from hydrogen, alkyl, aryl, arylalkyl, heteroaryl, heteroarylalkyl, cycloalkyl, cycloalkylalkyl further optionally substituted, and
R⁶ and R⁸ of Formula (XXXIX) and (XL) are independently selected from hydrogen, optionally substituted alkyl or optionally substituted cycloalkyl.
each X of Formulas (XXXIX) and (XL) is independently selected from:

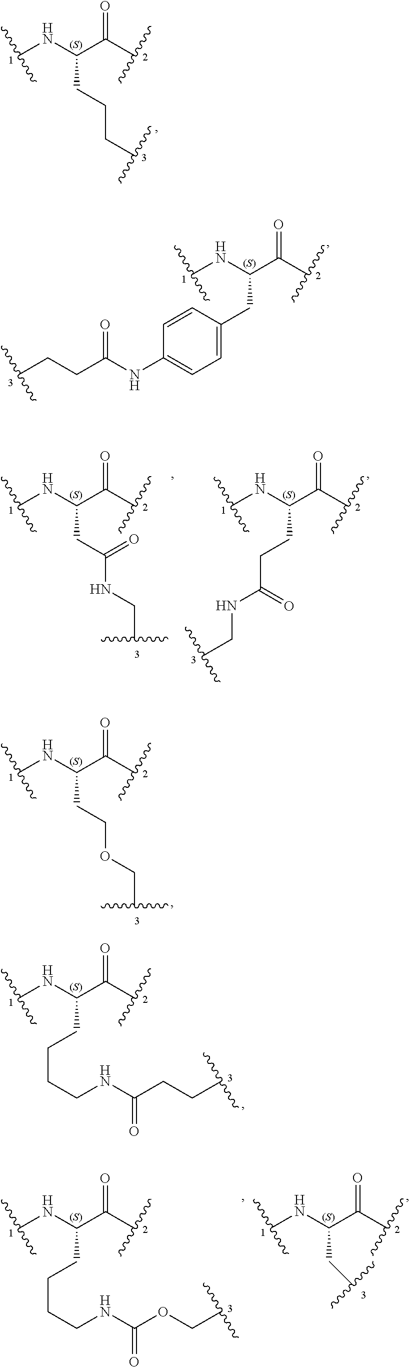

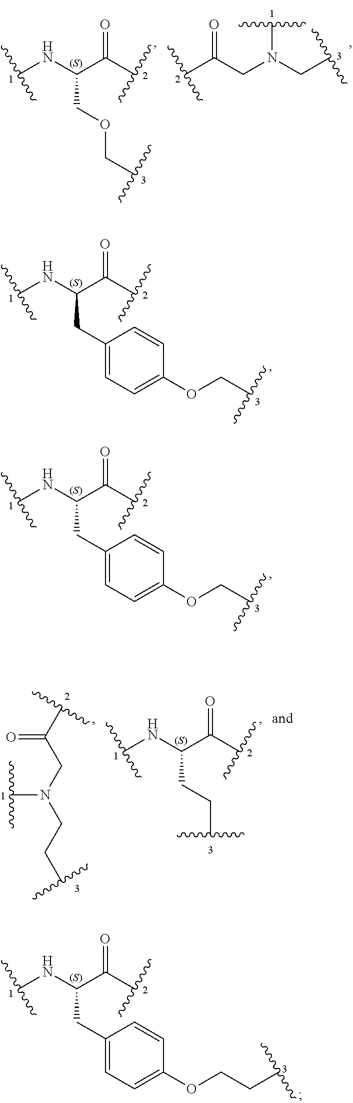

each Z of Formulas (XXXIX) and (XL) is selected from

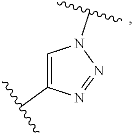 [SMILES: *c1cn(*)nn1]

wherein each [symbol] represents a point of attachment to the compound; and
each Y is selected from:

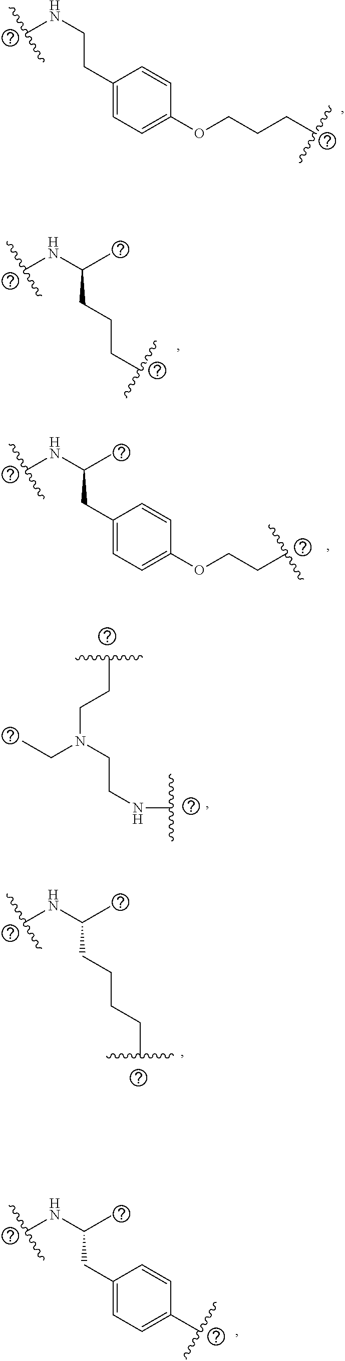

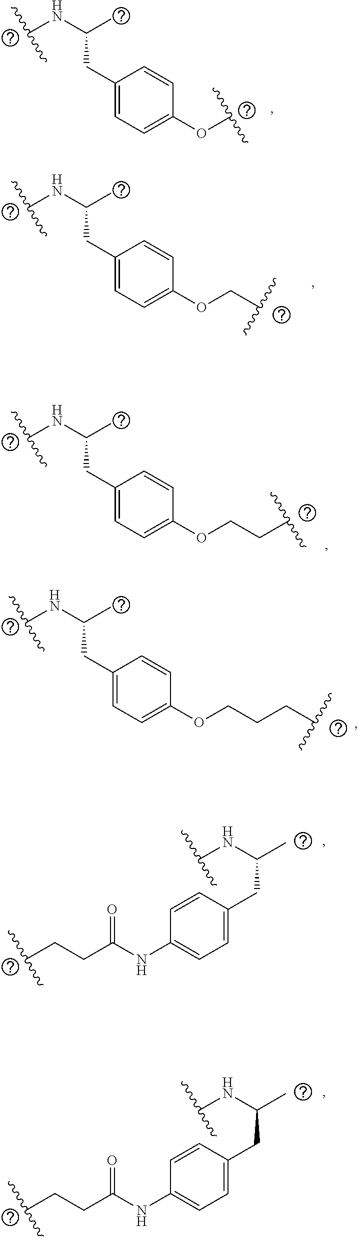 [SMILES: *N[C@H](C)Cc1ccc(O*)cc1 ; *COc1ccc(C[C@@H](C)N*)cc1 ; *CCOc1ccc(C[C@@H](C)N*)cc1 ; *CCCOc1ccc(C[C@@H](C)N*)cc1 ; *CCC(=O)Nc1ccc(C[C@@H](C)N*)cc1 ; *CCC(=O)Nc1ccc(C[C@H](C)N*)cc1]

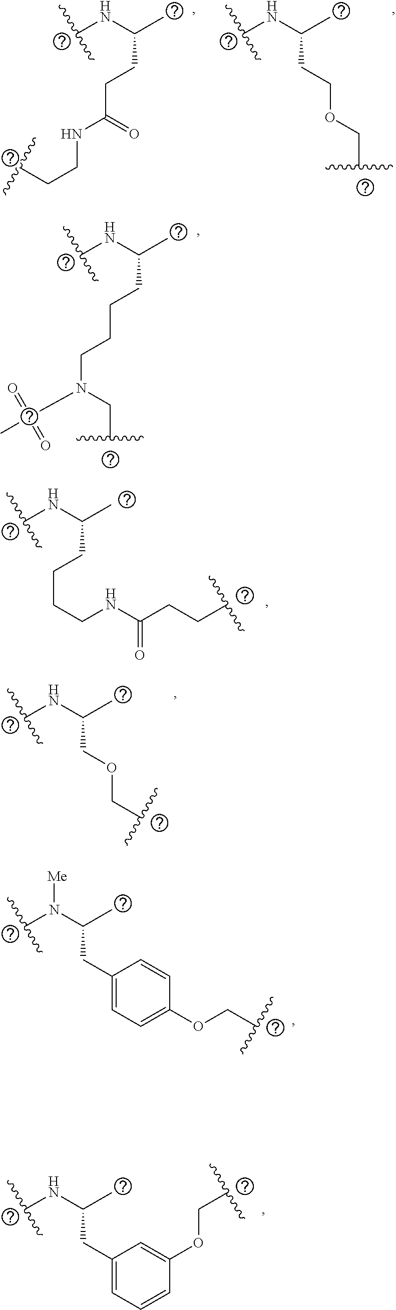

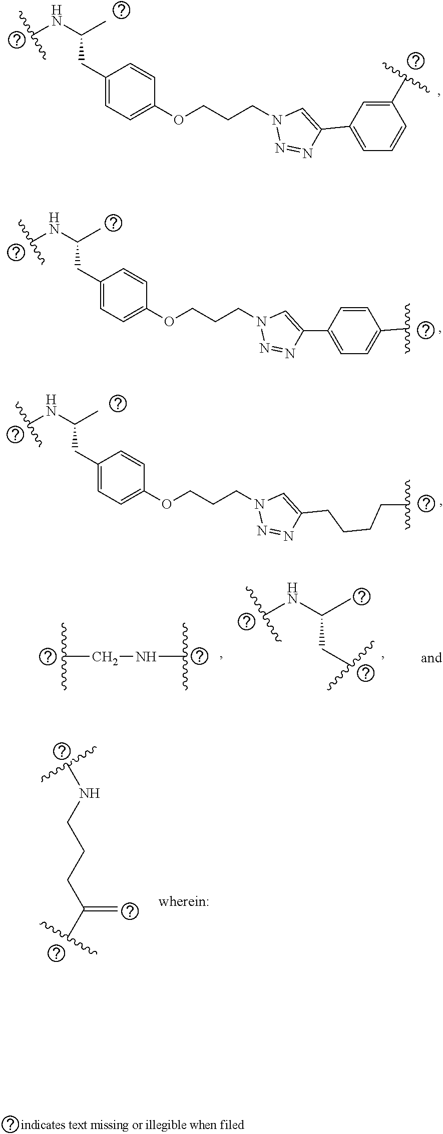

[symbol] represents a point of attachment to a —C═O portion of the compound;
[symbol] represents a point of attachment to an amino portion of the compound;
[symbol] represents a first point of attachment to Z;
[symbol] represents a second point of attachment to Z; and
A is selected from —C(O)R³ or

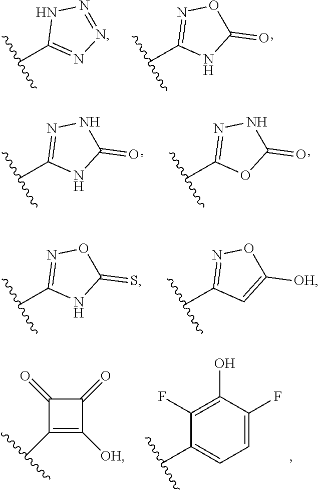

or a tautomeric form of any of the foregoing, wherein:
R³ of —C(O)R³ is selected from OH, NHCN, NHSO₂R¹⁰, NHOR¹¹ or N(R¹²)(R³);
R¹⁰ and R¹¹ of NHSO₂R¹⁰ and NHOR¹¹ are independently selected from —C₁-C₄ alkyl, cycloalkyl, aryl, heteroaryl, or heterocycloalkyl, any of which are optionally substituted, and hydrogen;
each of R¹² and R¹³ of N(R¹²)(R¹³) are independently selected from hydrogen, —C₁-C₄ alkyl, —(C₁-C₄ alkylene)-NH—(C₁-C₄ alkyl), benzyl, —(C₁-C₄ alkylene)-C(O)OH, —(C₁-C₄ alkylene)-C(O)CH3, —CH(benzyl)-COOH, —C₁-C₄ alkoxy, and —(C₁-C₄ alkylene)-O—(C₁-C₄ hydroxyalkyl); or R¹² and R¹³ of N(R¹²)(R¹³) are taken together with the nitrogen atom to which they are commonly bound to form a saturated heterocyclyl optionally comprising one additional heteroatom selected from N, O and S, and wherein the saturated heterocycle is optionally substituted with methyl.

In any of the compounds described herein, the ILM can have the structure of Formula (XLI), which are derived from the IAP ligands described in WO Pub. No. 2013/071039, or an unnatural mimetic thereof:

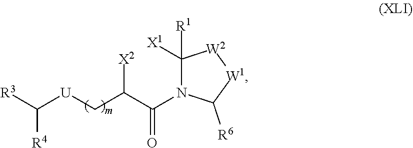

wherein:
W¹ of Formula (XLI) is selected from O, S, N—R^{A}, or C(R^{8a})(R^{8b});
W² of Formula (XLI) is selected from O, S, N—R^{A}, or C(R^{8c})(R^{8d}); provided that W¹ and W² are not both O, or both S;
R¹ of Formula (XLI) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ is selected from O, N—R^{A}, S, S(O), or S(O)₂, then X² is C(R^{2a}R^{2b});
or:
X¹ of Formula (XLI) is selected from CR^{2c}R^{2d} and X² is CR^{2a}R^{2b}, and R^{2c} and R^{2a} together form a bond;
or:
X¹ and X² of Formula (XLI) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring;
or:
X¹ of Formula (XLI) is selected from CH₂ and X² is C═O, C═C(R^{C})₂, or C═NR^{C}; where each R^{c} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl- (substituted or unsubstituted aryl), or —C₁-C₆alkyl-(substituted or unsubstituted heteroaryl);
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d} of CR^{2c}R^{2d} and CR^{2a}R^{2b} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), 1-C₆alkyl- (substituted or unsubstituted aryl), i-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), 1-C₆alkyl- (substituted or unsubstituted aryl), i-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl- (substituted or unsubstituted C₃-C₆cycloalkyl), 1-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
m of Formula (XLI) is selected from 0, 1 or 2;
—U— of Formula (XLI) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLI) is selected from C₁-C₃alkyl, or C₁-C₃fluoroalkyl;
R⁴ of Formula (XLI) is selected from —NHR⁵, —N(R⁵)2, —N+(R⁵)3 or —OR⁵;
each R⁵ of —NHR⁵, —N(R⁵)2, —N+(R⁵)3 and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLI) together with the atoms to which they are attached form a substituted or unsubstituted 5-7 membered ring;
or:
R³ of Formula (XLI) is bonded to a nitrogen atom of U to form a substituted or unsubstituted 5-7 membered ring;
R⁶ of Formula (XLI) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl- (substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH (substituted or unsubstituted aryl)2, —(CH₂)ₚ—CH(substituted or unsubstituted heteroaryl)2, —(CH₂)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)₂, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLII), which are derived from the IAP ligands described in WO Pub. No. 2013/071039, or an unnatural mimetic thereof:

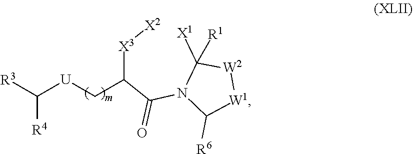

wherein:
W¹ of Formula (XLII) is O, S, N—R^{A}, or C(R^{8a})(R^{8b});
W² of Formula (XLII) is O, S, N—R^{A}, or C(R^{8c})(R^{8d}); provided that W¹ and W² are not both O, or both S;
R¹ of Formula (XLII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XLII) is N—R^{A}, then X² is C═O, or CR^{2c}R^{2d}, and X³ is CR^{2a}R^{2b};
or:
when X¹ of Formula (XLII) is selected from S, S(O), or S(O)₂, then X² is CR^{2c}R^{2d}, and X³ is CR^{2a}R^{2b}.
or:
when X¹ of Formula (XLII) is 0, then X² is CR^{2c}R^{2d} and N—R^{A} and X³ is CR^{2a}R^{2b};
or:
when X¹ of Formula (XLII) is CH₃, then X² is selected from O, N—R^{A}, S, S(O), or S(O)₂, and X³ is CR^{2a}R^{2b};
when X¹ of Formula (XLII) is CR^{2e}R^{2f} and X2 is CR^{2c}R^{2d}, and R^{2e} and R^{2c} together form a bond, and X³ of Formula (VLII) is CR^{2a}R^{2b};
or:
X¹ and X³ of Formula (XLII) are both CH₂ and X² of Formula (XLII) is C═O, C═C(R^{C})2, or C═NR^{C}; where each R^{C} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
or:
X¹ and X² of Formula (XLII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ is CR^{2a}R^{2b};
or:
X² and X³ of Formula (XLII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (VLII) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C1-C6alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl- (substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl- (substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
m of Formula (XLII) is selected from 0, 1 or 2;
—U— of Formula (XLII) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLII) is selected from C₁-C₃alkyl, or C₁-C₃fluoroalkyl;
R⁴ of Formula (XLII) is selected from —NHR⁵, —N(R⁵)2, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLII) together with the atoms to which they are attached form a substituted or unsubstituted 5-7 membered ring;
or:
R³ of Formula (XLII) is bonded to a nitrogen atom of U to form a substituted or unsubstituted 5-7 membered ring;
R⁶ of Formula (XLII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl- (substituted or unsubstituted C2-C10heterocycloalkyl, —C1-C6alkyl-(substituted or unsubstituted aryl), —C₁-C₆alkyl-(substituted or unsubstituted heteroaryl), —(CH2)p-CH(substituted or unsubstituted aryl)2, —(CH2)ₚ—CH(substituted or unsubstituted heteroaryl)2, —(CH2)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)2, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)2, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLIII), which is derived from the IAP ligands described in WO Pub. No. 2013/071039, or an unnatural mimetic thereof:

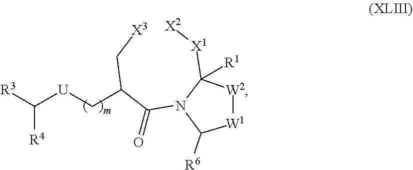

wherein:
W¹ of Formula (XLIII) is selected from O, S, N—R^{A}, or C(R^{8a})(R^{8b});
W² of Formula (XLIII) is selected from O, S, N—R^{A}, or C(R^{8c})(R^{8d}); provided that W¹ and W² are not both O, or both S;
R¹ of Formula (XLIII) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XLIII) is selected from N—R^{A}, S, S(O), or S(O)₂, then X² of Formula (XLIII) is CR^{2c}R^{2d}, and X³ of Formula (XLIII) is CR^{2a}R^{2b};
or:
when X¹ of Formula (XLIII) is 0, then X² of Formula (XLIII) is selected from O, N—R^{A}, S, S(O), or S(O)₂, and X³ of Formula (XLIII) is CR^{2a}R^{2b};
or:
when X¹ of Formula (XLIII) is CR^{2e}R^{2f} and X² of Formula (XLIII) is CR^{2c}R^{2d}, and R^{2e} and R^{2c} together form a bond, and X³ of Formula (XLIII) is CR^{2a}R^{2b};
or:
X¹ and X² of Formula (XLIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XLIII) is CR^{2a}R^{2b};
or:
X² and X³ of Formula (XLIII) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X¹ of Formula (VLII) is CR^{2e}R^{2f};
R^{A} of N—R^{A} is H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl- (substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl- (substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
m of Formula (XLIII) is 0, 1 or 2;
—U— of Formula (XLIII) is —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLIII) is C₁-C₃alkyl, or C₁-C₃fluoroalkyl;
R⁴ of Formula (XLIII) is —NHR⁵, —N(R⁵)₂, —N+(R⁵)₃ or —OR⁵;
each R⁵ of —NHR⁵, —N(R⁵)2, —N+(R⁵)₃ and —OR⁵ is independently selected from H, C₁-C₃alkyl, C₁-C₃haloalkyl, C₁-C₃heteroalkyl and —C₁-C₃alkyl-(C₃-Cscycloalkyl);
or:
R³ and R⁵ of Formula (XLIII) together with the atoms to which they are attached form a substituted or unsubstituted 5-7 membered ring;
or:
R³ of Formula (XLIII) is bonded to a nitrogen atom of U to form a substituted or unsubstituted 5-7 membered ring;
R⁶ of Formula (XLIII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C3-C10cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl- (substituted or unsubstituted C2-C10heterocycloalkyl, —C₁-C₆alkyl-(substituted or unsubstituted aryl), —C₁-C₆alkyl-(substituted or unsubstituted heteroaryl), —(CH2)p-CH(substituted or unsubstituted aryl)2, —(CH2)ₚ—CH(substituted or unsubstituted heteroaryl)2, —(CH2)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is 0, 1 or 2;
R^{8a}, R^{8b}, R^{8c}, and R^{8d} of C(R^{8a})(R^{8b}) and C(R^{8c})(R^{8d}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a} and R^{8d} are as defined above, and R^{8b} and R^{8c} together form a bond;
or:
R^{8a} and R^{8d} are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c} and R^{8d} are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a} and R^{8b} are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c} and R^{8d} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)2, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)2, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLIV), which is derived from the IAP ligands described in WO Pub. No. 2013/071039, or an unnatural mimetic thereof:

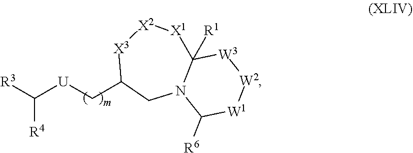

wherein:
W¹ of Formula (XLIV) is selected from O, S, N—R^{A}, or C(R^{8a})(R^{8b});
W² of Formula (XLIV) is selected from O, S, N—R^{A}, or C(R^{8c})(R^{8d}); provided that W¹ and W² are not both O, or both S;
W³ of Formula (XLIV) is selected from O, S, N—R^{A}, or C(R^{8e})(R^{8f}), providing that the ring comprising W¹, W², and W³ does not comprise two adjacent oxygen atoms or sulfur atoms;
R¹ of Formula (XLIV) is selected from H, C₁-C₆alkyl, C₃-C₆cycloalkyl, —C₁-C₆alkyl-(substituted or unsubstituted C₃-C₆cycloalkyl), substituted or unsubstituted aryl, substituted or unsubstituted heteroaryl, —C₁-C₆alkyl-(substituted or unsubstituted aryl), or —C₁-C₆alkyl-(substituted or unsubstituted heteroaryl);
when X¹ of Formula (XLIV) is O, then X² of Formula (XLIV) is selected from CR^{2c}R^{2d} and N—R^{A}, and X³ of Formula (XLIV) is CR^{2a}R^{2b};
or:
when X¹ of Formula (XLIV) is CH₂, then X² of Formula (XLIV) is selected from O, N—R^{A}, S, S(O), or S(O)₂, and X³ of Formula (XLIV) is CR^{2a}R^{2b};
or:
when X¹ of Formula (XLIV) is CR^{2e}R^{2f} and X² of Formula (XLIV) is CR^{2c}R^{2d}, and R^{2e} and R^{2c} together form a bond, and X³ of Formula (VLIV) is CR^{2a}R^{2b};
or:
X¹ and X³ of Formula (XLIV) are both CH₂ and X² of Formula (XLII) is C═P, C═C(R^{C})₂, or C═NR^{C}; where each R^{C} is independently selected from H, —CN, —OH, alkoxy, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
or:
X¹ and X² of Formula (XLIV) are independently selected from C and N, and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and X³ of Formula (XLIV) is CR^{2a}R^{2b};
or:
X² and X³ of Formula (XLIV) are independently selected from C and N. and are members of a fused substituted or unsubstituted saturated or partially saturated 3-10 membered cycloalkyl ring, a fused substituted or unsubstituted saturated or partially saturated 3-10 membered heterocycloalkyl ring, a fused substituted or unsubstituted 5-10 membered aryl ring, or a fused substituted or unsubstituted 5-10 membered heteroaryl ring, and Xj of Formula (VLIV) is CR^{2e}R^{2f};
R^{A} of N—R^{A} is selected from H, C₁-C₆alkyl, —C(═O)C₁-C₂alkyl, substituted or unsubstituted aryl, or substituted or unsubstituted heteroaryl;
R^{2a}, R^{2b}, R^{2c}, R^{2d}, R^{2e}, and R^{2f} of CR^{2c}R^{2d}, CR^{2a}R^{2b} and CR^{2e}R^{2f} are independently selected from H, substituted or unsubstituted C₁-C₆alkyl, substituted or unsubstituted C₁-C₆heteroalkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), —C₁-C₆alkyl-(substituted or unsubstituted heteroaryl) and —C(═O)R^{B};
R^{B} of —C(═O)R^{B} is selected from substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl-(substituted or unsubstituted C₃-C₆cycloalkyl), —C₁-C₆alkyl-(substituted or unsubstituted C₂-C₅heterocycloalkyl), —C₁-C₆alkyl- (substituted or unsubstituted aryl), —C₁-C₆alkyl-(substituted or unsubstituted heteroaryl), or —NR^{D}R^{E};
R^{D} and R^{E} of NR^{D}R^{E} are independently selected from H, substituted or unsubstituted C₁-C₆alkyl, substituted or unsubstituted C₃-C₆cycloalkyl, substituted or unsubstituted C₂-C₅heterocycloalkyl, substituted or unsubstituted aryl, substituted or unsubstituted heteroaryl, —C₁-C₆alkyl- (substituted or unsubstituted C₃-C₆cycloalkyl), —C₁-C₆alkyl-(substituted or unsubstituted C₂-C₅heterocycloalkyl), —C₁-C₆alkyl-(substituted or unsubstituted aryl), or —C₁-C₆alkyl- (substituted or unsubstituted heteroaryl);
m of Formula (XLIV) is selected from 0, 1 or 2;
—U— of Formula (XLIV) is selected from —NHC(═O)—, —C(═O)NH—, —NHS(═O)₂—, —S(═O)₂NH—, —NHC(═O)NH—, —NH(C═O)O—, —O(C═O)NH—, or —NHS(═O)₂NH—;
R³ of Formula (XLIV) is selected from C₁-C₃alkyl, or C₁-C₃fluoroalkyl;
R⁴ of Formula (XLIV) is selected from —NHR⁵, —N(R⁵)2, —N+(R⁵)₃ or —OR⁵;
each R⁵ of —NHR⁵, —N(R⁵)₂, —N+(R⁵)₃ and —OR⁵ is independently selected from H, C₁-C₃alkyl, C₁-C₃haloalkyl, C₁-C₃heteroalkyl and —C₁-C₃alkyl-(C₃-C₅cycloalkyl);
or:
R³ and R⁵ of Formula (XLIV) together with the atoms to which they are attached form a substituted or unsubstituted 5-7 membered ring;
or:
R³ of Formula (XLIII) is bonded to a nitrogen atom of U to form a substituted or unsubstituted 5-7 membered ring;
R⁶ of Formula (XLIII) is selected from —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)₂NHR⁷; —(C₁-C₃alkyl)-NHC(═O)NHR⁷, —(C₁-C₃alkyl)-NHS(═O)₂NHR⁷, substituted or unsubstituted C₂-C₁₀heterocycloalkyl, or substituted or unsubstituted heteroaryl;
each R⁷ of —NHC(═O)R⁷, —C(═O)NHR⁷, —NHS(═O)2R⁷, —S(═O)₂NHR⁷; —NHC(═O)NHR⁷, —NHS(═O)₂NHR⁷, —(C₁-C₃alkyl)-NHC(═O)R⁷, —(C₁-C₃alkyl)-C(═O)NHR⁷, —(C₁-C₃alkyl)-NHS(═O)2R⁷, —(C₁-C₃alkyl)-S(═O)2NHR⁷; —(C₁-C₃alkyl)-NHC(═O)NHR⁷, —(C₁-C₃alkyl)-NHS(═O)2NHR⁷ is independently selected from C₁-C₆alkyl, C₁-C₆haloalkyl, C₁-C₆heteroalkyl, a substituted or unsubstituted C₃-C₁₀cycloalkyl, a substituted or unsubstituted C₂-C₁₀heterocycloalkyl, a substituted or unsubstituted aryl, a substituted or unsubstituted heteroaryl, —C₁-C₆alkyl-(substituted or unsubstituted C₃-C₁₀cycloalkyl), —C₁-C₆alkyl- (substituted or unsubstituted C₂-C₁₀heterocycloalkyl, —C₁-C₆alkyl-(substituted or unsubstituted aryl), —C₁-C₆alkyl-(substituted or unsubstituted heteroaryl), —(CH2)p-CH(substituted or unsubstituted aryl)2, —(CH2)ₚ—CH(substituted or unsubstituted heteroaryl)2, —(CH2)_{P}—CH(substituted or unsubstituted aryl)(substituted or unsubstituted heteroaryl), -(substituted or unsubstituted aryl)-(substituted or unsubstituted aryl), -(substituted or unsubstituted aryl)-(substituted or unsubstituted heteroaryl), -(substituted or unsubstituted heteroaryl)-(substituted or unsubstituted aryl), or -(substituted or unsubstituted heteroaryl)-(substituted or unsubstituted heteroaryl);
p of R⁷ is selected from 0, 1 or 2;
R^{8a}, R^{8b}, R^{8c}, R^{8d}, R^{8e}, and R^{8f} of C(R^{8a})(R^{8b}), C(R^{8c})(R^{8d}) and C(R^{8e})(R^{8f}) are independently selected from H, C₁-C₆alkyl, C₁-C₆fluoroalkyl, C₁-C₆ alkoxy, C₁-C₆heteroalkyl, and substituted or unsubstituted aryl;
or:
R^{8a}, R^{8d}, R^{8e}, and R⁸¹ of C(R^{8a})(R^{8b}), C(R^{8c})(R^{8d}) and C(R^{8e})(R^{8f}) are as defined above, and R^{8b} and R^{8c} together form a bond;
or:
R^{8a}, R^{8b}, R^{8d}, and R^{8f} of C(R^{8a})(R^{8b}), C(R^{8c})(R^{8d}) and C(R^{8e})(R^{8f}) are as defined above, and R^{8c} and R^{8e} together form a bond;
or:
R^{8a}, R^{8d}, R^{8e}, and R^{8f} of C(R^{8a})(R^{8b}), C(R^{8c})(R^{8d}) and C(R^{8e})(R^{8f}) are as defined above, and R^{8b} and R^{8c} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8a}, R^{8b}, R^{8d}, and R⁸ of C(R^{8a})(R^{8b}), C(R^{8c})(R^{8d}) and C(R^{8e})(R^{8f}) are as defined above, and R^{8c} and R^{8e} together with the atoms to which they are attached form a substituted or unsubstituted fused 5-7 membered saturated, or partially saturated carbocyclic ring or heterocyclic ring comprising 1-3 heteroatoms selected from S, O and N, a substituted or unsubstituted fused 5-10 membered aryl ring, or a substituted or unsubstituted fused 5-10 membered heteroaryl ring comprising 1-3 heteroatoms selected from S, O and N;
or:
R^{8c}, R^{8d}, R^{8e}, and R^{8f} of C(R^{8c})(R^{8d}) and C(R^{8e})(R^{8f}) are as defined above, and R^{8a} and R^{8b} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8e}, and R^{8f} of C(R^{8a})(R^{8b}) and C(R^{8e})(R^{8f}) are as defined above, and R^{8c} and R^{8d} together with the atoms to which they are attached form a substituted or unsubstituted saturated, or partially saturated 3-7 membered spirocycle or heterospirocycle comprising 1-3 heteroatoms selected from S, O and N;
or:
R^{8a}, R^{8b}, R^{8c}, and R^{8d} of C(R^{8a})(R^{8b}) and C(R^{8c})(R^{8d}) are as defined above, and R^{8e} and R^{8f} together with the atoms to which they are attached form a substituted or unsubstituted saturated, or partially saturated 3-7 membered spirocycle or heterospirocycle comprising 1-3 heteroatoms selected from S, O and N;
or:
where each substituted alkyl, heteroalkyl, fused ring, spirocycle, heterospirocycle, cycloalkyl, heterocycloalkyl, aryl or heteroaryl is substituted with 1-3 R⁹; and
each R⁹ of R^{8a}, R^{8b}, R^{8c}, R^{8d}, R^{8e}, and R^{8f} is independently selected from halogen, —OH, —SH, (C═O), CN, C₁-C₄alkyl, C₁-C₄fluoroalkyl, C₁-C₄ alkoxy, C₁-C₄ fluoroalkoxy, —NH₂, —NH(C₁-C₄alkyl), —NH(C₁-C₄alkyl)₂, —C(═O)OH, —C(═O)NH₂, —C(═O)C₁-C₃alkyl, —S(═O)₂CH₃, —NH(C₁-C₄alkyl)-OH, —NH(C₁-C₄alkyl)-O—(C—C₄alkyl), —O(C₁-C₄alkyl)-NH2; —O(C₁-C₄alkyl)-NH—(C₁-C₄alkyl), and —O(C₁-C₄alkyl)-N—(C₁-C₄alkyl)2, or two R⁹ together with the atoms to which they are attached form a methylene dioxy or ethylene dioxy ring substituted or unsubstituted with halogen, —OH, or C₁-C₃alkyl.

In any of the compounds described herein, the ILM can have the structure of Formula (XLV), (XLVI) or (XLVII), which is derived from the IAP ligands described in Vamos, M., et al., Expedient synthesis of highly potent antagonists of inhibitor of apoptosis proteins (IAPs) with unique selectivity for ML-IAP, ACS Chem. Biol., 8(4), 725-32 (2013), or an unnatural mimetic thereof:

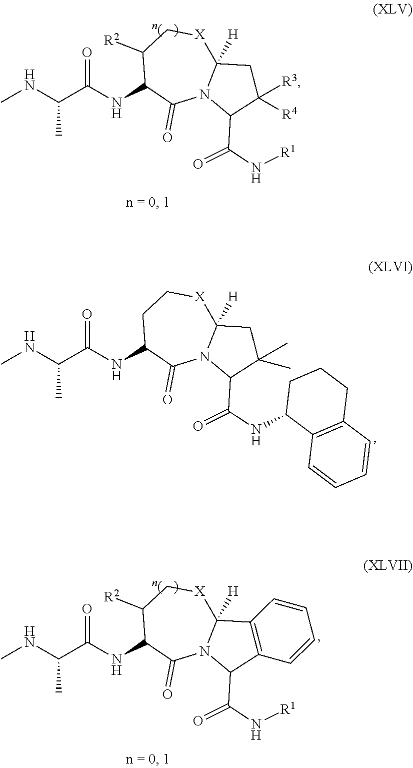

wherein:
R², R³ and R⁴ of Formula (XLV) are independently selected from H or ME;
X of Formula (XLV) is independently selected from O or S; and
R¹ of Formula (XLV) is selected from:

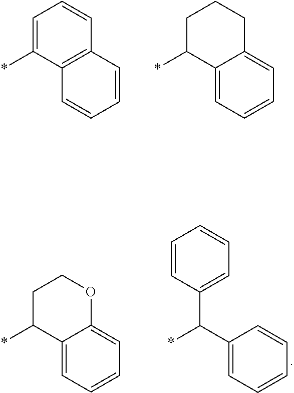

In a particular embodiment, the ILM has a structure according to Formula (XLVIII):

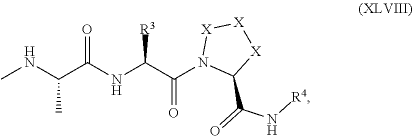

wherein R³ and R⁴ of Formula (XLVIII) are independently selected from H or ME;

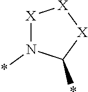

is a 5-member heterocycle selected from:

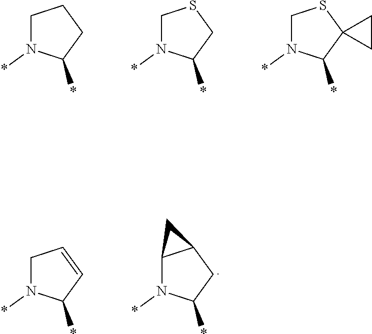 [SMILES: *[C@@H]1CCCN1* ; *[C@@H]1CSCN1* ; *[C@H]1N(*)CSC12CC2 ; *[C@@H]1C=CCN1* ; *[C@@H]1C[C@@H]2C[C@@H]2N1*]

In a particular embodiment, the

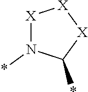

### of Formula XLVIII) is

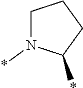 [SMILES: *[C@@H]1CCCN1*]

In a particular embodiment, the ILM has a structure and attached to a linker group L as shown below:

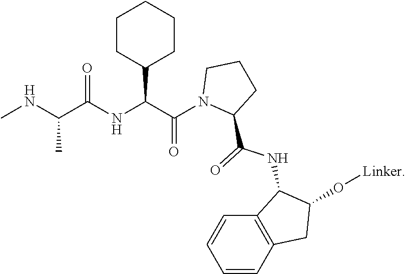

In a particular embodiment, the ILM has a structure according to Formula (XLIX), (L), or (LI):

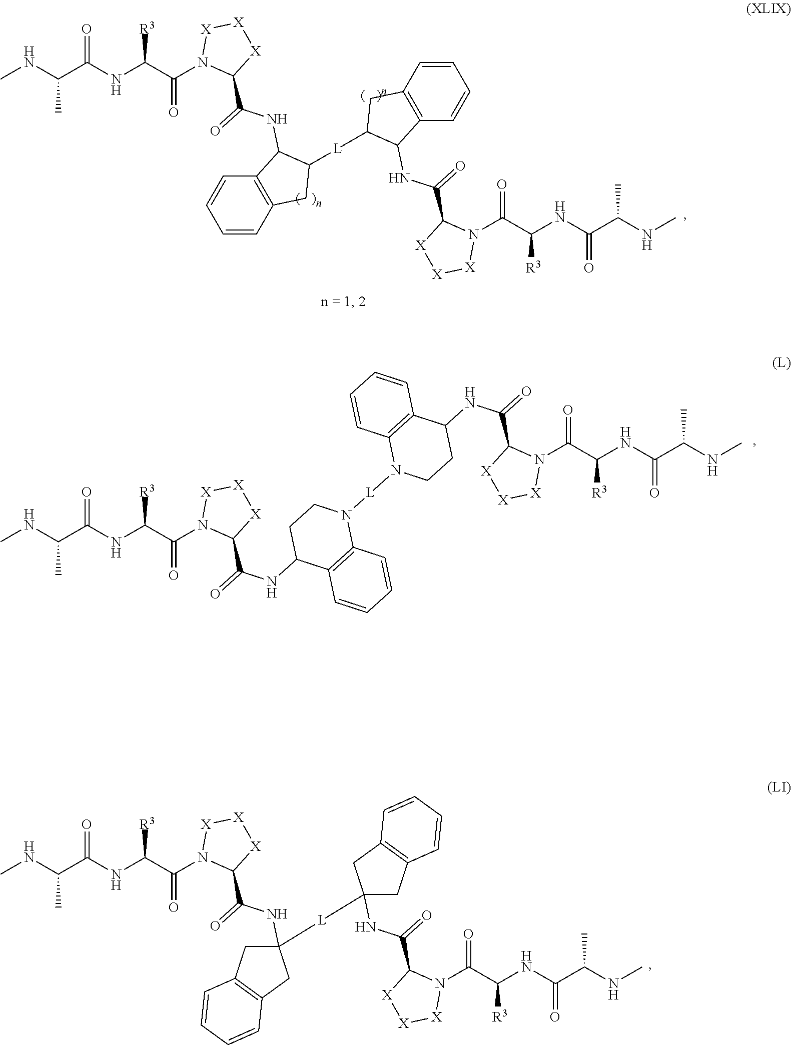

wherein:
R³ of Formula (XLIX), (L) or (LI) are independently selected from H or ME;

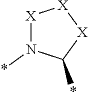

is a 5-member heterocycle selected from:

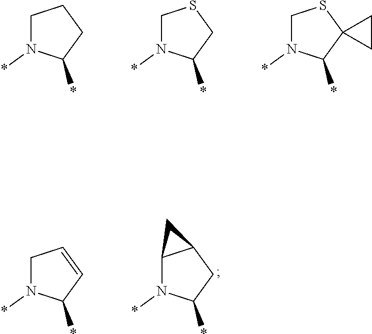 [SMILES: *[C@@H]1CCCN1* ; *[C@@H]1CSCN1* ; *[C@H]1N(*)CSC12CC2 ; *[C@@H]1C=CCN1* ; *[C@@H]1C[C@@H]2C[C@@H]2N1*]

and L of Formula (XLIX), (L) or (LI) is selected from:

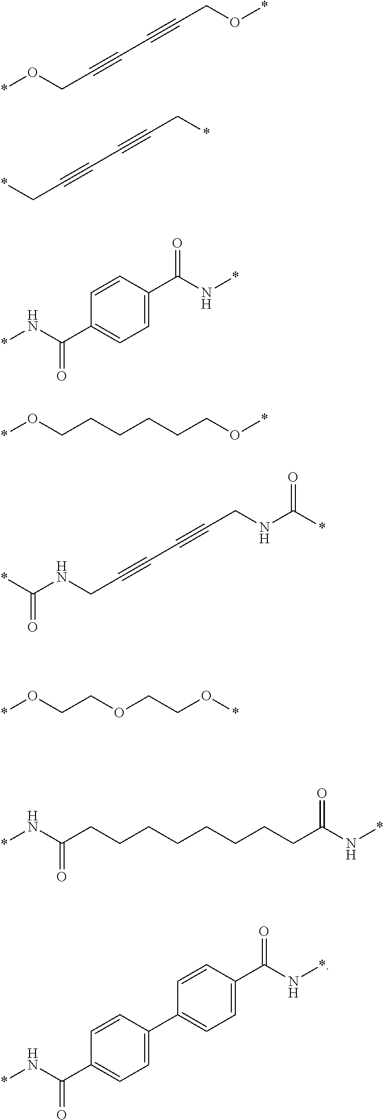

In a particular embodiment L of Formula (XLIX), (L), or (LI)

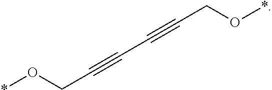

In a particular embodiment, the ILM has a structure according to Formula (LII):

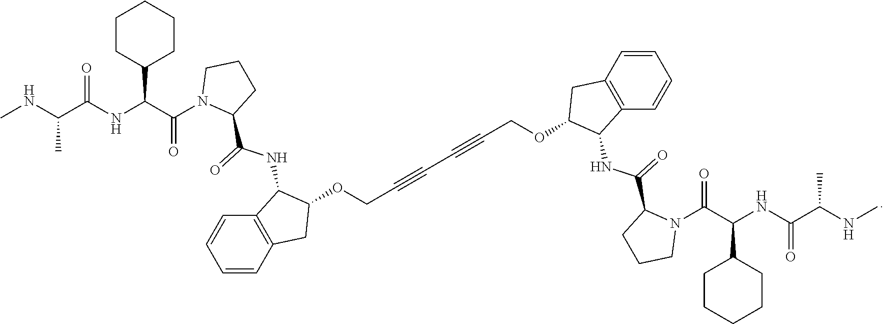

In a particular embodiment, the ILM according to Formula (LII) is chemically linked to the linker group L in the area denoted with [symbol] , and as shown below:

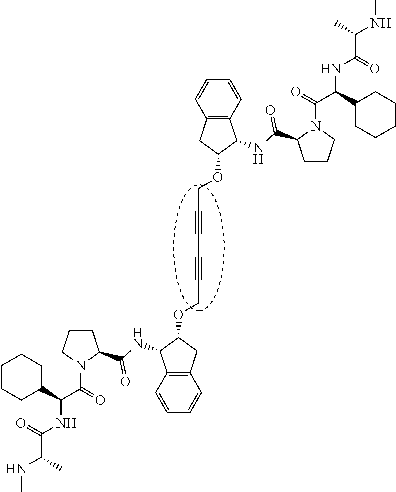 [SMILES: CN[C@@H](C)C(=O)N[C@H](C(=O)N1CCC[C@H]1C(=O)N[C@H]1c2ccccc2C[C@H]1OCC#CC#CCO[C@@H]1Cc2ccccc2[C@@H]1NC(=O)[C@@H]1CCCN1C(=O)[C@@H](NC(=O)[C@H](C)NC)C1CCCCC1)C1CCCCC1]

In some exemplary embodiment, a compound containing a PTM, L, and a ILM is selected from the group consisting of:

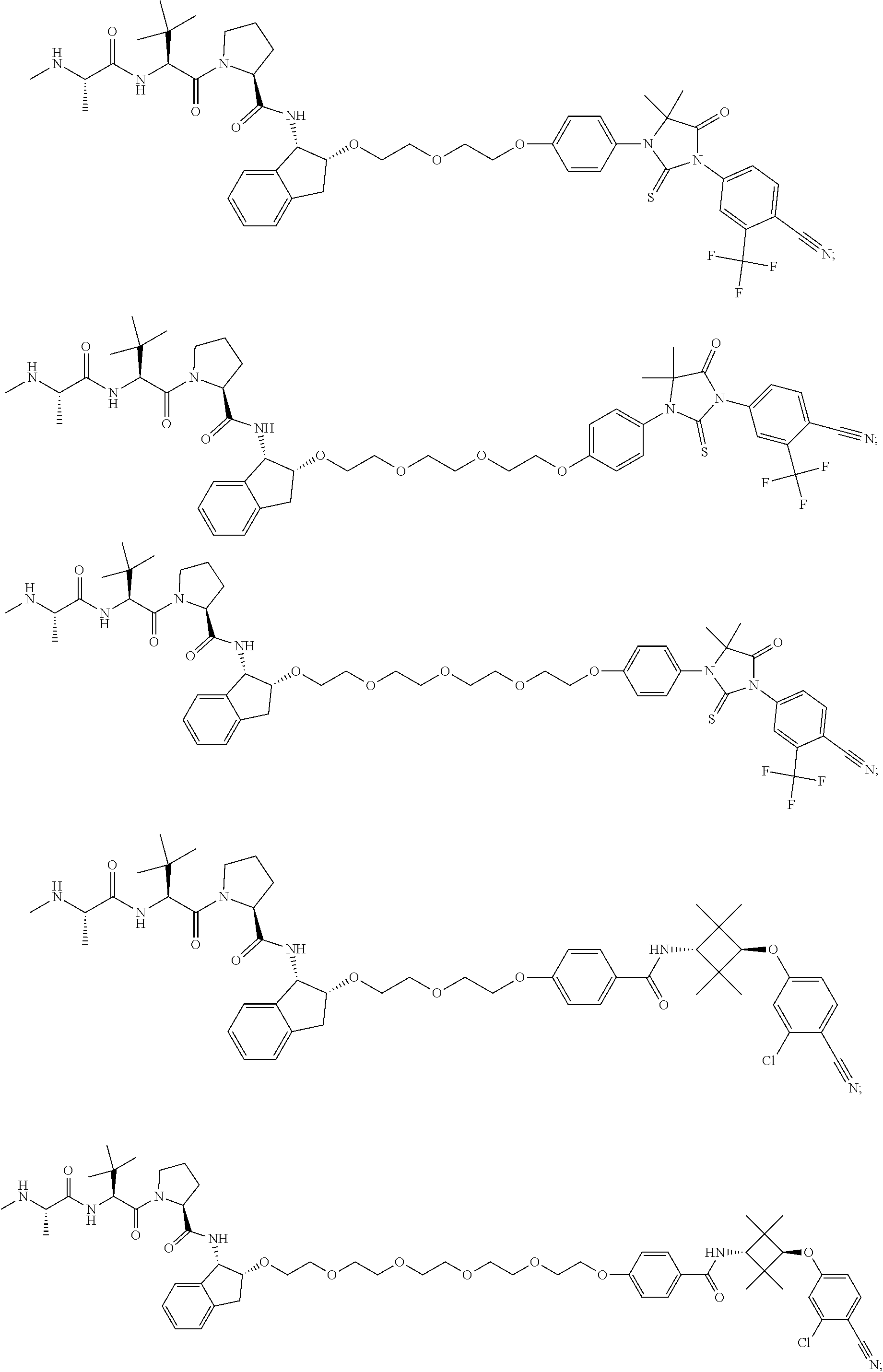

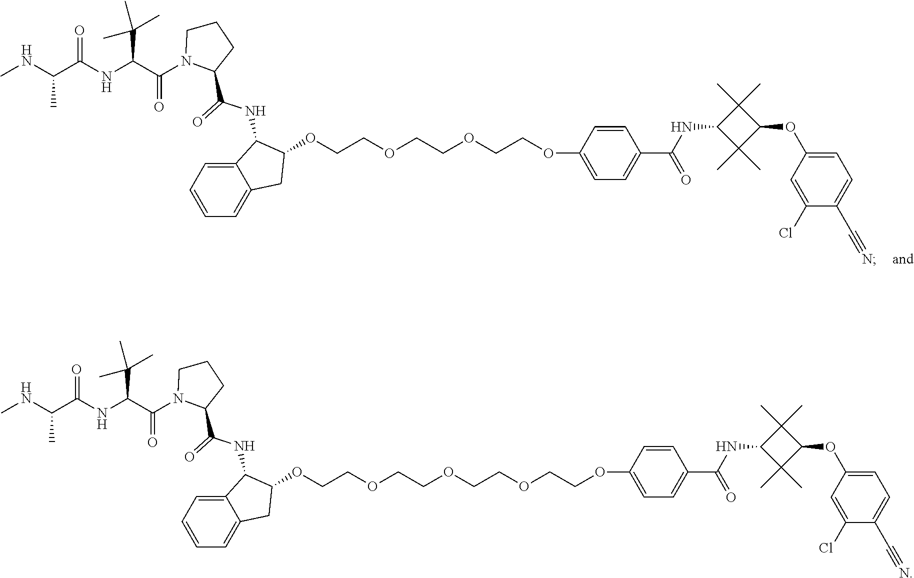 [SMILES: CN[C@@H](C)C(=O)N[C@H](C(=O)N1CCC[C@H]1C(=O)N[C@H]1c2ccccc2C[C@H]1OCCOCCOCCOc1ccc(C(=O)N[C@H]2C(C)(C)[C@H](Oc3ccc(C#N)c(Cl)c3)C2(C)C)cc1)C(C)(C)C ; CN[C@@H](C)C(=O)N[C@H](C(=O)N1CCC[C@H]1C(=O)N[C@H]1c2ccccc2C[C@H]1OCCOCCOCCOCCOc1ccc(C(=O)N[C@H]2C(C)(C)[C@H](Oc3ccc(C#N)c(Cl)c3)C2(C)C)cc1)C(C)(C)C]

In any of the compounds described herein, the ILM can have the structure of Formula (LIII) or (LIV), which is based on the IAP ligands described in Hennessy, E J, et al., Discovery of aminopiperidine-based Smac mimetics as IAP antagonists, Bioorg. Med. Chem. Lett., 22(4), 1960-4 (2012), or an unnatural mimetic thereof:

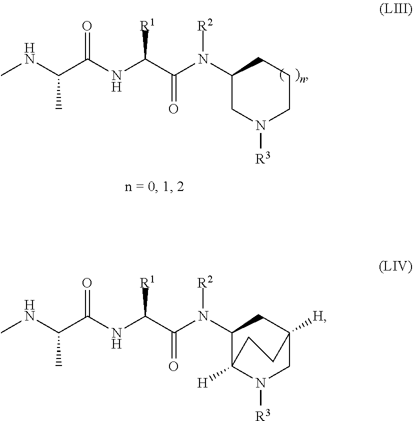

wherein:
R¹ of Formulas (LIII) and (LIV) is selected from:

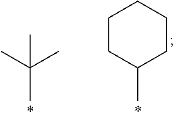

R² of Formulas (LIII) and (LIV) is selected from H or Me;
R³ of Formulas (LIII) and (LIV) is selected from:

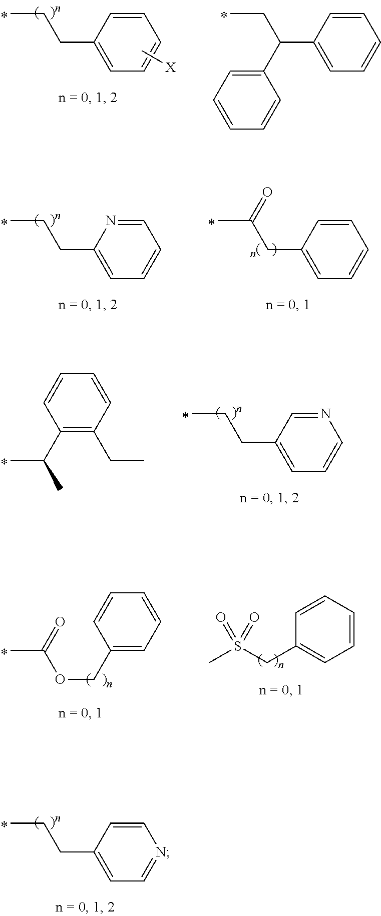

X of is selected from H, halogen, methyl, methoxy, hydroxy, nitro or trifluoromethyl.

In any of the compounds described herein, the ILM can have the structure of and be chemically linked to the linker as shown in Formula (LV) or (LVI), or an unnatural mimetic thereof:

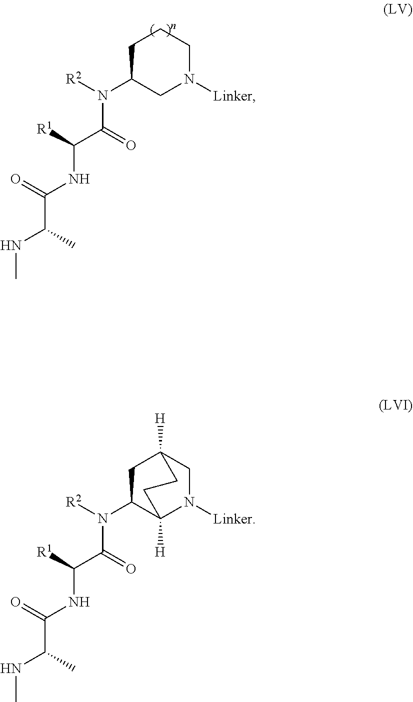

In any of the compounds described herein, the ILM can have the structure of Formula (LVII), which is based on the IAP ligands described in Cohen, F, et al., Orally bioavailable antagonists of inhibitor of apoptosis proteins based on an azabicyclooctane scaffold, J. Med. Chem., 52(6), 1723-30 (2009), or an unnatural mimetic thereof:

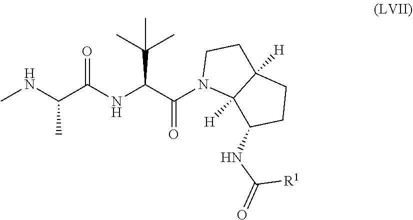

wherein:
R¹ of Formulas (LVII) is selected from:

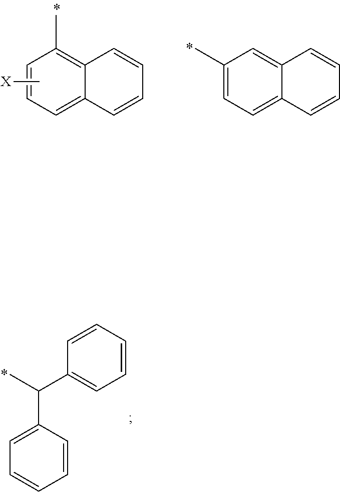

### X of

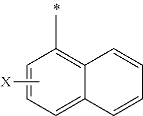

is selected from H, fluoro, methyl or methoxy.

In a particular embodiment, the ILM is represented by the following structure:

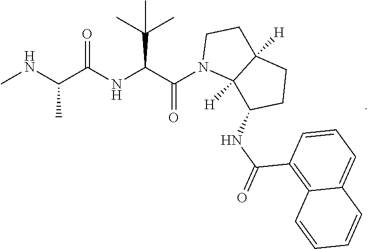 [SMILES: CN[C@@H](C)C(=O)N[C@H](C(=O)N1CC[C@H]2CC[C@H](NC(=O)c3cccc4ccccc34)[C@H]21)C(C)(C)C]

In a particular embodiment, the ILM is selected from the group consisting of, and which the chemical link between the ILM and linker group L is shown:

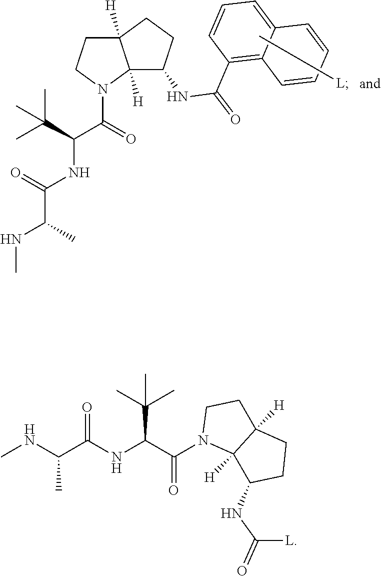

In any of the compounds described herein, the ILM is selected from the group consisting of the structures below, which are based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

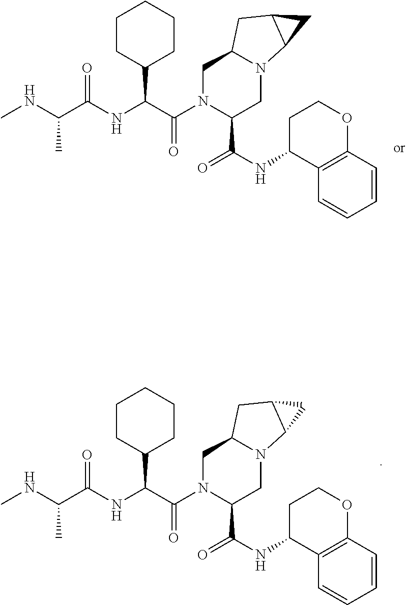 [SMILES: CN[C@@H](C)C(=O)N[C@H](C(=O)N1C[C@H]2C[C@H]3C[C@H]3N2C[C@H]1C(=O)N[C@@H]1CCOc2ccccc21)C1CCCCC1 ; CN[C@@H](C)C(=O)N[C@H](C(=O)N1C[C@H]2C[C@@H]3C[C@@H]3N2C[C@H]1C(=O)N[C@@H]1CCOc2ccccc21)C1CCCCC1]

In a particular embodiment, the ILM is selected from the group consisting of, and which the chemical link between the ILM and linker group L is shown:

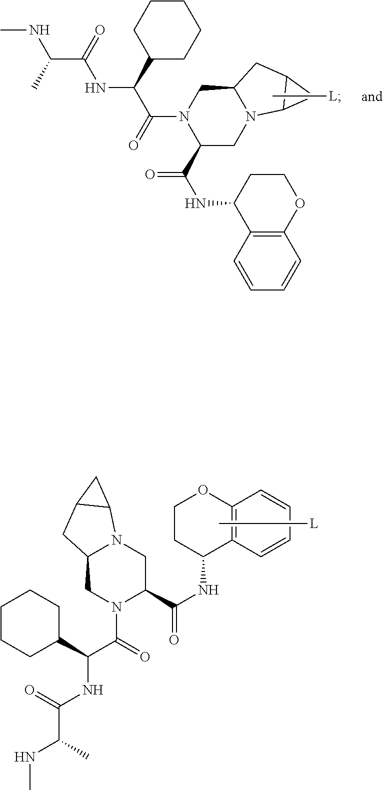

In any of the compounds described herein, the ILM can have the structure of Formula (LVIII), which is based on the IAP ligands described in Asano, M, et al., Design, sterioselective synthesis, and biological evaluation of novel tri-cyclic compounds as inhibitor of apoptosis proteins (IAP) antagonists, Bioorg. Med. Chem., 21(18): 5725-37 (2013), or an unnatural mimetic thereof:

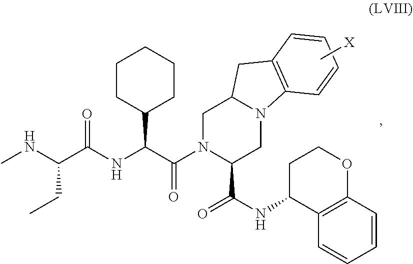

wherein X of Formula (LVIII) is one or two substituents independently selected from H, halogen or cyano.

In any of the compounds described herein, the ILM can have the structure of and be chemically linked to the linker group L as shown in Formula (LIX) or (LX), or an unnatural mimetic thereof:

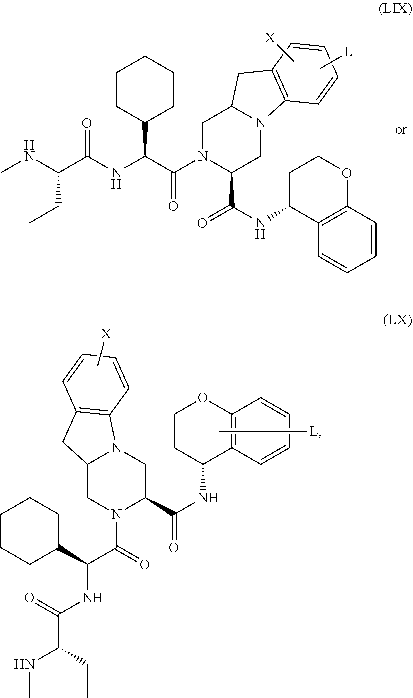

wherein X of Formula (LIX) and (LX) is one or two substituents independently selected from H, halogen or cyano, and; and L of Formulas (LIX) and (LX) is a linker group as described herein.

In any of the compounds described herein, the ILM can have the structure of Formula (LXI), which is based on the IAP ligands described in Ardecky, R J, et al., Design, sysnthesis and evaluation of inhibitor of apoptosis (IAP) antagonists that are highly selective for the BIR2 domain of XIAP, Bioorg. Med. Chem., 23(14): 4253-7 (2013), or an unnatural mimetic thereof:

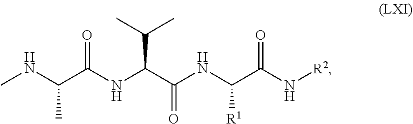

wherein:

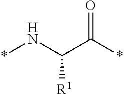

of Formula (LXI) is a natural or unnatural amino acid; and
R² of Formula (LXI) is selected from:

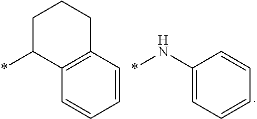

In any of the compounds described herein, the ILM can have the structure of and be chemically linked to the linker group L as shown in Formula (LXII) or (LLXIII), or an unnatural mimetic thereof:

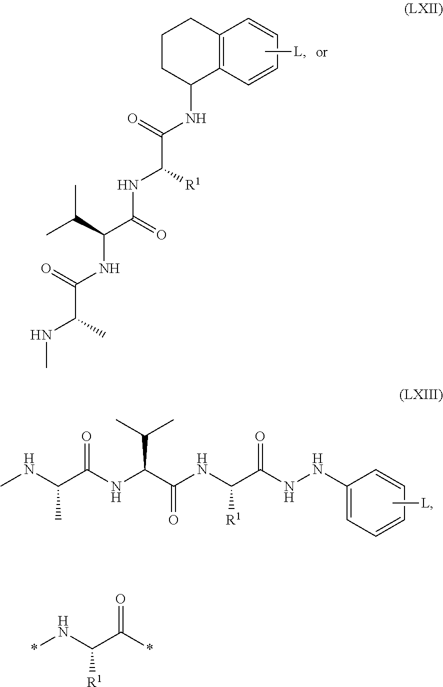

of Formula (LXI) is a natural or unnatural amino acid; and L of Formula (LXI) is a linker group as described herein.

In any of the compounds described herein, the ILM can have the structure selected from the group consisting of, which is based on the IAP ligands described in Wang, J, et al., Discovery of novel second mitochondrial-derived activator of caspase mimetics as selective inhibitor or apoptosis protein inhibitors, J. Pharmacol. Exp. Ther., 349(2): 319-29 (2014), or an unnatural mimetic thereof:

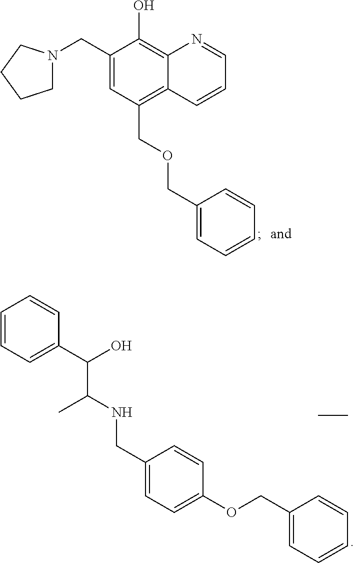 [SMILES: Oc1c(CN2CCCC2)cc(COCc2ccccc2)c2cccnc12 ; CC(NCc1ccc(OCc2ccccc2)cc1)C(O)c1ccccc1]

In any of the compounds described herein, the ILM has a structure according to Formula (LIX), which is based on the IAP ligands described in Hird, A W, et al., Structure-based design and synthesis of tricyclic IAP (Inhibitors of Apoptosis Proteins) inhibitors, Bioorg. Med. Chem. Lett., 24(7): 1820-4 (2014), or an unnatural mimetic thereof:

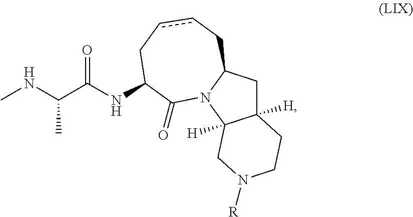

wherein R of Formula LIX is selected from the group consisting of:

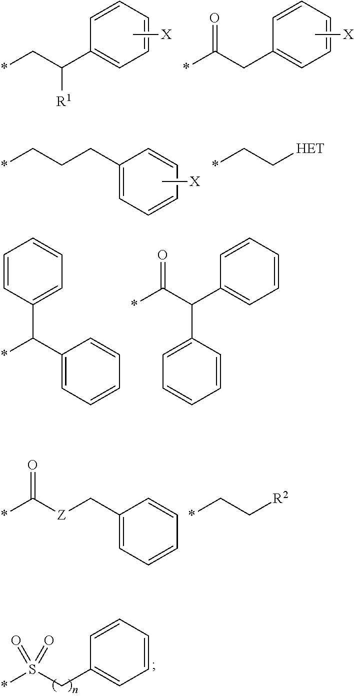

### R¹ of

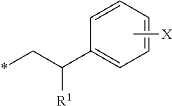

is selected from H or Me;

### R² of

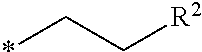

is selected from alkyl or cycloalkyl;

### X of

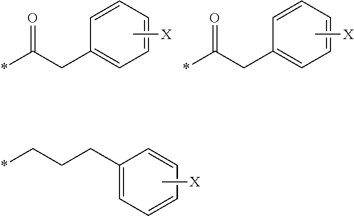

is 1-2 substitutents independently selected from halogen, hydroxy, methoxy, nitro and trifluoromethyl

### Z of

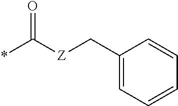

### is O or NH;

### HET of

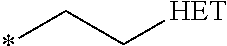

is mono- or fused bicyclic heteroaryl; and
--- of Formula (LIX) is an optional double bond.

In a particular embodiment, the ILM of the compound has a chemical structure as represented by:

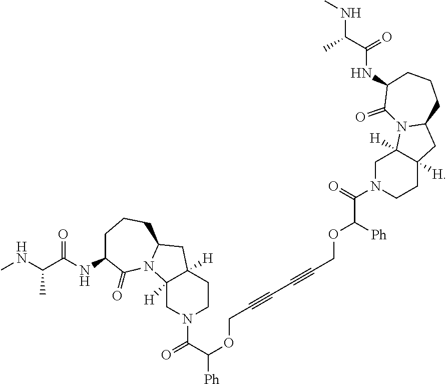

In a particular embodiment, the ILM of the compound has a chemical structure selected from the group consisting of:

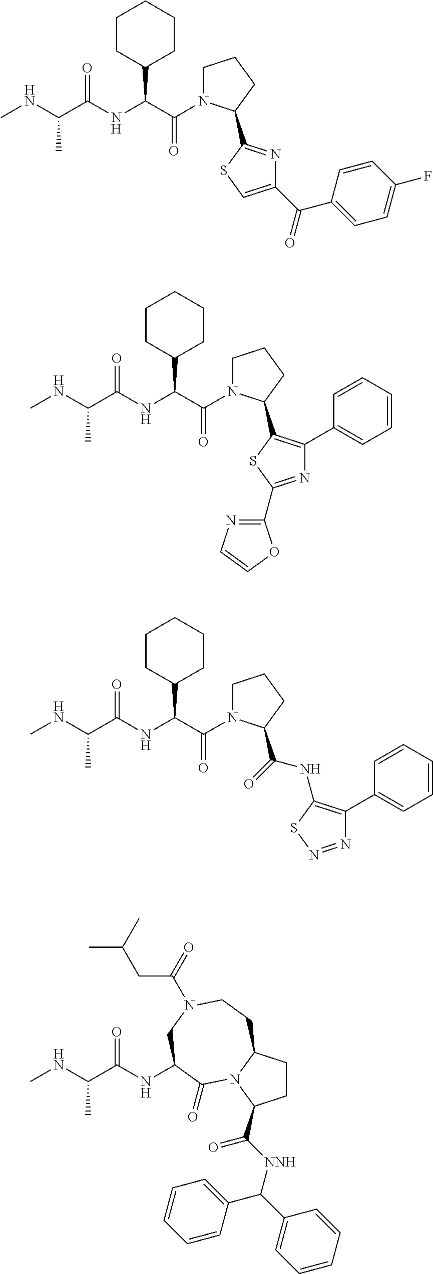

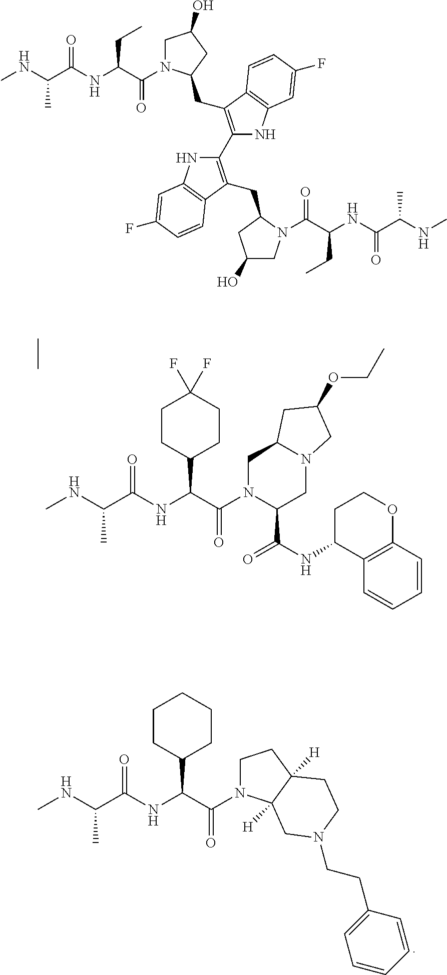

The term “independently” is used herein to indicate that the variable, which is independently applied, varies independently from application to application.

The term “alkyl” shall mean within its context a linear, branch-chained or cyclic fully saturated hydrocarbon radical or alkyl group, preferably a C₁-C₁₀, more preferably a C₁-C₆, alternatively a C₁-C₃ alkyl group, which may be optionally substituted. Examples of alkyl groups are methyl, ethyl, n-butyl, sec-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopen-tylethyl, cyclohexylethyl and cyclohexyl, among others. In certain embodiments, the alkyl group is end-capped with a halogen group (At, Br, Cl, F, or I). In certain preferred embodiments, compounds according to the present invention which may be used to covalently bind to dehalogenase enzymes. These compounds generally contain a side chain (often linked through a polyethylene glycol group) which terminates in an alkyl group which has a halogen substituent (often chlorine or bromine) on its distal end which results in covalent binding of the compound containing such a moiety to the protein.

The term “Alkenyl” refers to linear, branch-chained or cyclic C₂-C₁₀ (preferably C₂-C₆) hydrocarbon radicals containing at least one C═C bond.

The term “Alkynyl” refers to linear, branch-chained or cyclic C₂-C₁₀ (preferably C₂-C₆) hydrocarbon radicals containing at least one C≡C bond.

The term “alkylene” when used, refers to a —(CH₂)ₙ— group (n is an integer generally from 0-6), which may be optionally substituted. When substituted, the alkylene group preferably is substituted on one or more of the methylene groups with a C₁-C₆ alkyl group (including a cyclopropyl group or a t-butyl group), but may also be substituted with one or more halo groups, preferably from 1 to 3 halo groups or one or two hydroxyl groups, O—(C₁-C₆ alkyl) groups or amino acid sidechains as otherwise disclosed herein. In certain embodiments, an alkylene group may be substituted with a urethane or alkoxy group (or other group) which is further substituted with a polyethylene glycol chain (of from 1 to 10, preferably 1 to 6, often 1 to 4 ethylene glycol units) to which is substituted (preferably, but not exclusively on the distal end of the polyethylene glycol chain) an alkyl chain substituted with a single halogen group, preferably a chlorine group. In still other embodiments, the alkylene (often, a methylene) group, may be substituted with an amino acid sidechain group such as a sidechain group of a natural or unnatural amino acid, for example, alanine, β-alanine, arginine, asparagine, aspartic acid, cysteine, cystine, glutamic acid, glutamine, glycine, phenylalanine, histidine, isoleucine, lysine, leucine, methionine, proline, serine, threonine, valine, tryptophan or tyrosine.

The term “unsubstituted” shall mean substituted only with hydrogen atoms. A range of carbon atoms which includes C₀ means that carbon is absent and is replaced with H. Thus, a range of carbon atoms which is C₀-C₆ includes carbons atoms of 1, 2, 3, 4, 5 and 6 and for C₀, H stands in place of carbon.

The term “substituted” or “optionally substituted” shall mean independently (i.e., where more than substituent occurs, each substituent is independent of another substituent) one or more substituents (independently up to five substitutents, preferably up to three substituents, often 1 or 2 substituents on a moiety in a compound according to the present invention and may include substituents which themselves may be further substituted) at a carbon (or nitrogen) position anywhere on a molecule within context, and includes as substituents hydroxyl, thiol, carboxyl, cyano (C≡N), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or C₁), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present invention may include, for example —SiR₁R₂R₃ groups where each of R₁ and R₂ is as otherwise described herein and R₃ is H or a C₁-C₆alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteraryl moiety) through an optionally substituted —(CH₂)ₘ or alternatively an optionally substituted —(OCH₂)ₘ—, —(OCH₂CH₂)ₘ or —(CH₂CH₂O)ₘ group, which may be substituted with any one or more of the above-described substituents. Alkylene groups —(CH2)ₘ or —(CH₂)ₙ— groups or other chains such as ethylene glycol chains, as identified above, may be substituted anywhere on the chain. Preferred substitutents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sideshain of an amino acid as otherwise described herein and optionally substituted amide (preferably carboxamide substituted as described above) or urethane groups (often with one or two C₀-C₆ alkyl substitutents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present invention, a moiety in a molecule may be optionally substituted with up to five substituents, preferably up to three substituents. Most often, in the present invention moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R¹)—C(O)—O—R¹, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂),OH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R², —S(O)₂—R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present invention at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably substituted with a linker group attached to a PTM group, including a ULM group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

“Carboxyl” denotes the group —C(O)OR, where R is hydrogen, alkyl, substituted alkyl, aryl, substituted aryl, heteroaryl or substituted heteroaryl, whereas these generic substituents have meanings which are identical with definitions of the corresponding groups defined herein.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

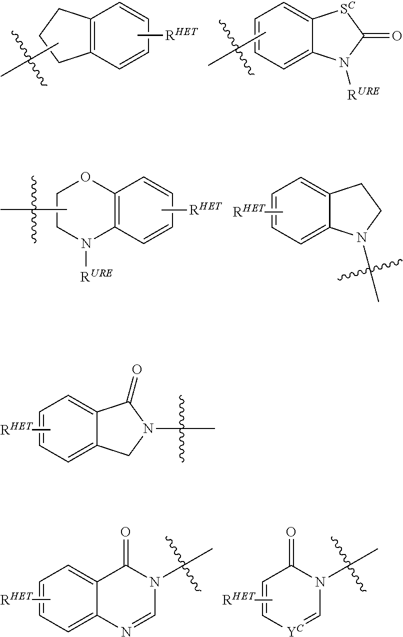

wherein
S^{c} is CHR^{SS}, NR^{URE}, or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and
Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl).

The terms “aralkyl” and “heteroarylalkyl” refer to groups that comprise both aryl or, respectively, heteroaryl as well as alkyl and/or heteroalkyl and/or carbocyclic and/or heterocycloalkyl ring systems according to the above definitions.

The term “arylalkyl” as used herein refers to an aryl group as defined above appended to an alkyl group defined above. The arylalkyl group is attached to the parent moiety through an alkyl group wherein the alkyl group is one to six carbon atoms. The aryl group in the arylalkyl group may be substituted as defined above.

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroirnidazolyl, dihydropyranyl, dihydrofuranyl, dioxanyl, dioxolanyl, ethyleneurea, 1,3-dioxolane, 1,3-dioxane, 1,4-dioxane, furyl, homnopiperidinyl, inidazolyl, inidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidiiyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyL, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO-heteroaryl, —SO2-alkyl, —SO2-substituted alkyl, —SO2-aryl, oxo (═O), and —SO2-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S or P. “Substituted heterocycloalkyl” refers to a monocyclic or polycyclic alkyl group in which at least one ring carbon atom of its cyclic structure being replaced with a heteroatom selected from the group consisting of N, O, S or P and the group is containing one or more substituents selected from the group consisting of halogen, alkyl, substituted alkyl, carbyloxy, carbylmercapto, aryl, nitro, mercapto or sulfo, whereas these generic substituent group have meanings which are identical with definitions of the corresponding groups as defined in this legend.

The term “hydrocarbyl” shall mean a compound which contains carbon and hydrogen and which may be fully saturated, partially unsaturated or aromatic and includes aryl groups, alkyl groups, alkenyl groups and alkynyl groups.

In any of the embodiments described herein, the W, X, Y, Z, G, G′, R, R′, R″, Q1-Q4, A, and Rn can independently be covalently coupled to a linker and/or a linker to which is attached one or more PTM, ULM, ILM or ILM′ groups.

### Exemplary Linkers

In certain embodiments, the compounds as described herein can be chemically linked or coupled via a chemical linker (L). In certain embodiments, the linker group L is a group comprising one or more covalently connected structural units of A (e.g., -A_{1 . . .} A_{q}-), wherein A₁ is a group coupled to at least one of a ULM, a PTM, or a combination thereof. In certain embodiments, A₁ links a ULM, a PTM, or a combination thereof directly to another ULM, PTM, or combination thereof. In other embodiments, A₁ links a ULM, a PTM, or a combination thereof indirectly to another ULM, PTM, or combination thereof through A_{q}.

In certain embodiments, A₁ links a ULM, a PTM, or a combination thereof directly to another ULM, PTM, or combination thereof. In other embodiments, A₁ links a ULM, a PTM, or a combination thereof indirectly to another ULM, PTM, or combination thereof through A_{q}. In a particular embodiment, A₁ to A_{q} are, each independently, a bond, CR^{L1}R^{L2}, O, S, SO, SO₂, NR³, SO₂NR^{L3}, SONR^{L3}, CONR^{L3}, NR^{L3}CONR^{L4}, NR^{L3}SO₂NR^{L4}, CO, CR^{L1}αCR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heterocyclyl optionally substituted with 0-6 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently, can be linked to other A groups to form cycloalkyl and/or heterocyclyl moeity which can be further substituted with 0-4 R^{L5} groups; wherein

R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)2, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NHSO₂NH(C₁₋₈alkyl), NHSO₂N(C₁₋₈alkyl)₂, NHSO₂NH₂.

In certain embodiments, q is an integer greater than or equal to 0. In certain embodiments, q is an integer greater than or equal to 1.

In certain embodiments, e.g., where q is greater than 2, A_{q} is a group which is connected to a ULM or ULM′ moiety, and A₁ and A_{q} are connected via structural units of A (number of such structural units of A: q-2).

In certain embodiments, e.g., where q is 2, A_{q} is a group which is connected to A₁ and to a ULM or ULM′ moiety.

In certain embodiments, e.g., where q is 1, the structure of the linker group L is -A₁-, and A₁ is a group which is connected to a ULM or ULM′ moiety and a PTM moiety.

In additional embodiments, q is an integer from 1 to 100, 1 to 90, 1 to 80, 1 to 70, 1 to 60, 1 to 50, 1 to 40, 1 to 30, 1 to 20, or 1 to 10.

In certain embodiments, the linker (L) is selected from the group consisting of):

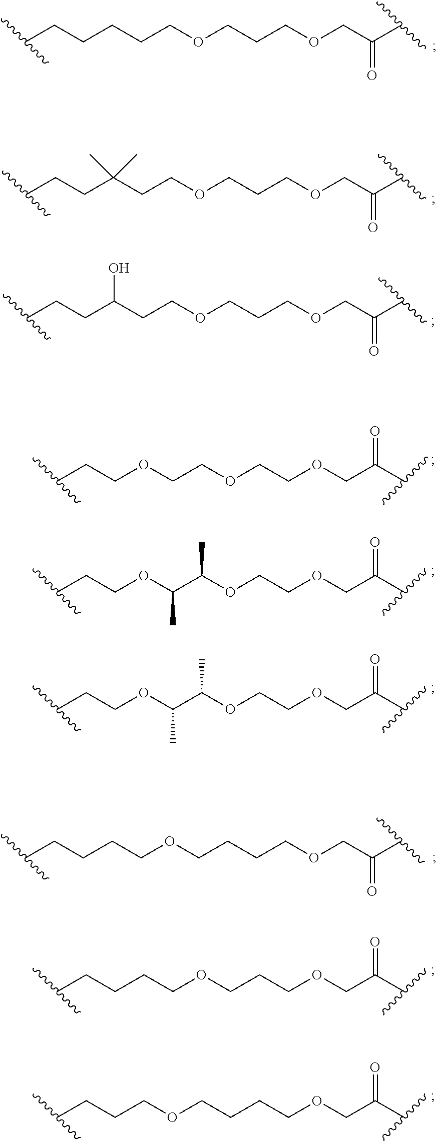

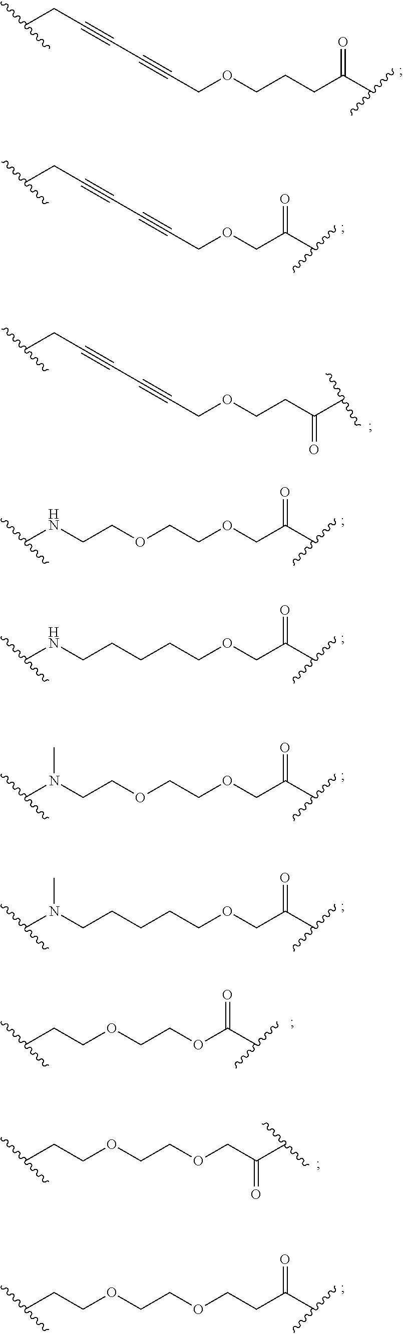

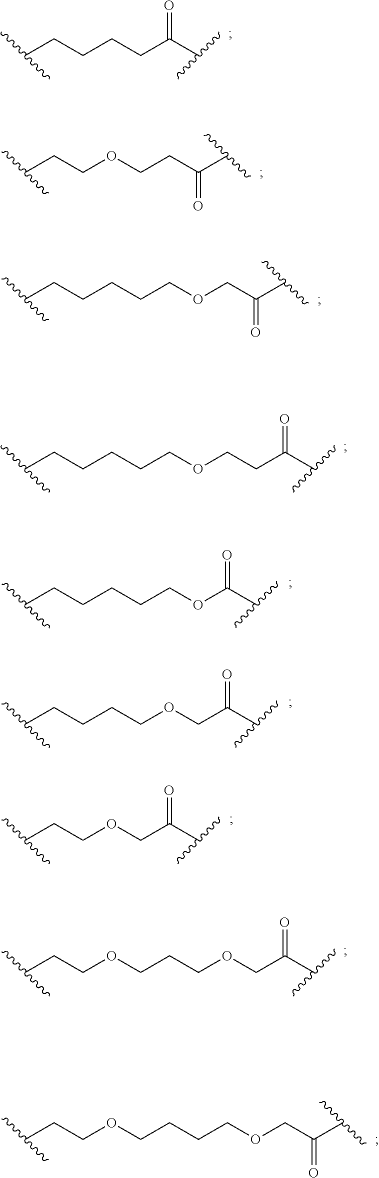

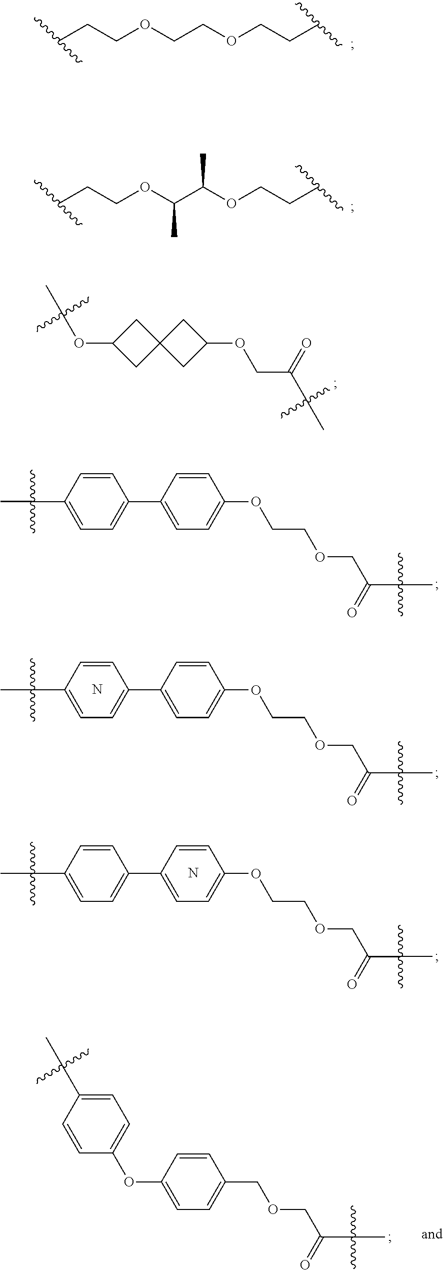

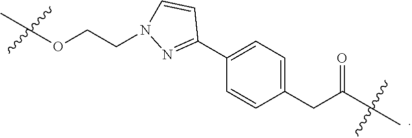 [SMILES: *OCCn1ccc(-c2ccc(CC(*)=O)cc2)n1]

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical.

In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

In certain embodiments, the linker group L is a group comprising one or more covalently connected structural units independently selected from the group consisting of:

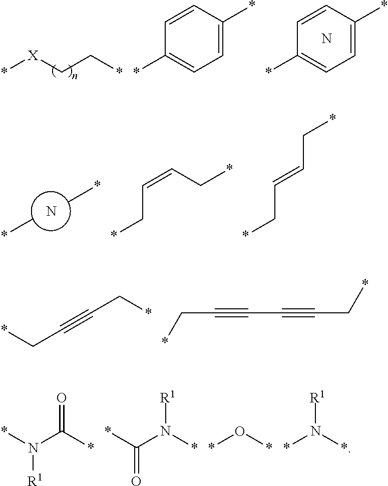

The X is selected from the group consisting of O, N, S, S(O) and SO₂; n is integer from 1-5, 5; R^{L1} is hydrogen or alkyl,

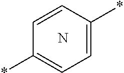

is a mono- or bicyclic aryl or heteroaryl optionally substituted with 1-3 substituents selected from alkyl, halogen, haloalkyl, hydroxy, alkoxy or cyano;

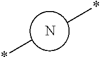

is a mono- or bicyclic cycloalkyl or a heterocycloalkyl optionally substituted with 1-3 substituents selected from alkyl, halogen, haloalkyl, hydroxy, alkoxy or cyano; and the phenyl ring fragment can be optionally substituted with 1, 2 or 3 substituents selected from the grou consisting of alkyl, halogen, haloalkyl, hydroxy, alkoxy and cyano. In an embodiment, the linker group L comprises up to 10 covalently connected structural units, as described above.

Although the ILM (or ULM) group and PTM group may be covalently linked to the linker group through any group which is appropriate and stable to the chemistry of the linker, in preferred aspects of the present invention, the linker is independently covalently bonded to the ILM group and the PTM group preferably through an amide, ester, thioester, keto group, carbamate (urethane), carbon or ether, each of which groups may be inserted anywhere on the ILM group and PTM group to provide maximum binding of the ILM group on the ubiquitin ligase and the PTM group on the target protein to be degraded. (It is noted that in certain aspects where the PTM group is a ULM group, the target protein for degradation may be the ubiquitin ligase itself). In certain preferred aspects, the linker may be linked to an optionally substituted alkyl, alkylene, alkene or alkyne group, an aryl group or a heterocyclic group on the ILM and/or PTM groups.

### Exemplary PTMs

In preferred aspects of the invention, the PTM group is a group, which binds to target proteins. Targets of the PTM group are numerous in kind and are selected from proteins that are expressed in a cell such that at least a portion of the sequences is found in the cell and may bind to a PTM group. The term “protein” includes oligopeptides and polypeptide sequences of sufficient length that they can bind to a PTM group according to the present invention. Any protein in a eukaryotic system or a microbial system, including a virus, bacteria or fungus, as otherwise described herein, are targets for ubiquitination mediated by the compounds according to the present invention. Preferably, the target protein is a eukaryotic protein. In certain aspects, the protein binding moiety is a haloalkane (preferably a C₁-C₁₀ alkyl group which is substituted with at least one halo group, preferably a halo group at the distal end of the alkyl group (i.e., away from the linker or ILM group), which may covalently bind to a dehalogenase enzyme in a patient or subject or in a diagnostic assay.

PTM groups according to the present invention include, for example, include any moiety which binds to a protein specifically (binds to a target protein) and includes the following non-limiting examples of small molecule target protein moieties: Hsp90 inhibitors, kinase inhibitors, HDM2 & MDM2 inhibitors, compounds targeting Human BET Bromodomain-containing proteins, HDAC inhibitors, human lysine methyltransferase inhibitors, angiogenesis inhibitors, nuclear hormone receptor compounds, immunosuppressive compounds, and compounds targeting the aryl hydrocarbon receptor (AHR), among numerous others. The compositions described below exemplify some of the members of these nine types of small molecule target protein binding moieties. Such small molecule target protein binding moieties also include pharmaceutically acceptable salts, enantiomers, solvates and polymorphs of these compositions, as well as other small molecules that may target a protein of interest. These binding moieties are linked to the ubiquitin ligase binding moiety preferably through a linker in order to present a target protein (to which the protein target moiety is bound) in proximity to the ubiquitin ligase for ubiquitination and degradation.

Any protein, which can bind to a protein target moiety or PTM group and acted on or degraded by an ubiquitin ligase is a target protein according to the present invention. In general, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catrabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eurkaryotes and prokaryotes including humans as targets for drug therapy, other animals, including domesticated animals, microbials for the determination of targets for antibiotics and other antimicrobials and plants, and even viruses, among numerous others.

In still other embodiments, the PTM group is a haloalkyl group, wherein said alkyl group generally ranges in size from about 1 or 2 carbons to about 12 carbons in length, often about 2 to 10 carbons in length, often about 3 carbons to about 8 carbons in length, more often about 4 carbons to about 6 carbons in length. The haloalkyl groups are generally linear alkyl groups (although branched-chain alkyl groups may also be used) and are end-capped with at least one halogen group, preferably a single halogen group, often a single chloride group. Haloalkyl PT, groups for use in the present invention are preferably represented by the chemical structure —(CH₂)ᵥ-Halo where v is any integer from 2 to about 12, often about 3 to about 8, more often about 4 to about 6. Halo may be any halogen, but is preferably Cl or Br, more often Cl.

In another embodiment, the present invention provides a library of compounds. The library comprises more than one compound wherein each composition has a formula of A-B, wherein A is a ubiquitin pathway protein binding moiety (preferably, an E3 ubiquitin ligase moiety as otherwise disclosed herein) and B is a protein binding member of a molecular library, wherein A is coupled (preferably, through a linker moiety) to B, and wherein the ubiquitin pathway protein binding moiety recognizes an ubiquitin pathway protein, in particular, an E3 ubiquitin ligase, such as cereblon. In a particular embodiment, the library contains a specific cereblon E3 ubiquitin ligase binding moiety bound to random target protein binding elements (e.g., a chemical compound library). As such, the target protein is not determined in advance and the method can be used to determine the activity of a putative protein binding element and its pharmacological value as a target upon degradation by ubiquitin ligase.

The present invention may be used to treat a number of disease states and/or conditions, including any disease state and/or condition in which proteins are dysregulated and where a patient would benefit from the degradation of proteins.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer. In certain additional embodiments, the disease is multiple myeloma.

In alternative aspects, the present invention relates to a method for treating a disease state or ameliorating the symptoms of a disease or condition in a subject in need thereof by degrading a protein or polypeptide through which a disease state or condition is modulated comprising administering to said patient or subject an effective amount, e.g., a therapeutically effective amount, of at least one compound as described hereinabove, optionally in combination with a pharmaceutically acceptable carrier, additive or excipient, and optionally an additional bioactive agent, wherein the composition is effective for treating or ameliorating the disease or disorder or symptom thereof in the subject. The method according to the present invention may be used to treat a large number of disease states or conditions including cancer, by virtue of the administration of effective amounts of at least one compound described herein. The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

In another aspect, the description provides methods for identifying the effects of the degradation of proteins of interest in a biological system using compounds according to the present invention.

The term “target protein” is used to describe a protein or polypeptide, which is a target for binding to a compound according to the present invention and degradation by ubiquitin ligase hereunder. Such small molecule target protein binding moieties also include pharmaceutically acceptable salts, enantiomers, solvates and polymorphs of these compositions, as well as other small molecules that may target a protein of interest. These binding moieties are linked to ILM or ULM groups through linker groups L.

Target proteins which may be bound to the protein target moiety and degraded by the ligase to which the ubiquitin ligase binding moiety is bound include any protein or peptide, including fragments thereof, analogues thereof, and/or homologues thereof. Target proteins include proteins and peptides having any biological function or activity including structural, regulatory, hormonal, enzymatic, genetic, immunological, contractile, storage, transportation, and signal transduction. In certain embodiments, the target proteins include structural proteins, receptors, enzymes, cell surface proteins, proteins pertinent to the integrated function of a cell, including proteins involved in catalytic activity, aromatase activity, motor activity, helicase activity, metabolic processes (anabolism and catrabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eurkaryotes and prokaryotes, including microbes, viruses, fungi and parasites, including humans, microbes, viruses, fungi and parasites, among numerous others, as targets for drug therapy, other animals, including domesticated animals, microbials for the determination of targets for antibiotics and other antimicrobials and plants, and even viruses, among numerous others.

More specifically, a number of drug targets for human therapeutics represent protein targets to which protein target moiety may be bound and incorporated into compounds according to the present invention. These include proteins which may be used to restore function in numerous polygenic diseases, including for example B7.1 and B7, TINFRlm, TNFR2, NADPH oxidase, BclIBax and other partners in the apotosis pathway, C₅a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases, CD23, CD124, tyrosine kinase p56 lck, CD4, CD5, IL-2 receptor, IL-1 receptor, TNF-alphaR, ICAM1, Cat+channels, VCAM, VLA-4 integrin, selectins, CD40/CD40L, newokinins and receptors, inosine monophosphate dehydrogenase, p38 MAP Kinase, RaslRaflMEWERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, estrogen receptors, androgen receptors, adenosine receptors, adenosine kinase and AMP deaminase, purinergic receptors (P2Y1, P2Y2, P2Y4, P2Y6, P2X1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase. Additional protein targets include, for example, ecdysone 20-monooxygenase, ion channel of the GABA gated chloride channel, acetylcholinesterase, voltage-sensitive sodium channel protein, calcium release channel, and chloride channels. Still further target proteins include Acetyl-CoA carboxylase, adenylosuccinate synthetase, protoporphyrinogen oxidase, and enolpyruvylshikimate-phosphate synthase.

Haloalkane dehalogenase enzymes are another target of specific compounds according to the present invention. Compounds according to the present invention which contain chloroalkane peptide binding moieties (C₁-C₁₂ often about C₂-C₁₀ alkyl halo groups) may be used to inhibit and/or degrade haloalkane dehalogenase enzymes which are used in fusion proteins or related dioagnostic proteins as described in PCT/US2012/063401 filed Dec. 6, 2011 and published as WO 2012/078559 on Jun. 14, 2012, the contents of which is incorporated by reference herein.

These various protein targets may be used in screens that identify compound moieties which bind to the protein and by incorporation of the moiety into compounds according to the present invention, the level of activity of the protein may be altered for therapeutic end result.

The term “protein target moiety” or PTM is used to describe a small molecule which binds to a target protein or other protein or polypeptide of interest and places/presents that protein or polypeptide in proximity to an ubiquitin ligase such that degradation of the protein or polypeptide by ubiquitin ligase may occur. Non-limiting examples of small molecule target protein binding moieties include Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among numerous others. The compositions described below exemplify some of the members of these nine types of small molecule target protein.

Exemplary protein target moieties according to the present disclosure include, haloalkane halogenase inhibitors, Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR).

The compositions described below exemplify some of the members of these types of small molecule target protein binding moieties. Such small molecule target protein binding moieties also include pharmaceutically acceptable salts, enantiomers, solvates and polymorphs of these compositions, as well as other small molecules that may target a protein of interest. References which are cited herein below are incorporated by reference herein in their entirety.

### I. Heat Shock Protein 90 (HSP90) Inhibitors:

HSP90 inhibitors as used herein include, but are not limited to:

1. The HSP90 inhibitors identified in Vallee, et al., “Tricyclic Series of Heat Shock Protein 90 (HSP90) Inhibitors Part I: Discovery of Tricyclic Imidazo[4,5-C]Pyridines as Potent Inhibitors of the HSP90 Molecular Chaperone (2011) J. Med. Chem. 54: 7206, including YKB (N-[4-(3H-imidazo[4,5-C]Pyridin-2-yl)-9H-Fluoren-9-yl]-succinamide):

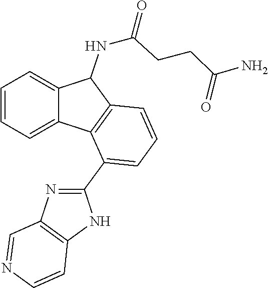 [SMILES: NC(=O)CCC(=O)NC1c2ccccc2-c2c(-c3nc4cnccc4[nH]3)cccc21]

derivatized where a linker group L or a -(L-ILM) group is attached, for example, via the terminal amide group;

2. The HSP90 inhibitor p54 (modified) (8-[(2,4-dimethylphenyl)sulfanyl]-3]pent-4-yn-1-yl-3H-purin-6-amine):

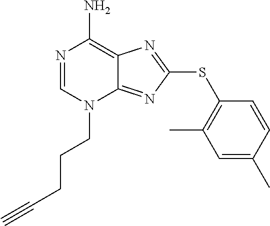 [SMILES: C#CCCCn1cnc(N)c2nc(Sc3ccc(C)cc3C)nc1-2]

where a linker group L or a -(L-ILM) group is attached, for example, via the terminal acetylene group;

3. The HSP90 inhibitors (modified) identified in Brough, et al., “4,5-Diarylisoxazole HSP90 Chaperone Inhibitors: Potential Therapeutic Agents for the Treatment of Cancer”, J. MED. CHEM. vol: 51, pag: 196 (2008), including the compound 2GJ (5-[2,4-dihydroxy-5-(1-methylethyl)phenyl]-n-ethyl-4-[4-(morpholin-4-ylmethyl)phenyl]isoxazole-3-carboxamide) having the structure:

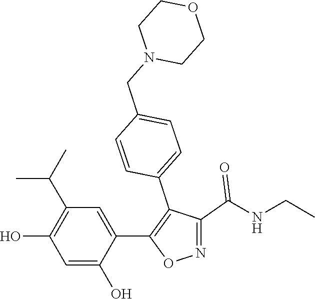 [SMILES: CCNC(=O)c1noc(-c2cc(C(C)C)c(O)cc2O)c1-c1ccc(CN2CCOCC2)cc1]

derivatized, where a linker group L or a -(L-ILM) group is attached, for example, via the amide group (at the amine or at the alkyl group on the amine);

4. The HSP90 inhibitors (modified) identified in Wright, et al., Structure-Activity Relationships in Purine-Based Inhibitor Binding to HSP90 Isoforms, Chem Biol. 2004 June; 11(6):775-85, including the HSP90 inhibitor PU3 having the structure:

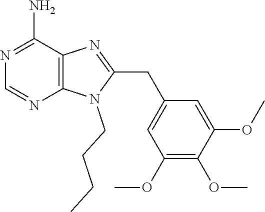 [SMILES: CCCCn1c(Cc2cc(OC)c(OC)c(OC)c2)nc2c(N)ncnc21]

where a linker group L or -(L-ILM) is attached, for example, via the butyl group; and

5. The HSP90 inhibitor geldanamycin ((4E,6Z,8S,9S,10E,12S,13R,14S,16R)-13-hydroxy-8,14,19-trimethoxy-4,10,12,16-tetramethyl-3,20,22-trioxo-2-azabicyclo[16.3.1](derivatized) or any of its derivatives (e.g. 17-alkylamino-17-desmethoxygeldanamycin (“17-AAG”) or 17-(2-dimethylaminoethyl)amino-17-desmethoxygeldanamycin (“17-DMAG”)) (derivatized, where a linker group L or a-(L-ILM) group is attached, for example, via the amide group).

### II. Kinase and Phosphatase Inhibitors:

Kinase inhibitors as used herein include, but are not limited to:

1. Erlotinib Derivative Tyrosine Kinase Inhibitor:

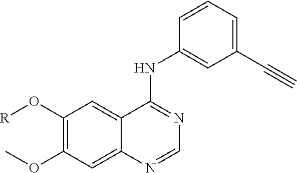

where R is a linker group L or a -(L-ILM) group attached, for example, via the ether group;

2. The kinase inhibitor sunitinib (derivatized):

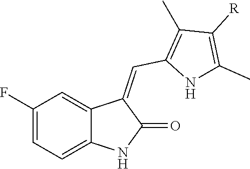

(derivatized where R is a linker group L or a -(L-ILM) group attached, for example, to the pyrrole moiety);

3. Kinase Inhibitor sorafenib (derivatized):

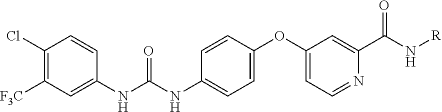

(derivatized where R is a linker group L or a -(L-ILM) group attached, for example, to the amide moiety);

4. The kinase inhibitor desatinib (derivatized):

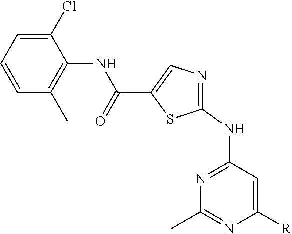

(derivatized where R is a linker group Lor a-(L-ILM) attached, for example, to the pyrimidine);

5. The kinase inhibitor lapatinib (derivatized):

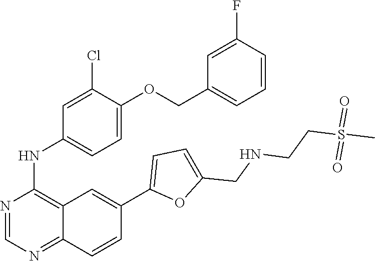 [SMILES: CS(=O)(=O)CCNCc1ccc(-c2ccc3ncnc(Nc4ccc(OCc5cccc(F)c5)c(Cl)c4)c3c2)o1]

(derivatized where a linker group L or a-(L-ILM) group is attached, for example, via the terminal methyl of the sulfonyl methyl group);

6. The kinase inhibitor U09-CX-5279 (derivatized):

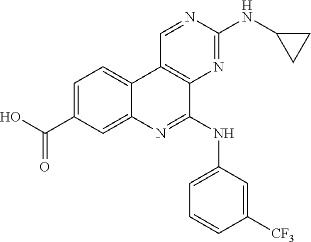 [SMILES: O=C(O)c1ccc2c(c1)nc(Nc1cccc(C(F)(F)F)c1)c1nc(NC3CC3)ncc12]

derivatized where a linker group L or a -(L-ILM) group is attached, for example, via the amine (aniline), carboxylic acid or amine alpha to cyclopropyl group, or cyclopropyl group;

7. The kinase inhibitors identified in Millan, et al., Design and Synthesis of Inhaled P38 Inhibitors for the Treatment of Chronic Obstructive Pulmonary Disease, J. MED. CHEM. vol:54, pag:7797 (2011), including the kinase inhibitors Y1W and Y1X (Derivatized) having the structures:

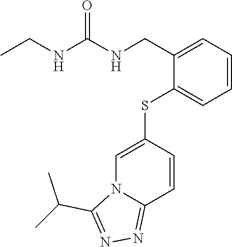 [SMILES: CCNC(=O)NCc1ccccc1Sc1ccc2nnc(C(C)C)n2c1]

YIX (1-ethyl-3-(2-{[3-(1-methylethyl)[1,2,4]triazolo[4,3-a]pyridine-6-yl]sulfanyl}benzyl)urea
derivatized where a linker group L or a-(L-ILM) group is attached, for example, via the ⁱpropyl group;

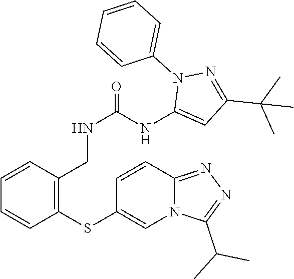 [SMILES: CC(C)c1nnc2ccc(Sc3ccccc3CNC(=O)Nc3cc(C(C)(C)C)nn3-c3ccccc3)cn12]

YIW 1-(3-tert-butyl-1-phenyl-1H-pyrazol-5-yl)-3-(2-{[3-(1-methylethyl)[1,2,4]triazolo[4,3-a]pyridin-6-yl]sulfanyl}benzyl)urea
derivatized where a linker group L or a -(L-ILM) group is attached, for example, preferably via either the i-propyl group or the t-butyl group;

8. The kinase inhibitors identified in Schenkel, et al., Discovery of Potent and Highly Selective Thienopyridine Janus Kinase 2 Inhibitors J. Med. Chem., 2011, 54 (24), pp 8440-8450, including the compounds 6TP and OTP (Derivatized) having the structures:

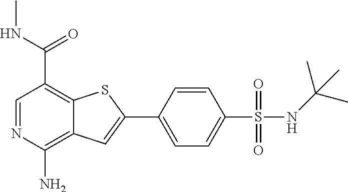 [SMILES: CNC(=O)c1cnc(N)c2cc(-c3ccc(S(=O)(=O)NC(C)(C)C)cc3)sc12]

6TP 4-amino-2-[4-(tert-butylsulfamoyl)phenyl]-N-methylthieno[3,2-c]pyridine-7-carboxamide Thienopyridine 19
derivatized where a linker group L or a -(L-ILM) group is attached, for example, via the terminal methyl group bound to amide moiety;

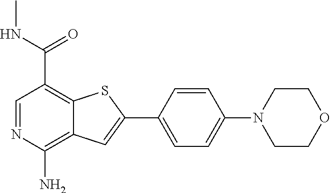 [SMILES: CNC(=O)c1cnc(N)c2cc(-c3ccc(N4CCOCC4)cc3)sc12]

OTP 4-amino-N-methyl-2-[4-(morpholin-4-yl)phenyl]thieno[3,2-c]pyridine-7-carboxamide Thienopyridine 8
derivatized where a linker group L or a -(L-ILM) group is attached, for example, via the terminal methyl group bound to the amide moiety;

9. The kinase inhibitors identified in Van Eis, et al., “2,6-Naphthyridines as potent and selective inhibitors of the novel protein kinase C isozymes”, Biorg. Med. Chem. Lett. 2011 Dec. 15; 21(24):7367-72, including the kinase inhibitor 07U having the structure:

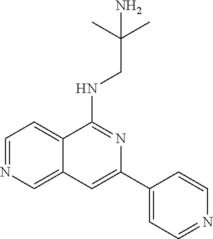 [SMILES: CC(C)(N)CNc1nc(-c2ccncc2)cc2cnccc12]

07U 2-methyl-N˜1˜-[3-(pyridin-4-yl)-2,6-naphthyridin-1-yl]propane-1,2-diamine
derivatized where a linker group L or a -(L-ILM) group is attached, for example, via the secondary amine or terminal amino group;

10. The kinase inhibitors identified in Lountos, et al., “Structural Characterization of Inhibitor Complexes with Checkpoint Kinase 2 (Chk2), a Drug Target for Cancer Therapy”, J. STRUCT. BIOL. vol:176, pag:292 (2011), including the kinase inhibitor YCF having the structure:

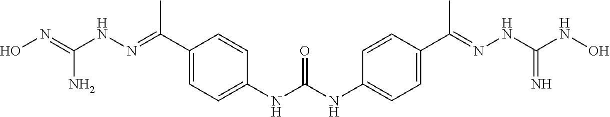 [SMILES: C/C(=N\NC(=N)NO)c1ccc(NC(=O)Nc2ccc(/C(C)=N/N/C(N)=N/O)cc2)cc1]

derivatized where a linker group L or a -(L-ILM) group is attached, for example, via either of the terminal hydroxyl groups;

11. The kinase inhibitors identified in Lountos, et al., “Structural Characterization of Inhibitor Complexes with Checkpoint Kinase 2 (Chk2), a Drug Target for Cancer Therapy”, J. STRUCT. BIOL. vol:176, pag:292 (2011), including the kinase inhibitors XK9 and NXP (derivatized) having the structures:

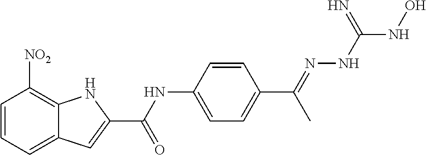 [SMILES: C/C(=N\NC(=N)NO)c1ccc(NC(=O)c2cc3cccc([N+](=O)[O-])c3[nH]2)cc1]

XK9 N-{4-[(1E)-N—(N-hydroxycarbamimidoyl)ethanehydrazonoyl]phenyl}-7-nitro-1H-indole-2-carboxamide;

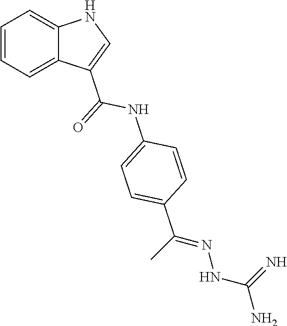 [SMILES: C/C(=N\NC(=N)N)c1ccc(NC(=O)c2c[nH]c3ccccc23)cc1]

### NXP N-{4-[(1E)-N-Carbamimidoylethanehydrazonoyl]Phenyl}-1H-Indole-3-Carboxamide

derivatized where a linker group L or a -(L-ILM) group is attached, for example, via the terminal hydroxyl group (XK9) or the hydrazone group (NXP);

12. The kinase inhibitor afatinib (derivatized) (N-[4-[(3-chloro-4-fluorophenyl)amino]-7-[[(3S)-tetrahydro-3-furanyl]oxy]-6-quinazolinyl]-4(dimethylamino)-2-butenamide) (Derivatized where a linker group L or a -(L-ILM) group is attached, for example, via the aliphatic amine group);

13. The kinase inhibitor fostamatinib (derivatized) ([6-({5-fluoro-2-[(3,4,5-trimethoxyphenyl)amino]pyrimidin-4-yl}amino)-2,2-dimethyl-3-oxo-2,3-dihydro-4H-pyrido[3,2-b]-1,4-oxazin-4-yl]methyl disodium phosphate hexahydrate) (Derivatized where a linker group L or a -(L-ILM) group is attached, for example, via a methoxy group);

14. The kinase inhibitor gefitinib (derivatized) (N-(3-chloro-4-fluoro-phenyl)-7-methoxy-6-(3-morpholin-4-ylpropoxy)quinazolin-4-amine):

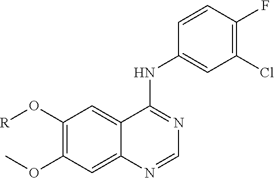

(derivatized where a linker group L or a -(L-ILM) group is attached, for example, via a methoxy or ether group);

15. The kinase inhibitor lenvatinib (derivatized) (4-[3-chloro-4-(cyclopropylcarbamoylamino)phenoxy]-7-methoxy-quinoline-6-carboxamide) (derivatized where a linker group L or a -(L-ILM) group is attached, for example, via the cyclopropyl group);

16. The kinase inhibitor vandetanib (derivatized) (N-(4-bromo-2-fluorophenyl)-6-methoxy-7-[(1-methylpiperidin-4-yl)methoxy]quinazolin-4-amine) (derivatized where a linker group L or a -(L-ILM) group is attached, for example, via the methoxy or hydroxyl group);

17. The kinase inhibitor vemurafenib (derivatized) (propane-1-sulfonic acid {3-[5-(4-chlorophenyl)-1H-pyrrolo[2,3-b]pyridine-3-carbonyl]-2,4-difluoro-phenyl}-amide) (derivatized where a linker group L or a -(L-ILM) group is attached, for example, via the sulfonyl propyl group);

18. The kinase inhibitor Gleevec (derivatized):

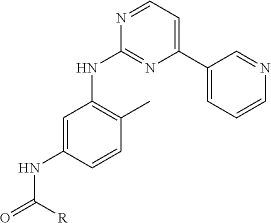

(derivatized where R as a linker group L or a-(L-ILM) group is attached, for example, via the amide group or via the aniline amine group);

19. The kinase inhibitor pazopanib (derivatized) (VEGFR3 inhibitor):

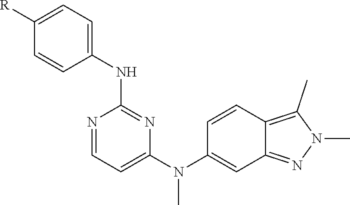

(derivatized where R is a linker group L or a -(L-ILM) group attached, for example, to the phenyl moiety or via the aniline amine group);

20. The kinase inhibitor AT-9283 (Derivatized) Aurora Kinase Inhibitor

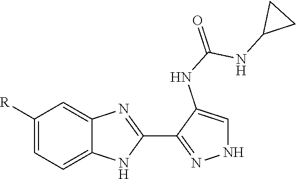

(where R is a linker group L or a -(L-ILM) group attached, for example, to the phenyl moiety);

21. The kinase inhibitor TAE684 (derivatized) ALK inhibitor

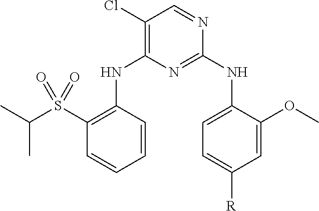

(where R is a linker group L or a -(L-ILM) group attached, for example, to the phenyl moiety);

22. The kinase inhibitor nilotanib (derivatized) Abl inhibitor:

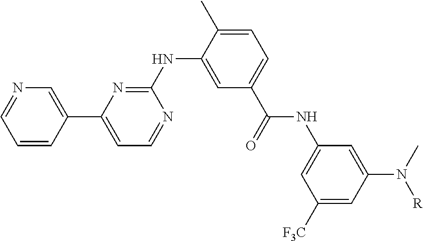

(derivatized where R is a linker group L or a -(L-ILM) group attached, for example, to the phenyl moiety or the aniline amine group);

23. Kinase Inhibitor NVP-BSK805 (derivatized) JAK2 Inhibitor

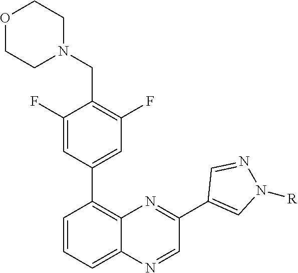

(derivatized where R is a linker group L or a -(L-ILM) group attached, for example, to the phenyl moiety or the diazole group);

24. Kinase Inhibitor crizotinib Derivatized Alk Inhibitor

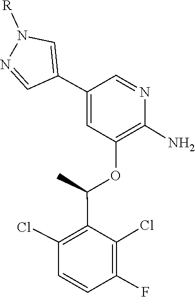

(derivatized where R is a linker group L or a -(L-ILM) group attached, for example, to the phenyl moiety or the diazole group);

25. Kinase Inhibitor JNJ FMS (derivatized) Inhibitor

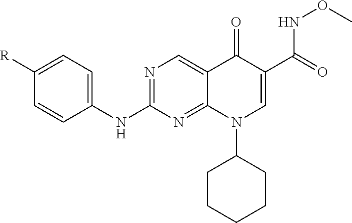

(derivatized where R is a linker group L or a -(L-ILM) group attached, for example, to the phenyl moiety);

26. The kinase inhibitor foretinib (derivatized) Met Inhibitor

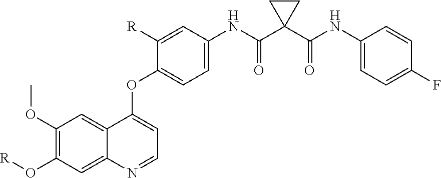

(derivatized where R is a linker group L or a -(L-ILM) group attached, for example, to the phenyl moiety or a hydroxyl or ether group on the quinoline moiety);

27. The allosteric Protein Tyrosine Phosphatase Inhibitor PTP1B (derivatized):

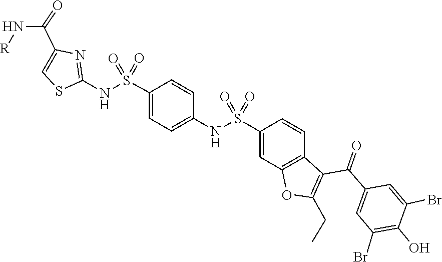

derivatized where a linker group L or a -(L-ILM) group is attached, for example, at R, as indicated;

28. The inhibitor of SHP-2 Domain of Tyrosine Phosphatase (derivatized):

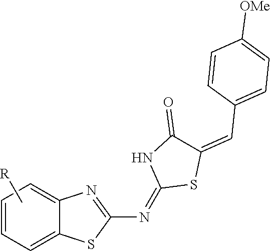

derivatized where a linker group L or a -(L-ILM) group is attached, for example, at R;

29. The inhibitor (derivatized) of BRAF (BRAF^{V600E})/MEK:

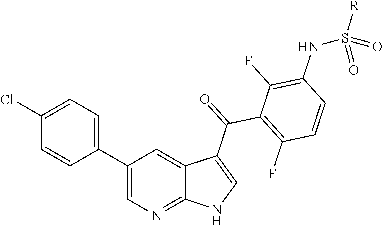

derivatized where a linker group L or a-(L-ILM) group is attached, for example, at R;

30. Inhibitor (derivatized) of Tyrosine Kinase ABL

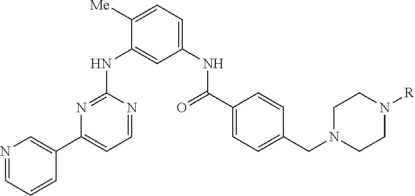

derivatized where a linker group L or a-(L-ILM) group is attached, for example, at R;

31. The kinase inhibitor OSI-027 (derivatized) mTORC1/2 inhibitor

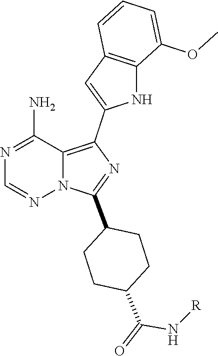

derivatized where a linker group L or a-(L-ILM) group is attached, for example, at R;

32. The kinase inhibitor OSI-930 (derivatized) c-Kit/KDR inhibitor

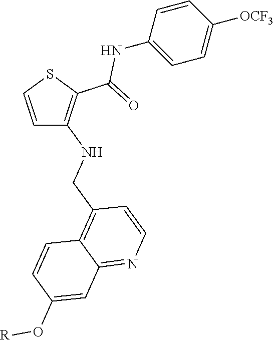

derivatized where a linker group L or a-(L-ILM) group is attached, for example, at R; and

33. The kinase inhibitor OSI-906 (derivatized) IGF1R/IR inhibitor

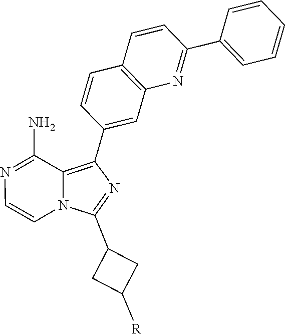

derivatized where a linker group L or a-(L-ILM) group is attached, for example, at R; (derivatized where “R” designates a site for attachment of a linker group L or a -(L-ILM) group on the piperazine moiety).

34. The kinase inhibitor EAI045 (derivatized) EGFR triple mutant

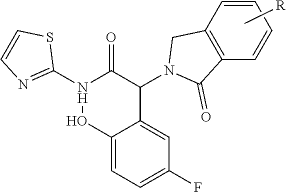

derivatized where a linker group L or a-(L-ILM) group is attached, for example, at R

35. The kinase inhibitor Compound 42 (JMC 1025, 8877) EGFR triple mutant

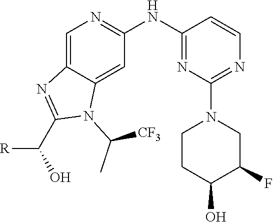

derivatized where a linker group L or a-(L-ILM) group is attached, for example, at R
The EGFR Δ20 insertion kinase inhibitors AEE788, TAK285, AP32788 and afatinib

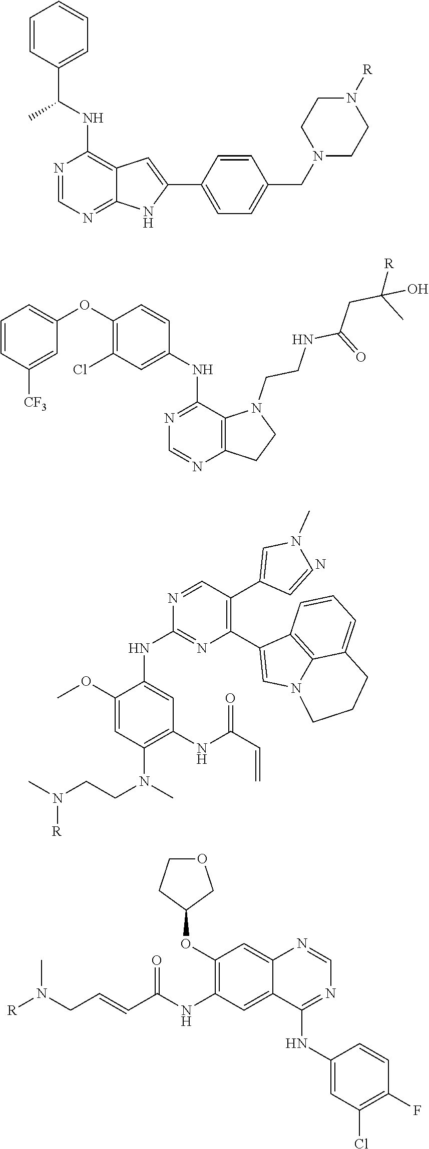

derivatized where a linker group L or a-(L-ILM) group is attached, for example, at R

36. The Flt3 inhibitors UNC-2025, Quizartinib, Cabozitinib, Pacrinitinib, AMG 925, G-749, AZD2932

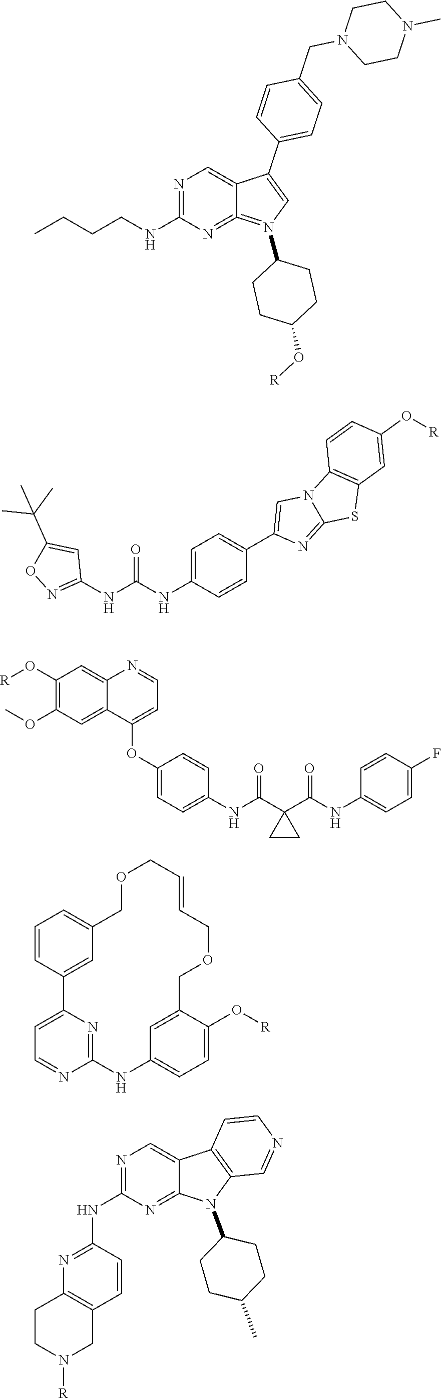

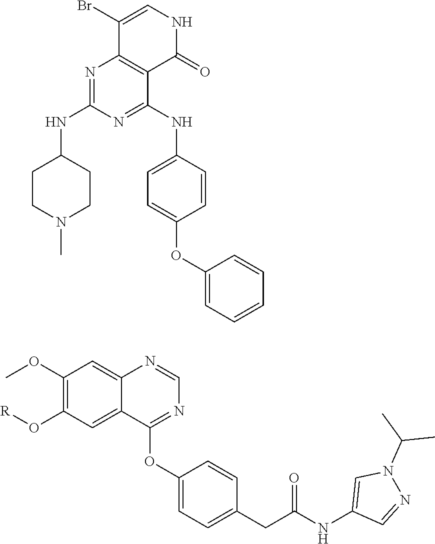

derivatized where a linker group L or a-(L-ILM) group is attached, for example, at R

37. The KSR inhibitors ASC65 and ASC24

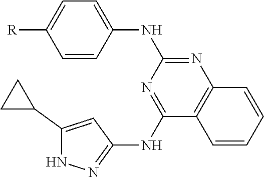

derivatized where a linker group L or a-(L-ILM) group is attached, for example, at R.

38. The JNK (c-Jun N-terminal kinase) inhibitors, such as those described by Koch, P. et al. in Journal of Medicinal Chemistry 2015, 58, 72-95, as well as those disclosed in WO 2007129195 and WO 2007125405

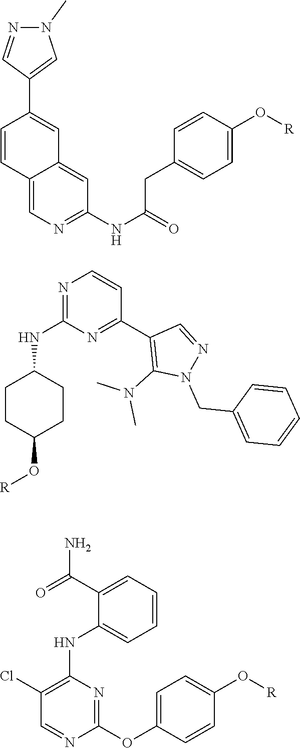

derivatized where a linker group L or a-(L-ILM) group is attached, for example, at R.

39. TNIK (TRAF2 and NCK-interacting protein kinase) ligands such as those described by Ho, K. et al. in Bioorganic and Medicinal Chemistry Letters 2013, 23, 569-573

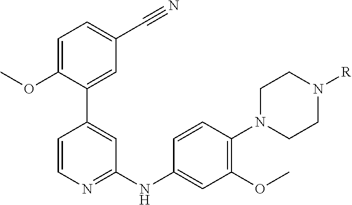

derivatized where a linker group L or a-(L-ILM) group is attached, for example, at R.

### III. HDM2/MDM2 Inhibitors:

HDM2/MDM2 inhibitors as used herein include, but are not limited to:

1. The HDM2/MDM2 inhibitors identified in Vassilev, et al., In vivo activation of the p53 pathway by small-molecule antagonists of MDM2, SCIENCE vol:303, pag:844-848 (2004), and Schneekloth, et al., Targeted intracellular protein degradation induced by a small molecule: En route to chemical proteomics, Bioorg. Med. Chem. Lett. 18 (2008) 5904-5908, including (or additionally) the compounds nutlin-3, nutlin-2, and nutlin-1 (derivatized) as described below, as well as all derivatives and analogs thereof:

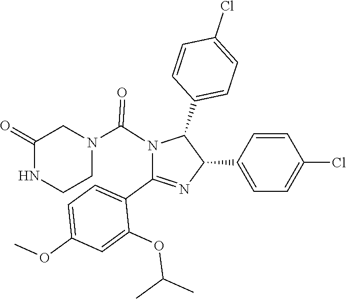 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

(derivatized where a linker group L or a -(L-ILM) group is attached, for example, at the methoxy group or as a hydroxyl group);

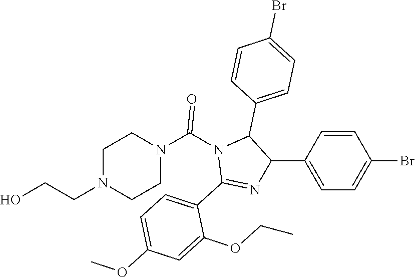 [SMILES: CCOc1cc(OC)ccc1C1=NC(c2ccc(Br)cc2)C(c2ccc(Br)cc2)N1C(=O)N1CCN(CCO)CC1]

(derivatized where a linker group L or a -(L-ILM) group is attached, for example, at the methoxy group or hydroxyl group);

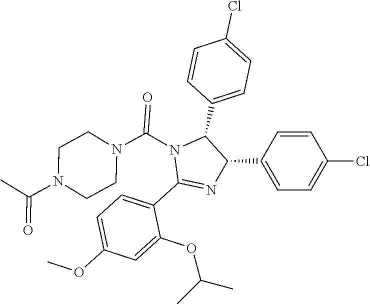 [SMILES: COc1ccc(C2=N[C@@H](c3ccc(Cl)cc3)[C@@H](c3ccc(Cl)cc3)N2C(=O)N2CCN(C(C)=O)CC2)c(OC(C)C)c1]

(derivatized where a linker group L or a -(L-ILM) group is attached, for example, via the methoxy group or as a hydroxyl group); and

2. Trans-4-Iodo-4′-Boranyl-Chalcone

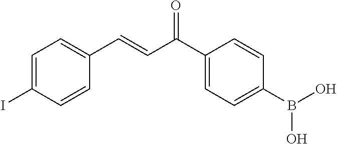 [SMILES: O=C(/C=C/c1ccc(I)cc1)c1ccc(B(O)O)cc1]

(derivatized where a linker group L or a a linker group L or a-(L-ILM) group is attached, for example, via a hydroxy group).

### IV. Compounds Targeting Human BET Bromodomain-Containing Proteins:

Compounds targeting Human BET Bromodomain-containing proteins include, but are not limited to the compounds associated with the targets as described below, where “R” designates a site for linker group L or a-(L-ILM) group attachment, for example:

1. JQ1, Filippakopoulos et al. Selective inhibition of BET bromodomains. Nature (2010):

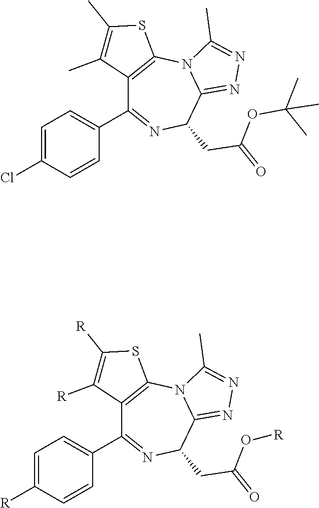

2. I-BET, Nicodeme et al. Supression of Inflammation by a Synthetic Histone Mimic. Nature (2010). Chung et al. Discovery and Characterization of Small Molecule Inhibitors of the BET Family Bromodomains. J. Med Chem. (2011):

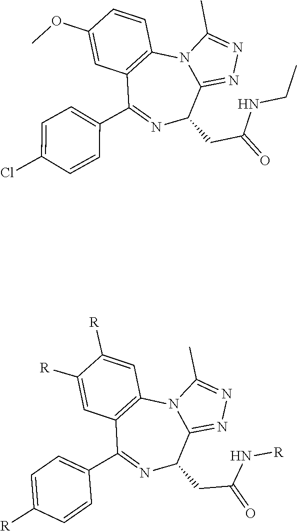

3. Compounds described in Hewings et al. 3,5-Dimethylisoxazoles Act as Acetyl-lysine Bromodomain Ligands. J. Med. Chem. (2011) 54 6761-6770.

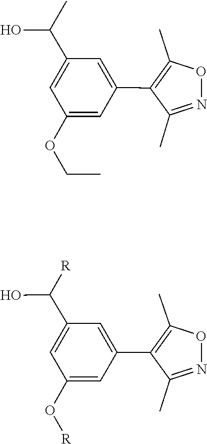

4. I-BET151, Dawson et al. Inhibition of BET Recruitment to Chromatin as an Effective Treatment for MLL-fusion Leukemia. Nature (2011):

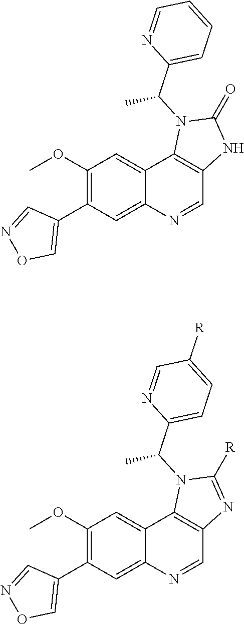

(Where R, in each instance, designates a site for attachment, for example, of a linker group L or a -(L-ILM) group).

### V. HDAC Inhibitors:

HDAC Inhibitors (derivatized) include, but are not limited to:

1. Finnin, M. S. et al. Structures of Histone Deacetylase Homologue Bound to the TSA and SAHA Inhibitors. Nature 40, 188-193 (1999).

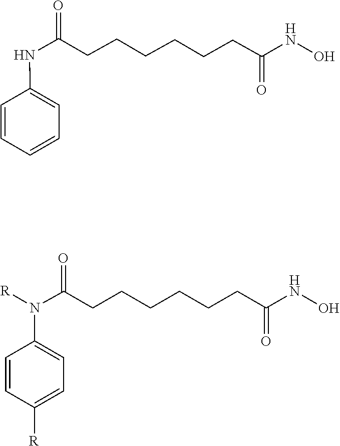

(Derivatized where “R” designates a site for attachment, for example, of a linker group L or a -(L-ILM) group); and

2. Compounds as defined by formula (I) of PCT WO0222577 (“DEACETYLASE INHIBITORS”) (Derivatized where a linker group L or a -(L-ILM) group is attached, for example, via the hydroxyl group);

### VI. Histone Lysine Methyltransferase Inhibitors:

Histone Lysine Methyltransferase inhibitors include, but are not limited to:

1. Chang et al. Structural Basis for G9a-Like protein Lysine Methyltransferase Inhibition by BIX-1294. Nat. Struct. Biol. (2009) 16(3) 312.

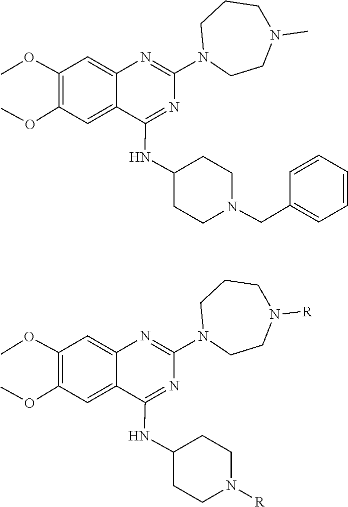

(Derivatized where “R” designates a site for attachment, for example, of a linker group L or a -(L-ILM) group);

2. Liu, F. et al Discovery of a 2,4-Diamino-7-aminoalkoxyquinazoline as a Potent and Selective Inhibitor of Histone Methyltransferase G9a. J. Med. Chem. (2009) 52(24) 7950.

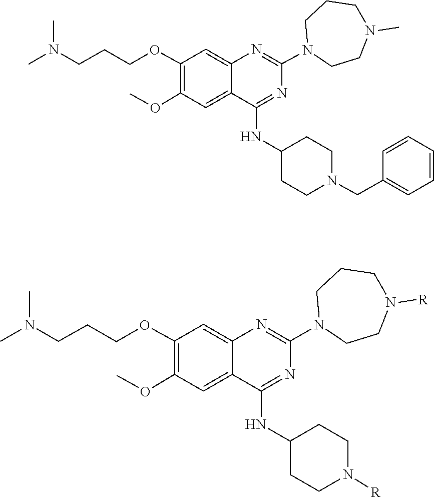

(Derivatized where “R” designates a potential site for attachment, for example, of a linker group L or a -(L-ILM) group);

3. Azacitidine (derivatized) (4-amino-1-β-D-ribofuranosyl-1,3,5-triazin-2(1H)-one) (Derivatized where a linker group L or a -(L-ILM) group is attached, for example, via the hydroxy or amino groups); and

4. Decitabine (derivatized) (4-amino-1-(2-deoxy-b-D-erythro-pentofuranosyl)-1, 3, 5-triazin-2(1H)-one) (Derivatized where a linker group L or a -(L-ILM) group is attached, for example, via either of the hydroxy groups or at the amino group).

5. Inhibitors of EZH2 (Enhancer of zeste homolog 2), a functional enzymatic component of the polycomb repressive complex 2 (PRC2), such as tazemetostat (EPZ-6438), GSK-126 and compounds disclosed in WO 2014123418

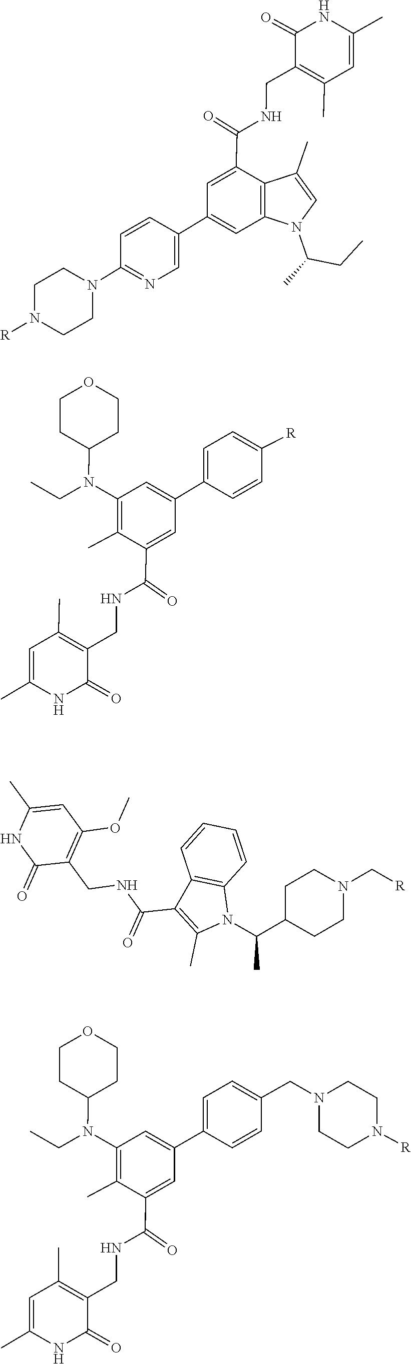

derivatized where a linker group L or a-(L-ILM) group is attached, for example, at R.

### VII. Angiogenesis Inhibitors:

Angiogenesis inhibitors include, but are not limited to:

1. GA-1 (derivatized) and derivatives and analogs thereof, having the structure(s) and binding to linkers as described in Sakamoto, et al., Development of Protacs to target cancer-promoting proteins for ubiquitination and degradation, Mol Cell Proteomics 2003 December; 2(12):1350-8;

2. Estradiol (derivatized), which may be bound to a linker group L or a -(L-ILM) group as is generally described in Rodriguez-Gonzalez, et al., Targeting steroid hormone receptors for ubiquitination and degradation in breast and prostate cancer, Oncogene (2008) 27, 7201-7211;

3. Estradiol, testosterone (derivatized) and related derivatives, including but not limited to DHT and derivatives and analogs thereof, having the structure(s) and binding to a linker group L or a -(L-ILM) group as generally described in Sakamoto, et al., Development of Protacs to target cancer-promoting proteins for ubiquitination and degradation, Mol Cell Proteomics 2003 December; 2(12):1350-8; and

4. Ovalicin, fumagillin (derivatized), and derivatives and analogs thereof, having the structure(s) and binding to a linker group L or a -(L-ILM) group as is generally described in Sakamoto, et al., Protacs: chimeric molecules that target proteins to the Skp1-Cullin-F box complex for ubiquitination and degradation Proc Natl Acad Sci USA. 2001 Jul. 17; 98(15):8554-9 and U.S. Pat. No. 7,208,157.

### VIII. Immunosuppressive Compounds:

Immunosuppressive compounds include, but are not limited to:

1. AP21998 (derivatized), having the structure(s) and binding to a linker group L or a -(L-ILM) group as is generally described in Schneekloth, et al., Chemical Genetic Control of Protein Levels: Selective in Vivo Targeted Degradation, J. AM. CHEM. SOC. 2004, 126, 3748-3754;

2. Glucocorticoids (e.g., hydrocortisone, prednisone, prednisolone, and methylprednisolone) (Derivatized where a linker group L or a -(L-ILM) group is to bound, e.g. to any of the hydroxyls) and beclometasone dipropionate (Derivatized where a linker group or a -(L-ILM) is bound, e.g. to a proprionate);

3. Methotrexate (Derivatized where a linker group or a -(L-ILM) group can be bound, e.g. to either of the terminal hydroxyls);

4. Ciclosporin (Derivatized where a linker group or a -(L-ILM) group can be bound, e.g. at any of the butyl groups);

5. Tacrolimus (FK-506) and rapamycin (Derivatized where a linker group L or a -(L-ILM) group can be bound, e.g. at one of the methoxy groups); and

6. Actinomycins (Derivatized where a linker group L or a -(L-ILM) group can be bound, e.g. at one of the isopropyl groups).

### IX. Compounds Targeting the Aryl Hydrocarbon Receptor (AHR):

Compounds targeting the aryl hydrocarbon receptor (AHR) include, but are not limited to:

1. Apigenin (Derivatized in a way which binds to a linker group L or a -(L-ILM) group as is generally illustrated in Lee, et al., Targeted Degradation of the Aryl Hydrocarbon Receptor by the PROTAC Approach: A Useful Chemical Genetic Tool, ChemBioChem Volume 8, Issue 17, pages 2058-2062, Nov. 23, 2007); and

2. SRi and LGC006 (derivatized such that a linker group L or a -(L-ILM) is bound), as described in Boitano, et al., Aryl Hydrocarbon Receptor Antagonists Promote the Expansion of Human Hematopoietic Stem Cells, Science 10 Sep. 2010: Vol. 329 no. 5997 pp. 1345-1348.

### X. Compounds Targeting RAF Receptor (Kinase):

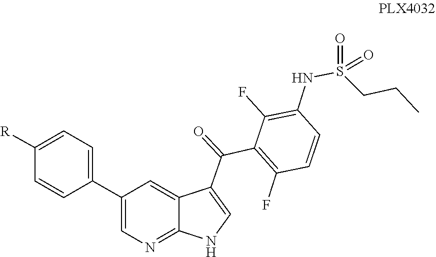

(Derivatized where “R” designates a site for linker group L or -(L-ILM) group attachment, for example).

### XI. Compounds Targeting FKBP:

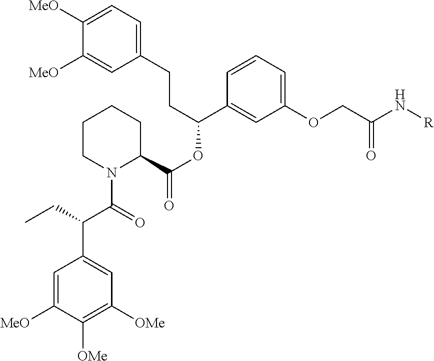

(Derivatized where “R” designates a site for a linker group L or a -(L-ILM) group attachment, for example).

### XII. Compounds Targeting Androgen Receptor (AR)

1. RU59063 Ligand (derivatized) of Androgen Receptor

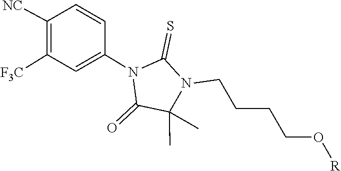

(Derivatized where “R” designates a site for a linker group L or a -(L-ILM) group attachment, for example).

2. SARM Ligand (derivatized) of Androgen Receptor

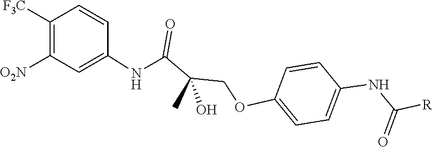

(Derivatized where “R” designates a site for a linker group L or a-(L-ILM) group attachment, for example).

3. Androgen Receptor Ligand DHT (derivatized)

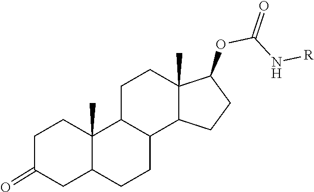

(Derivatized where “R” designates a site for a linker group L or -(L-ILM) group attachment, for example).

4. MDV3100 Ligand (derivatized)

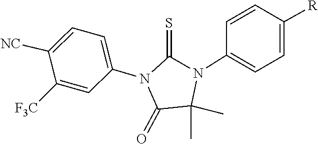

5. ARN-509 Ligand (derivatized)

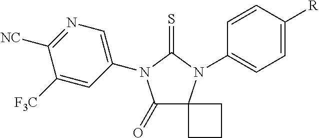

6. Hexahydrobenzisoxazoles

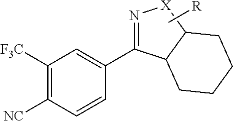

7. Tetramethylcyclobutanes

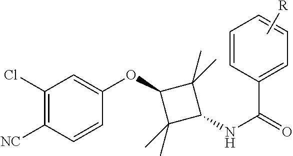

### XIII. Compounds Targeting Estrogen Receptor (ER) ICI-182780

1. Estrogen Receptor Ligand

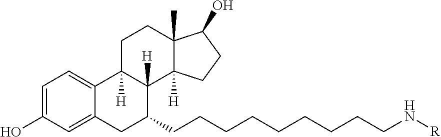

(Derivatized where “R” designates a site for linker group L or -(L-ILM) group attachment).

### XIV. Compounds Targeting Thyroid Hormone Receptor (TR)

1. Thyroid Hormone Receptor Ligand (derivatized)

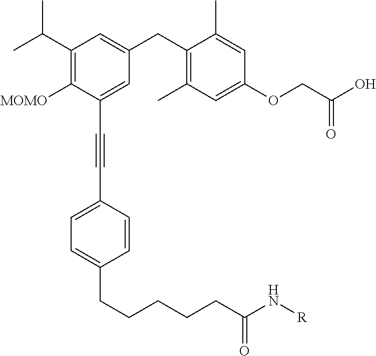

(Derivatized where “R” designates a site for linker group L or -(L-ILM) group attachment and MOMO indicates a methoxymethoxy group).

### XV. Compounds Targeting HIV Protease

1. Inhibitor of HIV Protease (derivatized)

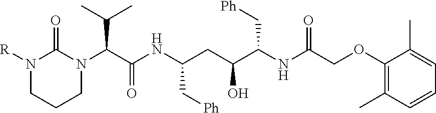

(Derivatized where “R” designates a site for linker group L or -(L-ILM) group attachment). See, J. Med. Chem. 2010, 53, 521-538.

2. Inhibitor of HIV Protease

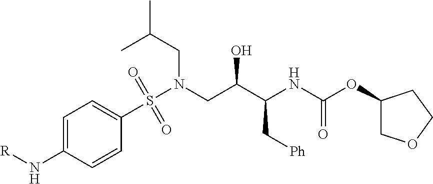

(Derivatized where “R” designates a potential site for linker group L or -(L-ILM) group attachment). See, J. Med. Chem. 2010, 53, 521-538.

### XVI. Compounds Targeting HIV Integrase

1. Inhibitor of HIV Integrase (derivatized)

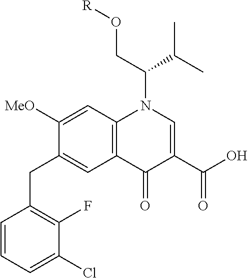

(Derivatized where “R” designates a site for linker group L or -(L-ILM) group attachment). See, J. Med. Chem. 2010, 53, 6466.

2. Inhibitor of HIV Integrase (derivatized)

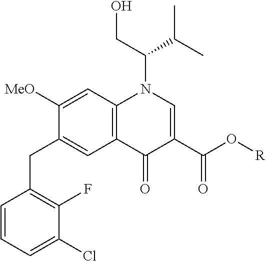

3. Inhibitor of HIV integrase Isetntress (derivatized)

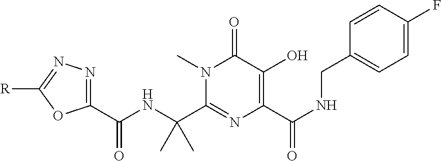

(Derivatized where “R” designates a site for linker group L or -(L-ILM) group attachment). See, J. Med. Chem. 2010, 53, 6466.

### XVII. Compounds Targeting HCV Protease

1. Inhibitors of HCV Protease (derivatized)

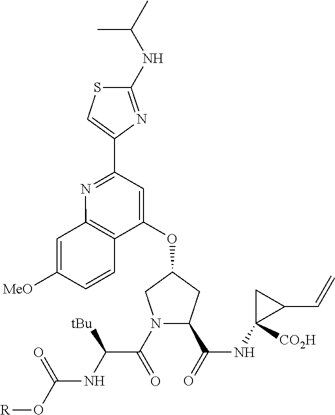

(Derivatized where “R” designates a site for linker group L or -(L-ILM) group attachment).
XVIII. Compounds targeting Acyl-protein Thioesterase-1 and -2 (APT1 and APT2)

1. Inhibitor of APT1 and APT2 (derivatized)

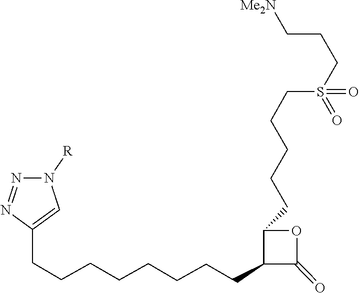

(Derivatized where “R” designates a site for linker group L or -(L-ILM) group attachment). See, Angew. Chem. Int. Ed. 2011, 50, 9838-9842, where L is a linker group as otherwise described herein and said ILM group is as otherwise described herein such that -(L-ILM) binds the ILM group to a PTMgroup as otherwise described herein.

### XIX. Compounds Targeting Ras (WT and G12C Mu)

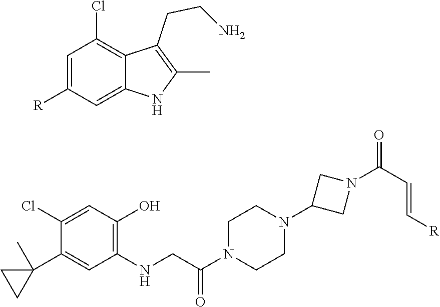

(Derivatized where “R” designates a site for linker group L or -(L-MLM) group attachment).

### XX. Compounds Targeting BRM/SMARCA2/4/PB1

The ligand PF-3

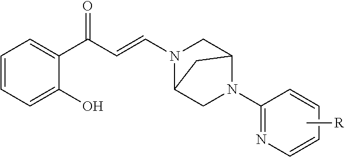

(Derivatized where “R” designates a site for linker group L or -(L-MLM) group attachment).

### XXI. Compounds Targeting Aggregation Proteins

Compounds include but are not limited to:

1. Ligands of tau protein including those described by Ariza, M. et al. in Journal of Medicinal Chemistry 2015, 58, 4365-4382

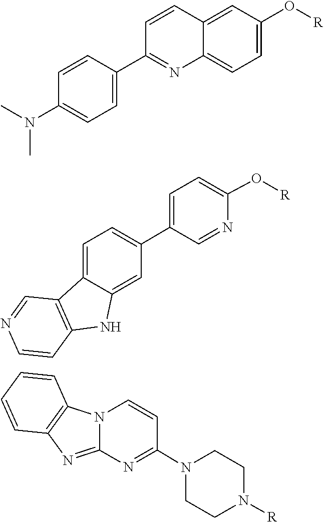

derivatized where R designates a site for linker group L or -(L-ILM) group attachment.

2. Ligands of α-synuclein protein

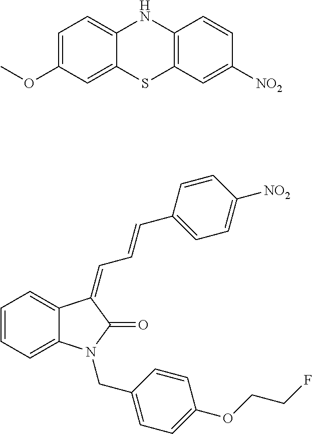 [SMILES: COc1ccc2c(c1)Sc1cc([N+](=O)[O-])ccc1N2 ; O=C1/C(=C\C=C\c2ccc([N+](=O)[O-])cc2)c2ccccc2N1Cc1ccc(OCCF)cc1]

3. Ligands of prion protein

### Therapeutic Compositions

Pharmaceutical compositions comprising combinations of an effective amount of at least one bifunctional compound as described herein, and one or more of the compounds otherwise described herein, all in effective amounts, in combination with a pharmaceutically effective amount of a carrier, additive or excipient, represents a further aspect of the present disclosure.

The present disclosure includes, where applicable, the compositions comprising the pharmaceutically acceptable salts, in particular, acid or base addition salts of compounds as described herein. The acids which are used to prepare the pharmaceutically acceptable acid addition salts of the aforementioned base compounds useful according to this aspect are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among numerous others.

Pharmaceutically acceptable base addition salts may also be used to produce pharmaceutically acceptable salt forms of the compounds or derivatives according to the present disclosure. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosureion may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions as described herein may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. In certain preferred aspects of the invention, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions as described herein may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition as described herein that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present invention.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present invention including pharmaceutically acceptable salts, solvates or polymorphs, thereof optionally in a pharmaceutically acceptable carrier or diluent, either alone, or in combination with other known erythopoiesis stimulating agents as otherwise identified herein.

These compounds can be administered by any appropriate route, for example, orally, parenterally, intravenously, intradermally, subcutaneously, or topically, including transdermally, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as erythropoietin stimulating agents, including EPO and darbapoietin alfa, among others. In certain preferred aspects of the invention, one or more compounds according to the present invention are coadministered with another bioactive agent, such as an erythropoietin stimulating agent or a would healing agent, including an antibiotic, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions which are modulated through the degraded protein.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Disease states or conditions, including cancer, which may be treated using compounds according to the present invention are set forth hereinabove.

The description provides therapeutic compositions as described herein for effectuating the degradation of proteins of interest for the treatment or amelioration of a disease, e.g., cancer. In certain additional embodiments, the disease is multiple myeloma. As such, in another aspect, the description provides a method of ubiquitinating/degrading a target protein in a cell. In certain embodiments, the method comprises administering a bifunctional compound as described herein comprising, e.g., a ILM and a PTM, preferably linked through a linker moiety, as otherwise described herein, wherein the ILM is coupled to the PTM and wherein the ILM recognizes a ubiquitin pathway protein (e.g., an ubiquitin ligase, preferably an E3 ubiquitin ligase such as, e.g., cereblon) and the PTM recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present invention.

In another embodiment, the present invention is directed to a method of treating a human patient in need for a disease state or condition modulated through a protein where the degradation of that protein will produce a therapeutic effect in that patient, the method comprising administering to a patient in need an effective amount of a compound according to the present invention, optionally in combination with another bioactive agent. The disease state or condition may be a disease caused by a microbial agent or other exogenous agent such as a virus, bacteria, fungus, protozoa or other microbe or may be a disease state, which is caused by overexpression of a protein, which leads to a disease state and/or condition

The term “disease state or condition” is used to describe any disease state or condition wherein protein dysregulation (i.e., the amount of protein expressed in a patient is elevated) occurs and where degradation of one or more proteins in a patient may provide beneficial therapy or relief of symptoms to a patient in need thereof. In certain instances, the disease state or condition may be cured.

Disease states of conditions which may be treated using compounds according to the present invention include, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, Turner syndrome.

Further disease states or conditions which may be treated by compounds according to the present invention include Alzheimer's disease, Amyotrophic lateral sclerosis (Lou Gehrig's disease), Anorexia nervosa, Anxiety disorder, Atherosclerosis, Attention deficit hyperactivity disorder, Autism, Bipolar disorder, Chronic fatigue syndrome, Chronic obstructive pulmonary disease, Crohn's disease, Coronary heart disease, Dementia, Depression, Diabetes mellitus type 1, Diabetes mellitus type 2, Epilepsy, Guillain-Barré syndrome, Irritable bowel syndrome, Lupus, Metabolic syndrome, Multiple sclerosis, Myocardial infarction, Obesity, Obsessive-compulsive disorder, Panic disorder, Parkinson's disease, Psoriasis, Rheumatoid arthritis, Sarcoidosis, Schizophrenia, Stroke, Thromboangiitis obliterans, Tourette syndrome, Vasculitis.

Still additional disease states or conditions which can be treated by compounds according to the present invention include aceruloplasminemia, Achondrogenesis type II, achondroplasia, Acrocephaly, Gaucher disease type 2, acute intermittent porphyria, Canavan disease, Adenomatous Polyposis Coli, ALA dehydratase deficiency, adenylosuccinate lyase deficiency, Adrenogenital syndrome, Adrenoleukodystrophy, ALA-D porphyria, ALA dehydratase deficiency, Alkaptonuria, Alexander disease, Alkaptonuric ochronosis, alpha 1-antitrypsin deficiency, alpha-1 proteinase inhibitor, emphysema, amyotrophic lateral sclerosis Alström syndrome, Alexander disease, Amelogenesis imperfecta, ALA dehydratase deficiency, Anderson-Fabry disease, androgen insensitivity syndrome, Anemia Angiokeratoma Corporis Diffusum, Angiomatosis retinae (von Hippel-Lindau disease) Apert syndrome, Arachnodactyly (Marfan syndrome), Stickler syndrome, Arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare-Stevenson cutis gyrata syndrome, Mediterranean fever, familial, Benjamin syndrome, beta-thalassemia, Bilateral Acoustic Neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dub6 syndrome, Brittle bone disease (osteogenesis imperfecta), Broad Thumb-Hallux syndrome (Rubinstein-Taybi syndrome), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, Cardiomyopathy (Noonan syndrome), Cri du chat, CAVD (congenital absence of the vas deferens), Caylor cardiofacial syndrome (CBAVD), CEP (congenital erythropoietic porphyria), cystic fibrosis, congenital hypothyroidism, Chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, Congenital heart disease, Methemoglobinemia/Congenital methaemoglobinaemia, achondroplasia, X-linked sideroblastic anemia, Connective tissue disease, Conotruncal anomaly face syndrome, Cooley's Anemia (beta-thalassemia), Copper storage disease (Wilson's disease), Copper transport disease (Menkes disease), hereditary coproporphyria, Cowden syndrome, Craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare-Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria Erythroid 5-aminolevulinate synthetase deficiency, Erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, Giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, Kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, Movement disorders, Mowat-Wilson syndrome, cystic fibrosis, Muenke syndrome, Multiple neurofibromatosis, Nance-Insley syndrome, Nance-Sweeney chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington) (Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, Thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alstram syndrome), Vrolik disease, Waardenburg syndrome, Warburg Sjo Fledelius Syndrome, Weissenbacher-Zweymiiller syndrome, Wolf-Hirschhorn syndrome, Wolff Periodic disease, Weissenbacher-Zweymiiller syndrome and Xeroderma pigmentosum, among others.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “bioactive agent” is used to describe an agent, other than a compound according to the present invention, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer agent, which may be combined with compounds according to the present invention to treat cancer. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS—100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1Cl1, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS—214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS—275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “anti-HIV agent” or “additional anti-HIV agent” includes, for example, nucleoside reverse transcriptase inhibitors (NRTI), other non-nucloeoside reverse transcriptase inhibitors (i.e., those which are not representative of the present invention), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development.

Other anti-HIV agents which may be used in coadministration with compounds according to the present invention include, for example, other NNRTI's (i.e., other than the NNRTI's according to the present invention) may be selected from the group consisting of nevirapine (BI-R6-587), delavirdine (U-901525/T), efavirenz (DMP-266), UC-781 (N-[4-chloro-3-(3-methyl-2-butenyloxy)phenyl]-2methyl3-furancarbothiamide), etravirine (TMC125), Trovirdine (Ly300046.HCl), MKC-442 (emivirine, coactinon), HI-236, HI-240, HI-280, HI-281, rilpivirine (TMC-278), MSC-127, HBY 097, DMP266, Baicalin (TJN-151) ADAM-II (Methyl 3′,3′-dichloro-4′,4″-dimethoxy-5′,5″-bis(methoxycarbonyl)-6,6-diphenylhexenoate), Methyl 3-Bromo-5-(1-5-bromo-4-methoxy-3-(methoxycarbonyl)phenyl)hept-1-enyl)-2-methoxybenzoate (Alkenyldiarylmethane analog, Adam analog), (5-chloro-3-(phenylsulfinyl)-2′-indolecarboxamide), AAP-BHAP (U-104489 or PNU-104489), Capravirine (AG-1549, S—1153), atevirdine (U-87201E), aurin tricarboxylic acid (SD-095345), 1-[(6-cyano-2-indolyl)carbonyl]-4-[3-(isopropylamino)-2-pyridinyl]piperazine, 1-[5-[[N-(methyl)methylsulfonylamino]-2-indolylcarbonyl-4-[3-(isopropylamino)-2-pyridinyl]piperazine, 1-[3-(Ethylamino)-2-[pyridinyl]-4-[(5-hydroxy-2-indolyl)carbonyl]piperazine, 1-[(6-Formyl-2-indolyl)carbonyl]-4-[3-(isopropylamino)-2-pyridinyl]piperazine, 1-[[5-(Methylsulfonyloxy)-2-indoyly)carbonyl]-4-[3-(isopropylamino)-2-pyridinyl]piperazine, U88204E, Bis(2-nitrophenyl)sulfone (NSC 633001), Calanolide A (NSC675451), Calanolide B, 6-Benzyl-5-methyl-2-(cyclohexyloxy)pyrimidin-4-one (DABO-546), DPC 961, E-EBU, E-EBU-dm, E-EPSeU, E-EPU, Foscarnet (Foscavir), HEPT (1-[(2-Hydroxyethoxy)methyl]-6-(phenylthio)thymine), HEPT-M (1-[(2-Hydroxyethoxy)methyl]-6-(3-methylphenyl)thio)thymine), HEPT-S (1-[(2-Hydroxyethoxy)methyl]-6-(phenylthio)-2-thiothymine), Inophyllum P, L-737,126, Michellamine A (NSC650898), Michellamine B (NSC649324), Michellamine F, 6-(3,5-Dimethylbenzyl)-1-[(2-hydroxyethoxy)methyl]-5-isopropyluracil, 6-(3,5-Dimethylbenzyl)-1-(ethyoxymethyl)-5-isopropyluracil, NPPS, E-BPTU (NSC 648400), Oltipraz (4-Methyl-5-(pyrazinyl)-3H-1,2-dithiole-3-thione), N-{2-(2-Chloro-6-fluorophenethyl]-N′-(2-thiazolyl)thiourea (PETT Cl, F derivative), N-{2-(2,6-Difluorophenethyl]-N′-[2-(5-bromopyridyl)]thiourea {PETT derivative), N-{2-(2,6-Difluorophenethyl]-N′-[2-(5-methylpyridyl)]thiourea {PETT Pyridyl derivative), N-[2-(3-Fluorofuranyl)ethyl]-N′-[2-(5-chloropyridyl)]thiourea, N-[2-(2-Fluoro-6-ethoxyphenethyl)]—N′-[2-(5-bromopyridyl)]thiourea, N-(2-Phenethyl)-N′-(2-thiazolyl)thiourea (LY-73497), L-697,639, L-697,593, L-697,661, 3-[2-(4,7-Difluorobenzoxazol-2-yl)ethyl}-5-ethyl-6-methyl(pypridin-2(1H)-thione (2-Pyridinone Derivative), 3-[[(2-Methoxy-5,6-dimethyl-3-pyridyl)methyl]amine]-5-ethyl-6-methyl(pypridin-2(1H)-thione, R82150, R82913, R87232, R88703, R89439 (Loviride), R90385, S-2720, Suramin Sodium, TBZ (Thiazolobenzimidazole, NSC 625487), Thiazoloisoindol-5-one, (+)(R)-9b-(3,5-Dimethylphenyl-2,3-dihydrothiazolo[2,3-a]isoindol-5(9bH)-one, Tivirapine (R86183), UC-38 and UC-84, among others.

The term “pharmaceutically acceptable salt” is used throughout the specification to describe, where applicable, a salt form of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present invention.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### General Synthetic Approach

The synthetic realization and optimization of the bifunctional molecules as described herein may be approached in a step-wise or modular fashion. For example, identification of compounds that bind to the target molecules can involve high or medium throughput screening campaigns if no suitable ligands are immediately available. It is not unusual for initial ligands to require iterative design and optimization cycles to improve suboptimal aspects as identified by data from suitable in vitro and pharmacological and/or ADMET assays. Part of the optimization/SAR campaign would be to probe positions of the ligand that are tolerant of substitution and that might be suitable places on which to attach the linker chemistry previously referred to herein. Where crystallographic or NMR structural data are available, these can be used to focus such a synthetic effort.

In a very analogous way one can identify and optimize ligands for an E3 Ligase, i.e. ULMs/ILMs.

With PTMs and ULMs (e.g. ILMs) in hand, one skilled in the art can use known synthetic methods for their combination with or without a linker moiety. Linker moieties can be synthesized with a range of compositions, lengths and flexibility and functionalized such that the PTM and ULM groups can be attached sequentially to distal ends of the linker. Thus a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

The following examples are used to assist in describing the present invention, but should not be seen as limiting the present invention in any way.

### Specific Embodiments of the Present Disclosure

The present disclosure encompasses the following specific embodiments. These following embodiments may include all of the features recited in a proceeding embodiment, as specified. Where applicable, the following embodiments may also include the features recited in any proceeding embodiment inclusively or in the alternative (e.g., embodiment (8) may include the features recited in embodiment (1), as recited, and/or the features of any of embodiments (2) to (7).

### Exemplary PROTACs

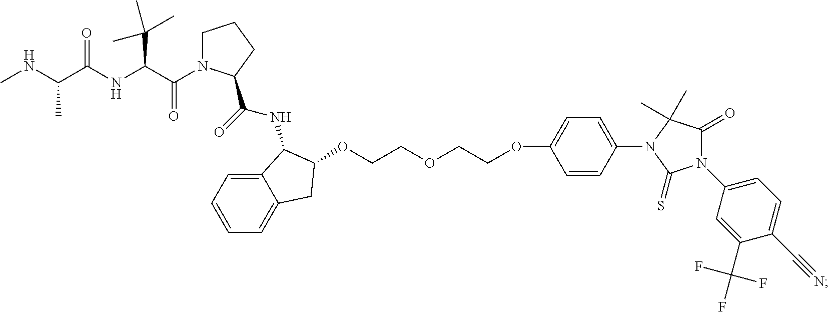 [SMILES: CN[C@@H](C)C(=O)N[C@H](C(=O)N1CCC[C@H]1C(=O)N[C@H]1c2ccccc2C[C@H]1OCCOCCOc1ccc(N2C(=S)N(c3ccc(C#N)c(C(F)(F)F)c3)C(=O)C2(C)C)cc1)C(C)(C)C]

(2S)—N-[(1S,2R)-2-{2-[2-(4-{3-[4-cyano-3-(trifluoromethyl)phenyl]-5,5-dimethyl-4-oxo-2-sulfanylideneimidazolidin-1-yl}phenoxy)ethoxy]ethoxy}-2,3-dihydro-1H-inden-1-yl]-1-[(2S)-3,3-dimethyl-2-[(2S)-2-(methylamino)propanamido]butanoyl]pyrrolidine-2-carboxamide
Androgen receptor (AR) degradation in VCaP cells: 29% @ 1 μM

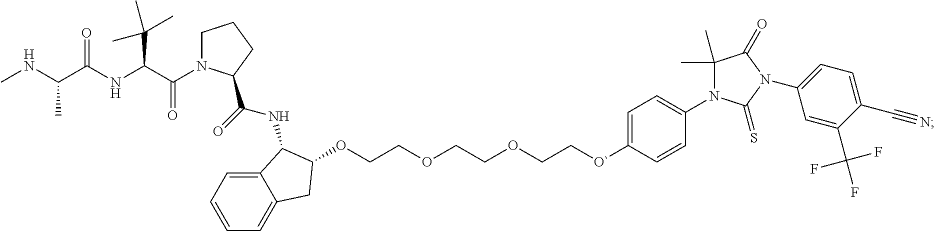

(2S)—N-[(1S,2R)-2-(2-{2-[2-(4-{3-[4-cyano-3-(trifluoromethyl)phenyl]-5,5-dimethyl-4-oxo-2-sulfanylideneimidazolidin-1-yl}phenoxy)ethoxy]ethoxy}ethoxy)-2,3-dihydro-1H-inden-1-yl]-1-[(2 S)-3,3-dimethyl-2-[(2S)-2-(methylamino)propanamido]butanoyl]pyrrolidine-2-carboxamide
Androgen receptor (AR) degradation in VCaP cells: 9% @ 1 M

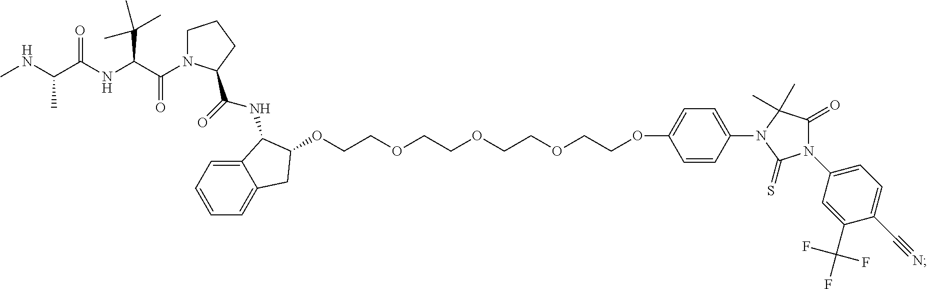 [SMILES: CN[C@@H](C)C(=O)N[C@H](C(=O)N1CCC[C@H]1C(=O)N[C@H]1c2ccccc2C[C@H]1OCCOCCOCCOCCOc1ccc(N2C(=S)N(c3ccc(C#N)c(C(F)(F)F)c3)C(=O)C2(C)C)cc1)C(C)(C)C]

(2S)—N-[(1S,2R)-2-{[1-(4-{3-[4-cyano-3-(trifluoromethyl)phenyl]-5,5-dimethyl-4-oxo-2-sulfanylideneimidazolidin-1-yl}phenyl)-1,4,7,10-tetraoxadodecan-12-yl]oxy}-2,3-dihydro-1H-inden-1-yl]-1-[(2S)-3,3-dimethyl-2-[(2S)-2-(methylamino)propanamido]butanoyl]pyrrolidine-2-carboxamide
Androgen receptor (AR) degradation in VCaP cells: 9% @ 1 μM

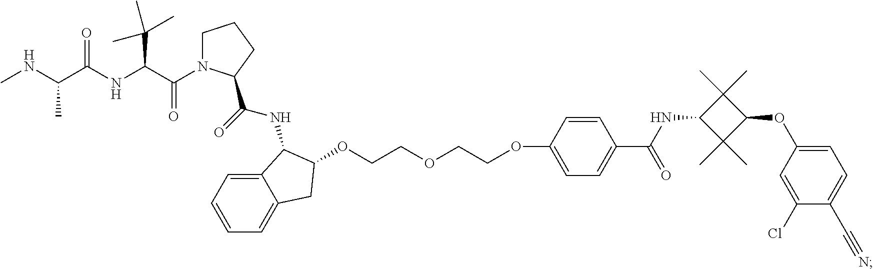 [SMILES: CN[C@@H](C)C(=O)N[C@H](C(=O)N1CCC[C@H]1C(=O)N[C@H]1c2ccccc2C[C@H]1OCCOCCOc1ccc(C(=O)N[C@H]2C(C)(C)[C@H](Oc3ccc(C#N)c(Cl)c3)C2(C)C)cc1)C(C)(C)C]

(2S)-1-[(2S)-3,3-dimethyl-2-[(2S)-2-(methylamino)propanamido]butanoyl]-N-[(1S,2R)-2-{2-[2-(4-{[(1r,3r)-3-(3-chloro-4-cyanophenoxy)-2,2,4,4-tetramethylcyclobutyl]carbamoyl}phenoxy)eth oxy]ethoxy}-2,3-dihydro-1H-inden-1-yl]pyrrolidine-2-carboxamide

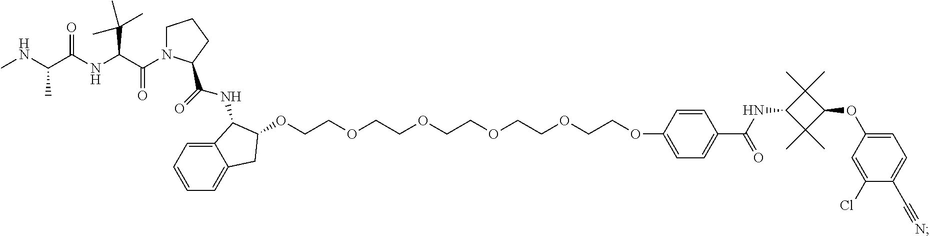 [SMILES: CN[C@@H](C)C(=O)N[C@H](C(=O)N1CCC[C@H]1C(=O)N[C@H]1c2ccccc2C[C@H]1OCCOCCOCCOCCOCCOc1ccc(C(=O)N[C@H]2C(C)(C)[C@H](Oc3ccc(C#N)c(Cl)c3)C2(C)C)cc1)C(C)(C)C]

(2S)-1-[(2S)-3,3-dimethyl-2-[(2S)-2-(methylamino)propanamido]butanoyl]-N-[(1S,2R)-2-{[1-(4-1{[(1r,3r)-3-(3-chloro-4-cyanophenoxy)-2,2,4,4-tetramethylcyclobutyl]carbamoyl}phenyl)-1,4,7,10,13-pentaoxapentadecan-15-yl]oxy}-2,3-dihydro-1H-inden-1-yl]pyrrolidine-2-carboxamide
Androgen receptor (AR) degradation in VCaP cells: 38% @ 1 μM

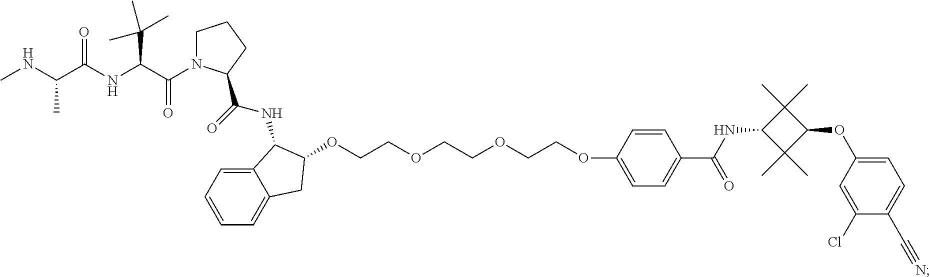 [SMILES: CN[C@@H](C)C(=O)N[C@H](C(=O)N1CCC[C@H]1C(=O)N[C@H]1c2ccccc2C[C@H]1OCCOCCOCCOc1ccc(C(=O)N[C@H]2C(C)(C)[C@H](Oc3ccc(C#N)c(Cl)c3)C2(C)C)cc1)C(C)(C)C]

(2S)-1-[(2S)-3,3-dimethyl-2-[(2S)-2-(methylamino)propanamido]butanoyl]-N-[(1S,2R)-2-(2-{2-[2-(4-{[(1r,3r)-3-(3-chloro-4-cyanophenoxy)-2,2,4,4-tetramethylcyclobutyl]carbamoyl}phenoxy)ethoxy]ethoxy}ethoxy)-2,3-dihydro-1H-inden-1-yl]pyrrolidine-2-carboxamide
Androgen receptor (AR) degradation in VCaP cells: 18% @ 1 μM and

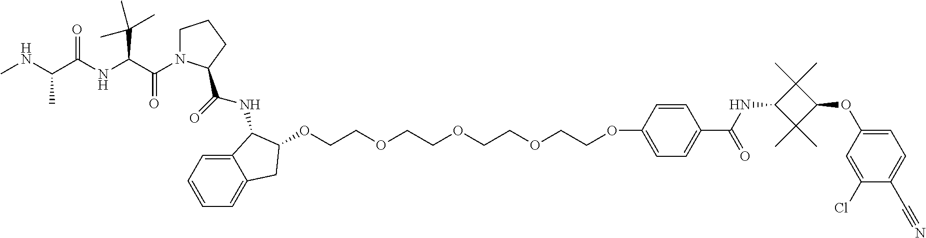 [SMILES: CN[C@@H](C)C(=O)N[C@H](C(=O)N1CCC[C@H]1C(=O)N[C@H]1c2ccccc2C[C@H]1OCCOCCOCCOCCOc1ccc(C(=O)N[C@H]2C(C)(C)[C@H](Oc3ccc(C#N)c(Cl)c3)C2(C)C)cc1)C(C)(C)C]

(2S)-1-[(2S)-3,3-dimethyl-2-[(2S)-2-(methylamino)propanamido]butanoyl]-N-[(1S,2R)-2-{[1-(4-1{[(1r,3r)-3-(3-chloro-4-cyanophenoxy)-2,2,4,4-tetramethylcyclobutyl]carbamoyl}phenyl)-1,4,7,10-tetraoxadodecan-12-yl]oxy}-2,3-dihydro-1H-inden-1-yl]pyrrolidine-2-carboxamide.
Androgen receptor (AR) degradation in VCaP cells: 33% @ 1 M

Synthetic Procedures

Compounds claimed in this document can be synthesized using synthetic methods known in the art of organic chemistry. The following examples are representatives of claimed compounds.

### Intermediate 1

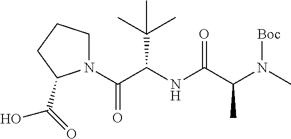 [SMILES: C[C@@H](C(=O)N[C@H](C(=O)N1CCC[C@H]1C(=O)O)C(C)(C)C)N(C)C(=O)OC(C)(C)C]

Intermediate 1 was prepared as described previously by Oost, T. K. et al. in the Journal of Medicinal Chemistry 2004, 47, 4417-4426.

### Intermediate 2

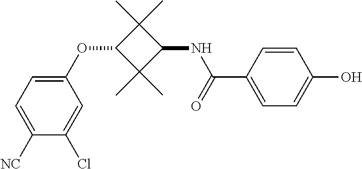

### Step 1

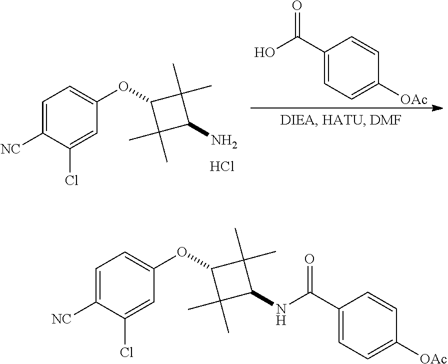

Into a 25-mL round-bottom flask, was placed a solution of 4-(acetyloxy)benzoic acid (100.0 mg, 0.56 mmol, 1.00 equiv) in N,N-dimethylformamide (10 mL), 2-chloro-4-[(1r,3r)-3-amino-2,2,4,4-tetramethylcyclobutoxy]benzonitrile [prepared as described previously by Crew, A. P. et al. in US 20150291562] (190.0 mg, 0.68 mmol, 1.10 equiv), HATU (253.0 g, 665.39 mmol, 1.20 equiv), DIEA (0.5 mL, 5.00 equiv). The resulting solution was stirred for 2 h at room temperature. The reaction was then quenched by the addition of water (10 mL). The resulting solution was extracted with ethyl acetate (10 mL×3) and the organic layers combined. The resulting mixture was washed with brine (10 mL×1). The mixture was dried over anhydrous sodium sulfate and concentrated under vacuum. This resulted in 230.0 mg (94%) of 4-[[(1r,3r)-3-(3-chloro-4-cyanophenoxy)-2,2,4,4-tetramethylcyclobutyl]carbamoyl]phenyl acetate as a light brown solid.

LC-MS (ES⁺): m/z 441.00 [MH⁺]

### Step 2

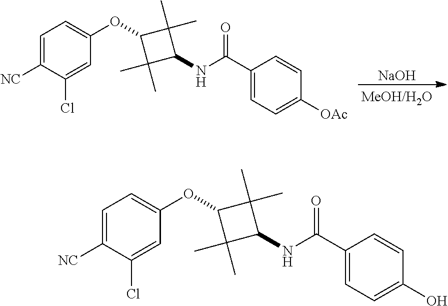

Into a 50-mL round-bottom flask, was placed 4-[[(1r,3r)-3-(3-chloro-4-cyanophenoxy)-2,2,4,4-tetramethylcyclobutyl]carbamoyl]phenyl acetate (230.0 mg, 0.52 mmol, 1.00 equiv) and sodium hydroxide (100.0 mg, 2.50 mmol, 3.00 equiv). The methanol solution was stirred for 2 h at 40° C. in an oil bath. The resulting mixture was concentrated under vacuum. The resulting solution was diluted with water (10 mL). The pH value of the solution was adjusted to 4-5 with hydrogen chloride (1 mol/L). The resulting solution was extracted with ethyl acetate (10 mL×3) and the organic layers combined. The resulting mixture was washed with brine (10 mL×1). The resulting mixture was concentrated under vacuum. The residue was applied onto a silica gel column with ethyl acetate/petroleum ether (1:1). This resulted in 200.0 mg (96%) of 4-hydroxy-N-[(1r,3r)-3-(3-chloro-4-cyanophenoxy)-2,2,4,4-tetramethylcyclobutyl]benzamide as light yellow oil.

LC-MS (ES⁺): m/z 398.95 [MH⁺]

### Intermediate 3

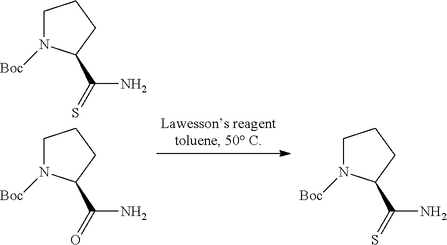

Into a 250-mL round-bottom flask, was placed tert-butyl (2S)-2-carbamoylpyrrolidine-1-carboxylate (4.2 g, 19.60 mmol, 1.00 equiv) in toluene (50 mL). Lawesson's reagent (4.1 g, 0.50 equiv) was added. The resulting solution was stirred for 2 h at 50° C. The reaction mixture was cooled. The solids were filtered out. The resulting mixture was concentrated under vacuum. The residue was applied onto a silica gel column eluting with ethyl acetate/petroleum ether (1:2). This resulted in 1 g (22%) of tert-butyl (2S)-2-carbamothioylpyrrolidine-1-carboxylate as a yellow solid.

### Intermediate 4

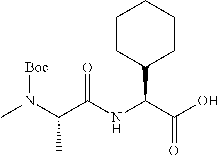 [SMILES: C[C@@H](C(=O)N[C@H](C(=O)O)C1CCCCC1)N(C)C(=O)OC(C)(C)C]

Intermediate 4 was prepared as described previously by Hennessy, E. J. et al. in the Journal of Medicinal Chemistry 2013, 56, 9897-9919.

### Intermediate 5

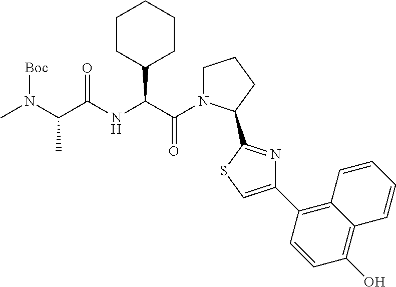 [SMILES: C[C@@H](C(=O)N[C@H](C(=O)N1CCC[C@H]1c1nc(-c2ccc(O)c3ccccc23)cs1)C1CCCCC1)N(C)C(=O)OC(C)(C)C]

### Step 1

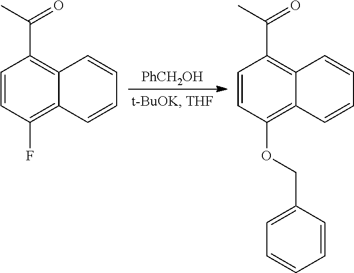 [SMILES: "PhCH2OH" ; CC(=O)c1ccc(F)c2ccccc12 ; CC(=O)c1ccc(OCc2ccccc2)c2ccccc12]

Into a 50-mL round-bottom flask, was placed phenylmethanol (86 mg, 0.80 mmol, 1.50 equiv) in tetrahydrofuran (5 mL), t-BuOK (1 M in tetrahydrofuran) (0.8 mL, 1.50 equiv). The resulting solution was stirred for 10 min at room temperature. Then 1-(4-fluoronaphthalen-1-yl)ethan-1-one (100 mg, 0.53 mmol, 1.00 equiv) was added. The reaction mixture was stirred for 2 h at room temperature. The resulting solution was extracted with 2×20 mL of EA. The combined organic layers were washed with brine, dried over anhydrous sodium sulfate and concentrated under vacuum. This resulted in 150 mg (crude) of 1-(4-(benzyloxy)naphthalene-1-yl)ethanone.

### Step 2

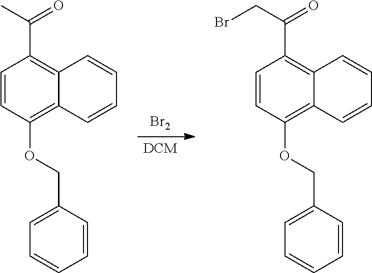

Into a 50-mL round-bottom flask purged and maintained with an inert atmosphere of nitrogen, was placed 1-[4-(benzyloxy)naphthalen-1-yl]ethan-1-one (70 mg, 0.25 mmol, 1.00 equiv) in dichloromethane (5 mL). This was followed by the addition of bromine (44 mg, 0.28 mmol, 1.09 equiv) dropwise with stirring. The resulting solution was stirred for 1 h at room temperature. The resulting solution was extracted with 2×20 mL of dichloromethane and the organic layers combined. The resulting mixture was washed with 20 mL of Na₂S₂O₃ solution, dried over anhydrous sodium sulfate and concentrated under vacuum. This resulted in 100 mg (crude) of 1-[4-(benzyloxy)naphthalen-1-yl]-2-bromoethan-1-one as yellow oil. LC-MS (ES⁺): m/z 355.25, 357.25 [MH⁺]

### Step 3

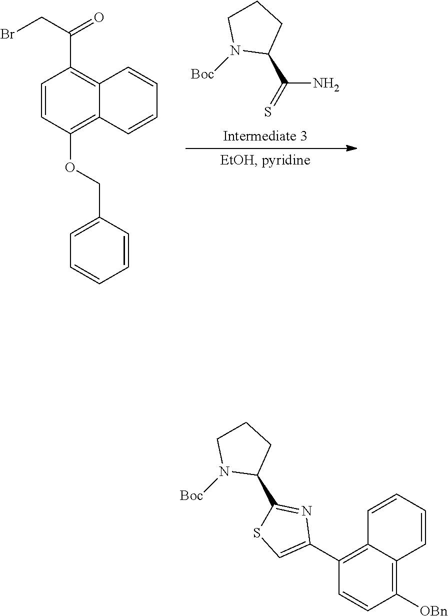

Into a 250-mL round-bottom flask, was placed 1-[4-(benzyloxy)naphthalen-1-yl]-2-bromoethan-1-one (4.9 g, 13.79 mmol, 1.00 equiv), tert-butyl (2S)-2-carbamothioylpyrrolidine-1-carboxylate (intermediate 3) (4.8 g, 20.84 mmol, 1.50 equiv), pyridine (990 mg, 0.90 equiv), ethanol (100 mL). The resulting solution was stirred for 1 h at 80° C. The reaction mixture was quenched with 25 ml of water. The resulting solution was extracted with 2×30 mL of ethyl acetate and the combined organic layers were washed with 50 mL of brine, dried over anhydrous sodium sulfate and concentrated under vacuum. The residue was applied onto a silica gel column eluting with ethyl acetate/petroleum ether (1:1). This resulted in 1.9 g (28%) of tert-butyl (2S)-2-[4-[4-(benzyloxy)naphthalen-1-yl]-1,3-thiazol-2-yl]pyrrolidine-1-carboxylate as yellow oil. LC-MS (ES⁺): m/z 487.35 [MH⁺]

### Step 4

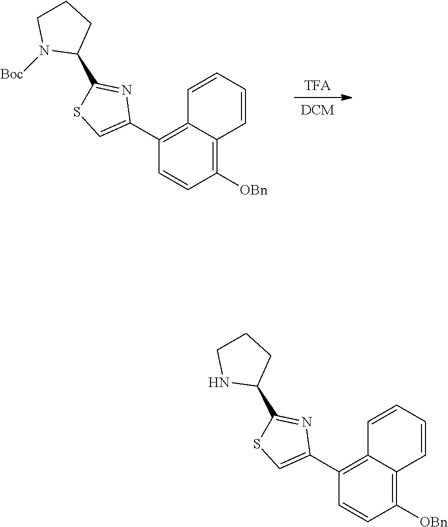

Into a 50-mL round-bottom flask, was placed a solution of tert-butyl (2S)-2-[4-[4-(benzyloxy)naphthalen-1-yl]-1,3-thiazol-2-yl]pyrrolidine-1-carboxylate (1.7 g, 3.49 mmol, 1.00 equiv) and trifluoroacetic acid (10 mL) in dichloromethane (30 mL) at room temperature. The resulting solution was stirred for 2 h at room temperature. The reaction mixture was concentrated under vacuum and this resulted in 1.4 g (83%) of 4-[4-(benzyloxy)naphthalen-1-yl]-2-[(2S)-pyrrolidin-2-yl]-1,3-thiazole, trifluoroacetic acid as yellow oil. LC-MS (ES⁺): m/z 387.50 [M+H]⁺

### Step 5

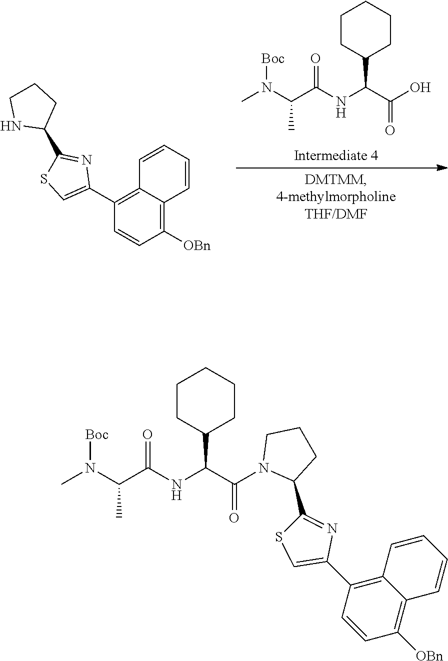 [SMILES: c1ccc(COc2ccc(-c3csc([C@@H]4CCCN4)n3)c3ccccc23)cc1 ; C[C@@H](C(=O)N[C@H](C(=O)O)C1CCCCC1)N(C)C(=O)OC(C)(C)C ; "DMTMM,4-methylmorpholineTHF/DMF" ; "Intermediate 4" ; C[C@@H](C(=O)N[C@H](C(=O)N1CCC[C@H]1c1nc(-c2ccc(OCc3ccccc3)c3ccccc23)cs1)C1CCCCC1)N(C)C(=O)OC(C)(C)C]

Into a 250-mL round-bottom flask, was placed (2S)-2-[(2S)-2-[[(tert-butoxy)carbonyl](methyl)amino]propanamido]-2-cyclohexylacetic acid (1.0 g, 2.92 mmol, 1.00 equiv), 4-[4-(benzyloxy)naphthalen-1-yl]-2-[(2S)-pyrrolidin-2-yl]-1,3-thiazole trifluoroacetic acid salt (1.4 g, 2.80 mmol, 1.20 equiv), 4-methylmorpholine (1.2 g, 11.86 mmol, 4.00 equiv) in tetrahydrofuran (50 mL)/N,N-dimethylformamide (5 mL). DMTMM (1.7 g, 26.92 mmol, 2.00 equiv) was added. The resulting solution was stirred for 2 h at room temperature. The resulting solution was extracted with 3×50 mL of ethyl acetate and the organic layers combined. The resulting mixture was washed with 50 mL of brine, dried over anhydrous sodium sulfate and concentrated under vacuum. The residue was applied onto a silica gel column eluting with ethyl acetate/petroleum ether (1:2). This resulted in 1.4 g (67%) of tert-butyl N-[(1S)-1-[[(1S)-2-[(2S)-2-[4-[4-(benzyloxy)naphthalen-1-yl]-1,3-thiazol-2-yl]pyrrolidin-1-yl]-1-cyclohexyl-2-oxoethyl]carbamoyl]ethyl]-N-methylcarbamate as yellow oil. LC-MS (ES⁺): m/z 711.35 [MH⁺]

### Step 6

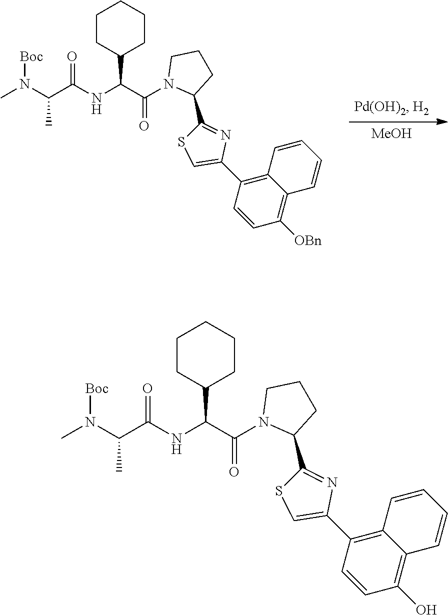

Into a 100-mL round-bottom flask, was placed tert-butyl N-[(1S)-1-[[(1S)-2-[(2S)-2-[4-[4-(benzyloxy)naphthalen-1-yl]-1,3-thiazol-2-yl]pyrrolidin-1-yl]-1-cyclohexyl-2-oxoethyl]carbamoyl]ethyl]-N-methylcarbamate (1.4 g, 1.97 mmol, 1.00 equiv), Pd(OH)₂ (1 g, 7.12 mmol, 3.62 equiv), ethanol (10 mL). The reaction flask was vacuumed and fitted with a hydrogen balloon. The resulting mixture was stirred overnight at room temperature under hydrogen atmosphere. The solids were filtered off. The filtrate was concentrated under vacuum. This resulted in 920 mg (75%) of tert-butyl N-[(1S)-1-[[(1S)-1-cyclohexyl-2-[(2S)-2-[4-(4-hydroxynaphthalen-1-yl)-1,3-thiazol-2-yl]pyrrolidin-1-yl]-2-oxoethyl]carbamoyl]ethyl]-N-methylcarbamate as a white solid. LC-MS (ES⁺): m/z 621.80 [MH⁺]

### Intermediate 6

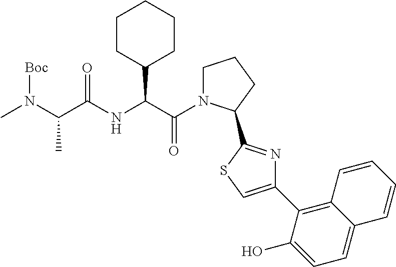 [SMILES: C[C@@H](C(=O)N[C@H](C(=O)N1CCC[C@H]1c1nc(-c2c(O)ccc3ccccc23)cs1)C1CCCCC1)N(C)C(=O)OC(C)(C)C]

### Step 1

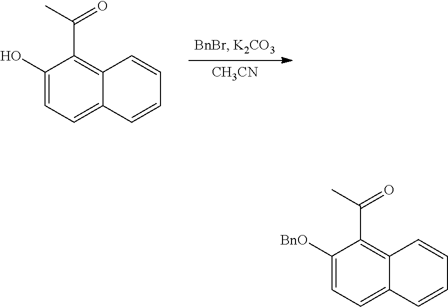

A mixture of 1-(2-hydroxynaphthalen-1-yl)ethanone (11.0 g, 59 mmol), benzyl bromide (12.1 g, 71 mmol) and potassium carbonate (16.3 g, 118 mmol) in acetonitrile (200 ml) was refluxed for 3 hours. TLC showed the reaction was complete. After cooling to room temperature, the mixture was concentrated to 50 ml, and the residue was partitioned between ethyl acetate (250 ml) and water (90 ml). The organic layer was collected, dried over anhydrous sodium sulfate, and concentrated to give a crude residue which was purified by silica gel flash chromatography (eluted with 10-20% ethyl acetate in hexane) to afford 1-(2-(benzyloxy)naphthalen-1-yl)ethanone (15.4 g, yield 94%) as yellow solid.

LC_MS: (ES⁺): m/z 277.1 [M+H]⁺.

### Step 2

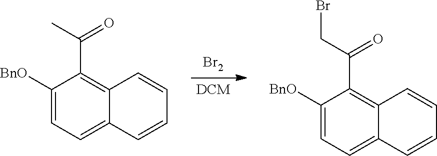

To a solution of 1-(2-(benzyloxy)-naphthalen-1-yl)-2-bromoethanone (15.4 g, 55.7 mmol) in anhydrous dichloromethane (120 ml) was added bromine (9.8 g, 61.3 mmol) 0° C., the resulting mixture was stirred at room temperature for 1 hour. TLC showed the reaction was complete. The mixture was quenched with 10% aqueous sodium thiosulfate (90 ml) and stirred vigorously for 20 min. The organic layer was collected, washed with saturated aqueous sodium bicarbonate (40 ml) and then brine (20 ml), dried over anhydrous sodium sulfate, and concentrated to give a crude product which was purified by silica gel flash chromatography (eluted with 5-10% ethyl acetate in hexane) to afford 1-(2-(benzyloxy)naphthalen-1-yl)-2-bromoethanone (8.5 g, crude) as yellow solid which was used in next step without further purification.

### Step 3

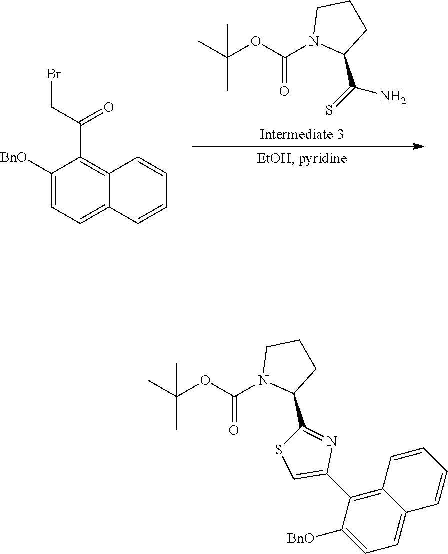

A mixture of (S)-tert-butyl 2-carbamothioylpyrrolidine-1-carboxylate (intermediate 3) (2.5 g, 10.9 mmol), 1-(2-(benzyloxy)naphthalen-1-yl)-2-bromoethanone (5.8 g, 16.3 mmol) and pyridine (0.95 ml, 10.9 mmol) in ethanol (25 ml) was stirred at 80° C. for 1 hour. TLC showed the reaction was complete. The volatiles were evaporated under reduced pressure to give a crude residue which was purified by silica gel flash chromatography (eluted with 0-5% ethyl acetate in dichloromethane) to afford (S)-tert-butyl-2-(4-(2-(benzyloxy)naphthalen-1-yl)thiazol-2-yl)pyrrolidine-1-carboxylate (2.1 g, yield 40%) as yellow oil.

LC_MS: (ES⁺): m/z 487.3 [M+H]⁺.

¹H NMR (400 MHz, CDCl₃): δ 1.30, 1.46 (two s, 9H), 1.67-2.01 (m, 3H), 2.23-2.33 (m, 1H), 3.38-3.43 (m, 1H), 3.52-3.56 (m, 1H), 5.06-5.16 (m, 3H), 7.14-7.33 (m, 8H), 7.39-7.48 (m, 2H), 7.73-7.75 (m, 1H), 7.84 (d, J=8.8 Hz, 1H).

Steps 4 through 6 were carried out as described for the synthesis of intermediate 5 above to afford intermediate 6.

### Intermediate 7

### tert-Butyl N-[(1S)-1-[[(1S)-1-cyclohexyl-2-[6-[2-(4-hydroxyphenyl)ethyl]-octahydro-1H-pyrrolo[2,3-c]pyridin-1-yl]-2-oxoethyl]carbamoyl]ethyl]-N-methylcarbamate

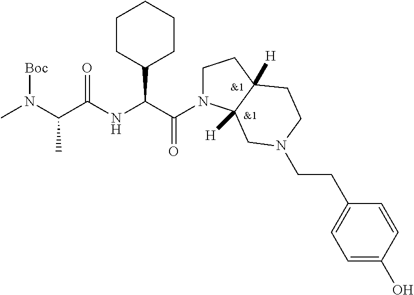 [SMILES: C[C@@H](C(=O)N[C@H](C(=O)N1CC[C@@H]2CCN(CCc3ccc(O)cc3)C[C@@H]21)C1CCCCC1)N(C)C(=O)OC(C)(C)C]

### Step 1

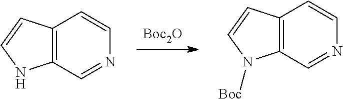 [SMILES: "Boc2O" ; c1cc2cc[nH]c2cn1 ; CC(C)(C)OC(=O)n1ccc2ccncc21]

Into a 250-mL round-bottom flask, was placed a solution of 1H-pyrrolo[2,3-c]pyridine (11.8 g, 99.88 mmol, 1.00 equiv), triethylamine (20.1 g, 198.64 mmol, 3.00 equiv), (Boc)₂O (43.6 g, 199.77 mmol, 2.00 equiv) in dichloromethane (100 mL). The resulting solution was stirred for 16 h at room temperature. The resulting solution was extracted with 2×100 mL of dichloromethane and the organic layers combined and concentrated under vacuum. The residue was applied onto a silica gel column eluting with ethyl acetate/petroleum ether (1:5). This resulted in 13 g (60%) of tert-butyl 1H-pyrrolo[2,3-c]pyridine-1-carboxylate as a yellow solid.

### Step 2

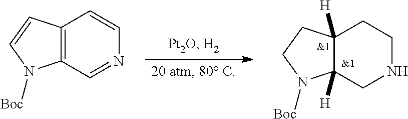

Into a 300-mL pressure tank reactor purged and maintained with an inert atmosphere of nitrogen, was placed tert-butyl 1H-pyrrolo[2,3-c]pyridine-1-carboxylate (3.0 g, 13.75 mmol, 1.00 equiv), acetic acid (150 mL), PtO₂ (1.5 g). The flask was then vacuumed and charged with hydrogen at 20 atm. The resulting solution was stirred for 48 h at 80° C. The solids were filtered out. The resulting mixture was concentrated under vacuum. This resulted in 3.05 g (98%) of tert-butyl octahydro-1H-pyrrolo[2,3-c]pyridine-1-carboxylate as red oil. LC-MS (ES⁺): m/z 227.15 [MH⁺]

### Step 3

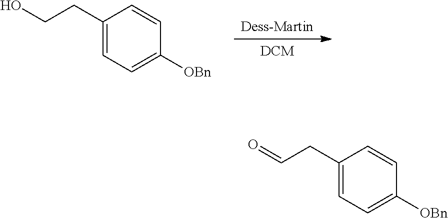

Into a 250-mL round-bottom flask, Dess-Martin periodinane (CAS #87413-090) (11.0 g, 1.20 equiv) was added into a solution of 2-[4-(benzyloxy)phenyl]ethan-1-ol (5.0 g, 21.90 mmol, 1.00 equiv) in dichloromethane (100 mL) at 0 degrees. The reaction mixture was stirred for 3 h at room temperature. The reaction was then quenched by the addition of water. The resulting solution was extracted with dichloromethane. The combined organic layers were dried over anhydrous sodium sulfate and concentrated under vacuum. The residue was applied onto a silica gel column eluting with ethyl acetate/petroleum ether (1:1). This resulted in 3.9 g (79%) of 2-[4-(benzyloxy)phenyl]acetaldehyde as a solid.

### Step 4

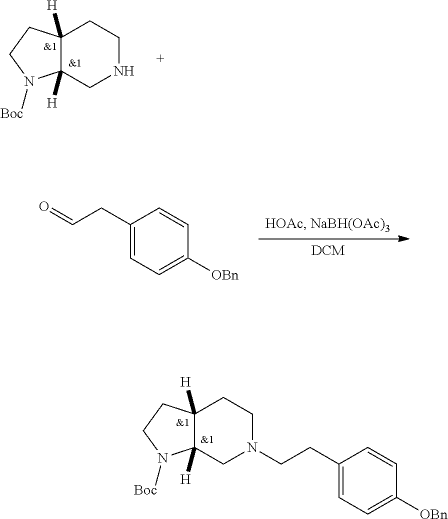

Into a 250-mL round-bottom flask, was placed tert-butyl octahydro-1H-pyrrolo[2,3-c]pyridine-1-carboxylate (3.0 g, 13.26 mmol, 1.00 equiv), 2-[4-(benzyloxy)phenyl]acetaldehyde (3.9 g, 17.24 mmol, 1.30 equiv), NaBH(OAc)₃ (3.77 g, 1.30 equiv) in dichloromethane (60 mL). The resulting solution was stirred for 12 h at room temperature. The reaction was then quenched by the addition of water. The resulting solution was extracted with dichloromethane. The combined organic layers was dried over anhydrous sodium sulfate and concentrated under vacuum. The residue was applied onto a silica gel column eluting with ethyl acetate/petroleum ether (1:1). This resulted in 3.1 g (54%) of tert-butyl 6-[2-[4-(benzyloxy)phenyl]ethyl]-octahydro-1H-pyrrolo[2,3-c]pyridine-1-carboxylate as red oil.

LC-MS (ES⁺): m/z 437.45 [MH⁺]

### Step 5

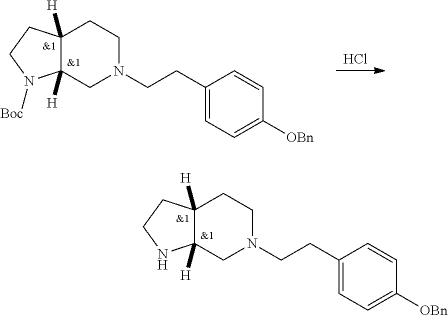 [SMILES: CC(C)(C)OC(=O)N1CC[C@@H]2CCN(CCc3ccc(OCc4ccccc4)cc3)C[C@@H]21 ; Cl ; c1ccc(COc2ccc(CCN3CC[C@H]4CCN[C@H]4C3)cc2)cc1]

Into a 250-mL round-bottom flask, HCl (gas) was introduced into a solution of tert-butyl 6-[2-[4-(benzyloxy)phenyl]ethyl]-octahydro-1H-pyrrolo[2,3-c]pyridine-1-carboxylate (3.1 g, 7.10 mmol, 1.00 equiv) in dioxane (100 mL) at room temperature. The resulting solution was stirred overnight at room temperature. The reaction mixture was concentrated under vacuum. This resulted in 2.4 g (crude) of 6-[2-[4-(benzyloxy)phenyl]ethyl]-octahydro-1H-pyrrolo[2,3-c]pyridine as a white solid.

LC-MS (ES⁺): m/z 337.40 [MH⁺]

Steps 6 and 7 were carried out as described for steps 5 and 6 of the synthesis of intermediate 5 above to afford intermediate 7.

### Intermediate 8

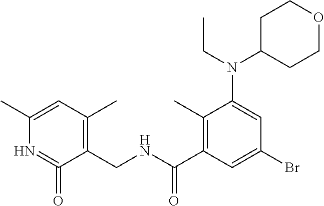

Intermediate 8 was prepared as described previously by Kuntz, K. W. et al. in the Journal of Medicinal Chemistry 2016, 59, 1556-1564.

### Intermediate 9

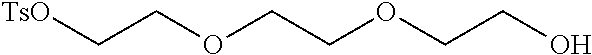 [SMILES: Cc1ccc(S(=O)(=O)OCCOCCOCCO)cc1]

Into a 250-mL round-bottom flask, was placed a solution of 2-[2-(2-hydroxyethoxy)ethoxy]-ethan-1-ol (1.5 g, 9.99 mmol, 1.00 equiv), Ag₂O (3.4 g, 14.98 mmol, 1.50 equiv), KI (0.5 g, 2.99 mmol, 0.30 equiv) in dichloromethane (50 mL) at room temperature. This was followed by the addition of TsCl (2.3 g, 12.06 mmol, 1.20 equiv). The resulting solution was stirred overnight at room temperature. The reaction mixture was then quenched by the addition of water (40 mL). The insoluble solids in the reaction mixture were filtered out and the filtrate was extracted with dichloromethane (40 ml×3). The organic layers were combined, washed with brine, dried over anhydrous sodium sulfate and concentrated under reduced pressure. The residue was applied onto a silica gel column eluting with dichloromethane/methanol (1:1). This resulted in 1 g (33%) of 14-[[(4-methylbenzene)sulfonyl]oxy]-3,6,9,12-tetraoxatetradecan-1-ol as yellow oil.

### Intermediate 10

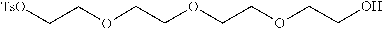 [SMILES: Cc1ccc(S(=O)(=O)OCCOCCOCCOCCO)cc1]

The intermediate was prepared from 1,11-dihydroxy-3,6,9-trioxaundecane (CAS #112-60-7) using procedure described above for intermediate 9.

### Example 1

### 1-[3,3-dimethyl-(2S)-2-[(2S)-2-(methylamino)propanamido]butanoyl]-N-[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10,13-pentaoxapentadecan-15-yl]oxy]-2,3-dihydro-1H-inden-1-yl]pyrrolidine-2-carboxamide

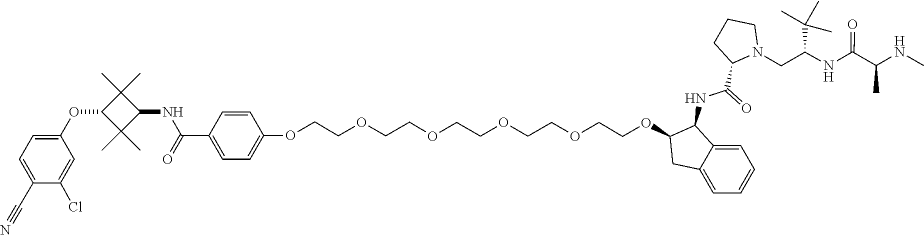 [SMILES: CN[C@@H](C)C(=O)N[C@H](CN1CCC[C@H]1C(=O)N[C@H]1c2ccccc2C[C@H]1OCCOCCOCCOCCOCCOc1ccc(C(=O)N[C@H]2C(C)(C)[C@H](Oc3ccc(C#N)c(Cl)c3)C2(C)C)cc1)C(C)(C)C]

### Step 1. 1-phenyl-2,5,8,11,14-pentaoxahexadecan-16-ol

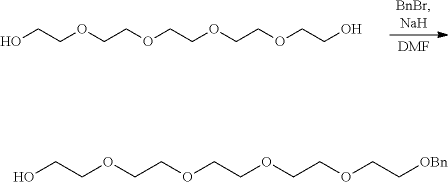

Into a 100-mL 3-necked round-bottom flask purged and maintained with an inert atmosphere of nitrogen, was placed a solution of 3,6,9,12-tetraoxatetradecane-1,14-diol (5.0 g, 20.98 mmol, 1.00 equiv) in N,N-dimethylformamide (10 mL). This was followed by the addition of sodium hydride (920.0 mg, 38.33 mmol, 1.10 equiv), in portions at 0° C. in 5 min. To this was added a solution of (bromomethyl)benzene (3.75 g, 21.93 mmol, 1.05 equiv) in N,N-dimethylformamide (5.0 mL) at 0° C. in 10 min. The resulting solution was stirred overnight at room temperature. The resulting solution was diluted with water (100 mL). The resulting solution was extracted with ethyl acetate (3×100 mL) and the organic layers combined. The residue was applied onto a silica gel column with ethyl acetate/petroleum ether (1/1). This resulted in 1.3 g (19%) of 1-phenyl-2,5,8,11,14-pentaoxahexadecan-16-ol as colorless oil.

### Step 2. 1-phenyl-2,5,8,11,14-pentaoxahexadecan-16-yl 4-methylbenzene-1-sulfonate

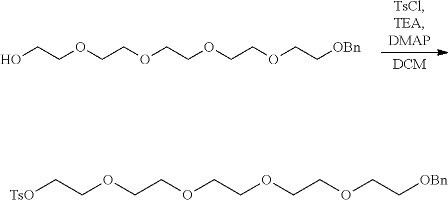

Into a 100-mL round-bottom flask, was placed 1-phenyl-2,5,8,11,14-pentaoxahexadecan-16-ol (1.3 g, 3.96 mmol, 1.00 equiv), 4-toluene sulfonyl chloride (1.1 g, 5.77 mmol, 1.50 equiv), N,N-dimethylpyridine (50.0 mg, 0.41 mmol, 0.10 equiv), trimethylamine (1.67 mL), dichloromethane (20.0 mL). The resulting solution was stirred overnight at room temperature. The resulting solution was diluted with water (100 mL). The resulting solution was extracted with dichloromethane (10×20 mL) and the organic layers combined. The residue was applied onto a silica gel column with ethyl acetate/petroleum ether (1/5). This resulted in 1.6 g (84%) of 1-phenyl-2,5,8,11,14-pentaoxahexadecan-16-yl 4-methylbenzene-1-sulfonate as colorless oil.

### Step 3. (1S,2R)-2-[(1-phenyl-2,5,8,11,14-pentaoxahexadecan-16-yl)oxy]-2,3-dihydro-1H-inden-1-amine

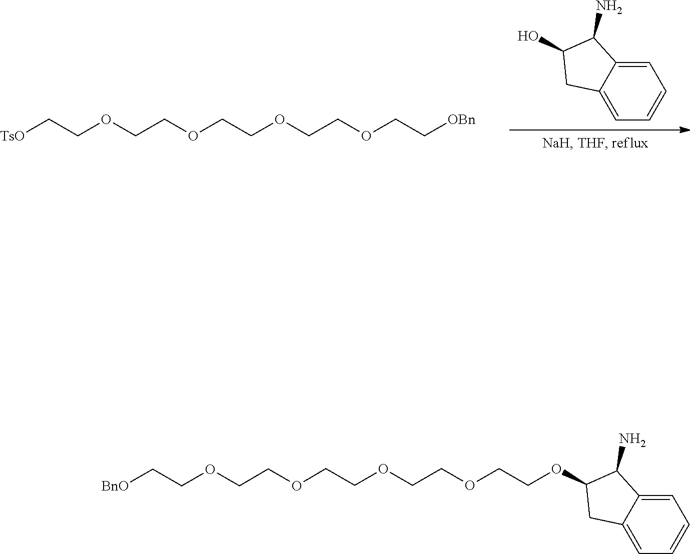 [SMILES: Cc1ccc(S(=O)(=O)OCCOCCOCCOCCOCCOCc2ccccc2)cc1 ; N[C@H]1c2ccccc2C[C@H]1O ; N[C@H]1c2ccccc2C[C@H]1OCCOCCOCCOCCOCCOCc1ccccc1]

Into a 100-mL 3-necked round-bottom flask purged and maintained with an inert atmosphere of nitrogen, was placed (1S,2R)-1-amino-2,3-dihydro-1H-inden-2-ol (500.0 mg, 3.35 mmol, 1.00 equiv), tetrahydrofuran (30.0 mL). This was followed by the addition of sodium hydride (268 mg, 11.17 mmol, 2.00 equiv), in portions at 0° C. After 30 min, to this was added a solution of 1-phenyl-2,5,8,11,14-pentaoxahexadecan-16-yl 4-methylbenzene-1-sulfonate (1.6 g, 3.32 mmol, 1.00 equiv) in tetrahydrofuran (5.0 mL) dropwise with stirring. The resulting solution was stirred for 5.0 h at 70° C. The reaction mixture was cooled to room temperature with a water bath. The reaction was then quenched by the addition of water (50 mL). The resulting solution was extracted with ethyl acetate (3×50 mL) and the organic layers combined. The residue was applied onto a silica gel column with dichloromethane/methanol (10/1). This resulted in 929 mg (60%) of (1S,2R)-2-[(1-phenyl-2,5,8,11,14-pentaoxahexadecan-16-yl)oxy]-2,3-dihydro-1H-inden-1-amine as black oil.

### Step 4

### tert-Butyl N-[(1S)-1-[[(2S)-3,3-dimethyl-1-oxo-1-[(2S)-2-[[(1S,2R)-2-[(1-phenyl-2,5,8,11,14-pentaoxahexadecan-16-yl)oxy]-2,3-dihydro-1H-inden-1-yl]carbamoyl]pyrrolidin-1-yl]butan-2-yl]carbamoyl]ethyl]-N-methylcarbamate

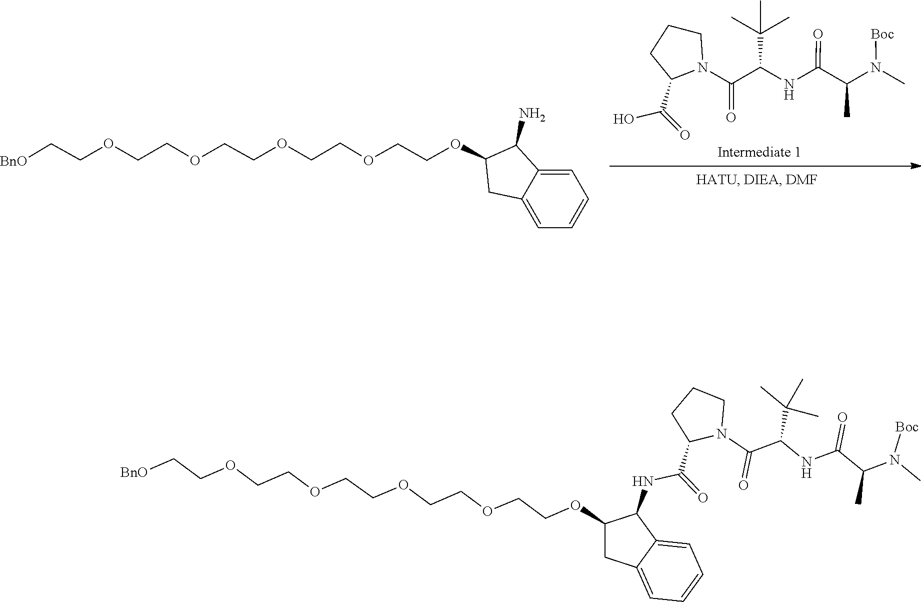 [SMILES: N[C@H]1c2ccccc2C[C@H]1OCCOCCOCCOCCOCCOCc1ccccc1 ; C[C@@H](C(=O)N[C@H](C(=O)N1CCC[C@H]1C(=O)O)C(C)(C)C)N(C)C(=O)OC(C)(C)C ; C[C@@H](C(=O)N[C@H](C(=O)N1CCC[C@H]1C(=O)N[C@H]1c2ccccc2C[C@H]1OCCOCCOCCOCCOCCOCc1ccccc1)C(C)(C)C)N(C)C(=O)OC(C)(C)C]

Into a 100-mL round-bottom flask purged and maintained with an inert atmosphere of nitrogen, was placed (1S,2R)-2-[(1-phenyl-2,5,8,11,14-pentaoxahexadecan-16-yl)oxy]-2,3-dihydro-1H-inden-1-amine (220.0 mg, 0.48 mmol, 1.00 equiv), N,N-dimethylformamide (5.0 mL), 0-(7-Azabenzotriazol-1-yl)-N,N,N,N-tetramethyluronium Hexafluorophosphate (218.0 mg, 0.57 mmol, 1.20 equiv), (2S)-1-[(2S)-2-[(2S)-2-[[(tert-butoxy)carbonyl](methyl)amino]-propanamido]-3,3-dimethylbutanoyl]pyrrolidine-2-carboxylic acid (200.0 mg, 0.48 mmol, 1.00 equiv), N,N-Diisopropylethylamine (0.2 mL). The resulting solution was stirred for 1 h at room temperature. The resulting solution was diluted with water (10 mL). The resulting solution was extracted with ethyl acetate (3×20 mL) and the organic layers combined. The residue was applied onto a silica gel column with dichloromethane/methanol (10/1). This resulted in 267 mg (65%) of tert-butyl N-[(1S)-1-[[(2S)-3,3-dimethyl-1-oxo-1-[(2S)-2-[[(1S,2R)-2-[(1-phenyl-2,5,8,11,14-pentaoxahexadecan-16-yl)oxy]-2,3-dihydro-1H-inden-1-yl]carbamoyl]pyrrolidin-1-yl]butan-2-yl]carbamoyl]ethyl]-N-methylcarbamate as brown oil.

LC-MS (ES⁺): m/z 855.40 [MH]⁺.

### Step 5

### tert-Butyl (S)-1-((S)-1-((S)-2-((1S,2R)-2-(14-hydroxy-3,6,9,12-tetraoxatetradecyloxy)-2,3-dihydro-1H-inden-1-yl-carbamoyl)pyrrolidin-1-yl)-3,3-dimethyl-1-oxobutan-2-ylamino)-1-oxopropan-2-yl(methyl)carbamate

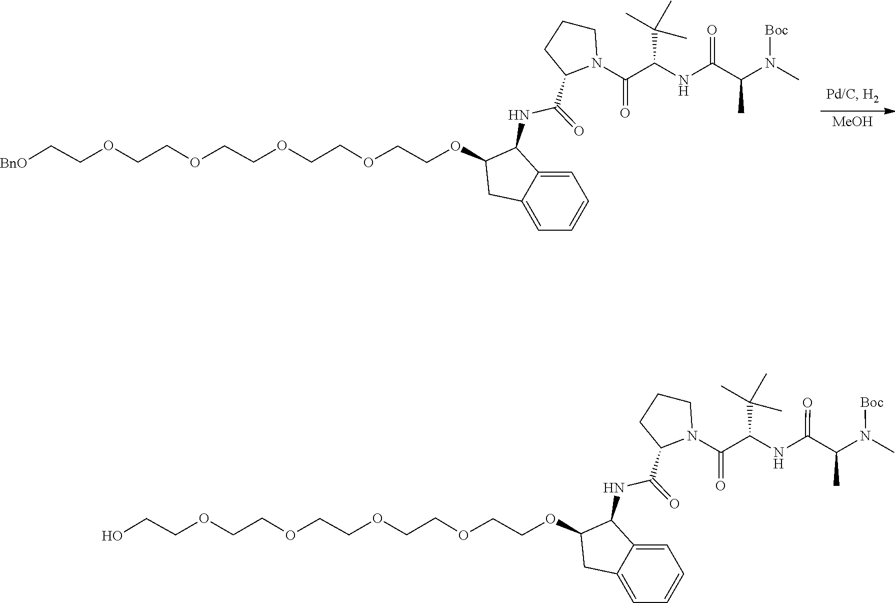 [SMILES: "MeOH" ; C[C@@H](C(=O)N[C@H](C(=O)N1CCC[C@H]1C(=O)N[C@H]1c2ccccc2C[C@H]1OCCOCCOCCOCCOCCOCc1ccccc1)C(C)(C)C)N(C)C(=O)OC(C)(C)C ; C[C@@H](C(=O)N[C@H](C(=O)N1CCC[C@H]1C(=O)N[C@H]1c2ccccc2C[C@H]1OCCOCCOCCOCCOCCO)C(C)(C)C)N(C)C(=O)OC(C)(C)C]

Into a 100-mL round-bottom flask purged and maintained with an inert atmosphere of nitrogen, was placed tert-butyl N-[(1S)-1-[[(2S)-3,3-dimethyl-1-oxo-1-[(2S)-2-[[(1S,2R)-2-[(1-phenyl-2,5,8,11,14-pentaoxahexadecan-16-yl)oxy]-2,3-dihydro-1H-inden-1-yl]carbamoyl]pyrrolidin-1-yl]butan-2-yl]carbamoyl]ethyl]-N-methylcarbamate (267.0 mg, 0.48 mmol, 1.00 equiv), Pd/C (200 mg) in methanol (10 mL) was hydrogenated under 1 atm at room temperature for 15 h. The resulting solution was filtrated and the filtrate was concentrated. This resulted in 210 mg (90%) of tert-butyl (S)-1-((S)-1-((S)-2-((1S,2R)-2-(14-hydroxy-3,6,9,12-tetraoxatetradecyloxy)-2,3-dihydro-1H-inden-1-yl-carbamoyl)pyrrolidin-1-yl)-3,3-dimethyl-1-oxobutan-2-ylamino)-1-oxopropan-2-yl(methyl)carbamate as brown oil.

### Step 6

### tert-butyl N-[(1S)-1-[[(2S)-3,3-dimethyl-1-(2-[[(1S,2R)-2-[(14-[[(4-methylbenzene)-sulfonyl]oxy]-3,6,9,12-tetraoxatetradecan-1-yl)oxy]-2,3-dihydro-1H-inden-1-yl]carbamoyl]pyrrolidin-1-yl)-1-oxobutan-2-yl]carbamoyl]ethyl]-N-methylcarbamate

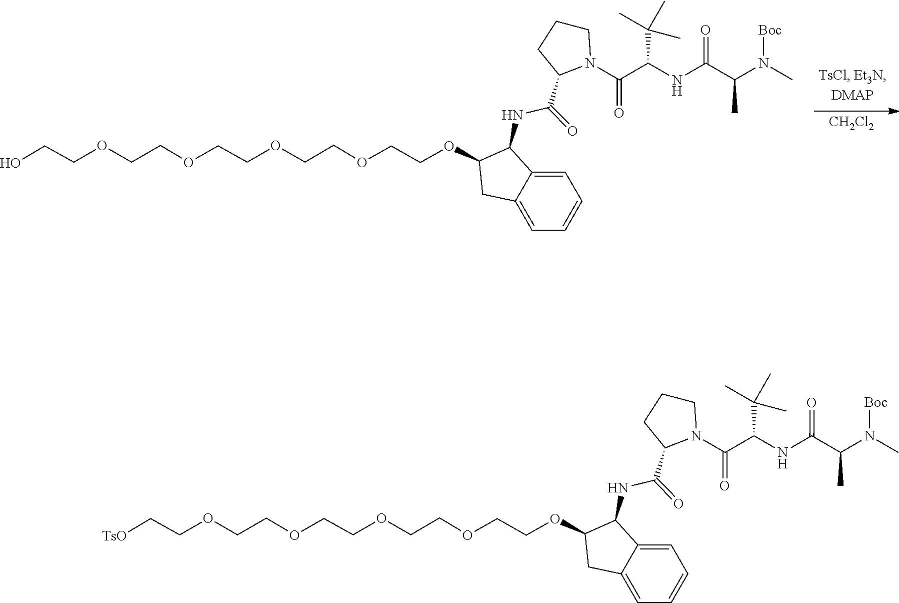

Into a 100-mL round-bottom flask, was placed tert-butyl N-[(1S)-1-[[1-(2-[[(1S,2R)-2-[(14-hydroxy-3,6,9,12-tetraoxatetradecan-1-yl)oxy]-2,3-dihydro-1H-inden-1-yl]carbamoyl]pyrrolidin-1-yl)-3,3-dimethyl-1-oxobutan-2-yl]carbamoyl]ethyl]-N-methylcarbamate (375 mg, 0.49 mmol, 1.00 equiv), 4-toluene sulfonyl chloride (139 mg, 0.73 mmol, 1.50 equiv), dichloromethane (20 mL), trimethylamine (0.2 mL), N,N-dimethylpyridine (6.0 mg, 0.05 mmol, 0.10 equiv). The resulting solution was stirred for 24 h at room temperature. The resulting mixture was washed with water (2×20 mL). The mixture was dried over anhydrous sodium sulfate. The residue was applied onto a silica gel column with dichloromethane/methanol (10/1). This resulted in 309.0 mg (69%) of tert-butyl N-[(1S)-1-[[(2S)-3,3-dimethyl-1-(2-[[(1S,2R)-2-[(14-[[(4-methylbenzene)sulfonyl]oxy]-3,6,9,12-tetraoxatetradecan-1-yl)oxy]-2,3-dihydro-1H-inden-1-yl]carbamoyl]pyrrolidin-1-yl)-1-oxobutan-2-yl]carbamoyl]ethyl]-N-methylcarbamate as colorless oil.

LC-MS (ES⁺): m/z 919.30 [MH]⁺.

### Step 7

### tert-Butyl N-[(1S)-1-[[(2S)-3,3-dimethyl-1-oxo-1-(2-[[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10,13-pentaoxapentadecan-15-yl]oxy]-2,3-dihydro-1H-inden-1-yl]carbamoyl]pyrrolidin-1-yl)butan-2-yl]carbamoyl]ethyl]-N-methylcarbamate

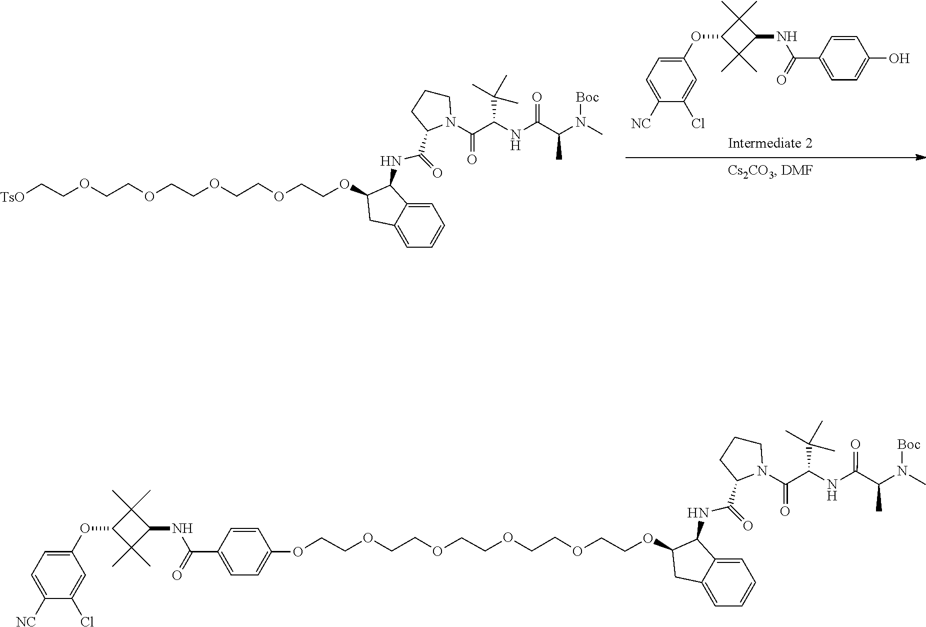

Into a 100-mL round-bottom flask, was placed tert-butyl N-[(1S)-1-[[3,3-dimethyl-1-(2-[[(1S,2R)-2-[(14-[[(4-methylbenzene)sulfonyl]oxy]-3,6,9,12-tetraoxatetradecan-1-yl)oxy]-2,3-dihydro-1H-inden-1-yl]carbamoyl]pyrrolidin-1-yl)-1-oxobutan-2-yl]carbamoyl]ethyl]-N-methylcarbamate (115.0 mg, 0.13 mmol, 1.00 equiv), 4-hydroxy-N-[(1r,3r)-3-(3-chloro-4-cyanophenoxy)-2,2,4,4-tetramethylcyclobutyl]benzamide (50.0 mg, 0.13 mmol, 1.00 equiv), potassium carbonate (34.0 mg, 0.25 mmol, 2.00 equiv), N,N-dimethylformamide (10 mL). The resulting solution was stirred for 2 h at 80° C. The resulting solution was diluted with 20 mL of water. The resulting solution was extracted with ethyl acetate (2×20 mL) and the organic layers combined and dried over anhydrous sodium sulfate and concentrated under vacuum. This resulted in 110.0 mg (77%) of tert-butyl N-[(1S)-1-[[(2S)-3,3-dimethyl-1-oxo-1-(2-[[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10,13-pentaoxapentadecan-15-yl]oxy]-2,3-dihydro-1H-inden-1-yl]carbamoyl]pyrrolidin-1-yl)butan-2-yl]carbamoyl]ethyl]-N-methylcarbamate as brown oil.

### Step 8

### 1-[3,3-dimethyl-(2S)-2-[(2S)-2-(methylamino)propanamido]butanoyl]-N-[(1S,2R)-2-[[1-(4-[[(1r,3r)-3-(3-chloro-4-cyanophenoxy)-2,2,4,4-tetramethylcyclobutyl]-carbamoyl]phenyl)-1,4,7,10,13-pentaoxapentadecan-15-yl]oxy]-2,3-dihydro-1H-inden-1-yl]pyrrolidine-2-carboxamide

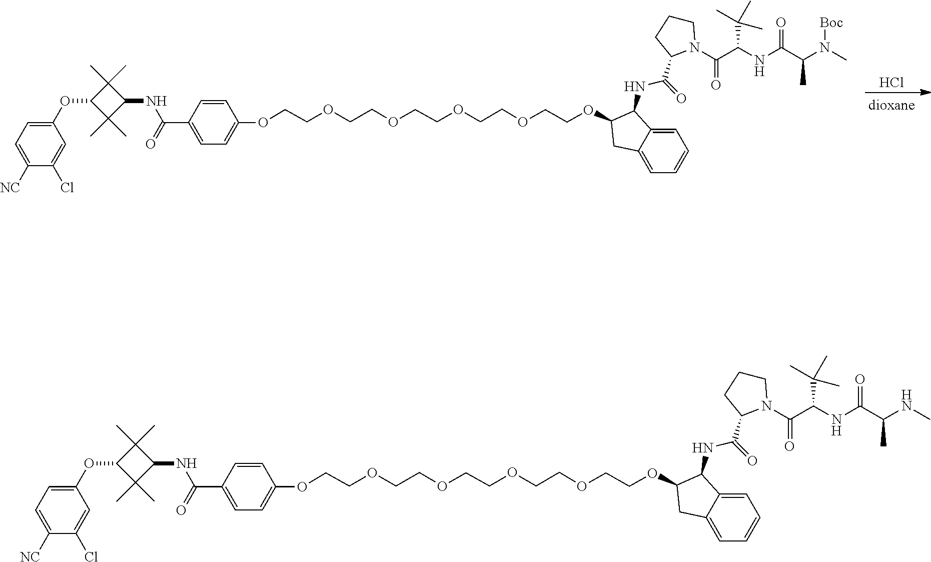

Into a 100-mL round-bottom flask, was placed tert-butyl N-[(1S)-1-[[3,3-dimethyl-1-oxo-1-(2-[[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10,13-pentaoxapentadecan-15-yl]oxy]-2,3-dihydro-1H-inden-1-yl]carbamoyl]pyrrolidin-1-yl)butan-2-yl]carbamoyl]ethyl]-N-methylcarbamate (110.0 mg, 0.10 mmol, 1.00 equiv), hydrogen chloride/dioxnae (20 mL). The resulting solution was stirred for 1 h at room temperature. The resulting mixture was concentrated under vacuum. The crude product was purified by Prep-HPLC with the following conditions (2#-AnalyseHPLC-SHIMADZU (HPLC-10)): Column, XBridge Shield RP18 OBD Column, 5 um, 19*150 mm; mobile phase, Water with 10 mmolNH4HCO3 and ACN (50.0% ACN up to 65.0% in 10 min); Detector, UV 254 nm. This resulted in 49.5 mg (49%) of 1-[3,3-dimethyl-(2S)-2-[(2S)-2-(methylamino)propanamido]butanoyl]-N-[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10,13-pentaoxapentadecan-15-yl]oxy]-2,3-dihydro-1H-inden-1-yl]pyrrolidine-2-carboxamide as a white solid.

¹H NMR (300 MHz, CD₃OD): δ7.75-7.70 (m, 2H), 7.69-7.65 (m, 1H), 7.37-7.32 (m, 1H), 7.25-7.06 (m, 4H), 6.98-6.90 (m, 3H), 5.36-5.32 (m, 1H), 4.60 (s, 1H), 4.55-43 (m, 1H), 4.30-4.20 (m, 2H), 4.18-4.01 (m, 3H), 3.99-3.86 (m, 1H), 3.85-3.83 (m, 2H), 3.81-3.54 (m, 18H), 3.22-3.10 (m, 1H), 3.08-2.98 (m, 2H), 2.30 (s, 3H), 2.20-1.80 (m, 4H), 1.26-1.25 (m, 6H), 1.24-1.26 (m, 8H), 1.05 (s, 8H), 0.97 (s, 1H); LC-MS (ES⁺): m/z, 1045.14 [MH⁺].

Using procedures analogous to those described above for Example 1, the following compounds have been prepared:

### Example 2

### 1-[(2S)-3,3-dimethyl-2-[(2S)-2-(methylamino)propanamido]butanoyl]-N-[(1S,2R)-2-(2-[2-[2-(4-[[(1r,3r)-3-(3-chloro-4-cyanophenoxy)-2,2,4,4-tetramethylcyclobutyl]carbamoyl]phenoxy)ethoxy]ethoxy]ethoxy)-2,3-dihydro-1H-inden-1-yl]pyrrolidine-2-carboxamide

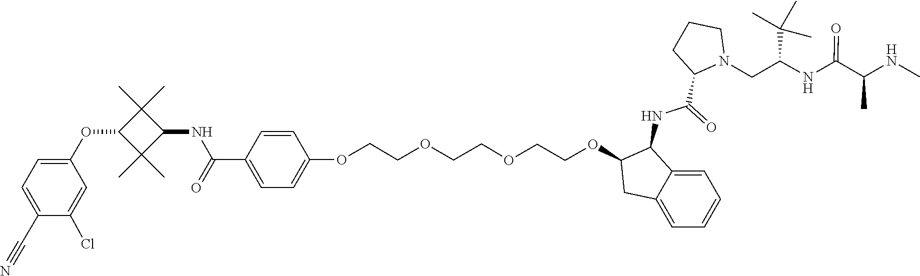 [SMILES: CN[C@@H](C)C(=O)N[C@H](CN1CCC[C@H]1C(=O)N[C@H]1c2ccccc2C[C@H]1OCCOCCOCCOc1ccc(C(=O)N[C@H]2C(C)(C)[C@H](Oc3ccc(C#N)c(Cl)c3)C2(C)C)cc1)C(C)(C)C]

¹H NMR (300 MHz, CD₃OD): δ7.75-7.70 (m, 2H), 7.69-7.65 (m, 1H), 7.37-7.32 (m, 1H), 7.25-7.06 (m, 4H), 6.98-6.90 (m, 3H), 5.36-5.32 (m, 1H), 4.70-43 (m, 3H), 4.30-4.20 (m, 2H), 4.18-4.01 (m, 3H), 3.92-3.86 (m, 3H), 3.85-3.54 (m, 9H), 3.22-3.10 (m, 1H), 3.08-2.98 (m, 2H), 2.30 (s, 3H), 2.20-1.80 (m, 4H), 1.26-1.25 (m, 6H), 1.24-1.26 (m, 8H), 1.05 (s, 8H), 0.97 (s, 1H); LC-MS (ES⁺): m/z, 957.14 [MH⁺]

### Example 3

### 1-[(2S)-3,3-dimethyl-2-[(2S)-2-(methylamino)propanamido]butanoyl]-N-[(1S,2R)-2-[[1-(4-[[(1r,3r)-3-(3-chloro-4-cyanophenoxy)-2,2,4,4-tetramethylcyclobutyl]carbamoyl]phenyl)-1,4,7,10-tetraoxadodecan-12-yl]oxy]-2,3-dihydro-1H-inden-1-yl]pyrrolidine-2-carboxamide

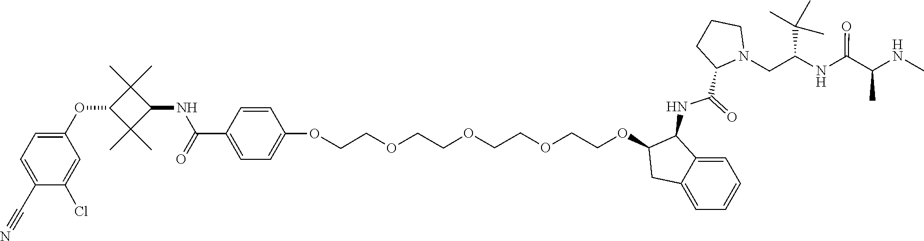 [SMILES: CN[C@@H](C)C(=O)N[C@H](CN1CCC[C@H]1C(=O)N[C@H]1c2ccccc2C[C@H]1OCCOCCOCCOCCOc1ccc(C(=O)N[C@H]2C(C)(C)[C@H](Oc3ccc(C#N)c(Cl)c3)C2(C)C)cc1)C(C)(C)C]

¹H NMR (300 MHz, CD₃OD): δ7.75-7.70 (m, 2H), 7.69-7.65 (m, 1H), 7.37-7.32 (m, 1H), 7.25-7.06 (m, 4H), 6.98-6.90 (m, 3H), 5.36-5.32 (m, 1H), 4.60 (s, 1H), 4.56-4.53 (m, 1H), 4.30-4.20 (m, 2H), 4.18-4.01 (m, 3H), 3.92-3.83 (m, 1H), 3.82-3.75 (m, 2H), 3.74-3.50 (m, 14H), 3.22-3.10 (m, 1H), 3.08-2.98 (m, 2H), 2.30 (s, 3H), 2.20-1.80 (m, 4H), 1.26-1.25 (m, 6H), 1.24-1.26 (m, 8H), 1.05 (s, 8H), 0.97 (s, 1H); LC-MS (ES⁺): m/z, 1001.12 [MH⁺].

### Example 4

### (2S)—N-[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]-2-(methylamino)propanamide

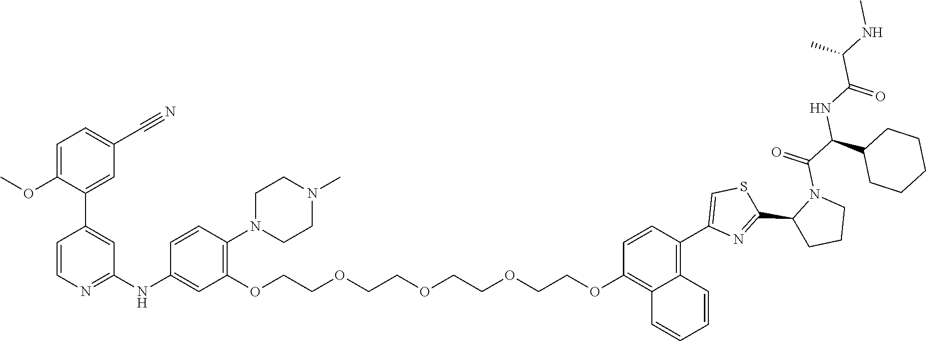 [SMILES: CN[C@@H](C)C(=O)N[C@H](C(=O)N1CCC[C@H]1c1nc(-c2ccc(OCCOCCOCCOCCOc3cc(Nc4cc(-c5cc(C#N)ccc5OC)ccn4)ccc3N3CCN(C)CC3)c3ccccc23)cs1)C1CCCCC1]

### Step 1. 2-(Allyloxy)-1-chloro-4-nitrobenzene

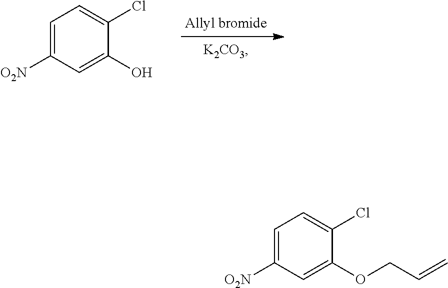

A mixture of 2-chloro-5-nitrophenol (5.0 g, 28.8 mmol), allyl bromide (3.5 g, 28.8 mmol) and potassium carbonate (6.0 g, 43.2 mmol) in N,N-dimethylformamide (50 ml) was stirred at 70° C. for 12 hours. TLC showed the reaction was complete. The mixture was partitioned between ethyl acetate (100 ml) and water (100 ml). The organic layer was collected, and the aqueous layer was extracted with ethyl acetate (50 ml×2). The combined organic layers were washed with brine (50 ml×2), dried over anhydrous sodium sulfate, and concentrated under reduced pressure to give a crude residue which was purified by silica gel flash chromatography (eluted 10-30% ethyl acetate in hexane) to afford 2-(allyloxy)-1-chloro-4-nitrobenzene (5.0 g, 23.5 mmol, yield 81%) as yellow solid.

### Step 2. 1-(2-(Allyloxy)-4-nitrophenyl)-4-methylpiperazine

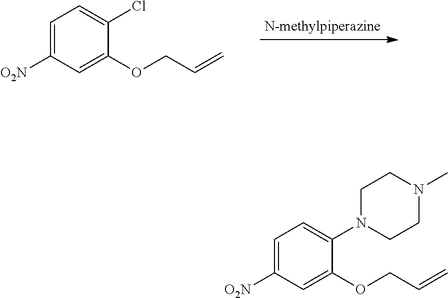 [SMILES: C=CCOc1cc([N+](=O)[O-])ccc1Cl ; C=CCOc1cc([N+](=O)[O-])ccc1N1CCN(C)CC1]

A mixture of 2-(allyloxy)-1-chloro-4-nitrobenzene (5.0 g, 23.5 mmol), 1-methylpiperazine (3.5 g, 35.2 mmol) and potassium carbonate (6.5 g, 47.0 mmol) in 1-methylpyrrolidin-2-one (20 ml) was stirred at 120° C. for 12 hours. TLC showed the reaction was complete. The mixture was partitioned between ethyl acetate (50 ml) and water (50 ml). The organic layer was collected, and the aqueous layer was extracted with ethyl acetate (50 ml×3). The combined organic layers were washed with brine (50 ml×2), dried over anhydrous sodium sulfate, and concentrated under reduced pressure to give a crude residue which was purified by silica gel flash chromatography (eluted 20-40% ethyl acetate in hexane) to afford 1-(2-(allyloxy)-4-nitrophenyl)-4-methylpiperazine (2.5 g, 9.0 mmol, yield 38%) as yellow solid.

### Step 3. 3-(Allyloxy)-4-(4-methylpiperazin-1-yl)aniline

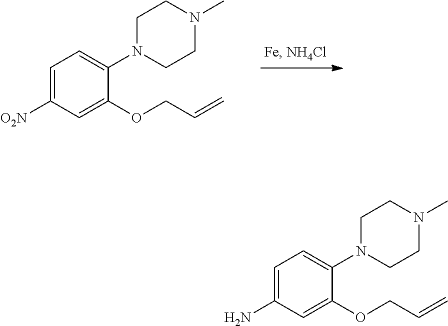 [SMILES: C=CCOc1cc([N+](=O)[O-])ccc1N1CCN(C)CC1 ; C=CCOc1cc(N)ccc1N1CCN(C)CC1]

A mixture of 1-(2-(allyloxy)-4-nitrophenyl)-4-methylpiperazine (2.5 g, 9.0 mmol), iron powder (2.5 g, 45.1 mmol) and ammonium chloride (4.8 g, 90.1 mmol) in ethanol (30 ml)-water (5 ml) was refluxed for 2 hours. TLC showed the reaction was complete. Iron powder was removed through filtration and the filter cake was washed with ethanol (20 ml×2). The combined filtrates were concentrated under reduced pressure to give a residue which was purified by silica gel flash chromatography (eluted with 1-5% methanol in dichloromethane) to afford 3-(allyloxy)-4-(4-methylpiperazin-1-yl)aniline (1.8 g, 7.4 mmol, yield 82%) as yellow solid.

LC_MS: (ES⁺): m/z 278.1 [M+H]⁺

### Step 4. 3-Bromo-4-methoxybenzonitrile

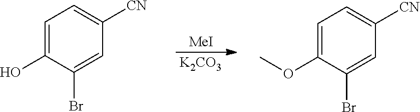

A mixture of 3-bromo-4-hydroxybenzonitrile (5.0 g, 25.3 mmol), potassium carbonate (7.0 g, 50.1 mmol) and iodomethane (3.9 g, 27.8 mmol) in acetonitrile (20 ml) was stirred at 25° C. for 6 hours. TLC showed the reaction was complete. The mixture was partitioned between ethyl acetate (100 ml) and water (30 ml). The organic layer was collected, washed with brine (20 ml), dried over anhydrous sodium sulfate, and concentrated under reduced pressure to give a crude residue which was purified by silica gel flash chromatography (eluted 30% ethyl acetate in hexane) to afford 3-bromo-4-methoxybenzonitrile (4.8 g, 22.6 mmol, yield 89%).

### Step 5. 4-Methoxy-3-(4,4,5,5-tetramethyl-1,3,2-dioxaborolan-2-yl)benzonitrile

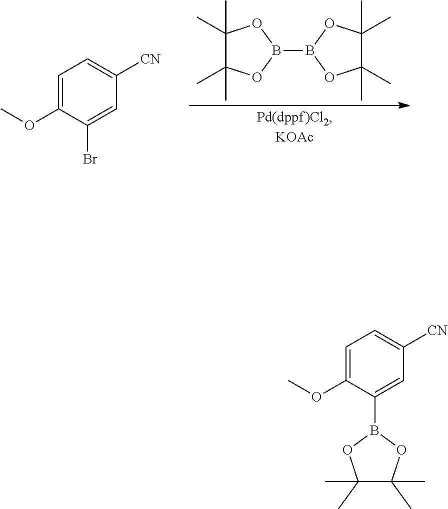

To a stirred solution of 3-bromo-4-methoxybenzonitrile (5.0 g, 23.6 mmol), 4,4,4′,4′,5,5,5′,5′-octamethyl-2,2′-bi(1,3,2-dioxaborolane) (9.0 g, 35.4 mmol), and potassium acetate (6.9 g, 70.1 mmol) in dioxane (50 ml)-dimethyl sulfoxide (1 ml) was added 1,1′-bis(diphenylphosphino)ferrocene palladium(II)dichloride (1.7 g, 2.3 mmol) at room temperature under nitrogen atmosphere; the mixture was degassed with nitrogen three times. The resulting mixture was refluxed for 2 hours. TLC showed the reaction was complete. The cooled reaction mixture was partitioned between ethyl acetate (100 ml) and water (80 ml). The organic layer was collected, and the aqueous layer was extracted with ethyl acetate (50 ml×2). The combined organic layers were washed with brine (40 ml×2), dried over anhydrous sodium sulfate, and concentrated under reduced pressure to give a crude residue which was purified by silica gel flash chromatography (eluted with 10-33% ethyl acetate in hexane) to afford 4-methoxy-3-(4,4,5,5-tetramethyl-1,3,2-dioxaborolan-2-yl)benzonitrile (4.2 g, 16.2 mmol, yield 68%) as white solid.

LC_MS: (ES⁺): m/z 260.0 [M+H]⁺

### Step 6. 3-(2-Fluoropyridin-4-yl)-4-methoxybenzonitrile

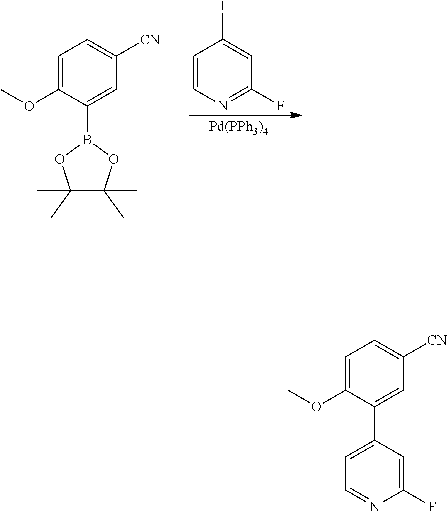 [SMILES: COc1ccc(C#N)cc1B1OC(C)(C)C(C)(C)O1 ; Fc1cc(I)ccn1 ; COc1ccc(C#N)cc1-c1ccnc(F)c1 ; "Pd(PPh3)4"]

To stirred mixture of 4-methoxy-3-(4,4,5,5-tetramethyl-1,3,2-dioxaborolan-2-yl)benzonitrile (2.5 g, 9.6 mmol), 2-fluoro-4-iodopyridine (2.1 g, 9.6 mmol) and potassium acetate (1.9 g, 19.4 mmol) in dioxane (32 ml)-water (8 ml) was added 1,1′-bis(diphenylphosphino)ferrocene-palladium(II)dichloride (700 mg, 0.96 mmol) at room temperature under nitrogen; the mixture was degassed with nitrogen three times. The resulting mixture was refluxed for 2 hours. TLC showed the reaction was complete. The cooled reaction mixture was partitioned between ethyl acetate (50 ml) and water (30 ml). The organic layer was collected, and the aqueous layer was extracted with ethyl acetate (50 ml×2). The combined organic layers were washed with brine (40 ml×2), dried over anhydrous sodium sulfate, and concentrated under reduced pressure to give a crude residue which was purified by silica gel flash chromatography (eluted with 2-10% ethyl acetate in hexane) to afford 3-(2-fluoropyridin-4-yl)-4-methoxybenzonitrile (1.4 g, 6.1 mmol, yield 64%) as white solid.

¹HNMR (400 MHz, CDCl₃): δ 3.93 (s, 3H), 7.08-7.10 (m, 2H), 7.29-7.31 (m, 1H), 7.63 (d, J=2.0 Hz, 1H), 7.72-7.74 (m, 1H), 8.28 (d, J=5.2 Hz, 1H).

### Step 7. 3-(2-((3-(Allyloxy)-4-(4-methylpiperazin-1-yl)phenyl)amino)pyridin-4-yl)-4-methoxybenzonitrile

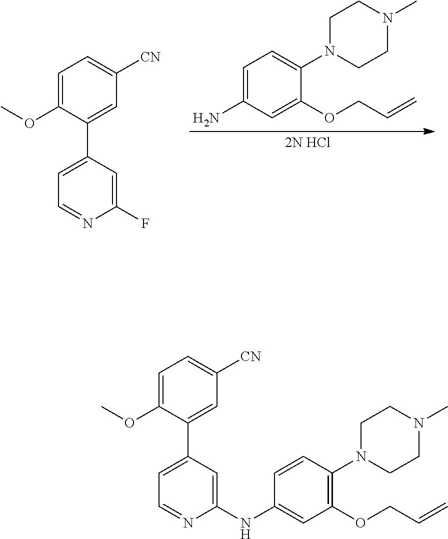

A mixture of 3-(2-fluoropyridin-4-yl)-4-methoxybenzonitrile (923 mg, 4.0 mmol) and 3-(allyloxy)-4-(4-methylpiperazin-1-yl)aniline (1.0 g, 4.0 mmol) in dioxane (10 ml)-water (2 ml)-diluted hydrochloride acid (2N, 2 ml) was stirred in sealed tube at 120° C. for 12 hours. TLC showed the reaction was complete. The cooled reaction mixture was partitioned between ethyl acetate (50 ml) and water (30 ml). The organic layer was collected, and the aqueous layer was extracted with ethyl acetate (50 ml×2). The combined organic layers were washed with brine (40 ml×2), dried over anhydrous sodium sulfate, and concentrated under reduced pressure to give a crude residue which was purified by silica gel flash chromatography (eluted with 20-50% ethyl acetate in hexane) to afford 3-(2-((3-(allyloxy)-4-(4-methylpiperazin-1-yl)phenyl)amino)pyridin-4-yl)-4-methoxybenzonitrile (450 mg, 1.0 mmol, yield 25%).

¹HNMR (400 MHz, CD₃OD): δ 2.99 (s, 3H), 3.07-3.19 (m, 2H), 3.32-3.40 (m, 2H), 3.55-3.69 (m, 4H), 3.97 (s, 3H), 4.65-4.67 (m, 2H), 5.29-5.32 (m, 1H), 5.44-5.49 (m, 1H), 6.10-6.20 (m, 1H), 7.01-7.02 (m, 1H), 7.06 (d, J=0.8 Hz, 2H), 7.09 (s, 1H), 7.25 (s, 1H), 7.32 (d, J=8.8 Hz, 1H), 7.76-7.77 (m, 1H), 7.82-7.85 (m, 1H), 8.02 (d, J=5.6 Hz, 1H).

LC_MS: (ES⁺): m/z 456.2 [M+H]⁺

### Step 8. 3-(2-((3-Hydroxy-4-(4-methylpiperazin-1-yl)phenyl)amino)pyridin-4-yl)-4-methoxybenzonitrile

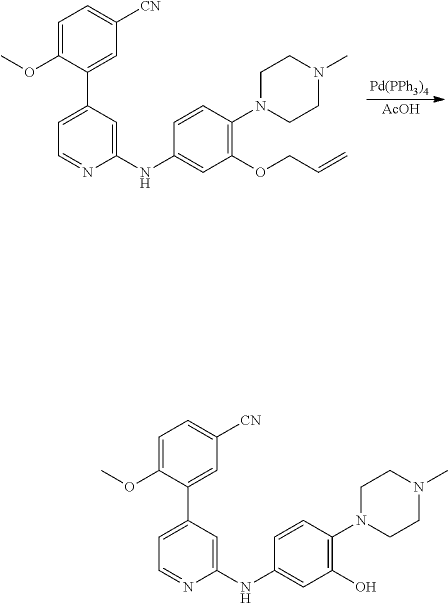

To stirred mixture of 3-(2-((3-(allyloxy)-4-(4-methylpiperazin-1-yl)phenyl)amino)pyridin-4-yl)-4-methoxybenzonitrile (450 mg, 1.0 mmol) in acetic acid (5 ml) was added tetrakis(triphenylphosphine)palladium (380 mg, 0.33 mmol) at room temperature under nitrogen; the mixture was degassed with nitrogen three times. The resulting mixture was refluxed for 1 hour. TLC showed the reaction was complete. The volatiles were evaporated under reduced pressure and the residue was taken-up with methanol (5 ml)-dichloromethane (50 ml). The solid was removed through filtration and the filter cake was washed with methanol (2 ml)-dichloromethane (20 ml). The combined filtrates were concentrated under reduced pressure to give a crude residue which was purified by silica gel flash chromatography (eluted with 2-10% methanol in dichloromethane) to afford 3-(2-((3-hydroxy-4-(4-methylpiperazin-1-yl)phenyl)amino)pyridin-4-yl)-4-methoxybenzonitrile (330 mg, 0.79 mmol, yield 79%) as white solid.

¹HNMR (400 MHz, CD₃OD): δ 2.44 (s, 3H), 2.76 (br, 4H), 3.03 (br, 4H), 3.95 (s, 3H), 6.84-6.89 (m, 2H), 6.95 (s, 1H), 7.02-7.04 (m, 1H), 7.16 (d, J=2.4 Hz, 1H), 7.27-7.29 (m, 1H), 7.70-7.79 (m, 2H), 8.10 (d, J=5.6 Hz, 1H).

LC_MS: (ES⁺): m/z 416.3 [M+H]⁺

### Step 9. 3-(2-[[3-(2-[2-[2-(2-hydroxyethoxy)ethoxy]ethoxy]ethoxy)-4-(4-methylpiperazin-1-yl)phenyl]amino]pyridin-4-yl)-4-methoxybenzonitrile

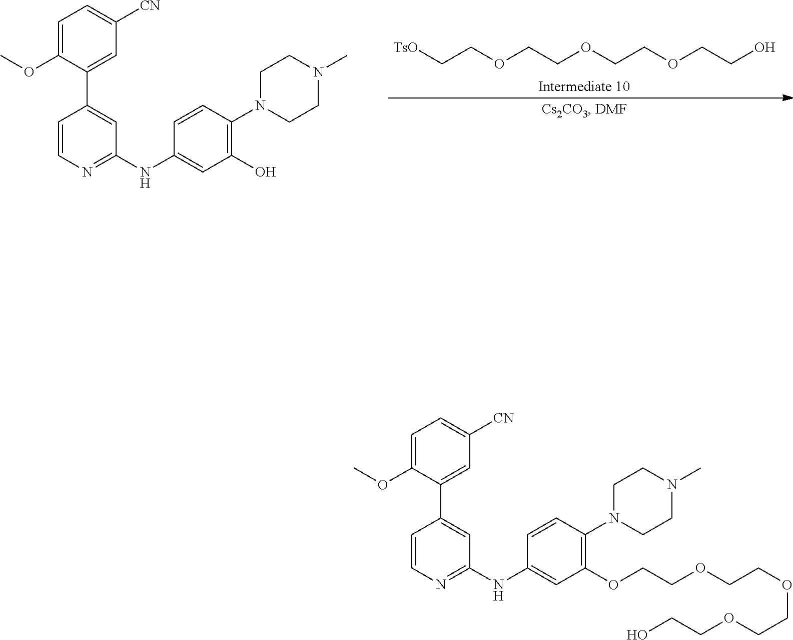

Into a 50-mL round-bottom flask, was placed 3-(2-[[3-hydroxy-4-(4-methylpiperazin-1-yl)phenyl]amino]pyridin-4-yl)-4-methoxybenzonitrile (260.0 mg, 0.63 mmol, 1.00 equiv), 2-[2-[2-(2-[[(4-methylbenzene)sulfonyl]oxy]ethoxy)ethoxy]ethoxy]ethan-1-ol (218.0 mg, 0.63 mmol, 1.00 equiv), Cs₂CO₃ (407.0 mg, 1.25 mmol, 2.00 equiv) in N,N-dimethylformamide (5 mL). The resulting solution was stirred for 2 h at 60° C. The resulting solution was extracted with ethyl acetate (20 mL×2) and washed with brine (30 mL), dried over anhydrous sodium sulfate and concentrated under vacuum. This resulted in 180.0 mg (crude) of 3-(2-[[3-(2-[2-[2-(2-hydroxyethoxy)ethoxy]ethoxy]ethoxy)-4-(4-methylpiperazin-1-yl)phenyl]amino]pyridin-4-yl)-4-methoxybenzonitrile as yellow oil. LC-MS (ES⁺): m/z 592.50 [MH⁺]

### Step 10. 1-(5-[[4-(5-cyano-2-methoxyphenyl)pyridin-2-yl]amino]-2-(4-methylpiperazin-1-yl)phenyl)-1,4,7,10-tetraoxadodecan-12-yl 4-methylbenzene-1-sulfonate

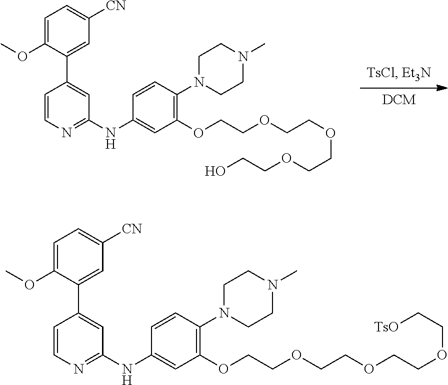

Into a 50-mL round-bottom flask, was placed 3-(2-[[3-(2-[2-[2-(2-hydroxyethoxy)ethoxy]-ethoxy]ethoxy)-4-(4-methylpiperazin-1-yl)phenyl]amino]pyridin-4-yl)-4-methoxybenzonitrile (180.0 mg, 0.30 mmol, 1.00 equiv), TEA (61.0 mg, 0.60 mmol, 2.00 equiv), TsCl (69.0 mg, 0.36 mmol, 1.20 equiv), 4-dimethylaminopyridine (7.4 mg, 0.06 mmol, 0.20 equiv) in dichloromethane (2 mL). Reaction mixture was stirred for 3 h at room temperature and subjected to aqueous work-up with dichloromethane extraction (20 mL×2) and washing of organic phase with saturated sodium chloride (30 mL). The combined organic extracts were dried over anhydrous sodium sulfate and concentrated under vacuum. The residue was applied onto a silica gel column eluting with dichloromethane/methanol (v:v=10:1). The collected fractions were combined and concentrated under vacuum. This resulted in 165.0 mg (73%) of 1-(5-[[4-(5-cyano-2-methoxyphenyl)pyridin-2-yl]amino]-2-(4-methylpiperazin-1-yl)phenyl)-1,4,7,10-tetraoxadodecan-12-yl 4-methylbenzene-1-sulfonate as yellow oil. LC-MS (ES⁺): m/z 746.35 [MH⁺]

### Step 11. tert-Butyl N-[(1S)-1-[[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]carbamoyl]ethyl]-N-methylcarbamate

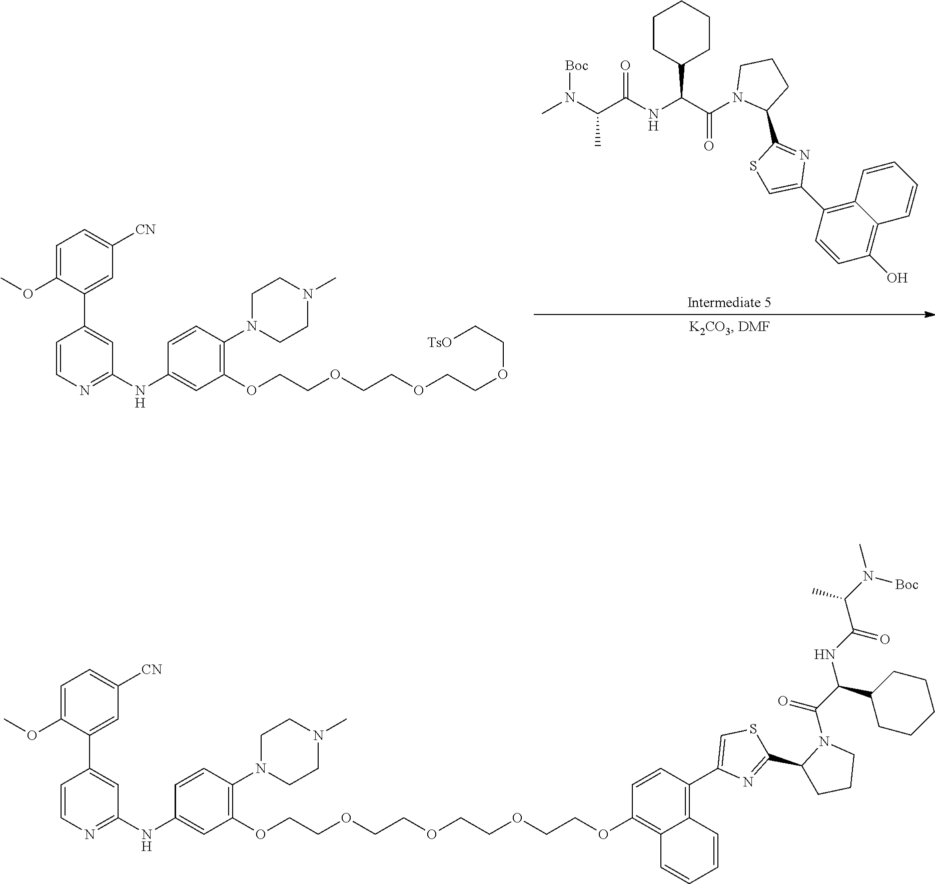

Into a 50-mL round-bottom flask, was placed 1-(5-[[4-(5-cyano-2-methoxyphenyl)pyridin-2-yl]amino]-2-(4-methylpiperazin-1-yl)phenyl)-1,4,7,10-tetraoxadodecan-12-yl 4-methylbenzene-1-sulfonate (85.0 mg, 0.11 mmol, 1.00 equiv), tert-butyl N-[(1S)-1-[[(1S)-1-cyclohexyl-2-[(2S)-2-[4-(4-hydroxynaphthalen-1-yl)-1,3-thiazol-2-yl]pyrrolidin-1-yl]-2-oxoethyl]carbamoyl]ethyl]-N-methylcarbamate (71.0 mg, 0.11 mmol, 1.00 equiv), K₂CO₃ (74.0 mg, 0.23 mmol, 2.00 equiv) in N,N-dimethylformamide (2 mL). The resulting solution was stirred for 4 h at 70° C. The resulting solution was extracted with ethyl acetate (20 mL×2) and washed with saturated sodium chloride (30 mL). The mixture was dried over anhydrous sodium sulfate and concentrated under vacuum. The residue was applied onto a silica gel column eluting with dichloromethane/methanol (v:v=5:1). The collected fractions were combined and concentrated under vacuum. This resulted in 95.0 mg (70%) of tert-butyl N-[(1S)-1-[[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]carbamoyl]ethyl]-N-methylcarbamate as yellow oil.

LC-MS (ES⁺): m/z 1194.85 [MH⁺]

### Step 12. (2S)—N-[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]-2-(methylamino)propanamide

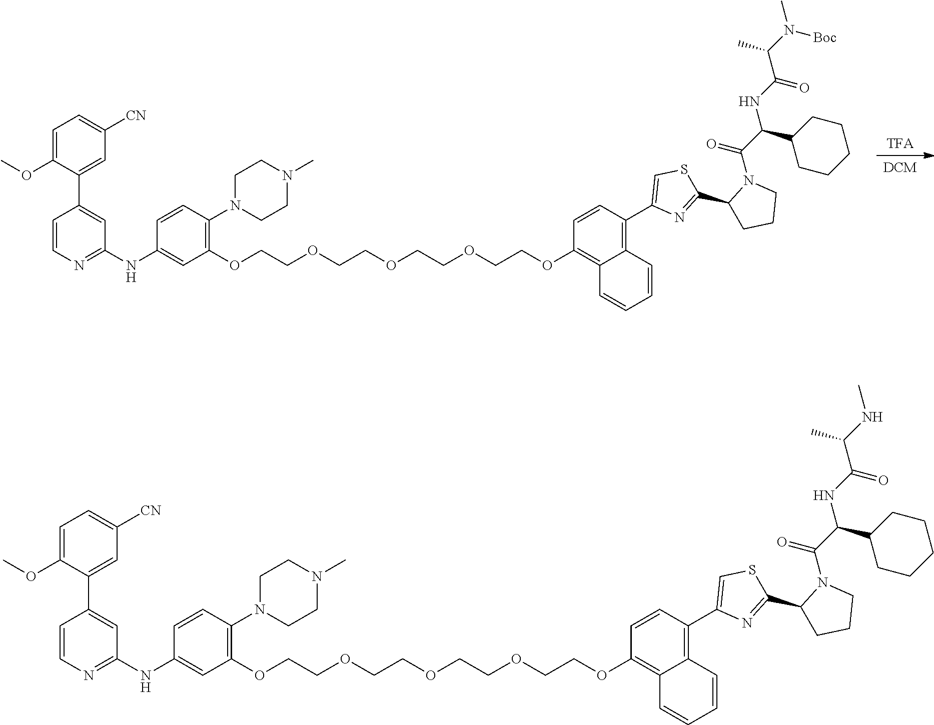

Into a 50-mL round-bottom flask, trifluoroacetic acid (1 mL) was added to a solution of tert-butyl N-[(1S)-1-[[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]carbamoyl]ethyl]-N-methylcarbamate (95.0 mg, 0.08 mmol, 1.00 equiv) in dichloromethane (2 mL). The resulting solution was stirred for 2 h at room temperature. The resulting mixture was concentrated under vacuum. The crude product was purified by Prep-HPLC with the following conditions: Column, XBridge Shield RP18 OBD Column, 5 um, 19*150 mm; mobile phase, Waters (0.05% NH3H2O) and ACN (55.0% ACN up to 75.0% in 8 min); Detector, UV 220 nm. This resulted in 29.4 mg (34%) of (2S)—N-[(1S)-2-[(2S)-2-[4-(4-[[1-(5-[[4-(5-cyano-2-methoxyphenyl)pyridin-2-yl]amino]-2-(4-methylpiperazin-1-yl)phenyl)-1,4,7,10-tetraoxadodecan-12-yl]oxy]naphthalen-1-yl)-1,3-thiazol-2-yl]pyrrolidin-1-yl]-1-cyclohexyl-2-oxoethyl]-2-(methylamino)propanamide as a light yellow solid.

1H NMR (400 MHz, CD₃OD) δ 8.32 (d, J=8.4 Hz, 1H), 8.07 (m, 2H), 7.71 (d, J=8.4 Hz, 1H), 7.64 (s, 1H), 7.49-7.41 (m, 3H), 7.39 (s, 1H), 7.20 (m, 2H), 6.99-6.93 (m, 1H), 6.89-6.81 (m, 3H), 6.80-6.78 (m, 1H), 5.55 (m, 1H), 4.59 (m, 1H), 4.33 (m, 2H), 4.16 (m, 2H), 4.01-3.64 (m, 14H), 3.19-3.03 (s, 3H), 2.98-2.86 (m, 4H), 2.56 (m, 6H), 2.39-2.02 (m, 4H), 1.88-1.54 (m, 6H), 1.42 (d, J=8.4 Hz, 3H), 1.23-1.06 (m, 6H); LC-MS (ES+): m/z 1094.55 [MH+]

Using procedures analogous to those described above for Example 4, the following compounds were prepared:

### Example 5 (2S)—N-[(1S)-2-[(2S)-2-[4-[4-(2-[2-[2-(5-[[4-(5-cyano-2-methoxyphenyl)pyridin-2-yl]amino]-2-(4-methylpiperazin-1-yl)phenoxy)ethoxy]ethoxy]ethoxy)naphthalen-1-yl]-1,3-thiazol-2-yl]pyrrolidin-1-yl]-1-cyclohexyl-2-oxoethyl]-2-(methylamino)propanamide

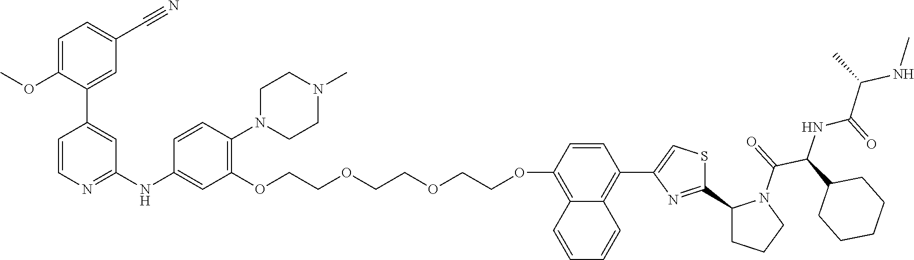

1H NMR (400 MHz, CD₃OD) δ8.54-8.46 (m, 1H), 8.32 (d, J=8.4 Hz, 1H), 8.07 (m, 2H), 7.71 (d, J=8.4 Hz, 1H), 7.64 (s, 1H), 7.49-7.41 (m, 4H), 7.26 (s, 1H), 7.20 (d, J=8.4 Hz, 1H), 6.99-6.80 (m, 5H), 5.55 (d, J=8.4 Hz, 1H), 4.59 (m, 1H), 4.33 (m, 2H), 4.16 (m, 2H), 4.01-3.64 (m, 14H), 3.20-3.08 (m, 3H), 2.98 (m, 4H), 2.56 (s, 6H), 2.39-2.02 (m, 4H), 1.88-1.54 (m, 6H), 1.42 (d, J=8.4 Hz, 3H), 1.23-1.06 (m, 6H); LC-MS (ES⁺): m/z 1050.52 [MH⁺]

### Example 6

### (2S)—N-[(1S)-2-{6-[2-(4-{[1-(5-{[4-(5-cyano-2-methoxyphenyl)pyridin-2-yl]amino}-2-(4-meth ylpiperazin-1-yl)phenyl)-1,4,7,10-tetraoxadodecan-12-yl]oxy}phenyl)ethyl]-octahydro-1H-pyrrolo[2,3-c]pyridin-1-yl}-1-cyclohexyl-2-oxoethyl]-2-(methylamino)propanamide

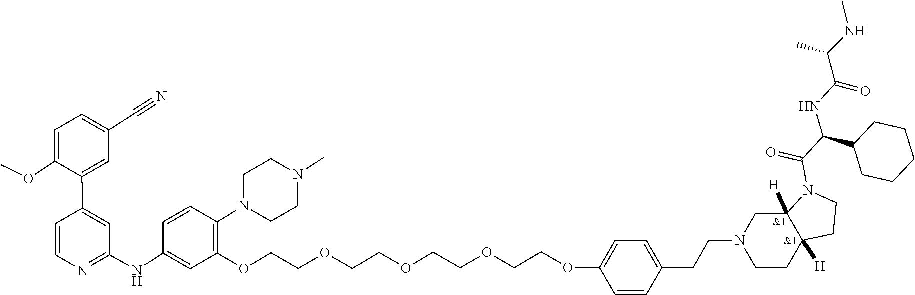 [SMILES: CN[C@@H](C)C(=O)N[C@H](C(=O)N1CC[C@@H]2CCN(CCc3ccc(OCCOCCOCCOCCOc4cc(Nc5cc(-c6cc(C#N)ccc6OC)ccn5)ccc4N4CCN(C)CC4)cc3)C[C@@H]21)C1CCCCC1]

1H NMR (400 MHz, CD3OD) δ 8.60-8.49 (m, 1H), 8.12 (d, J=8.4 Hz, 1H), 7.88 (d, J=8.4 Hz, 1H), 7.71 (s, 1H), 7.33 (s, 1H), 7.26 (d, J=8.4 Hz, 1H), 7.13-7.01 (m, 3H), 6.99-6.80 (m, 5H), 4.39 (d, J=8.4 Hz, 1H), 4.16 (m, 2H), 4.05 (m, 2H), 3.98-3.79 (m, 9H), 3.83-3.68 (m, 10H), 3.51-3.14 (m, 9H), 3.08-2.80 (m, 8H), 2.62 (s, 3H), 2.58-2.32 (m, 2H), 2.29-2.08 (m, 2H), 1.91-1.67 (m, 8H), 1.48-1.38 (m, 3H), 1.35-1.01 (m, 5H); LC-MS (ES⁺): m/z 1044.80 [MH⁺]

### Example 7 (2S)—N-[(1S)-2-[6-[2-[4-(2-[2-[2-(5-[[4-(5-cyano-2-methoxyphenyl)-pyridin-2-yl]amino]-2-(4-methylpiperazin-1-yl)phenoxy)ethoxy]ethoxy]ethoxy)-phenyl]ethyl]-octahydro-1H-pyrrolo[2,3-c]pyridin-1-yl]-1-cyclohexyl-2-oxoethyl]-2-(methylamino)propanamide

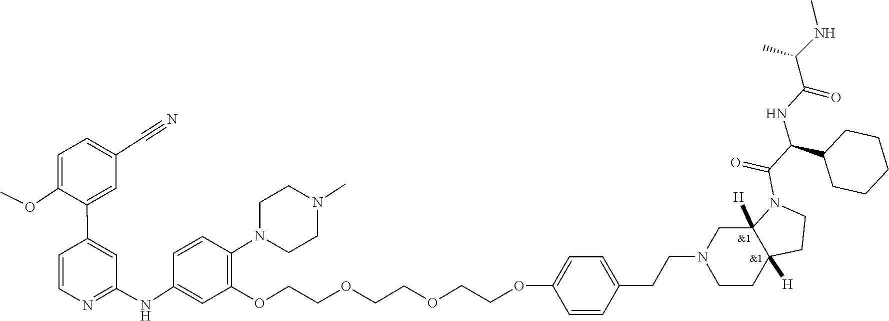 [SMILES: CN[C@@H](C)C(=O)N[C@H](C(=O)N1CC[C@@H]2CCN(CCc3ccc(OCCOCCOCCOc4cc(Nc5cc(-c6cc(C#N)ccc6OC)ccn5)ccc4N4CCN(C)CC4)cc3)C[C@@H]21)C1CCCCC1]

1H NMR (400 MHz, MeOD) δ8.12 (d, J=8.4 Hz, 1H), 7.88 (d, J=8.4 Hz, 1H), 7.71 (m, 1H), 7.33 (m, 1H), 7.26 (d, J=8.4 Hz, 1H), 7.13-7.01 (m, 3H), 6.95-6.87 (m, 2H), 6.85-6.78 (m, 3H), 4.39 (d, J=8.4 Hz, 1H), 4.33-4.15 (m, 3H), 4.05 (m, 2H), 4.01-3.85 (m, 6H), 3.83-3.68 (m, 8H), 3.59-3.39 (m, 2H), 3.25 (s, 6H), 3.11-2.88 (m, 2H), 2.78 (d, J=8.4 Hz, 7H), 2.61 (m, 3H), 2.58-2.32 (m, 2H), 2.29-2.08 (m, 3H), 1.91-1.67 (m, 7H), 1.44-1.35 (m, 3H), 1.32-1.06 (m, 5H); LC-MS (ES+): m/z 1000.70 [MH+]

### Example 8 5-(4-[[1-(4-[2-[1-[(2S)-2-cyclohexyl-2-[(2S)-2-(methylamino)propanamido]acetyl]-octahydro-1H-pyrrolo[2,3-c]pyridin-6-yl]ethyl]phenyl)-1,4,7,10,13-pentaoxapentadecan-15-yl]oxy]phenyl)-N-[(4,6-dimethyl-2-oxo-1,2-dihydropyridin-3-yl)methyl]-3-[ethyl(oxan-4-yl)amino]-2-methylbenzamide

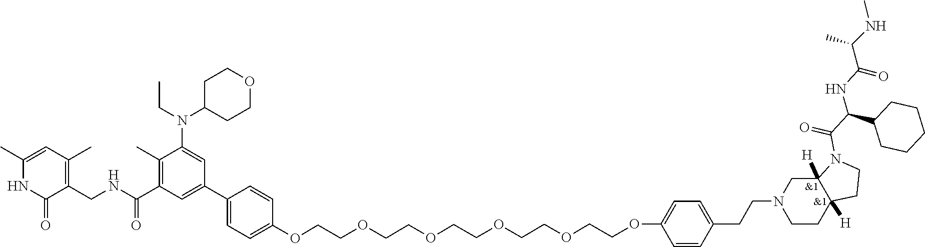 [SMILES: CCN(c1cc(-c2ccc(OCCOCCOCCOCCOCCOc3ccc(CCN4CC[C@H]5CCN(C(=O)[C@@H](NC(=O)[C@H](C)NC)C6CCCCC6)[C@H]5C4)cc3)cc2)cc(C(=O)NCc2c(C)cc(C)[nH]c2=O)c1C)C1CCOCC1]

### Step 1. 14-[[(4-methylbenzene)sulfonyl]oxy]-3,6,9,12-tetraoxatetradecan-1-ol

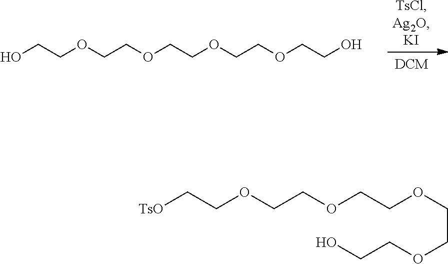

Into a 250-mL round-bottom flask, was placed a solution of 3,6,9,12-tetraoxatetradecane-1,14-diol (5.0 g, 20.98 mmol, 1.00 equiv) in dichloromethane (100 mL), Ag₂O (7.3 g, 31.50 mmol, 1.50 equiv), 4-methylbenzene-1-sulfonyl chloride (4.0 g, 20.99 mmol, 1.00 equiv), KI (695.0 mg, 4.19 mmol, 0.30 equiv). The resulting solution was stirred overnight at room temperature. The reaction was then quenched by the addition of water (80 mL). The solids were filtered out. The resulting solution was extracted with dichloromethane (60 ml×3) and the organic layers combined. The resulting mixture was washed with brine (60 ml×1), dried over anhydrous sodium sulfate and concentrated under reduced pressure. The residue was applied onto a silica gel column eluting with ethyl acetate/petroleum ether (1/1). This resulted in 4.2 g (51%) of 14-[[(4-methylbenzene)sulfonyl]oxy]-3,6,9,12-tetraoxatetradecan-1-ol as yellow oil.

### Step 2. 1-[4-(tetramethyl-1,3,2-dioxaborolan-2-yl)phenyl]-1,4,7,10,13-pentaoxapentadecan-15-ol

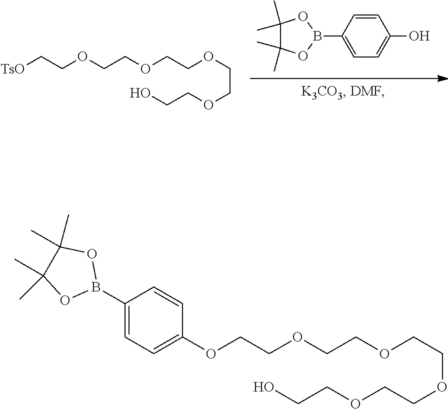

Into a 250-mL round-bottom flask, was placed a solution of 14-[[(4-methylbenzene)sulfonyl]oxy]-3,6,9,12-tetraoxatetradecan-1-ol (2.0 g, 5.10 mmol, 1.00 equiv) in N,N-dimethylformamide (150 mL), potassium carbonate (2.2 g, 15.92 mmol, 3.00 equiv), 4-(tetramethyl-1,3,2-dioxaborolan-2-yl)phenol (1.5 g, 6.82 mmol, 1.50 equiv). The resulting solution was stirred for 16 h at 60° C. in an oil bath. The reaction was then quenched by the addition of water (100 mL) and extracted with ethyl acetate (50 mL×3). The combined organic layers were washed with brine (80 mL×2), dried over anhydrous sodium sulfate and concentrated under reduced pressure. The residue was applied onto a silica gel column eluting with ethyl acetate/petroleum ether (1/1). This resulted in 1.2 g (53%) of 1-[4-(tetramethyl-1,3,2-dioxaborolan-2-yl)phenyl]-1,4,7,10,13-pentaoxapentadecan-15-ol as yellow oil.

### Step 3. N-[(4,6-dimethyl-2-oxo-1,2-dihydropyridin-3-yl)methyl]-3-[ethyl(oxan-4-yl)amino]-5-[4-[(14-hydroxy-3,6,9,12-tetraoxatetradecan-1-yl)oxy]phenyl]-2-methylbenzamide

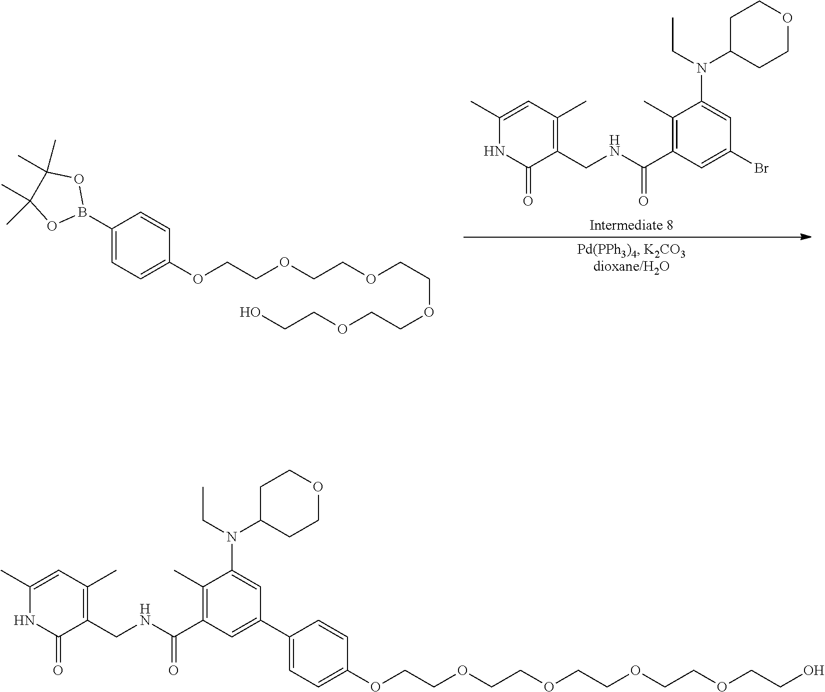

Into a 100-mL 3-necked round-bottom flask purged and maintained with an inert atmosphere of nitrogen, was placed a solution of 1-[4-(tetramethyl-1,3,2-dioxaborolan-2-yl)phenyl]-1,4,7,10,13-pentaoxapentadecan-15-ol (300.0 mg, 0.68 mmol, 1.00 equiv) in dioxane/water (40/10 mL), potassium carbonate (282.0 mg, 2.04 mmol, 3.00 equiv), 5-(4-bromophenyl)-N-[(4,6-dimethyl-2-oxo-1,2-dihydropyridin-3-yl)methyl]-3-[ethyl(oxan-4-yl)amino]-2-methylbenzamide (323.0 mg, 0.58 mmol, 1.00 equiv), Pd(PPh₃)₄ (79.0 mg, 0.07 mmol, 0.10 equiv). The resulting solution was stirred for 4 h at 100° C. in an oil bath. The reaction was then quenched by the addition of water (80 mL) and extracted with ethyl acetate (40 mL×3). The combined organic layers was washed with brine (60 mL×2), dried over anhydrous sodium sulfate and concentrated under vacuum. The residue was applied onto a silica gel column eluting with dichloromethane/methanol (10/1). This resulted in 400.0 mg (83%) of N-[(4,6-dimethyl-2-oxo-1,2-dihydropyridin-3-yl)methyl]-3-[ethyl(oxan-4-yl)amino]-5-[4-[(14-hydroxy-3,6,9,12-tetraoxatetradecan-1-yl)oxy]phenyl]-2-methylbenzamide as a yellow solid.

LC-MS (ES+): m/z 355.60 [MH+]

### Step 4. N-[[2-(benzyloxy)-4,6-dimethylpyridin-3-yl]methyl]-3-[ethyl(oxan-4-yl)amino]-5-[4-[(14-hydroxy-3,6,9,12-tetraoxatetradecan-1-yl)oxy]phenyl]-2-methylbenzamide

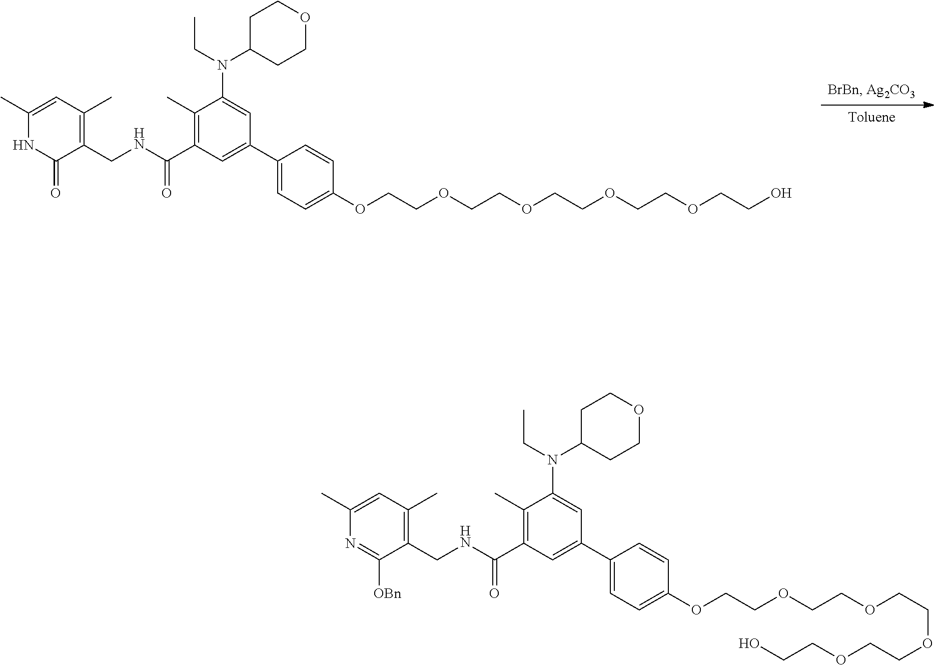

Into a 100-mL round-bottom flask, was placed a solution of N-[(4,6-dimethyl-2-oxo-1,2-dihydropyridin-3-yl)methyl]-3-[ethyl(oxan-4-yl)amino]-5-[4-[(14-hydroxy-3,6,9,12-tetraoxatetradecan-1-yl)oxy]phenyl]-2-methylbenzamide (350.0 mg, 0.49 mmol, 1.00 equiv) in methylbenzene (20 mL), Ag₂CO₃ (273.0 mg, 0.99 mmol, 2.00 equiv), BnBr (256.0 mg, 1.50 mmol, 3.00 equiv). The resulting solution was stirred for 2 h at 80° C. in an oil bath. The reaction was then quenched by the addition of water (20 mL) and extracted with ethyl acetate (20 mL×3). The combined organic layers was washed with brine (20 mL×2), dried over anhydrous sodium sulfate and concentrated under vacuum. The residue was applied onto a silica gel column eluting with dichloromethane/methanol (10/1). This resulted in 380.0 mg (92%) of N-[[2-(benzyloxy)-4,6-dimethylpyridin-3-yl]methyl]-3-[ethyl(oxan-4-yl)amino]-5-[4-[(14-hydroxy-3,6,9,12-tetraoxatetradecan-1-yl)oxy]phenyl]-2-methylbenzamide as yellow oil.

### Step 5. 1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl 4-methylbenzene-1-sulfonate

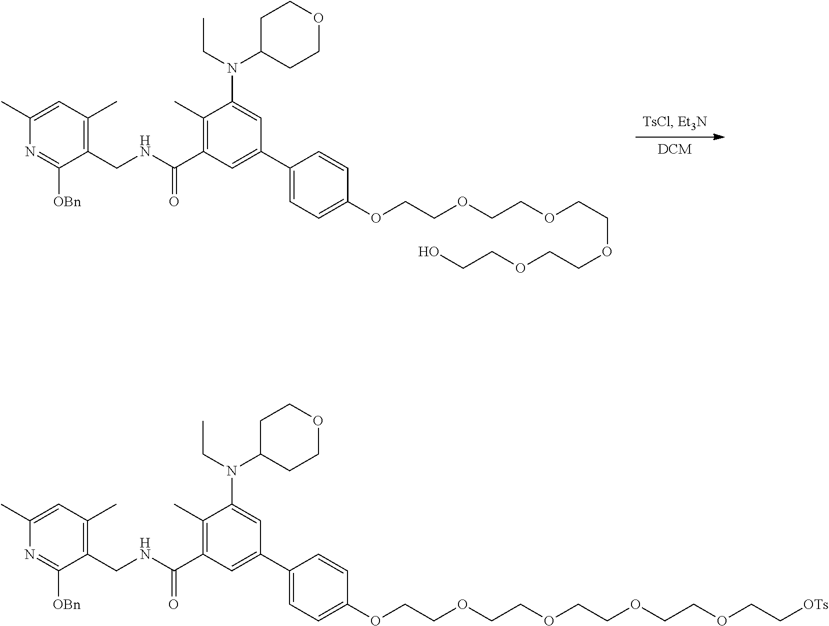

Into a 100-mL round-bottom flask, was placed a solution of N-[[2-(benzyloxy)-4,6-dimethylpyridin-3-yl]methyl]-3-[ethyl(oxan-4-yl)amino]-5-[4-[(14-hydroxy-3,6,9,12-tetraoxatetradecan-1-yl)oxy]phenyl]-2-methylbenzamide (400.0 mg, 0.50 mmol, 1.00 equiv) in dichloromethane (20 mL), triethylamine (152.0 mg, 1.50 mmol, 3.00 equiv), TsCl (192.0 mg, 1.01 mmol, 2.00 equiv), 4-dimethylaminopyridine (8.0 mg, 0.07 mmol, 0.10 equiv). The resulting solution was stirred for 3 h at room temperature. The reaction was then quenched by the addition of water (30 mL) and extracted with dichloromethane (20 mL×3). The combined organic layers were washed with brine (20 mL×3), dried over anhydrous sodium sulfate and concentrated under vacuum. The residue was applied onto a silica gel column eluting with ethyl acetate. This resulted in 270.0 mg (57%) of 1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl 4-methylbenzene-1-sulfonate as yellow oil.

### Step 6. tert-Butyl N-[(1S)-1-[[(1S)-2-[(3aR,7aS)-6-(2-[4-[(1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl)oxy]phenyl]ethyl)-octahydro-1H-pyrrolo[2,3-c]pyridin-1-yl]-1-cyclohexyl-2-oxoethyl]carbamoyl]ethyl]-N-methylcarbamate

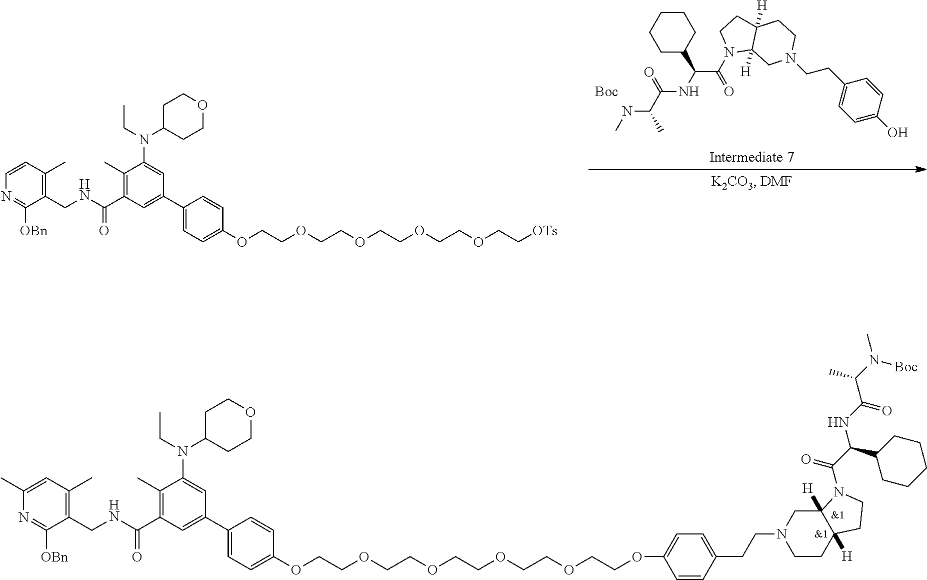

Into a 50-mL round-bottom flask, was placed a solution of 1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl 4-methylbenzene-1-sulfonate (100.0 mg, 0.10 mmol, 1.00 equiv) in N,N-dimethylformamide (15 mL), K₂CO₃ (103.0 mg, 0.32 mmol, 3.00 equiv), tert-butyl N-[(1S)-1-[[(1S)-2-[(3aR,7aS)-6-[2-(4-hydroxyphenyl)ethyl]-octahydro-1H-pyrrolo[2,3-c]pyridin-1-yl]-1-cyclohexyl-2-oxoethyl]carbamoyl]ethyl]-N-methylcarbamate (40.0 mg, 0.07 mmol, 1.00 equiv). The resulting solution was stirred for 4 h at 60° C. in an oil bath. The reaction was then quenched by the addition of water (15 mL) and extracted with ethyl acetate (15 mL×3). The combined organic layers were washed with brine (15 mL×2), dried over anhydrous sodium sulfate and concentrated under vacuum. The residue was applied onto a silica gel column eluting with dichloromethane/methanol (10/1). This resulted in 55.0 mg (39%) of tert-butyl N-[(1S)-1-[[(1S)-2-[6-(2-[4-[(1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl)oxy]phenyl]ethyl)-octahydro-1H-pyrrolo[2,3-c]pyridin-1-yl]-1-cyclohexyl-2-oxoethyl]carbamoyl]ethyl]-N-methylcarbamate as yellow oil.

### Step 7. tert-Butyl N-[(1S)-1-[[(1S)-1-cyclohexyl-2-[(2S)-2-[4-(4-[[1-(4-[3-[ethyl(oxan-4-yl)amino]-5-[[(2-hydroxy-4,6-dimethyl-1,2-dihydropyridin-3-yl)methyl]carbamoyl]-4-methylphenyl]phenyl)-1,4,7,10,13-pentaoxapentadecan-15-yl]oxy]naphthalen-1-yl)-1,3-thiazol-2-yl]pyrrolidin-1-yl]-2-oxoethyl]carbamoyl]ethyl]-N-methylcarbamate

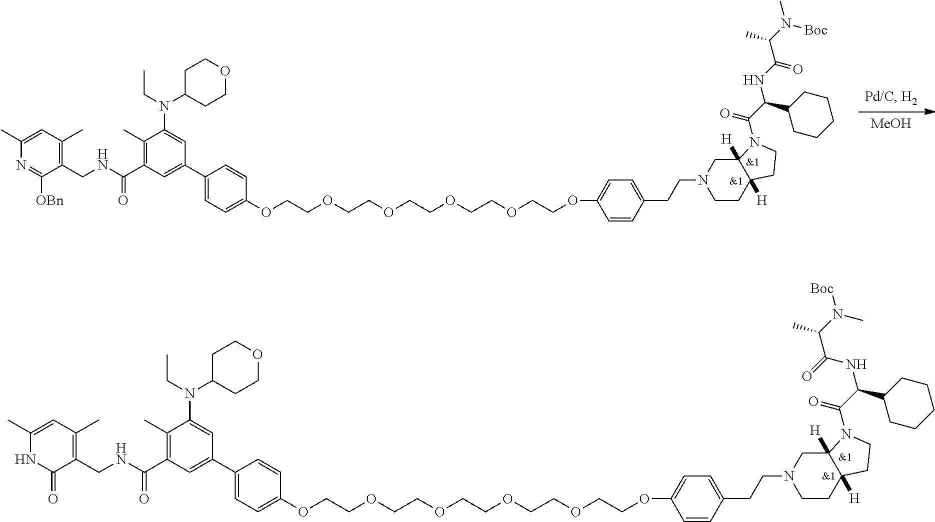 [SMILES: "MeOH" ; CCN(c1cc(-c2ccc(OCCOCCOCCOCCOCCOc3ccc(CCN4CC[C@H]5CCN(C(=O)[C@@H](NC(=O)[C@H](C)N(C)C(=O)OC(C)(C)C)C6CCCCC6)[C@H]5C4)cc3)cc2)cc(C(=O)NCc2c(C)cc(C)nc2OCc2ccccc2)c1C)C1CCOCC1 ; "Pd/C, H2" ; CCN(c1cc(-c2ccc(OCCOCCOCCOCCOCCOc3ccc(CCN4CC[C@H]5CCN(C(=O)[C@@H](NC(=O)[C@H](C)N(C)C(=O)OC(C)(C)C)C6CCCCC6)[C@H]5C4)cc3)cc2)cc(C(=O)NCc2c(C)cc(C)[nH]c2=O)c1C)C1CCOCC1]

In a 50 ml round bottom flask, Pd/C (10%, 200 mg) was added to a solution of tert-butyl N-[(1S)-1-[[(1S)-2-[(2S)-2-(4-[4-[(1-[4-[3-([[2-(benzyloxy)-4,6-dimethylpyridin-3-yl]methyl]carbamoyl)-5-[ethyl(oxan-4-yl)amino]-4-methylphenyl]phenyl]-1,4,7,10,13-pentaoxapentadecan-15-yl)oxy]naphthalen-1-yl]-1,3-thiazol-2-yl)pyrrolidin-1-yl]-1-cyclohexyl-2-oxoethyl]carbamoyl]ethyl]-N-methylcarbamate (55 mg) in 10 mL MeOH. The reaction flask was vacuumed and charged with a hydrogen balloon. The resulting mixture was stirred for 4 h at room temperature under hydrogen atmosphere. After the reaction was done, the reaction mixture was filtered through a Celite pad and the filtrate was concentrated under reduced pressure. This resulted in 30.0 mg (80%) of tert-butyl N-[(1S)-1-[[(1S)-1-cyclohexyl-2-[(2S)-2-[4-(4-[[1-(4-[3-[ethyl(oxan-4-yl)amino]-5-[[(2-hydroxy-4,6-dimethyl-1,2-dihydropyridin-3-yl)methyl]carbamoyl]-4-methylphenyl]phenyl)-1,4,7,10,13-pentaoxapentadecan-15-yl]oxy]naphthalen-1-yl)-1,3-thiazol-2-yl]pyrrolidin-1-yl]-2-oxoethyl]carbamoyl]ethyl]-N-methylcarbamate as yellow solid.

### Step 8. 5-(4-[[1-(4-[2-[1-[(2S)-2-cyclohexyl-2-[(2S)-2-(methylamino)propanamido]acetyl]-octahydro-1H-pyrrolo[2,3-c]pyridin-6-yl]ethyl]phenyl)-1,4,7,10,13-pentaoxapentadecan-15-yl]oxy]phenyl)-N-[(4,6-dimethyl-2-oxo-1,2-dihydropyridin-3-yl)methyl]-3-[ethyl(oxan-4-yl)amino]-2-methylbenzamide

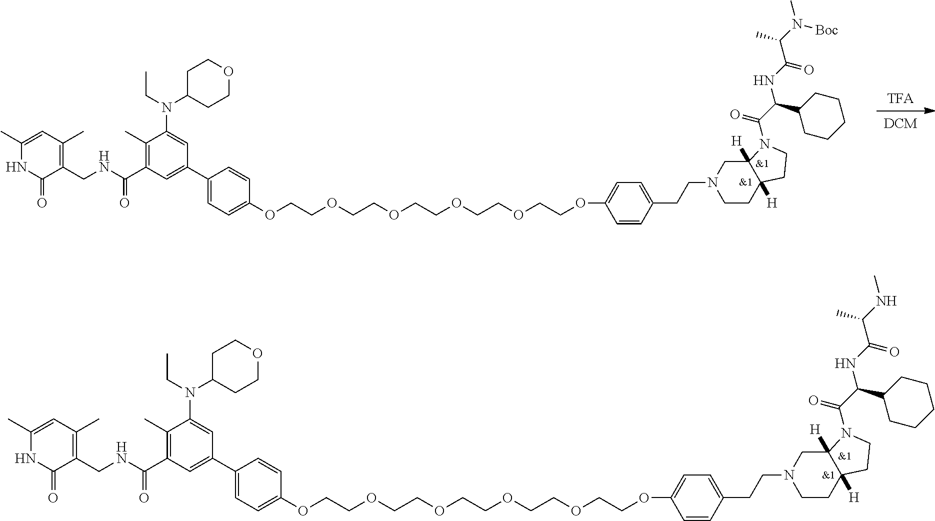

Into a 50-mL round-bottom flask, was placed a solution of tert-butyl N-[(1S)-1-[[(1S)-2-[(3aR,7aS)-6-[2-[4-([1-[4-(3-[[(4,6-dimethyl-2-oxo-1,2-dihydropyridin-3-yl)methyl]carbamoyl]-5-[ethyl(oxan-4-yl)amino]-4-methylphenyl)phenyl]-1,4,7,10,13-pentaoxapentadecan-15-yl]oxy)phenyl]ethyl]-octahydro-1H-pyrrolo[2,3-c]pyridin-1-yl]-1-cyclohexyl-2-oxoethyl]carbamoyl]ethyl]-N-methylcarbamate (30.0 mg, 0.02 mmol, 1.00 equiv) in dichloromethane (5 mL), trifluoroacetic acid (2.0 mL). The resulting solution was stirred for 2 h at room temperature. The resulting mixture was concentrated under vacuum. The crude product was purified by Prep-HPLC with the following conditions: Column, XBridge Shield RP18 OBD Column, 5 um, 19*150 mm; mobile phase, Water (0.05% NH3H2O) and ACN (42.0% ACN up to 55.0% in 10 min); Detector, UV 220 nm. This resulted in 11.2 mg (41%) of 5-(4-[[1-(4-[2-[1-[(2S)-2-cyclohexyl-2-[(2S)-2-(methylamino)propanamido]acetyl]-octahydro-1H-pyrrolo[2,3-c]pyridin-6-yl]ethyl]phenyl)-1,4,7,10,13-pentaoxapentadecan-15-yl]oxy]phenyl)-N-[(4,6-dimethyl-2-oxo-1,2-dihydropyridin-3-yl)methyl]-3-[ethyl(oxan-4-yl)amino]-2-methylbenzamide as a white solid.

¹H NMR (400 MHz, CD₃OD): δ7.60-7.50 (m, 2H), 7.41-7.40 (s, 1H), 7.27 (s, 1H), 7.19-6.98 (m, 4H), 6.90-6.83 (m, 2H), 6.11 (m, 1H), 4.88 (s, 2H), 4.49 (m, 1H), 4.14-4.05 (m, 5H), 4.05-3.79 (m, 7H), 3.70-3.64 (m, 13H), 3.35-3.30 (s, 1H), 3.15-3.00 (m, 4H), 2.80-2.60 (m, 3H), 2.60-2.50 (m, 3H), 2.45-2.35 (m, 3H), 2.30 (s, 6H), 2.24-2.10 (m, 5H), 2.10-1.95 (m, 2H), 1.90-1.50 (m, 14H), 1.40-1.15 (m, 10H), 0.91-0.85 (m, 3H); LC-MS (ES+): m/z 1162.75 [MH+]

Using procedures analogous to those described above for Example 8, the following compounds have been prepared:

### Example 9 5-[4-(2-[2-[2-(4-[2-[1-[(2S)-2-cyclohexyl-2-[(2S)-2-(methylamino)propanamido]acetyl]-octahydro-1H-pyrrolo[2,3-c]pyridin-6-yl]ethyl]phenoxy)ethoxy]ethoxy]ethoxy)phenyl]-N-[(4,6-dimethyl-2-oxo-1,2-dihydropyridin-3-yl)methyl]-3-[ethyl(oxan-4-yl)amino]-2-methylbenzamide

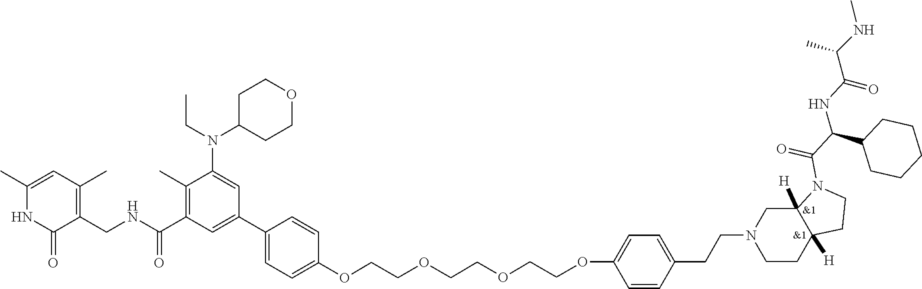 [SMILES: CCN(c1cc(-c2ccc(OCCOCCOCCOc3ccc(CCN4CC[C@H]5CCN(C(=O)[C@@H](NC(=O)[C@H](C)NC)C6CCCCC6)[C@H]5C4)cc3)cc2)cc(C(=O)NCc2c(C)cc(C)[nH]c2=O)c1C)C1CCOCC1]

1H NMR (400 MHz, CD₃OD): δ7.52-7.49 (d, J=8.8 Hz, 2H), 7.42 (d, J=1.6 Hz, 1H), 7.29 (s, 1H), 7.09-6.99 (m, 4H), 6.85-6.83 (m, 2H), 6.12 (s, 1H), 4.50 (s, 2H), 4.16-4.14 (m, 3H), 4.09-4.07 (m, 2H), 3.94-3.82 (m, 7H), 3.74 (m, 4H), 3.37-3.32 (m, 3H), 3.16-3.13 (m, 5H), 2.85-2.70 (m, 3H), 2.60-2.50 (m, 2H), 2.33-2.30 (m, 3H), 2.28-2.26 (m, 6H), 2.25 (s, 3H), 2.20-2.00 (m, 4H), 1.88-1.85 (m, 3H), 1.78-1.64 (m, 10H), 1.24-1.21 (d, J=14.0 Hz, 6H), 1.20-1.00 (m, 2H), 0.92-0.89 (t, J=7.0 Hz, 3H); LC-MS (ES⁺): m/z 1074.75 [MH⁺]

### Example 10: 5-[4-[2-(2-[2-[(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)oxy]ethoxy]ethoxy)ethoxy]phenyl]-N-[(4,6-dimethyl-2-oxo-1,2-dihydropyridin-3-yl)methyl]-3-[ethyl(oxan-4-yl)amino]-2-methylbenzamide

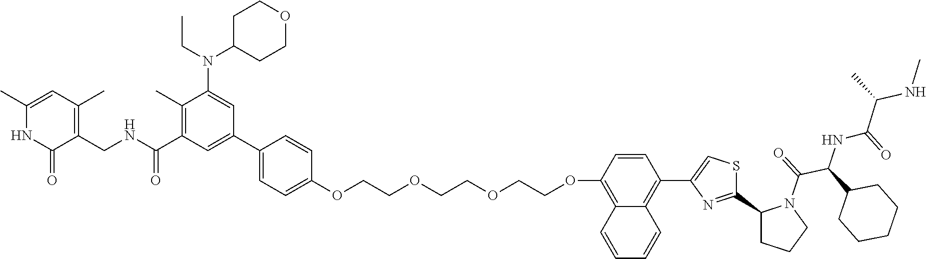

¹H NMR (400 MHz, CD₃OD): δ 8.35 (m, 1H), 8.10 (m, 1H), 7.50-7.43 (m, 6H), 7.38 (s, 1H), 7.25 (m, 1H), 6.95-6.92 (m, 3H), 6.09 (s, 1H), 5.50 (m, 1H), 4.60 (m, 1H), 4.48 (s, 2H), 4.34-4.32 (d, J=4.40 Hz, 2H), 4.10-4.08 (m, 2H), 4.03-4.01 (m, 2H), 3.90-3.76 (m, 10H), 3.32-3.30 (m, 3H), 3.20-3.10 (m, 4H), 2.38 (s, 3H), 2.33-2.30 (d, J=11.2 Hz, 6H), 2.22-2.16 (m, 4H), 1.80-1.59 (m, 11H), 1.28-1.24 (m, 5H), 1.13-1.11 (m, 4H), 0.88-0.85 (t, J=7.0 Hz, 3H);

LC-MS (ES⁺): m/z 1124.70 [MH⁺]

### Example 11 5-(4-[[1-(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)-1,4,7,10,13-pentaoxapentadecan-15-yl]oxy]phenyl)-N-[(4,6-dimethyl-2-oxo-1,2-dihydropyridin-3-yl)methyl]-3-[ethyl(oxan-4-yl)amino]-2-methylbenzamide

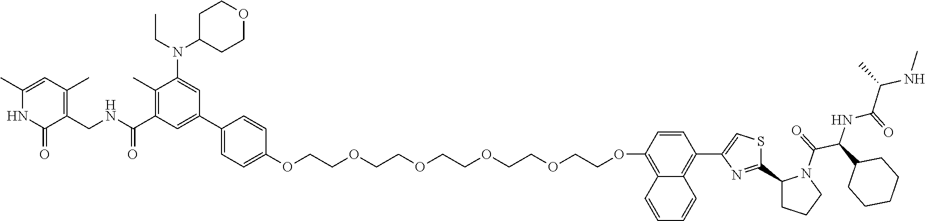

¹H NMR (400 MHz, CD₃OD): δ 8.40 (m, 1H), 8.10-8.00 (m, 1H), 7.60-7.45 (m, 7H), 7.39 (s, 1H), 7.26 (s, 1H), 6.95-6.93 (m, 3H), 6.09 (s, 1H), 5.50 (s, 1H), 4.60 (m, 1H), 4.48 (s, 2H), 4.32 (m, 2H), 4.08-4.07 (m, 2H), 4.00-3.99 (m, 3H), 3.98-3.90 (m, 3H), 3.78-3.77 (m, 5H), 3.76-3.75 (m, 3H), 3.69-3.60 (m, 10H), 3.30-3.12 (m, 4H), 2.40-2.38 (m, 7H), 2.30 (s, 4H), 2.22 (s, 6H), 2.15-2.00 (m, 2H), 1.90-1.50 (m, 12H), 0.89-0.85 (m, 4H); LC-MS (ES⁺): m/z 1212.7 [MH⁺]

### Example 12 (2S)—N-[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]-2-(methylamino)propanamide

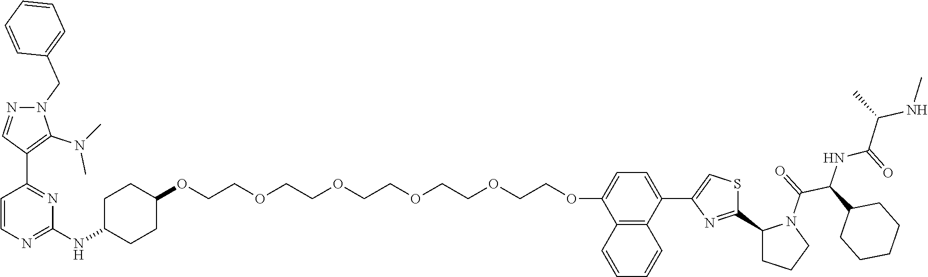 [SMILES: CN[C@@H](C)C(=O)N[C@H](C(=O)N1CCC[C@H]1c1nc(-c2ccc(OCCOCCOCCOCCOCCO[C@H]3CC[C@H](Nc4nccc(-c5cnn(Cc6ccccc6)c5N(C)C)n4)CC3)c3ccccc23)cs1)C1CCCCC1]

### Step 1. 2-[[(1r,4r)-4-(dibenzylamino)cyclohexyl]oxy]ethyl 4-methylbenzene-1-sulfonate

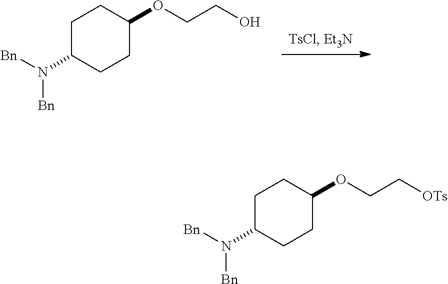

The experiment was run using procedure described for step 5 of Example 8. The starting 2-[[(1r,4r)-4-(dibenzylamino)cyclohexyl]oxy]ethanol was prepared as described previously by Takahashi, F. et al. in US 20130150364.

### Step 2. 1-[(1r,4r)-4-(dibenzylamino)cyclohexyl]-1,4,7,10,13-pentaoxapentadecan-15-ol

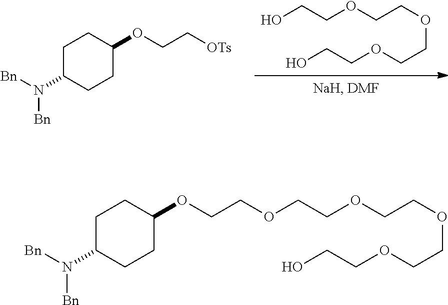

Into a 100-mL round-bottom flask, sodium hydride (72.9 mg, 3.04 mmol, 1.50 equiv) was added to a solution of 2-2-[2-(2-hydroxyethoxy)ethoxy]ethoxyethan-1-ol (785.4 mg, 4.04 mmol, 2.00 equiv) in N,N-dimethylformamide (10 mL) at 0° C. in a water/ice bath under nitrogen atmosphere. The mixture was stirred at room temperature for 30 min. Then 2-[[(1r,4r)-4-(dibenzylamino)cyclohexyl]oxy]ethyl 4-methylbenzene-1-sulfonate (1.0 g, 2.03 mmol, 1.00 equiv) was added. The resulting solution was stirred for 4 h at room temperature. The reaction was then quenched by water/ice (30 mL) and extracted with ethyl acetate (30 mL×3). The combined organic layer was washed with brine (30 mL), dried over anhydrous sodium sulfate and concentrated under vacuum. The residue was applied onto a silica gel column eluting with ethyl acetate/petroleum ether (v:v=1:1). This resulted in 480.0 mg (46%) of 1-[(1r,4r)-4-(dibenzylamino)cyclohexyl]-1,4,7,10,13-pentaoxapentadecan-15-ol as yellow oil.

LC-MS (ES⁺): m/z 516.35 [MH⁺]

### Step 3. 1-[(1r,4r)-4-aminocyclohexyl]-1,4,7,10,13-pentaoxapentadecan-15-ol

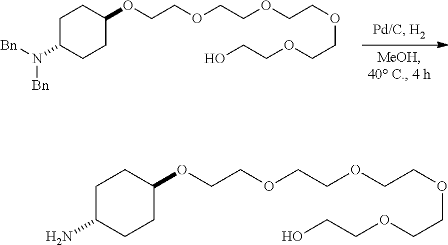 [SMILES: OCCOCCOCCOCCOCCO[C@H]1CC[C@H](N(Cc2ccccc2)Cc2ccccc2)CC1 ; "MeOH,40° C., 4 h" ; "Pd/C, H2" ; N[C@H]1CC[C@H](OCCOCCOCCOCCOCCO)CC1]

Into a 100-mL round-bottom flask, palladium carbon (10%, 700.0 mg) was added to a solution of 1-[(1r,4r)-4-(dibenzylamino)cyclohexyl]-1,4,7,10,13-pentaoxapentadecan-15-ol (720.0 mg, 1.40 mmol, 1.00 equiv) in methanol (20 mL). The reaction flask was vacuumed and charged with a hydrogen balloon. The resulting mixture was stirred for 4 h at 40° C. under hydrogen atmosphere. After the reaction was done, the reaction mixture was filtered through a Celite pad and the filtrate was concentrated under reduced pressure. This resulted in 480.0 mg (crude) of 1-[(1r,4r)-4-aminocyclohexyl]-1,4,7,10,13-pentaoxapentadecan-15-ol as yellow oil.

LC-MS (ES⁺): m/z 388.25 [MNa⁺]

### Step 4. 1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-ol

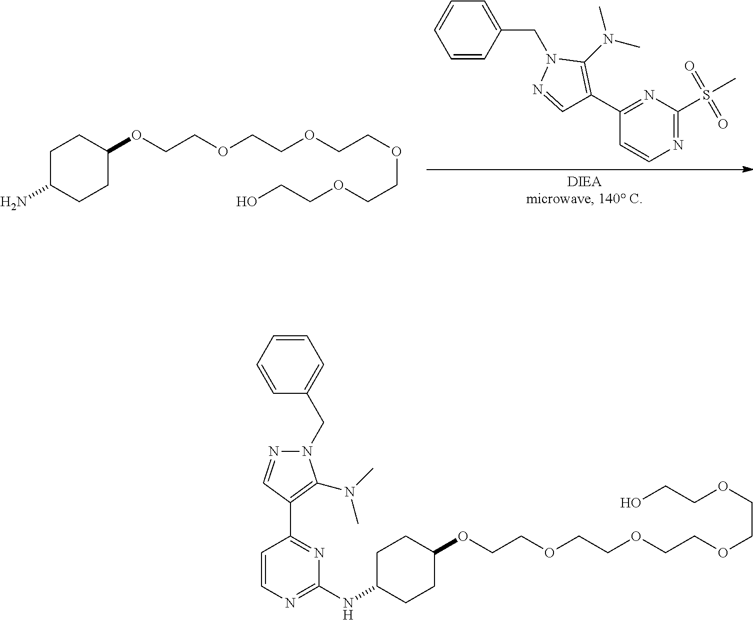 [SMILES: N[C@H]1CC[C@H](OCCOCCOCCOCCOCCO)CC1 ; CN(C)c1c(-c2ccnc(S(C)(=O)=O)n2)cnn1Cc1ccccc1 ; "DIEAmicrowave, 140° C." ; CN(C)c1c(-c2ccnc(N[C@H]3CC[C@H](OCCOCCOCCOCCOCCO)CC3)n2)cnn1Cc1ccccc1]

Into a 10-mL microwave vial, was placed 1-[(1r,4r)-4-aminocyclohexyl]-1,4,7,10,13-pentaoxapentadecan-15-ol (230.0 mg, 0.69 mmol, 1.00 equiv), 1-benzyl-4-(2-methanesulfonylpyrimidin-4-yl)-N,N-dimethyl-1H-pyrazol-5-amine [prepared as previously described by Peng, C. et al. in WO 2007129195] (245.1 mg, 0.69 mmol, 1.00 equiv), i-propanol (1.5 mL), DIEA (885.7 mg, 6.85 mmol, 10.00 equiv). The vial was irradiated in a microwave at 140° C. for 1 h. The reaction mixture was quenched by water (20 mL), extracted with ethyl acetate (30 mL×3). The combined organic layer was washed with brine (30 mL), dried over anhydrous sodium sulfate and concentrated under vacuum. The residue was applied onto a silica gel column eluting with ethyl acetate/petroleum ether (v:v=1:0). This resulted in 95.0 mg (23%) of 1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-ol as colorless oil.

LC-MS (ES⁺): m/z 613.40 [MH⁺]

### Step 5. 1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl 4-methylbenzene-1-sulfonate

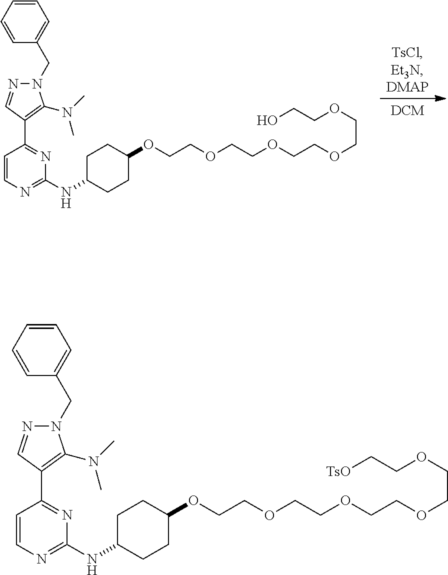

Into a 100-mL round-bottom flask, was placed 1-[(1r,4r)-4-([4-[l-benzyl-5-(dimethylamino)-1H-pyrazol-4-yl]pyrimidin-2-yl]amino)cyclohexyl]-1,4,7,10,13-pentaoxapentadecan-15-ol (95.0 mg, 0.15 mmol, 1.00 equiv), dichloromethane (5.0 mL), TsCl (59.2 mg, 0.31 mmol, 2.00 equiv), triethylamine (39.1 mg, 0.39 mmol, 2.50 equiv), 4-dimethylaminopyridine (5.7 mg, 0.05 mmol, 0.30 equiv). The resulting solution was stirred for 6 h at 40° C. in an oil bath. The reaction was then quenched by water (20 mL) and extracted with dichloromethane (20 mL×2). The combined organic layer was washed with brine (20 mL), dried over anhydrous sodium sulfate and concentrated under vacuum. The residue was applied onto a silica gel column eluting with ethyl acetate/petroleum ether (v:v=2:1). This resulted in 180.0 mg of 1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl 4-methylbenzene-1-sulfonate as light yellow oil.

LC-MS (ES⁺): m/z 767.45 [MH⁺]

### Step 6. tert-Butyl N-[(1S)-1-[[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]carbamoyl]ethyl]-N-methylcarbamate

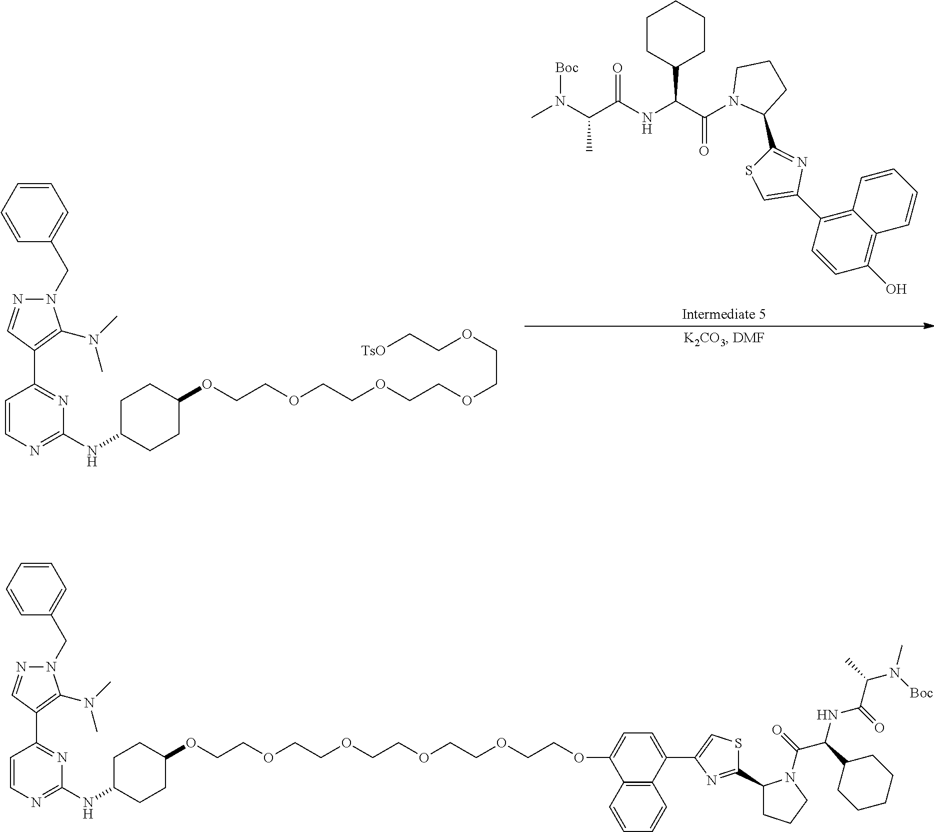

Into a 25-mL round-bottom flask, was placed 1-[(1r,4r)-4-([4-[l-benzyl-5-(dimethylamino)-1H-pyrazol-4-yl]pyrimidin-2-yl]amino)cyclohexyl]-1,4,7,10,13-pentaoxapentadecan-15-yl 4-methylbenzene-1-sulfonate (80.0 mg, 0.10 mmol, 1.00 equiv), potassium carbonate (43.2 mg, 0.31 mmol, 3.00 equiv), tert-butyl N-[(1S)-1-[(1S)-1-cyclohexyl-2-[(2S)-2-[4-(4-hydroxynaphthalen-1-yl)-1,3-thiazol-2-yl]pyrrolidin-1-yl]-2-oxoethyl]carbamoylethyl]-N-methylcarbamate (64.8 mg, 0.10 mmol, 1.00 equiv), N,N-dimethylformamide (2 mL). The resulting solution was stirred for 4 h at 80° C. in an oil bath. The reaction was then quenched by water (20 mL) and extracted with ethyl acetate (20 mL×2). The combined organic layers were washed with brine (20 mL), dried over anhydrous sodium sulfate and concentrated under vacuum. The residue was applied onto a silica gel column eluting with dichloromethane/methanol (v:v=10:1). This resulted in 91.3 mg (72%) of tert-butyl N-[(1S)-1-[[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]carbamoyl]ethyl]-N-methylcarbamate as yellow oil.

LC-MS (ES⁺): m/z 1215.70 [MH⁺]

### Step 7. (2S)—N-[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]-2-(methylamino)propanamide

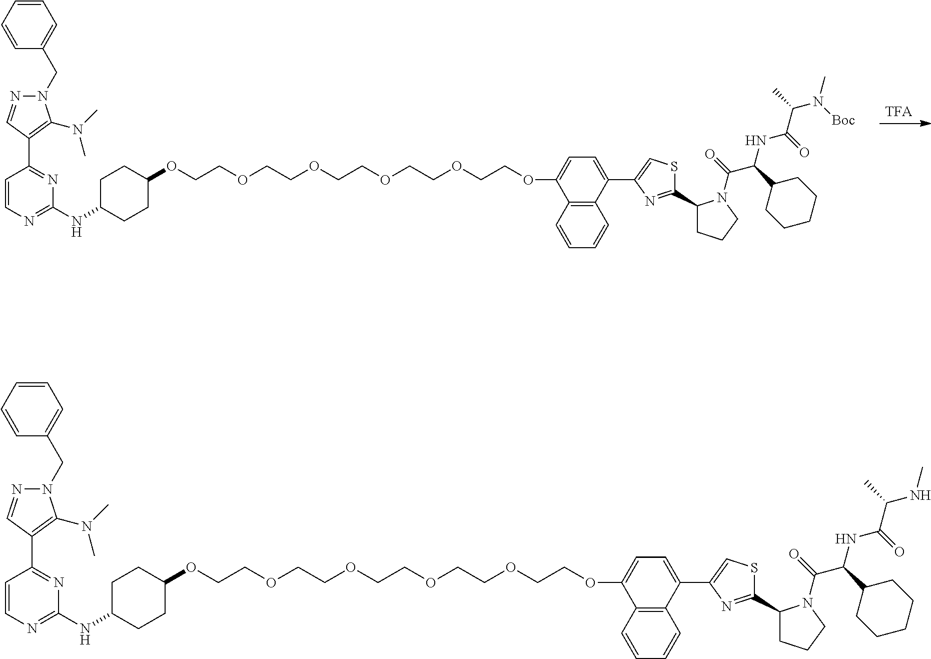

Into a 25-mL round-bottom flask, was placed tert-butyl N-[(1S)-1-[[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]carbamoyl]ethyl]-N-methylcarbamate (91.3 mg, 0.08 mmol, 1.00 equiv), dichloromethane (1 mL), trifluoroacetic acid (1 mL). The resulting solution was stirred for 2 h at room temperature. The resulting mixture was concentrated under vacuum. The crude product was purified by Prep-HPLC with the following conditions: Column, XBridge Shield RP18 OBD Column, 5 um, 19*150 mm; mobile phase, Waters (0.1% FA) and ACN (20.0% ACN up to 50.0% in 8 min); Detector, UV 220 nm. This resulted in 34.9 mg (42%) of (2S)—N-[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)naphthalen-1-yl]-1,3-thiazol-2-yl]pyrrolidin-1-yl]ethyl]-2-(methylamino)propanamide as a white solid.

¹H NMR (300 MHz, CD₃OD): δ 8.32 (m, 1H), 8.10-8.08 (m, 2H), 7.89 (s, 1H), 7.53-7.42 (m, 4H), 7.29-2.26 (m, 3H), 7.16-7.13 (m, 2H), 6.95 (m, 1H), 6.78 (m, 1H), 5.48 (m, 1H), 5.28 (s, 2H), 4.56 (m, 1H), 4.33-4.30 (m, 2H), 3.99-3.95 (m, 2H), 3.94-3.86 (m, 2H), 3.85-3.76 (m, 2H), 3.75-3.73 (m, 2H), 3.70-3.66 (m, 2H), 3.60-3.54 (m, 7H), 3.51 (m, 3H), 3.15 (m, 1H), 2.74 (s, 6H), 2.40-2.10 (m, 6H), 2.00-21.97 (m, 5H), 1.85-1.50 (m, 6H), 1.26-1.20 (m, 9H), 1.19-1.12 (m, 5H); LC-MS (ES⁺): m/z 1115.70 [MH⁺]

Using procedures analogous to those described above for Example 12, the following compounds were prepared:

### Example 13 (2S)—N-[(1S)-1-cyclohexyl-2-oxo-2-[(2S)-2-(4-[4-[2-(2-[[(1r,4r)-4-([4-[1-benzyl-5-(dimethylamino)-1H-pyrazol-4-yl]pyrimidin-2-yl]amino)cyclohexyl]oxy]ethoxy)ethoxy]naphthalen-1-yl]-1,3-thiazol-2-yl)pyrrolidin-1-yl]ethyl]-2-(methylamino)propanamide

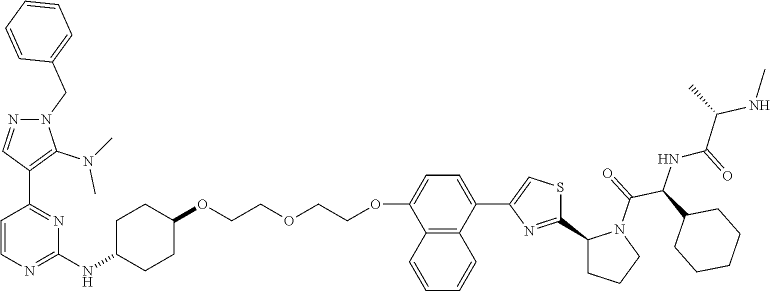 [SMILES: CN[C@@H](C)C(=O)N[C@H](C(=O)N1CCC[C@H]1c1nc(-c2ccc(OCCOCCO[C@H]3CC[C@H](Nc4nccc(-c5cnn(Cc6ccccc6)c5N(C)C)n4)CC3)c3ccccc23)cs1)C1CCCCC1]

¹H NMR (400 MHz, CD₃OD): δ 8.37-8.35 (m, 1H), 8.12-8.10 (m, 2H), 7.92 (s, 1H), 7.58-7.48 (m, 4H), 7.34-7.28 (m, 3H), 7.19-7.16 (m, 2H), 7.00-6.98 (m, 1H), 6.81-6.80 (m, 1H), 5.50 (m, 1H), 5.30 (s, 2H), 4.60-4.58 (m, 1H), 4.38-4.37 (m, 2H), 4.03-4.02 (m, 2H), 3.95-3.80 (m, 3H), 3.78-3.76 (m, 2H), 3.70-3.69 (m, 3H), 3.31-3.30 (s, 2H), 2.80-2.75 (m, 6H), 2.70 (s, 2H), 2.55-2.20 (m, 3H), 2.10-2.00 (m, 5H), 1.82-1.50 (m, 6H), 1.40-1.10 (m, 12H); LC-MS (ES⁺): m/z 983.50 [MH⁺]

### Example 14

### (S)—N—((S)-2-((S)-2-(4-(4-(2-(2-(2-((1r,4r)-4-(4-(1-benzyl-5-(dimethylamino)-1H-pyrazol-4-yl)pyrimidin-2-ylamino)cyclohexyloxy)ethoxy)ethoxy)ethoxy)naphthalen-1-yl)thiazol-2-yl)pyrrolidin-1-yl)-1-cyclohexyl-2-oxoethyl)-2-(methylamino)propanamide

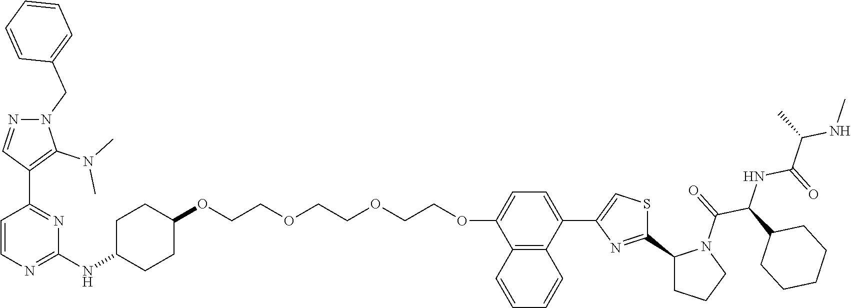

¹H NMR (300 MHz, Methanol-d4) δ 8.38-8.28 (m, 1H), 8.13-8.01 (m, 2H), 7.89 (s, 1H), 7.58-7.37 (m, 4H), 7.32-7.19 (m, 3H), 7.18-7.11 (m, 2H), 6.95 (d, J=8.0 Hz, 1H), 6.77 (d, J=5.4 Hz, 1H), 5.48 (dd, J=7.8, 2.8 Hz, 1H), 5.27 (s, 2H), 4.57 (d, J=6.9 Hz, 1H), 4.34 (dd, J=5.6, 3.5 Hz, 2H), 4.00 (dd, J=5.7, 3.3 Hz, 2H), 3.92 (dd, J=15.9, 8.7 Hz, 2H), 3.80-3.73 (m, 2H), 3.71-3.63 (m, 2H), 3.59-3.49 (m, 4H), 3.37 (q, J=6.9 Hz, 1H), 3.19 (m, 1H), 2.73 (d, J=1.8 Hz, 6H), 2.40 (s, 3H), 2.38-2.06 (m, 4H), 1.96-1.90 (m, 4H), 1.85-1.69 (m, 3H), 1.62-1.55 (d, J=21.9 Hz, 3H), 1.29 (d, J=6.9 Hz, 3H), 1.28-1.18 (t, J=9.6 Hz, 5H), 1.17-1.02 (m, 5H); LC-MS (ES⁺): m/z 1027.55 [MH⁺]

### Example 15

### (2S)—N-[(1S)-1-cyclohexyl-2-oxo-2-[(2S)-2-[4-[4-([1-[(1r,4r)-4-([4-[1-benzyl-5-(dimethylamino)-1H-pyrazol-4-yl]pyrimidin-2-yl]amino)cyclohexyl]-1,4,7,10-tetraoxadodecan-12-yl]oxy)naphthalen-1-yl]-1,3-thiazol-2-yl]pyrrolidin-1-yl]ethyl]-2-(methylamino)propanamide

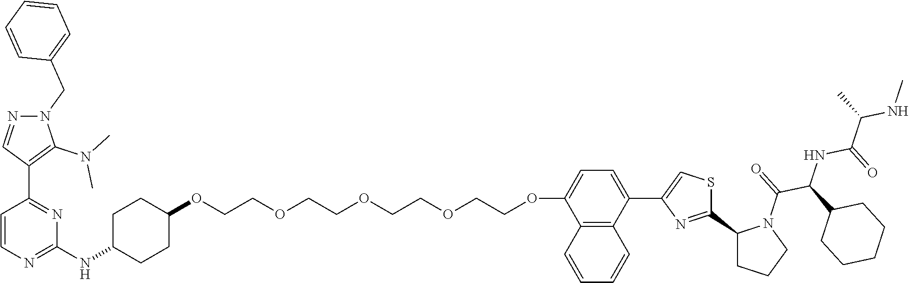

1H NMR (400 MHz, CD3OD) δ 8.39 (m, 1H), 8.14 (d, J=8.4 Hz, 2H), 7.94 (s, 1H), 7.57-7.46 (m, 4H), 7.33-7.19 (m, 5H), 6.99 (d, J=8.0 Hz, 1H), 6.82 (d, J=8.4 Hz, 1H), 5.58 (s, 1H), 5.33 (s, 2H), 4.62 (d, J=8.4 Hz, 1H), 4.38 (m, 2H), 4.06-3.81 (m, 7H), 3.73-3.51 (m, 11H), 3.33-3.22 (m, 2H), 2.78 (s, 6H), 2.41-2.35 (m, 4H), 2.28-2.02 (m, 7H), 1.84-1.59 (m, 6H), 1.30-1.14 (m, 11H); LC-MS (ES+): m/z 1071.65 [MH+]

### Example 16 (2S)—N-[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)phenyl]ethyl]-octahydro-1H-pyrrolo[2,3-c]pyridin-1-yl)ethyl]-2-(methylamino)propanamide

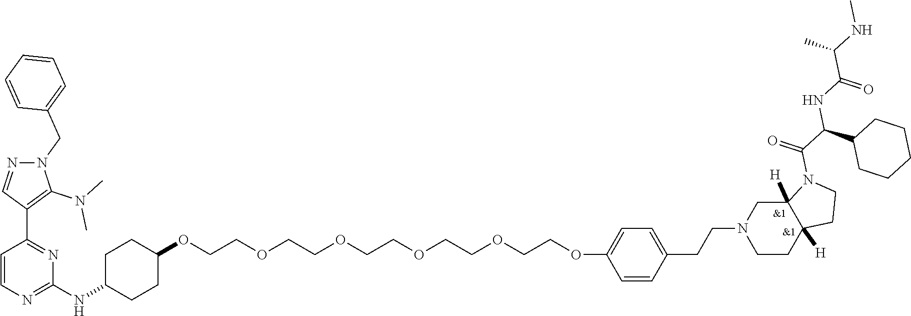

### Step 1. tert-Butyl N-[(1S)-1-[[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)phenyl]ethyl]-octahydro-1H-pyrrolo[2,3-c]pyridin-1-yl)ethyl]carbamoyl]ethyl]-N-methylcarbamate

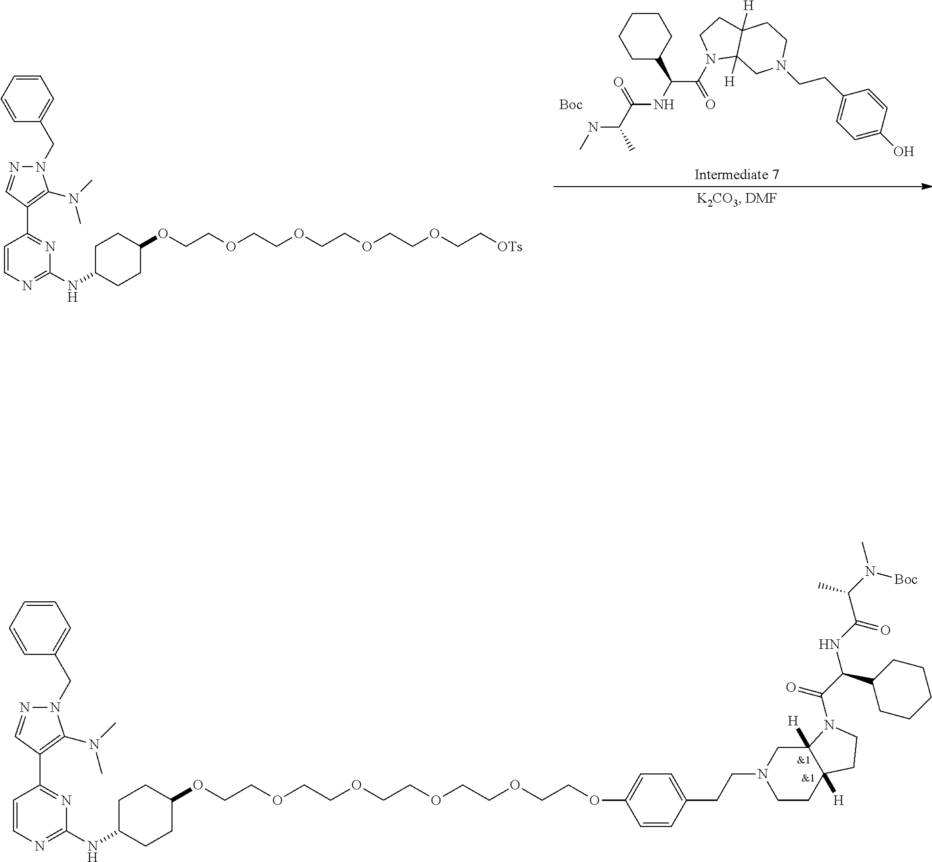

Into a 25-mL round-bottom flask, was placed a solution of tert-butyl N-[(1S)-1-[[(1S)-1-cyclohexyl-2-[6-[2-(4-hydroxyphenyl)ethyl]-octahydro-1H-pyrrolo[2,3-c]pyridin-1-yl]-2-oxoethyl]carbamoyl]ethyl]-N-methylcarbamate (57.0 mg, 0.10 mmol, 1.10 equiv) in N,N-dimethylformamide (10 mL), K₂CO₃ (90.0 mg, 0.28 mmol, 3.00 equiv), 1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl 4-methylbenzene-1-sulfonate [prepared as described in Example 12](70.0 mg, 0.09 mmol, 1.00 equiv). The resulting solution was stirred for 2 h at 80° C. in an oil bath. The reaction was then quenched by the addition of water (100 mL) and extracted with ethyl acetate (50 mL×3). The combined organic layers was washed with brine (80 mL×2), dried over anhydrous sodium sulfate and concentrated under reduced pressure. The residue was applied onto a silica gel column eluting with dichloromethane/methanol (10/1). This resulted in 60.0 mg (56%) of tert-butyl N-[(1S)-1-[[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)phenyl]ethyl]-octahydro-1H-pyrrolo[2,3-c]pyridin-1-yl)ethyl]carbamoyl]ethyl]-N-methylcarbamate as a yellow solid.

### Step 2. tert-Butyl N-[(1S)-1-[[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)phenyl]ethyl]-octahydro-1H-pyrrolo[2,3-c]pyridin-1-yl)ethyl]carbamoyl]ethyl]-N-methylcarbamate

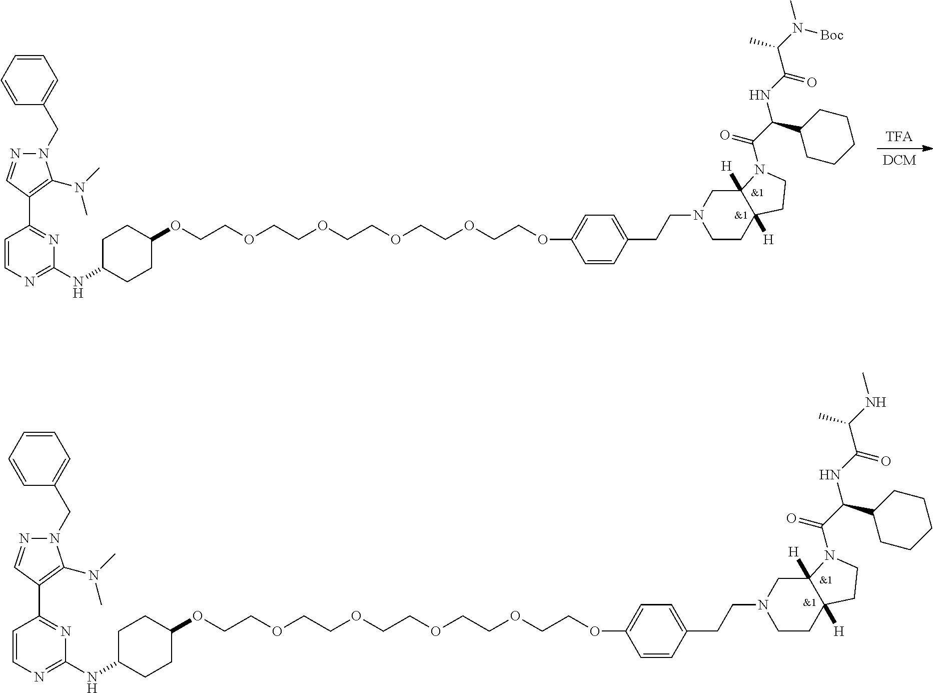

tert-Butyl N-[(1S)-1-[[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10,13-pentaoxapentadecan-15-yl]oxy)phenyl]ethyl]-octahydro-1H-pyrrolo[2,3-c]pyridin-1-yl)ethyl]carbamoyl]ethyl]-N-methylcarbamate was converted into the title compound using procedure of the step 7 of Example 12.

¹H NMR (300 MHz, CD₃OD): δ 8.15-8.13 (s, 1H), 7.93 (s, 1H), 7.33-7.06 (m, 7H), 6.86-6.81 (m, 3H), 5.33 (s, 2H), 4.45-4.40 (m, 1H), 4.30-4.10 (m, 1H), 4.08-4.07 (m, 2H), 4.00-3.90 (m, 1H), 3.83-3.82 (m, 2H), 3.69-3.55 (m, 18H), 3.14-3.12 (m, 1H), 2.80 (m, 7H), 2.76-2.70 (m, 2H), 2.67-2.57 (m, 2H), 2.30-2.29 (m, 4H), 2.20-2.00 (m, 7H), 1.90-1.50 (m, 10H), 1.45-1.20 (m, 13H); LC-MS (ES⁺): m/z 1065.60 [MH⁺]

Using procedures analogous to those described above for Example 16, the following compounds were prepared:

### Example 17 (2S)—N-[(1S)-1-cyclohexyl-2-oxo-2-[6-(2-[4-[2-(2-[[(1r,4r)-4-([4-[1-benzyl-5-(dimethylamino)-1H-pyrazol-4-yl]pyrimidin-2-yl]amino)cyclohexyl]oxy]ethoxy)ethoxy]phenyl]ethyl)-octahydro-1H-pyrrolo[2,3-c]pyridin-1-yl]ethyl]-2-(methylamino)propanamide

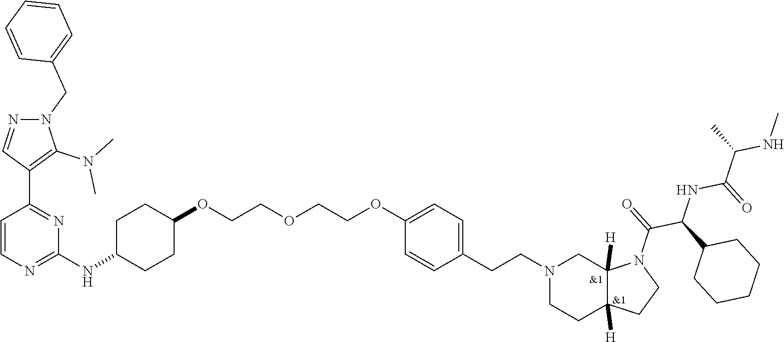

¹H NMR (400 MHz, CD₃OD): δ 8.12 (s, 1H), 7.93 (s, 1H), 7.32-7.07 (m, 7H), 6.87-6.80 (m, 3H), 5.32 (s, 2H), 4.60-4.40 (m, 1H), 4.30-4.10 (m, 3H), 4.09-3.83 (m, 1H), 3.82 (m, 2H), 3.81-3.66 (m, 5H), 3.50-3.35 (m, 2H), 3.30-3.11 (m, 1H), 2.90-2.69 (m, 9H), 2.60-2.40 (m, 2H), 2.30 (m, 4H), 2.29-1.90 (m, 8H), 1.82-1.55 (m, 9H), 1.36-0.09 (m, 13H); LC-MS (ES⁺): m/z 933.60 [MH⁺]

### Example 18 (2S)—N-[(1S)-1-cyclohexyl-2-oxo-2-[6-[2-(4-[2-[2-(2-[[(1r,4r)-4-([4-[1-benzyl-5-(dimethylamino)-1H-pyrazol-4-yl]pyrimidin-2-yl]amino)cyclohexyl]oxy]ethoxy)-ethoxy]ethoxy]phenyl)ethyl]-octahydro-1H-pyrrolo[2,3-c]pyridin-1-yl]ethyl]-2-(methylamino)propanamide

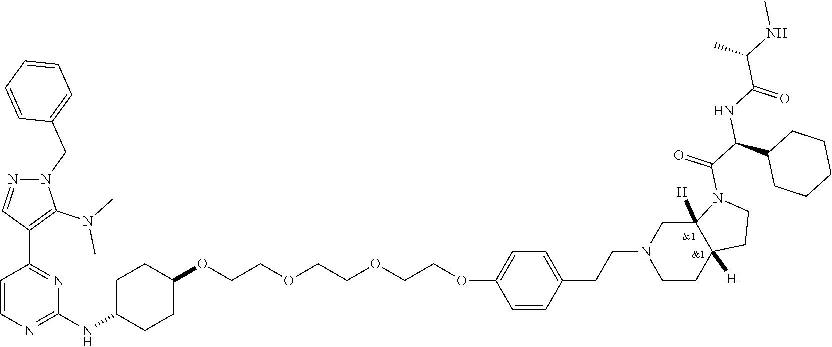

¹H NMR (300 MHz, CD₃OD): δ 8.15 (s, 1H), 7.95 (s, 1H), 7.35-7.20 (m, 7H), 6.88-6.83 (m, 3H), 5.34 (s, 2H), 4.70-4.45 (m, 1H), 4.40-4.15 (m, 1H), 4.11 (m, 2H), 4.10-3.90 (m, 1H), 3.85-3.64 (m, 11H), 3.55-3.35 (m, 2H), 3.30-3.20 (m, 1H), 3.15-3.10 (m, 1H), 2.95-2.81 (m, 9H), 2.65-2.50 (m, 2H), 2.45-2.30 (m, 4H), 2.25-2.00 (m, 7H), 1.95-1.55 (m, 9H), 1.50-1.15 (m, 11H), 1.10-090 (m, 2H); LC-MS (ES⁺): m/z 977.75 [MH⁺]

### Example 19 (2S)—N-[(1S)-1-cyclohexyl-2-oxo-2-(6-[2-[4-([1-[(1r,4r)-4-([4-[1-benzyl-5-(dimethylamino)-1H-pyrazol-4-yl]pyrimidin-2-yl]amino)cyclohexyl]-1,4,7,10-tetraoxadodecan-12-yl]oxy)phenyl]ethyl]-octahydro-1H-pyrrolo[2,3-c]pyridin-1-yl)ethyl]-2-(methylamino)propanamide

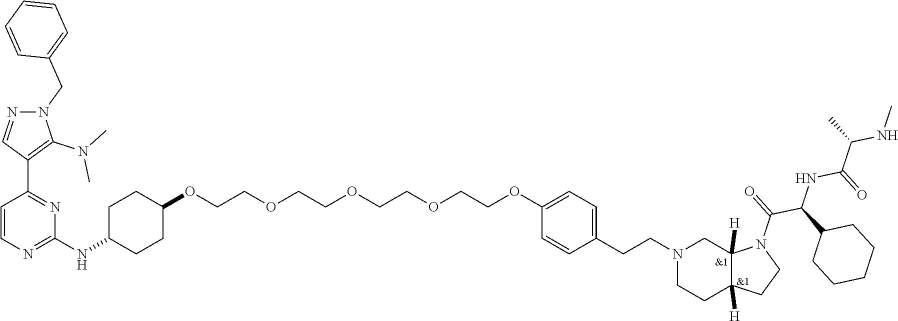

¹H NMR (300 MHz, CD₃OD): δ 8.15-8.13 (s, 1H), 7.93 (s, 1H), 7.33-7.06 (m, 7H), 6.88-6.81 (m, 3H), 5.33 (s, 2H), 4.60-4.45 (m, 1H), 4.09-4.08 (m, 2H), 3.90-3.84 (m, 1H), 3.83-3.81 (m, 2H), 3.69-3.54 (m, 13H), 3.20-3.13 (m, 1H), 2.95-2.67 (m, 10H), 2.59-2.53 (m, 2H), 2.31-2.30 (s, 3H), 2.29-2.06 (m, 7H), 1.90-1.50 (m, 10H), 1.45-0.95 (m, 15H); LC-MS (ES⁺): m/z 1021.60 [MH⁺]

### Example 20

### (2S)—N-[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]-2-(methylamino)propanamide

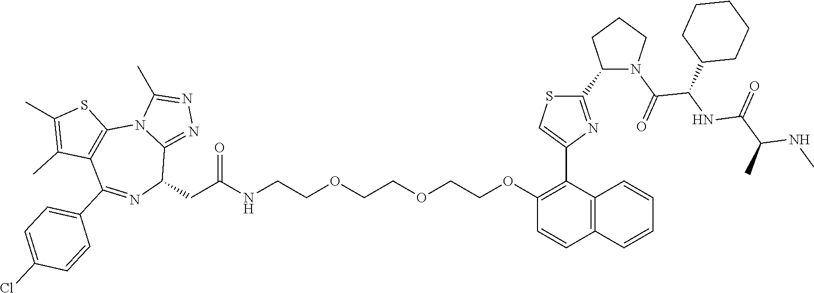 [SMILES: CN[C@@H](C)C(=O)N[C@H](C(=O)N1CCC[C@H]1c1nc(-c2c(OCCOCCOCCNC(=O)C[C@@H]3N=C(c4ccc(Cl)cc4)c4c(sc(C)c4C)-n4c(C)nnc43)ccc3ccccc23)cs1)C1CCCCC1]

### Step 1. 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-hydroxyethoxy)ethoxy]ethyl]acetamide

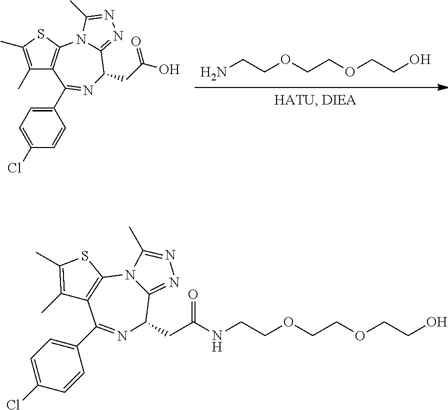

Into a 25-mL round-bottom flask, was placed a solution of 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetic acid (TFA salt) [prepared as previously described by Filippakopoulos, P. et al. in Nature 2010, 468, 1067-1073 and by Zengerle, M. et al. in ACS Chemical Biology 2015, 10, 1770-1777] (150.0 mg, 0.37 mmol, 1.00 equiv) in N,N-dimethylformamide (5 mL). This was followed by the addition of O-(7-Azabenzotriazol-1-yl)-N,N,N,N-tetramethyluronium Hexafluorophosphate (171.0 mg, 0.45 mmol, 1.20 equiv) at 0° C. N,N-Diisopropylethylamine 0.2 ml was added into at 0° C. To this was added 2-[2-(2-aminoethoxy)ethoxy]ethan-1-ol (168.0 mg, 1.13 mmol, 3.00 equiv) at 0° C. The resulting solution was stirred for 2 h at room temperature. The resulting solution was stirred for 1 h at 10° C. The reaction was then quenched by the addition of water (20 mL). The resulting solution was extracted with ethyl acetate (20 mL×3) and the organic layers combined. The resulting mixture was washed with brine (20 mL×1). The mixture was dried over anhydrous sodium sulfate and concentrated under vacuum. The residue was applied onto a silica gel column with dichloromethane/methanol (10:1). This resulted in 140.0 mg (70%) of 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-hydroxyethoxy)ethoxy]ethyl]acetamide as yellow oil.

### Step 2. 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-[[(4-methylbenzene)sulfonyl]oxy]ethoxy)ethoxy]ethyl]acetamide

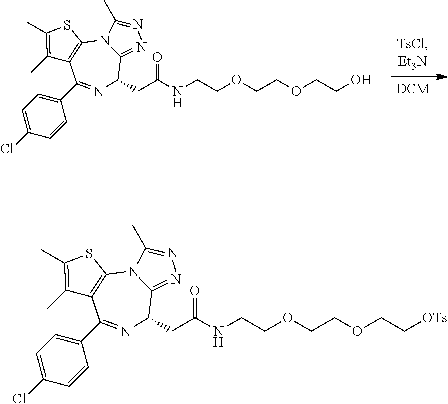

Into a 25-mL round-bottom flask, was placed 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-hydroxyethoxy)ethoxy]ethyl]acetamide (100.0 mg, 0.19 mmol, 1.00 equiv), dichloromethane (10 mL), 4-methylbenzene-1-sulfonyl chloride (53.67 mg, 0.28 mmol, 1.50 equiv), triethylamine (38.0 mg, 0.38 mmol, 2.00 equiv), 4-dimethylaminopyridine (2.29 mg, 0.02 mmol, 0.10 equiv). The resulting solution was stirred for 2 h at room temperature, was purified by TLC with dichloromethane/methanol (10:1). This resulted in 90.0 mg (70%) of 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-[[(4-methylbenzene)sulfonyl]oxy]ethoxy)ethoxy]ethyl]acetamide as light yellow oil.

LC-MS (ES⁺): m/z 686.15 [MH⁺]

### Step 3. tert-Butyl N-[(1S)-1-[[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0 \hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]carbamoyl]ethyl]-N-methylcarbamate

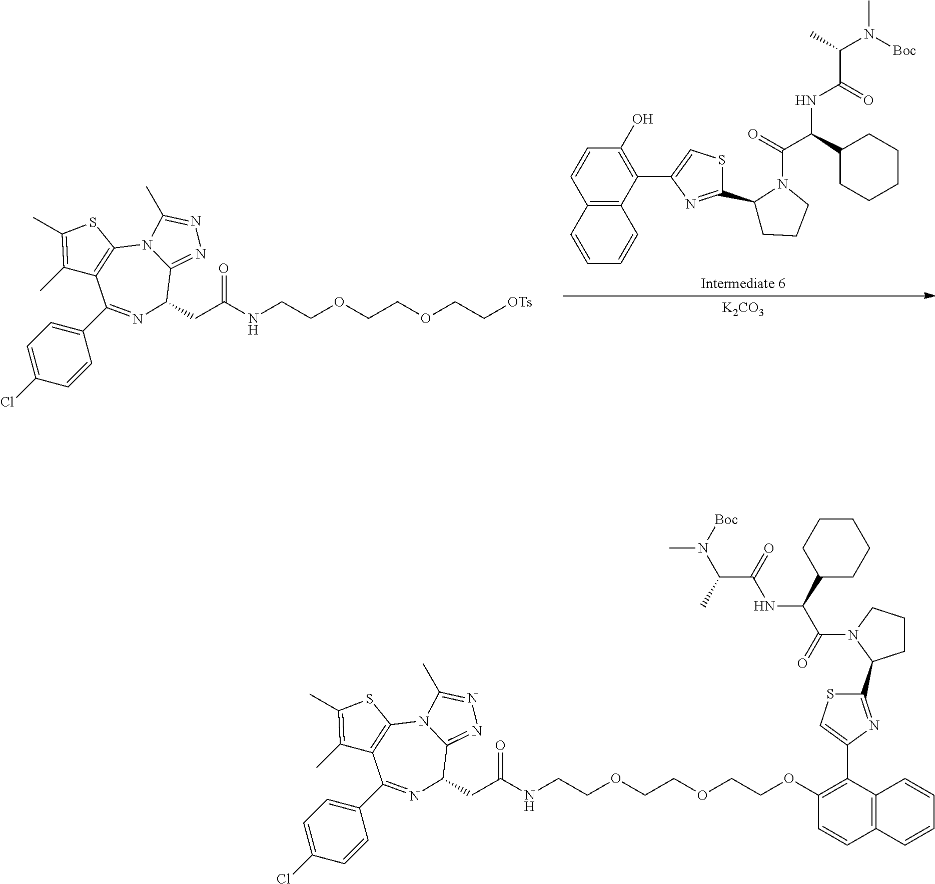 [SMILES: Cc1ccc(S(=O)(=O)OCCOCCOCCNC(=O)C[C@@H]2N=C(c3ccc(Cl)cc3)c3c(sc(C)c3C)-n3c(C)nnc32)cc1 ; C[C@@H](C(=O)N[C@H](C(=O)N1CCC[C@H]1c1nc(-c2c(O)ccc3ccccc23)cs1)C1CCCCC1)N(C)C(=O)OC(C)(C)C ; "K2CO3" ; "Intermediate 6" ; Cc1sc2c(c1C)C(c1ccc(Cl)cc1)=N[C@@H](CC(=O)NCCOCCOCCOc1ccc3ccccc3c1-c1csc([C@@H]3CCCN3C(=O)[C@@H](NC(=O)[C@H](C)N(C)C(=O)OC(C)(C)C)C3CCCCC3)n1)c1nnc(C)n1-2]

Into a 25-mL round-bottom flask, was placed 2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]-N-[2-[2-(2-[[(4-methylbenzene)sulfonyl]oxy]ethoxy)ethoxy]ethyl]acetamide (40.0 mg, 0.06 mmol, 1.00 equiv), N,N-dimethylformamide (5 mL), potassium carbonate (16.11 mg, 0.12 mmol, 2.00 equiv), tert-butyl N-[(1S)-1-[(1S)-1-cyclohexyl-2-[(2S)-2-[4-(2-hydroxynaphthalen-1-yl)-1,3-thiazol-2-yl]pyrrolidin-1-yl]-2-oxoethyl]carbamoylethyl]-N-methylcarbamate (36.2 mg, 0.06 mmol, 1.00 equiv). The resulting solution was stirred overnight at 80° C. in an oil bath. The reaction was then quenched by the addition of water (20 mL). The resulting solution was extracted with ethyl acetate (20 mL×3) and the organic layers combined. The resulting mixture was washed with brine (20 mL×1). The mixture was dried over anhydrous sodium sulfate and concentrated under vacuum. The residue was applied onto a silica gel column with dichloromethane/methanol (10:1). This resulted in 66.0 mg (100%) of tert-butyl N-[(1S)-1-[[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0^{A}[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]carbamoyl]ethyl]-N-methylcarbamate as light yellow oil.

LC-MS (ES⁺): m/z 1135.60/1137.60 [MH⁺]

### Step 4. (2S)—N-[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]-2-(methylamino)propanamide

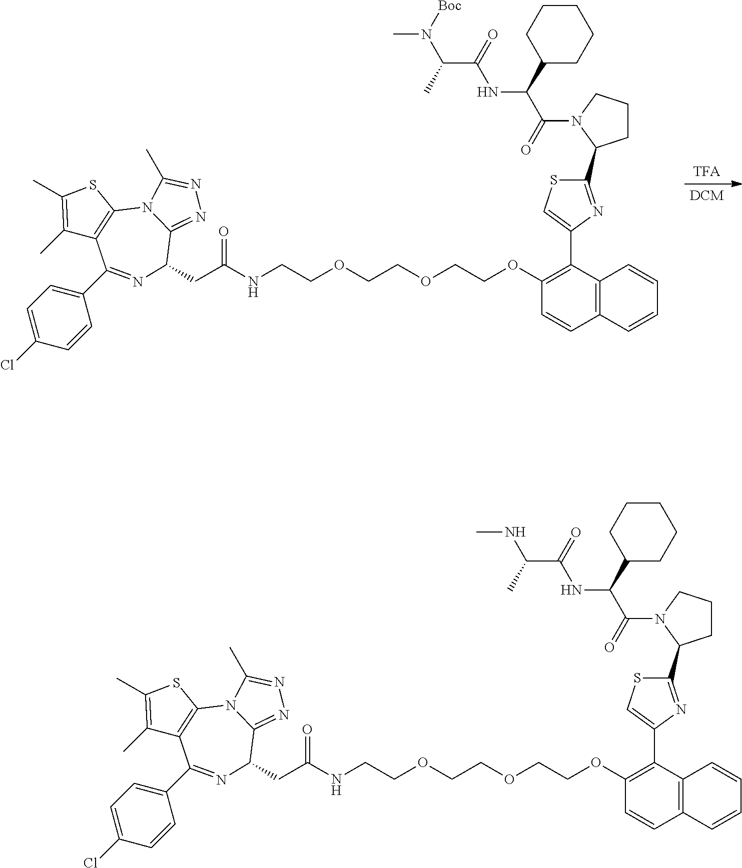

Into a 25-mL round-bottom flask, was placed tert-butyl N-[(1S)-1-[[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]carbamoyl]ethyl]-N-methylcarbamate (66.0 mg, 0.06 mmol, 1.00 equiv), dichloromethane (5.0 mL), trifluoroacetic acid (3.0 mL). The resulting solution was stirred for 2 h at room temperature. The resulting mixture was concentrated under vacuum, then purified by Prep-HPLC with Column: XBridge Shield RP18 OBD Column, 5 um, 19*150 mm; Mobile Phase A:Waters (10 mmol/L Bicarbonate amine), Mobile Phase B: acetonitrile; Flow rate: 20 mL/min; Gradient: 45% B to 61% B in 10 min; 220 nm. This resulted in 12.5 mg (21%) of (2S)—N-[(1S)-2-[(2S)-2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]-2-(methylamino)propanamide as a white solid.

¹H NMR (400 MHz, CD₃OD) δ 8.70-8.50 (b, 1H), 7.93-7.91 (d, J=8.8 Hz, 1H), 7.84-7.82 (d, J=8.8 Hz, 1H), 7.59-7.56 (m, 2H), 7.47-7.36 (m, 7H), 5.55-5.53 (m, 1H), 4.62-4.61 (m, 2H), 4.25-4.24 (m, 2H), 4.00-3.91 (m, 2H), 3.81-3.71 (m, 7H), 3.59-3.45 (m, 3H), 2.72 (s, 3H), 2.61 (s, 3H), 2.45 (s, 3H), 2.42-2.02 (m, 4H), 2.00-1.79 (m, 3H), 1.73-1.69 (m, 4H), 1.69-1.62 (m, 2H), 1.46-1.44 (m, 2H), 1.40-1.20 (m, 3H), 1.19-1.11 (m, 5H). LC-MS (ES⁺): m/z 1034.55/1036.55 [MH⁺]

Using procedures analogous to those described above for Example 20, the following compounds have been prepared:

### Example 21 (2S)—N-[(1S)-2-[(2S)-2-[4-(4-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]naphthalen-1-yl)-1,3-thiazol-2-yl]pyrrolidin-1-yl]-1-cyclohexyl-2-oxoethyl]-2-(methylamino)propanamide

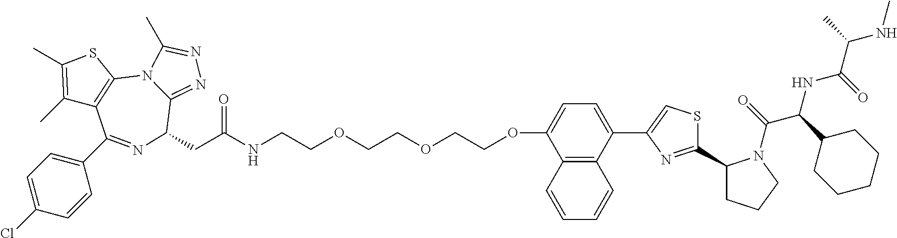 [SMILES: CN[C@@H](C)C(=O)N[C@H](C(=O)N1CCC[C@H]1c1nc(-c2ccc(OCCOCCOCCNC(=O)C[C@@H]3N=C(c4ccc(Cl)cc4)c4c(sc(C)c4C)-n4c(C)nnc43)c3ccccc23)cs1)C1CCCCC1]

¹H NMR (400 MHz, CD₃OD): δ 8.30 (m, 1H), 8.10 (m, 1H), 7.51-7.36 (m, 8H), 6.95-6.93 (d, J=8.0 Hz, 1H), 5.10 (m, 1H), 4.63-4.60 (m, 2H), 4.38-4.36 (t, J=4.6 Hz, 2H), 4.07-4.04 (m, 2H), 3.95 (m, 1H), 3.85-3.83 (m, 2H), 3.75-3.73 (m, 2H), 3.67-3.64 (t, J=5.4 Hz, 2H), 3.48-3.41 (m, 3H), 3.21-3.19 (m, 2H), 2.69 (s, 3H), 2.43 (s, 3H), 2.34 (s, 3H), 2.30-2.18 (m, 4H), 1.84-1.80 (m, 3H), 1.79-1.64 (m, 6H), 1.31-1.28 (m, 2H), 1.26 (m, 3H), 1.19-1.12 (m, 4H); LC-MS (ES⁺): m/z 1034.50/1036.50 [MH⁺]

### Example 22 (2S)—N-[(1S)-2-[(2S)-2-[4-[4-(2-[2-[2-(2-[2-[(9S)-7-(4-chlorophenyl)-4,5,13-trimethyl-3-thia-1,8,11,12-tetraazatricyclo[8.3.0.0 \hat{ }[2,6]]trideca-2(6),4,7,10,12-pentaen-9-yl]acetamido]ethoxy)ethoxy]ethoxy]ethoxy)naphthalen-1-yl]-1,3-thiazol-2-yl] pyrrolidin-1-yl]-1-cyclohexyl-2-oxoethyl]-2-(methylamino)propanamide

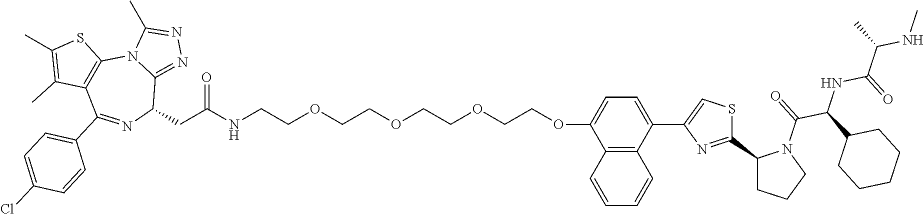 [SMILES: CN[C@@H](C)C(=O)N[C@H](C(=O)N1CCC[C@H]1c1nc(-c2ccc(OCCOCCOCCOCCNC(=O)C[C@@H]3N=C(c4ccc(Cl)cc4)c4c(sc(C)c4C)-n4c(C)nnc43)c3ccccc23)cs1)C1CCCCC1]

¹H NMR (400 MHz, CD₃OD): δ 8.35-8.33 (m, 1H), 8.15-8.13 (m, 1H), 7.49-7.35 (m, 8H), 6.93-6.91 (m, 1H), 5.55-5.52 (m, 1H), 4.61-4.59 (m, 2H), 4.37-4.35 (m, 2H), 4.03-4.00 (m, 4H), 3.81-3.80 (m, 2H), 3.79-3.56 (m, 7H), 3.62-3.56 (m, 5H), 2.66 (s, 3H), 2.54 (s, 3H), 2.42 (s, 3H), 2.42-2.41 (m, 1H), 2.32-2.22 (m, 2H), 2.12-2.00 (m, 1H), 1.99-1.80 (m, 3H), 1.71-1.55 (m, 6H), 1.41-1.40 (m, 3H), 1.21-1.01 (m, 6H); LC-MS (ES⁺): m/z 1078.60/1080.60 [MH⁺]

### Example 23 (2S)—N-[(1S)-2-[(2S)-2-(4-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]naphthalen-1-yl]-1,3-thiazol-2-yl)pyrrolidin-1-yl]-1-cyclohexyl-2-oxoethyl]-2-(methylamino)propanamide

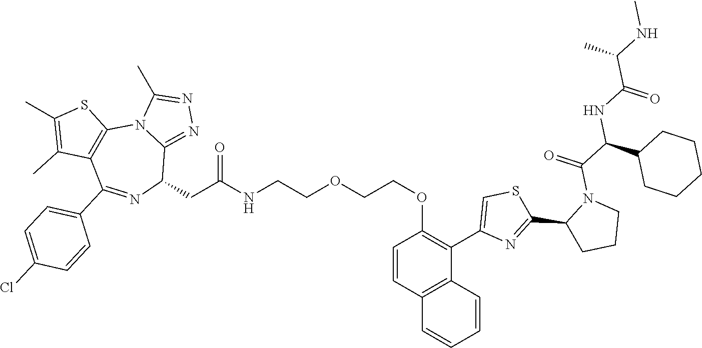 [SMILES: CN[C@@H](C)C(=O)N[C@H](C(=O)N1CCC[C@H]1c1nc(-c2c(OCCOCCNC(=O)C[C@@H]3N=C(c4ccc(Cl)cc4)c4c(sc(C)c4C)-n4c(C)nnc43)ccc3ccccc23)cs1)C1CCCCC1]

¹H NMR (400 MHz, CD₃OD): δ 7.95-7.93 (d, J=9.2 Hz, 1H), 7.85-7.82 (d, J=9.2 Hz, 1H), 7.59-7.57 (d, J=7.6 Hz, 1H), 7.48-7.35 (m, 8H), 5.55-5.53 (m, 1H), 4.63-4.58 (m, 2H), 4.26-4.24 (t, J=2.8 Hz, 2H), 4.00-3.85 (m, 2H), 3.78-3.76 (t, J=4.6 Hz, 2H), 3.56-3.54 (t, J=5.4 Hz, 2H), 3.44-3.33 (m, 3H), 3.32-3.19 (m, 2H), 2.69 (s, 3H), 2.68 (s, 3H), 2.34 (s, 3H), 2.24-2.05 (m, 3H), 1.84-1.80 (m, 3H), 1.68-1.60 (m, 6H), 1.30 (s, 1H), 1.25-1.24 (d, J=6.8 Hz, 3H), 1.17-1.12 (m, 5H); LC-MS (ES⁺): m/z 990.55/992.55 [MH⁺]

### Example 24 (2S)—N-[(1S)-2-[(2S)-2-[4-[2-(2-[2-[2-(2-[2-[(9S)-7-(4-chlorophenyl)-4,5,13-trimethyl-3-thia-1,8,11,12-tetraazatricyclo[8.3.0.0 \hat{ }[2,6]]trideca-2(6),4,7,10,12-pentaen-9-yl]acetamido]ethoxy)ethoxy]ethoxy]ethoxy)naphthalen-1-yl]-1,3-thiazol-2-yl]pyrrolidin-1-yl]-1-cyclohexyl-2-oxoethyl]-2-(methylamino)propanamide

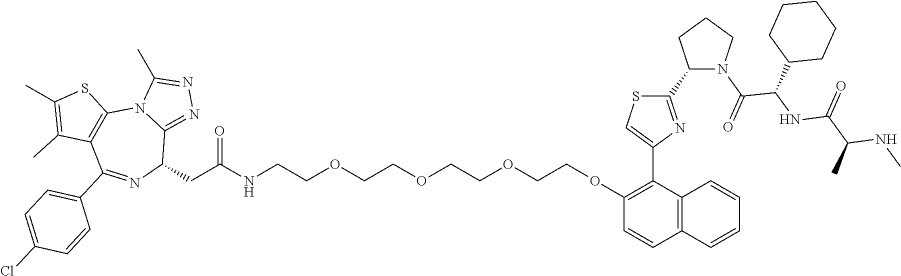 [SMILES: CN[C@@H](C)C(=O)N[C@H](C(=O)N1CCC[C@H]1c1nc(-c2c(OCCOCCOCCOCCNC(=O)C[C@@H]3N=C(c4ccc(Cl)cc4)c4c(sc(C)c4C)-n4c(C)nnc43)ccc3ccccc23)cs1)C1CCCCC1]

¹H NMR (400 MHz, CD₃OD): δ 7.88-7.80 (m, 2H), 7.56-7.54 (m, 2H), 7.44-7.34 (m, 7H), 5.61-5.52 (m, 1H), 4.62-4.58 (m, 2H), 4.22-4.20 (m, 2H), 4.00-3.88 (m, 2H), 3.82-3.75 (m, 3H), 3.56-3.55 (m, 10H), 3.42-3.41 (m, 3H), 3.41-3.39 (m, 1H), 2.67-2.63 (m, 6H), 2.47 (s, 3H), 2.41-2.27 (m, 3H), 2.11-2.00 (m, 1H), 1.92-1.72 (m, 3H), 1.63-1.51 (m, 6H), 1.59-1.56 (m, 3H), 1.32-1.15 (m, 5H); LC-MS (ES⁺): m/z 1078.65/1080.65 [MH⁺]

### Example 25 (2S)—N-[(1S)-2-[(2S)-2-(4-[4-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]naphthalen-1-yl]-1,3-thiazol-2-yl)pyrrolidin-1-yl]-1-cyclohexyl-2-oxoethyl]-2-(methylamino)propanamide

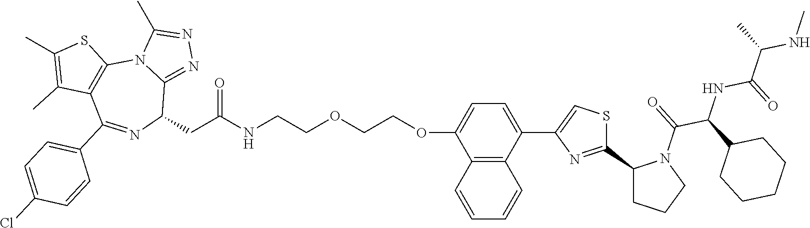 [SMILES: CN[C@@H](C)C(=O)N[C@H](C(=O)N1CCC[C@H]1c1nc(-c2ccc(OCCOCCNC(=O)C[C@@H]3N=C(c4ccc(Cl)cc4)c4c(sc(C)c4C)-n4c(C)nnc43)c3ccccc23)cs1)C1CCCCC1]

¹H NMR (400 MHz, CD₃OD): δ 8.36-8.33 (d, J=9.6 Hz, 1H), 8.11-8.09 (d, J=9.2 Hz, 1H), 7.56-7.54 (d, J=8.0 Hz, 1H), 7.51-7.43 (m, 5H), 7.37-7.34 (d, J=8.4 Hz, 2H), 6.99-6.97 (d, J=8.0 Hz, 1H), 5.55-5.54 (d, J=5.2 Hz, 1H), 4.63-4.58 (m, 2H), 4.39-4.37 (t, J=4.6 Hz, 2H), 4.06-3.90 (m, 4H), 3.81-3.79 (m, 2H), 3.57-3.40 (m, 3H), 3.33-3.20 (m, 2H), 2.68 (s, 3H), 2.43-2.34 (m, 8H), 2.18 (s, 1H), 1.84-1.80 (m, 3H), 1.67-1.62 (m, 6H), 1.27-1.26 (d, J=6.8 Hz, 3H), 1.19-1.11 (m, 5H); LC-MS (ES⁺): m/z 990.55/992.55 [MH⁺]

### Example 26 (2S)—N-[(1S)-2-[(3aS,7aR)-6-[2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]] trideca-2(6),4,7,10,12-pentaen-9-yl]acetamido] ethoxy)ethoxy]ethoxy]ethoxy)phenyl]ethyl]-octahydro-1H-pyrrolo[2,3-c]pyridin-1-yl]-1-cyclohexyl-2-oxoethyl]-2-(methylamino)propanamide

### (2S)—N-[(1S)-2-[(3aR,7aS)-6-[2-[4-(2-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]ethoxy)phenyl]ethyl]-octahydro-1H-pyrrolo[2,3-c]pyridin-1-yl]-1-cyclohexyl-2-oxoethyl]-2-(methylamino)propanamide

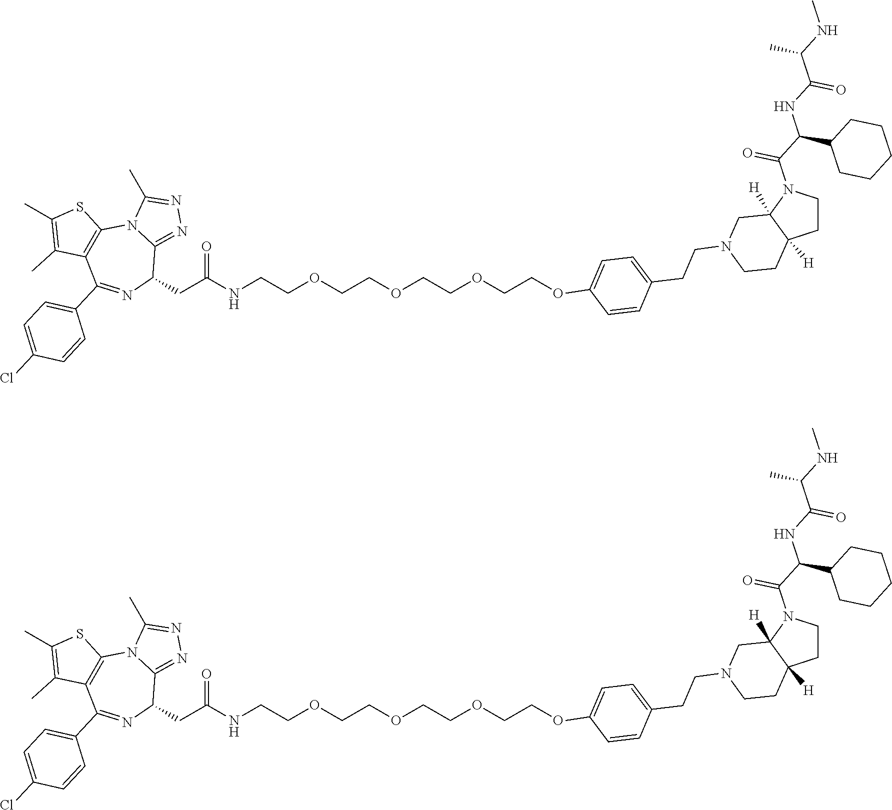 [SMILES: CN[C@@H](C)C(=O)N[C@H](C(=O)N1CC[C@H]2CCN(CCc3ccc(OCCOCCOCCOCCNC(=O)C[C@@H]4N=C(c5ccc(Cl)cc5)c5c(sc(C)c5C)-n5c(C)nnc54)cc3)C[C@H]21)C1CCCCC1 ; CN[C@@H](C)C(=O)N[C@H](C(=O)N1CC[C@@H]2CCN(CCc3ccc(OCCOCCOCCOCCNC(=O)C[C@@H]4N=C(c5ccc(Cl)cc5)c5c(sc(C)c5C)-n5c(C)nnc54)cc3)C[C@@H]21)C1CCCCC1]

The crude product was purified by Prep-HPLC with the following conditions: Column: Gemini-NX C18 AXAI Packed 21.2*150 mm Sum; Mobile Phase A:Waters (10.0 mmol/L NH₄HCO₃), Mobile Phase B: ACN; Flow rate: 20 mL/min; Gradient: 61% B to 90% B in 8 min. This resulted in 12.4 mg (23%) of isomer 1 and 10.9 mg (20%) of isomer 2 (two absolute stereo configurations of the octahydro-1H-pyrrolo[2,3-c]pyridine motif not assigned to the specific isomers).

Isomer 1. ¹H NMR (300 MHz, CD3OD) δ 8.91-8.18 (m, 2H), 7.55-7.28 (m, 4H), 7.07-7.00 (m, 2H), 6.80-6.71 (m, 2H), 4.75-4.50 (m, 1H), 4.48-4.25 (m, 2H), 4.10-4.00 (m, 2H), 3.95-3.90 (m, 1H), 3.80-3.70 (m, 3H), 3.77-3.50 (m, 8H), 3.50-3.40 (m, 3H), 3.35-3.30 (m, 3H), 3.28-3.10 (m, 2H), 3.00-2.76 (m, 5H), 2.66 (s, 3H), 2.60 (s, 3H), 2.54-2.30 (m, 5H), 2.20-2.00 (m, 2H), 1.98-1.70 (m, 6H), 1.65-1.50 (m, 5H), 1.49-1.30 (m, 3H), 1.29-0.92 (m, 6H); LC-MS (ES⁺): m/z 1050.50 [MNa⁺].

Isomer 2. ¹H NMR (300 MHz, CD₃OD) δ 8.81-8.18 (m, 1H), 7.64-7.26 (m, 4H), 7.10-7.00 (m, 2H), 6.80-6.72 (m, 2H), 4.75-4.40 (m, 2H), 4.38-4.10 (m, 1H), 4.09-3.90 (m, 2H), 3.85-3.70 (m, 3H), 3.69-3.50 (m, 9H), 3.49-3.40 (m, 2H), 3.38-3.30 (m, 1H), 3.25-3.20 (m, 3H), 3.18-3.00 (m, 1H), 3.00-2.60 (m, 8H), 2.50 (s, 3H), 2.45-2.35 (m, 4H), 2.30-1.90 (m, 4H), 1.89-1.70 (m, 5H), 1.69-1.50 (m, 6H), 1.45-1.40 (m, 3H), 1.30-0.90 (m, 6H); LC-MS (ES⁺): m/z 1050.50 [MNa⁺].

### Example 27 tert-Butyl N-[(1S)-1-[[(1S)-2-[6-[2-(4-[2-[2-(2-[2-[(9S)-7-(4-chlorophenyl)-4,5,13-trimethyl-3-thia-1,8,11,12-tetraazatricyclo[8.3.0.0\hat{ }[2,6]]trideca-2(6),4,7,10,12-pentaen-9-yl]acetamido]ethoxy)ethoxy]ethoxy]phenyl)ethyl]-octahydro-1H-pyrrolo[2,3-c]pyridin-1-yl]-1-cyclohexyl-2-oxoethyl]carbamoyl]ethyl]-N-methylcarbamate

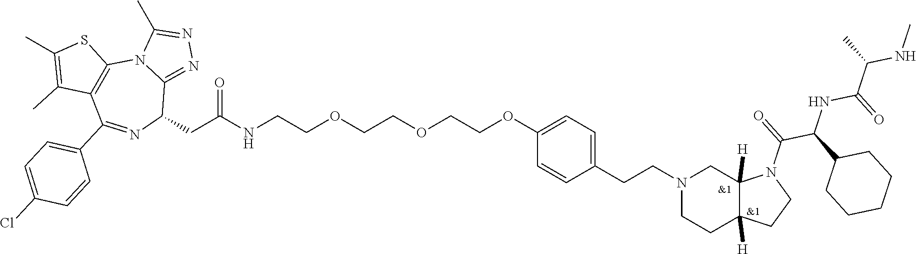 [SMILES: CN[C@@H](C)C(=O)N[C@H](C(=O)N1CC[C@@H]2CCN(CCc3ccc(OCCOCCOCCNC(=O)C[C@@H]4N=C(c5ccc(Cl)cc5)c5c(sc(C)c5C)-n5c(C)nnc54)cc3)C[C@@H]21)C1CCCCC1]

¹H NMR (300 MHz, CD₃OD): δ 8.51 (s, 4H), 7.40 (q, J=8.4 Hz, 4H), 7.09 (dd, J=8.2, 3.7 Hz, 2H), 6.83 (d, J=8.1 Hz, 2H), 4.59-4.36 (m, 1H), 4.11-4.01 (m, 2H), 4.01-4.92 (m, 2H), 3.91-3.73 (m, 4H), 3.72-3.53 (m, 6H), 3.50-3.42 (m, 1H), 3.41-3.33 (m, 3H), 3.12-2.94 (m, 2H), 2.93-2.79 (m, 4H), 2.67 (s, 4H), 2.59 (s, 3H), 2.51 (s, 5H), 2.13 (m, 2H), 2.04-1.91 (m, 1H), 1.74-1.67 (m, 2H), 1.67-1.41 (m, 3H), 1.42-1.24 (m, 5H), 1.35-1.32 (m, 3H) 1.30-0.90 (m, 6H); LC-MS (ES⁺): m/z 984.60 [MH⁺]

### Example 28

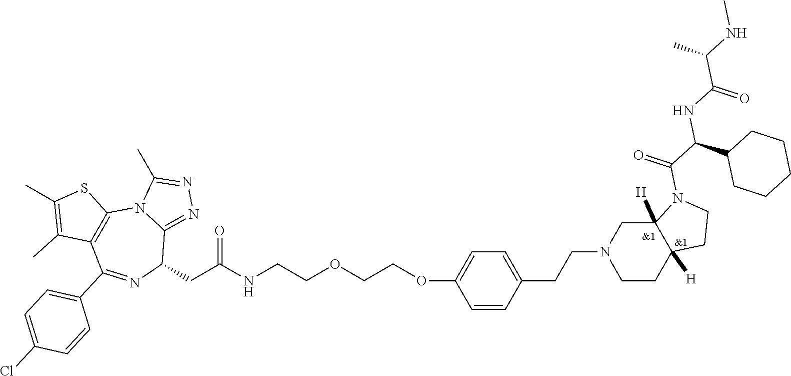 [SMILES: CN[C@@H](C)C(=O)N[C@H](C(=O)N1CC[C@@H]2CCN(CCc3ccc(OCCOCCNC(=O)C[C@@H]4N=C(c5ccc(Cl)cc5)c5c(sc(C)c5C)-n5c(C)nnc54)cc3)C[C@@H]21)C1CCCCC1]

### Example 29 4-[(2-[2-[(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)oxy]ethoxy]ethyl)amino]-N-[(1r,3r)-3-(3-chloro-4-cyanophenoxy)-2,2,4,4-tetramethylcyclobutyl]benzamide

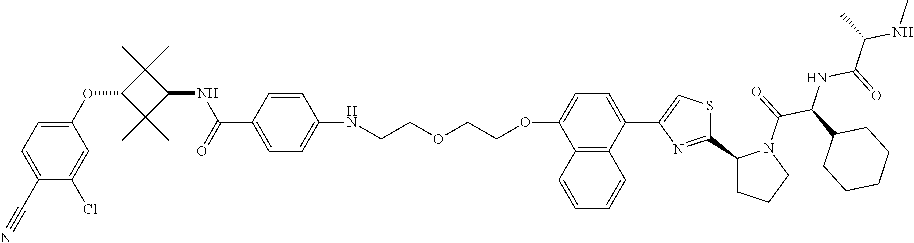

### Step 1. Methyl 4-[[2-(2-hydroxyethoxy)ethyl]amino]benzoate

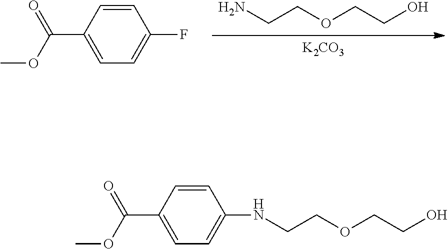

Into a 100-mL round-bottom flask, was placed a solution of methyl 4-fluorobenzoate (5.0 g, 32.44 mmol, 1.00 equiv) in NMP (50 mL), 2-(2-aminoethoxy)ethan-1-ol (4.1 g, 39.00 mmol, 1.20 equiv), potassium carbonate (5.4 g, 39.13 mmol, 1.20 equiv). The resulting solution was stirred for 12 h at 130° C. The reaction mixture was cooled. The reaction was then quenched by the addition of water (50 mL). The resulting solution was extracted with ethyl acetate (50 mL×3) and the organic layers combined. The resulting mixture was washed with brine (50 mL×3). The mixture was dried over anhydrous sodium sulfate and concentrated under vacuum. The residue was applied onto a silica gel column with ethyl acetate/petroleum ether (1/1). This resulted in 3.2 g (41%) of methyl 4-[[2-(2-hydroxyethoxy)ethyl]amino]benzoate as a yellow solid.

LC-MS (ES⁺): m/z 240.00 [MH⁺]

### Step 2. Methyl 4-([2-[2-(oxan-2-yloxy)ethoxy]ethyl]amino)benzoate

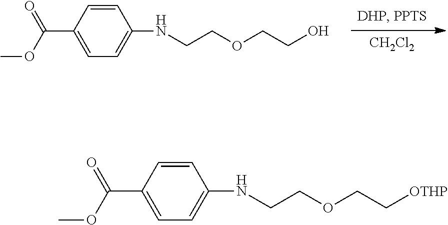

Into a 25-mL round-bottom flask, was placed a solution of methyl 4-[[2-(2-hydroxyethoxy)ethyl]amino]benzoate (700.0 mg, 2.93 mmol, 1.00 equiv) in dichloromethane (10 mL), DHP (246 mg, 2.92 mmol, 2.00 equiv), PPTS (10.0 mg, 0.04 mmol, 0.01 equiv). The resulting solution was stirred for 12 h at room temperature. The reaction was then quenched by the addition of water (5 mL). The resulting mixture was concentrated under vacuum. The residue was applied onto a silica gel column with ethyl acetate/petroleum ether (1/1). This resulted in 900.0 mg (95%) of methyl 4-([2-[2-(oxan-2-yloxy)ethoxy]ethyl]amino)benzoate as light yellow oil.

### Step 3. 4-([2-[2-(Oxan-2-yloxy)ethoxy]ethyl]amino)benzoic acid

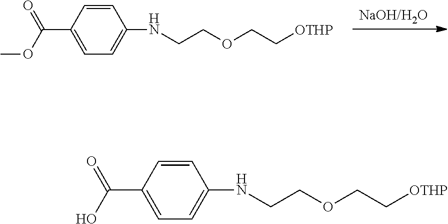

Into a 100-mL round-bottom flask, was placed a solution of methyl 4-([2-[2-(oxan-2-yloxy)ethoxy]ethyl]amino)benzoate (1000.0 mg, 3.09 mmol, 1.00 equiv) in methanol/water (20/10 mL), sodium hydroxide (495.0 mg, 12.38 mmol, 4.00 equiv). The resulting solution was stirred overnight at 50° C. The resulting mixture was concentrated under vacuum. The pH value of the solution was adjusted to 5-6 with 1 M hydrogen chloride. The resulting solution was extracted with dichloromethane (50 mL×3) and the organic layers combined. The mixture was dried over anhydrous sodium sulfate and concentrated under vacuum. This resulted in 910.0 mg (95%) of 4-([2-[2-(oxan-2-yloxy)ethoxy]ethyl]amino)benzoic acid as yellow oil.

LC-MS (ES⁺): m/z 310.00 [MH⁺]

### Step 4. 4-([2-[2-(Oxan-2-yloxy)ethoxy]ethyl]amino)-N-[(1r,3r)-3-(3-chloro-4-cyanophenoxy)-2,2,4,4-tetramethylcyclobutyl]benzamide

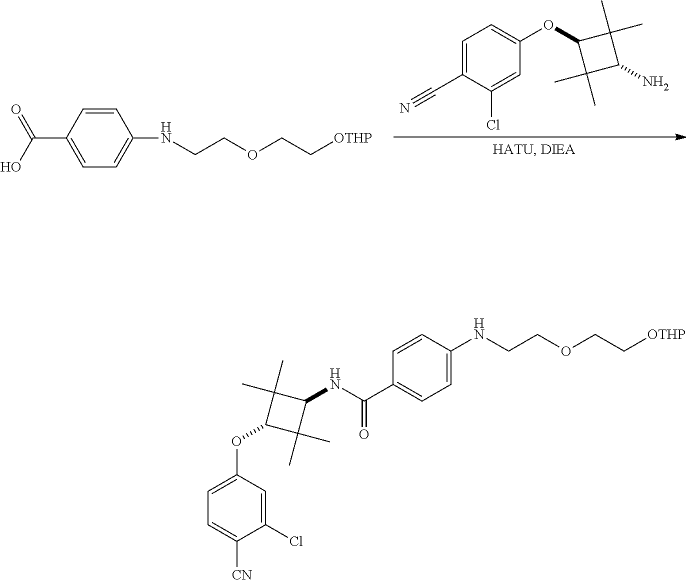

Into a 25-mL round-bottom flask, was placed a solution of 4-([2-[2-(oxan-2-yloxy)ethoxy]ethyl]amino)benzoic acid (246.0 mg, 0.80 mmol, 1.00 equiv) in N,N-dimethylformamide (10 mL). This was followed by the addition of HATU (363 mg, 0.95 mmol, 1.22 equiv). DIEA 0.5 mL was added into at 0° C. To this was added 2-chloro-4-[(1r,3r)-3-amino-2,2,4,4-tetramethylcyclobutoxy]benzonitrile hydrogen chloride [prepared as described previously by Crew, A. P. et al. in US 20150291562] (300.0 mg, 1.08 mmol, 1.20 equiv). The resulting solution was stirred for 2 h at room temperature. The reaction was then quenched by the addition of water (20 mL). The resulting solution was extracted with ethyl acetate (50 mL×3) and the organic layers combined. The resulting mixture was washed with brine (50 mL×3). The mixture was dried over sodium sulfate and concentrated under vacuum. The residue was applied onto a silica gel column with ethyl acetate/petroleum ether (1/1). This resulted in 414.0 mg (91%) of 4-([2-[2-(oxan-2-yloxy)ethoxy]ethyl]amino)-N-[(1r,3r)-3-(3-chloro-4-cyanophenoxy)-2,2,4,4-tetramethylcyclobutyl]benzamide as light yellow oil.

LC-MS (ES⁺): m/z 592.25/594.25 [MNa⁺]

### Step 5. 4-[[2-(2-hydroxyethoxy)ethyl]amino]-N-[(1r,3r)-3-(3-chloro-4-cyanophenoxy)-2,2,4,4-tetramethylcyclobutyl]benzamide

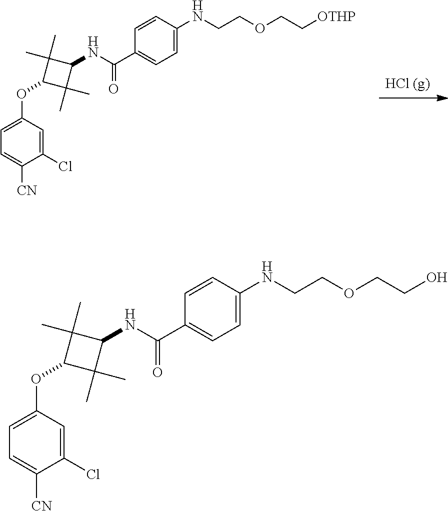

Into a 25-mL round-bottom flask, was placed 4-([2-[2-(oxan-2-yloxy)ethoxy]ethyl]amino)-N-[(1r,3r)-3-(3-chloro-4-cyanophenoxy)-2,2,4,4-tetramethylcyclobutyl]benzamide (231.0 mg, 0.41 mmol, 1.00 equiv), methanol (5.0 mL). To the above hydrogen chloride (g) was introduced in. The resulting solution was stirred for 2 h at room temperature. The resulting mixture was concentrated under vacuum. The pH value of the solution was adjusted to 9 with sodium bicarbonate (1 mol/L). The resulting solution was extracted with dichloromethane (50 mL×3) and the organic layers combined and dried over anhydrous sodium sulfate, concentrated under vacuo. This resulted in 172.0 mg (87%) of 4-[[2-(2-hydroxyethoxy)ethyl]amino]-N-[(1r,3r)-3-(3-chloro-4-cyanophenoxy)-2,2,4,4-tetramethylcyclobutyl]benzamide as light yellow oil.
Steps 6 through 8 were carried out using procedures described for steps 10-12 of Example 4 to afford the title compound, 4-[(2-[2-[(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)oxy]ethoxy]ethyl)amino]-N-[(1r,3r)-3-(3-chloro-4-cyanophenoxy)-2,2,4,4-tetramethylcyclobutyl]benzamide, as a white solid.

¹H NMR (400 MHz, CD₃OD): δ 8.35-8.33 (d, J=8.8 Hz, 1H), 8.13-8.11 (d, J=6.4 Hz, 1H), 7.75-7.73 (d, J=8.8 Hz, 1H), 7.65-7.63 (d, J=8.8 Hz, 2H), 7.57-7.48 (m, 4H), 7.14 (d, J=2.4 Hz, 1H), 7.01-6.98 (d, J=8.4 Hz, 2H), 6.67-6.65 (d, J=8.8 Hz, 2H), 5.55 (m, 1H), 4.63-4.61 (m, 1H), 4.41-4.38 (t, J=4.6 Hz, 2H), 4.29 (s, 1H), 4.13 (s, 1H), 4.06-4.00 (m, 4H), 3.88-3.85 (t, J=5.4 Hz, 2H), 3.45-3.42 (m, 2H), 3.21-3.19 (m, 1H), 2.34-2.20 (m, 5H), 2.18 (s, 2H), 1.85-1.84 (m, 3H), 1.64-1.63 (m, 3H), 1.27-1.18 (m, 20H); LC-MS (ES⁺): m/z 988.20/990.20 [MH⁺]

### Example 30: 4-[1-(4-[2-[(2S)-1-[(2S)-2-Cyclohexyl-2-[(2S)-2-(methylamino)propanamido]acetyl]pyrrolidin-2-yl]-1,3-thiazol-4-yl]naphthalen-1-yl)-1,4,7-trioxa-10-azadecan-10-yl]-N-[(1r,3r)-3-(3-chloro-4-cyanophenoxy)-2,2,4,4-tetramethylcyclobutyl]benzamide

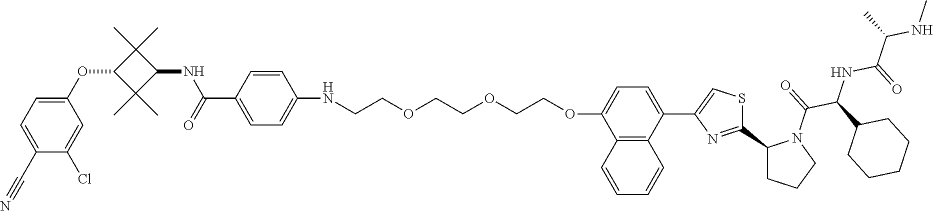 [SMILES: CN[C@@H](C)C(=O)N[C@H](C(=O)N1CCC[C@H]1c1nc(-c2ccc(OCCOCCOCCNc3ccc(C(=O)N[C@H]4C(C)(C)[C@H](Oc5ccc(C#N)c(Cl)c5)C4(C)C)cc3)c3ccccc23)cs1)C1CCCCC1]

### Step 1. Methyl 4-([2-[2-(2-hydroxyethoxy)ethoxy]ethyl]amino)benzoate

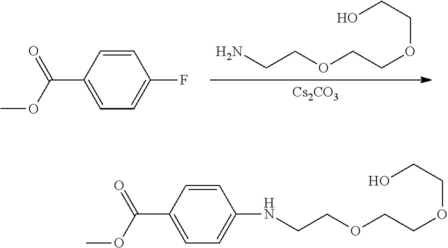

Into a 25-mL round-bottom flask, was placed methyl 4-fluorobenzoate (500.0 mg, 3.24 mmol, 1.00 equiv), NMP (10 mL), potassium carbonate (894.24 mg, 6.47 mmol, 2.00 equiv), 2-[2-(2-aminoethoxy)ethoxy]ethan-1-ol (580.0 mg, 3.89 mmol, 1.20 equiv). The resulting solution was stirred 12 h at 130° C. in an oil bath. The resulting solution was extracted with ethyl acetate (50 mL×3) and the organic layers combined. The resulting mixture was washed with brine (50 mL×3). The mixture was dried over anhydrous sodium sulfate. The residue was applied onto a silica gel column with ethyl acetate/petroleum ether (9/1). This resulted in 300.0 mg (33%) of methyl 4-([2-[2-(2-hydroxyethoxy)ethoxy]ethyl]amino)benzoate as light yellow oil.

LC-MS (ES⁺): m/z 284.05[MH⁺]

### Step 2. Methyl 4-([2-[2-(2-[[(4-methylbenzene)sulfonyl]oxy]ethoxy)ethoxy]ethyl]amino)benzoate

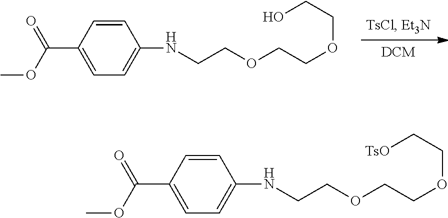

Into a 50-mL round-bottom flask purged and maintained with an inert atmosphere of nitrogen, was placed methyl 4-([2-[2-(2-hydroxyethoxy)ethoxy]ethyl]amino)benzoate (150.0 mg, 0.53 mmol, 1.00 equiv), dichloromethane (10 mL), 4-methylbenzene-1-sulfonyl chloride (131.0 mg, 0.69 mmol, 1.30 equiv), triethylamine (100.0 mg, 0.99 mmol, 2.00 equiv), 4-dimethylaminopyridine (20.0 mg, 0.16 mmol, 0.31 equiv). The resulting solution was stirred 16 h at room temperature. The residue was applied onto a silica gel column with ethyl acetate/petroleum ether (1:3). This resulted in 210.0 mg (91%) of methyl 4-([2-[2-(2-[[(4-methylbenzene)sulfonyl]oxy]ethoxy)ethoxy]ethyl]amino)benzoate as yellow oil.

### Step 3. Methyl 4-[1-(4-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoate

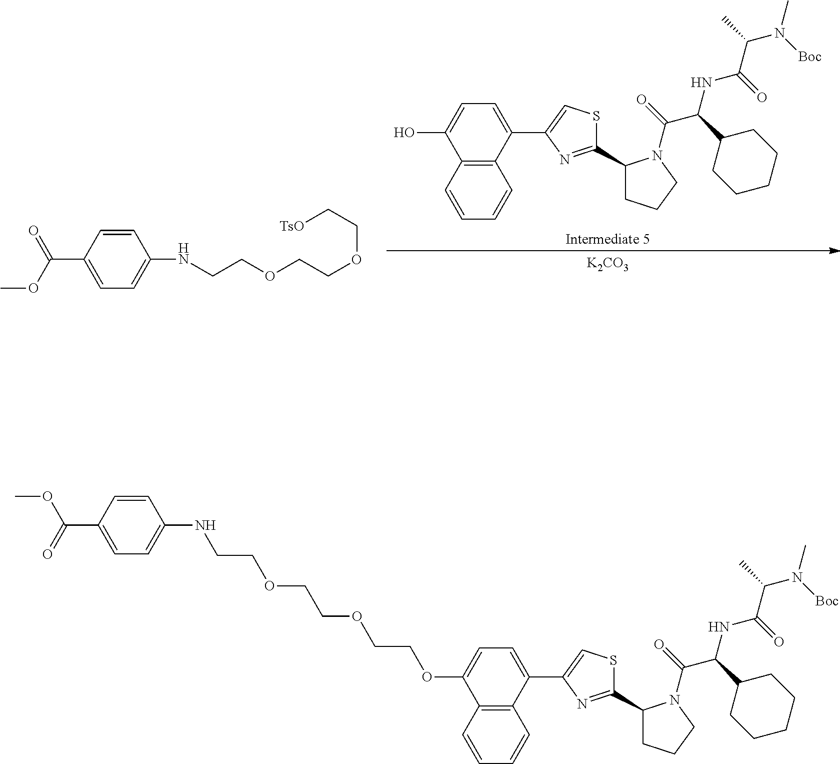

Into a 50-mL round-bottom flask, was placed tert-butyl N-[(1S)-1-[[(1S)-1-cyclohexyl-2-[(2S)-2-[4-(4-hydroxynaphthalen-1-yl)-1,3-thiazol-2-yl]pyrrolidin-1-yl]-2-oxoethyl]carbamoyl]ethyl]-N-methylcarbamate (50.0 mg, 0.08 mmol, 1.00 equiv), N,N-dimethylformamide (5.0 mL), potassium carbonate (22.25 mg, 0.16 mmol, 1.00 equiv), methyl 4-(2-[2-(2-[(4-methylbenzene)sulfonyl]oxyethoxy)ethoxy]ethylamino)benzoate (38.76 mg, 0.09 mmol, 1.10 equiv). The resulting solution was stirred for 5 h at 80° C. in an oil bath. The resulting solution was extracted with ethyl acetate (50 mL×3) and the organic layers combined. The resulting mixture was washed with brine (50 mL×3). The mixture was dried over anhydrous sodium sulfate. The residue was purified by TLC with ethyl acetate/petroleum ether (7/3). This resulted in 97.0 mg (crude) of methyl 4-[1-(4-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoate as light yellow oil.

LC-MS (ES⁺): m/z 908.45 [MNa⁺]

### Step 4. 4-[1-(4-[2-[(2S)-1-[(2S)-2-[(2S)-2-[[(tert-Butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoic acid

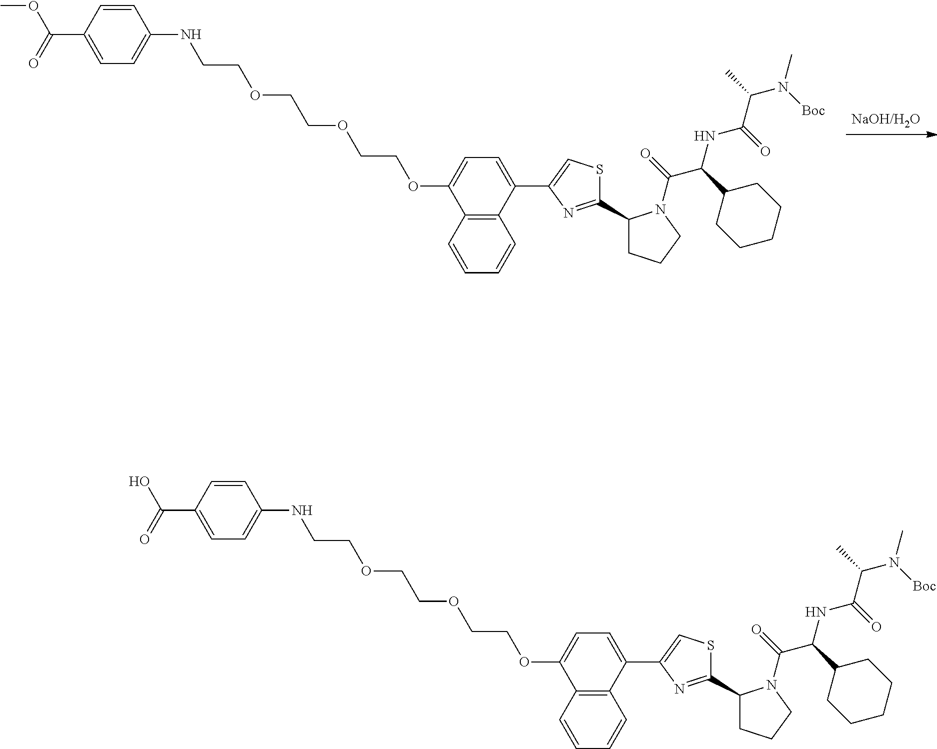 [SMILES: COC(=O)c1ccc(NCCOCCOCCOc2ccc(-c3csc([C@@H]4CCCN4C(=O)[C@@H](NC(=O)[C@H](C)N(C)C(=O)OC(C)(C)C)C4CCCCC4)n3)c3ccccc23)cc1 ; "NaOH/H2O" ; C[C@@H](C(=O)N[C@H](C(=O)N1CCC[C@H]1c1nc(-c2ccc(OCCOCCOCCNc3ccc(C(=O)O)cc3)c3ccccc23)cs1)C1CCCCC1)N(C)C(=O)OC(C)(C)C]

Into a 25-mL round-bottom flask, was placed methyl 4-[1-(4-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoate (97.0 mg, 0.11 mmol, 1.00 equiv), methanol (5.0 mL), a solution of sodium hydroxide (8.7 mg, 0.22 mmol, 2.00 equiv) in water (2.0 mL). The resulting solution was stirred overnight at 50° C. in an oil bath. The resulting mixture was concentrated under vacuum. The pH value of the solution was adjusted to 5. The resulting solution was extracted with ethyl acetate (50 mL×3) and the organic layers combined and dried over anhydrous sodium sulfate, concentrated under vacuo. This resulted in 95.0 mg of 4-[1-(4-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoic acid as light yellow oil.

LC-MS (ES⁺): m/z 872.40 [MH⁺]

### Step 5. tert-Butyl N-[(1S)-1-[[(1S)-1-cyclohexyl-2-oxo-2-[(2S)-2-(4-[4-[2-(2-[2-[(4-[[(1r,3r)-3-(3-chloro-4-cyanophenoxy)-2,2,4,4-tetramethylcyclobutyl]carbamoyl]phenyl)amino]ethoxy]ethoxy)ethoxy]naphthalen-1-yl]-1,3-thiazol-2-yl)pyrrolidin-1-yl]ethyl]carbamoyl]ethyl]-N-methylcarbamate

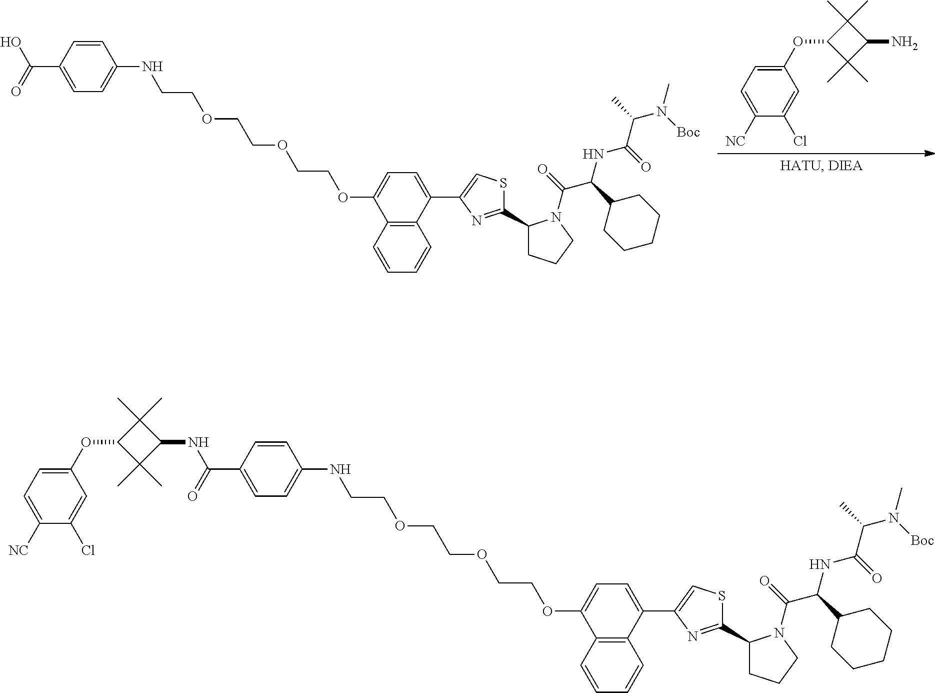

Into a 25-mL round-bottom flask, was placed 4-[1-(4-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazol-4-yl]naphthalen-1-yl)-1,4,7-trioxa-10-azadecan-10-yl]benzoic acid (80.0 mg, 0.09 mmol, 1.00 equiv), N,N-dimethylformamide (5.0 mL), 2-chloro-4-[(1r,3r)-3-amino-2,2,4,4-tetramethylcyclobutoxy]benzonitrile 4-methylbenzene-1-sulfonyl chloride (31.72 mg, 0.11 mmol, 1.10 equiv), HATU (38.39 mg, 0.10 mmol, 1.10 equiv), DIEA (47.39 mg, 0.37 mmol, 4.00 equiv) at 0° C. The resulting solution was stirred for 2 h at r.t. The resulting solution was extracted with ethyl acetate (50 mL×3) and the organic layers combined. The resulting mixture was washed with brine (50 mL×3). The mixture was dried over anhydrous sodium sulfate. The residue was purified by TLC with dichloromethane/methanol (15/1). This resulted in 55.0 mg (53%) of tert-butyl N-[(1S)-1-[[(1S)-1-cyclohexyl-2-oxo-2-[(2S)-2-(4-[4-[2-(2-[2-[(4-[[(1r,3r)-3-(3-chloro-4-cyanophenoxy)-2,2,4,4-tetramethylcyclobutyl]carbamoyl]phenyl)amino]ethoxy]ethoxy)ethoxy]naphthalen-1-yl]-1,3-thiazol-2-yl)pyrrolidin-1-yl]ethyl]carbamoyl]ethyl]-N-methylcarbamate as light yellow oil.

LC-MS (ES⁺): m/z 1132.50/1134.50 [MH⁺]

### Step 6. 4-[1-(4-[2-[(2S)-1-[(2S)-2-Cyclohexyl-2-[(2S)-2-(methylamino)propanamido]acetyl]pyrrolidin-2-yl]-1,3-thiazol-4-yl]naphthalen-1-yl)-1,4,7-trioxa-10-azadecan-10-yl]-N-[(1r,3r)-3-(3-chloro-4-cyanophenoxy)-2,2,4,4-tetramethylcyclobutyl]benzamide

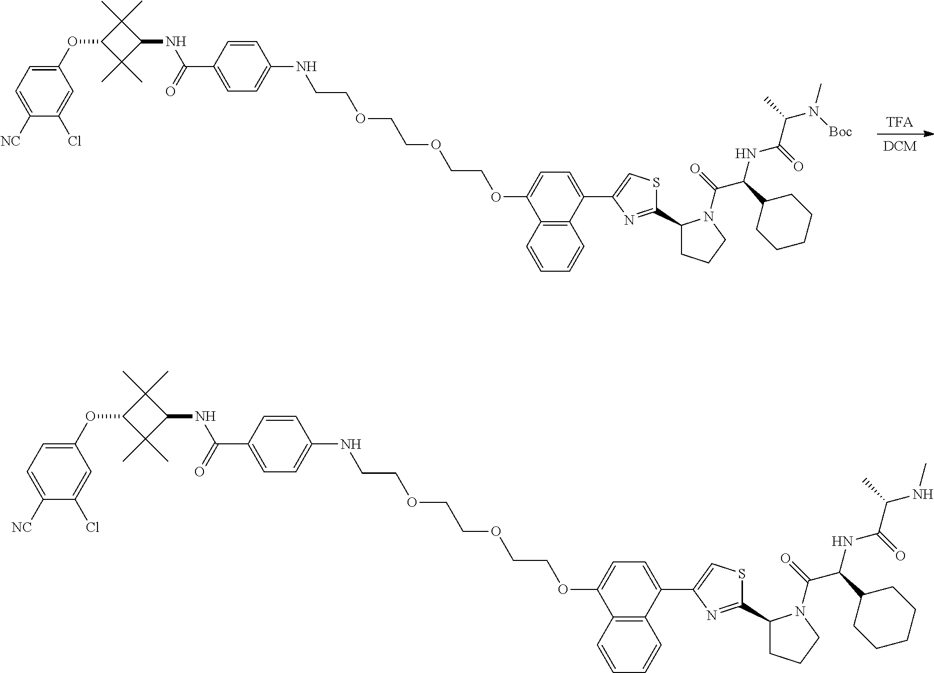

Into a 25-mL round-bottom flask, was placed tert-butyl N-[(1S)-1-[[(1S)-1-cyclohexyl-2-oxo-2-[(2S)-2-(4-[4-[2-(2-[2-[(4-[[(1r,3r)-3-(3-chloro-4-cyanophenoxy)-2,2,4,4-tetramethylcyclobutyl]carbamoyl]phenyl)amino]ethoxy]ethoxy)ethoxy]naphthalen-1-yl]-1,3-thiazol-2-yl)pyrrolidin-1-yl]ethyl]carbamoyl]ethyl]-N-methylcarbamate (55.0 mg, 0.05 mmol, 1.00 equiv), dichloromethane (5.0 mL), trifluoroacetic acid (3.0 mL). The resulting solution was stirred for 2 h at r.t. The resulting mixture was concentrated under vacuum. The crude product was purified by Prep-HPLC with the following conditions Column, XBridge C18 OBD Prep Column, 5 μm, 19 mm×250 mm; mobile phase, waters (10 mmol/L NH₄HCO₃) and acetonitrile (63.0% acetonitrile up to 75.0% in 10 min); Detector, UV 254 nm. This resulted in 25.0 mg (50%) of 4-[1-(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)-1,4,7-trioxa-10-azadecan-10-yl]-N-[(1r,3r)-3-(3-chloro-4-cyanophenoxy)-2,2,4,4-tetramethylcyclobutyl]benzamide as a white solid.

¹H NMR (400 MHz, CD₃OD): δ 8.38-8.36 (d, J=9.6 Hz, 1H), 8.12-8.11 (d, J=5.2 Hz, 1H), 7.75-7.73 (d, J=8.8 Hz, 1H), 7.64-7.61 (d, J=8.8 Hz, 2H), 7.57-7.48 (m, 4H), 7.14-7.13 (d, J=2.4 Hz, 1H), 7.00-6.97 (d, J=11.2 Hz, 2H), 6.64-6.62 (d, J=8.8 Hz, 2H), 5.55 (m, 1H), 4.63-4.61 (m, 1H), 4.38-4.36 (t, J=4.4 Hz, 2H), 4.28 (s, 1H), 4.12 (s, 1H), 4.06-3.90 (m, 4H), 3.84-3.83 (t, J=5.4 Hz, 2H), 3.74-3.71 (m, 4H), 3.21-3.19 (m, 1H), 2.37-2.20 (m, 4H), 2.30-2.10 (m, 3H), 1.84-1.63 (m, 6H), 1.31-1.13 (m, 22H); LC-MS (ES⁺): m/z 1032.10/1034.10 [MH⁺]

Using procedures analogous to those described above for Example 30, the following compounds have been prepared:

### Example 31 4-[1-(4-[2-[(2S)-1-[(2S)-2-cyclohexyl-2-[(2S)-2-(methylamino)propanamido]acetyl]pyrrolidin-2-yl]-1,3-thiazol-4-yl]naphthalen-1-yl)-1,4,7,10-tetraoxa-13-azatridecan-13-yl]-N-[(1r,3r)-3-(3-chloro-4-cyanophenoxy)-2,2,4,4-tetramethylcyclobutyl]benzamide

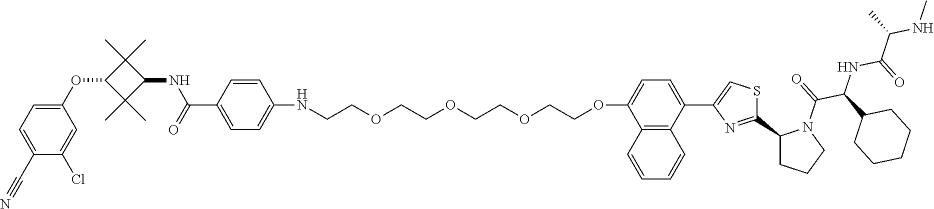 [SMILES: CN[C@@H](C)C(=O)N[C@H](C(=O)N1CCC[C@H]1c1nc(-c2ccc(OCCOCCOCCOCCNc3ccc(C(=O)N[C@H]4C(C)(C)[C@H](Oc5ccc(C#N)c(Cl)c5)C4(C)C)cc3)c3ccccc23)cs1)C1CCCCC1]

¹H NMR (400 MHz, CD₃OD): δ 8.38-8.36 (d, J=9.6 Hz, 1H), 8.12-8.11 (d, J=5.2 Hz, 1H), 7.74-7.72 (d, J=8.8 Hz, 1H), 7.66-7.63 (d, J=8.8 Hz, 2H), 7.56-7.48 (m, 4H), 7.14-7.13 (d, J=2.4 Hz, 1H), 7.00-6.95 (d, J=11.2 Hz, 2H), 6.65-6.63 (d, J=8.8 Hz, 2H), 5.55 (m, 1H), 4.63-4.61 (m, 1H), 4.37-4.35 (t, J=4.6 Hz, 2H), 4.27 (s, 1H), 4.12 (s, 1H), 4.05-3.90 (m, 4H), 3.83-3.81 (m, 2H), 3.74-3.61 (m, 8H), 3.32-3.30 (m, 2H), 3.28-3.27 (m, 1H), 2.34-2.00 (m, 7H), 1.84-1.63 (m, 6H), 1.28-1.17 (m, 20H); LC-MS (ES⁺): m/z 1076.20/1078.20 [MH⁺]

### Example 32 4-[1-[4-(2-[1-[(2S)-2-cyclohexyl-2-[(2S)-2-(methylamino)propanamido]acetyl]-octahydro-1H-pyrrolo[2,3-c]pyridin-6-yl]ethyl)phenyl]-1,4,7-trioxa-10-azadecan-10-yl]-N-[(1r,3r)-3-(3-chloro-4-cyanophenoxy)-2,2,4,4-tetramethylcyclobutyl]benzamide

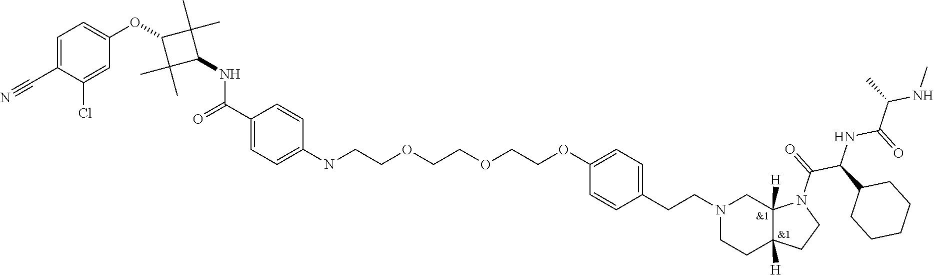

¹H NMR (400 MHz, CD₃OD): δ 7.74 (d, J=8.8 Hz, 1H), 7.66 (d, J=8.8 Hz, 2H), 7.21-7.14 (m, 3H), 7.01 (d, J=4.8 Hz, 1H), 6.99 (d, J=4.8 Hz, 2H), 6.72-6.65 (m, 2H), 4.99-4.96 (m, 2H), 4.55-4.41 (m, 1H), 4.31 (s, 1H), 4.20-4.03 (m, 3H), 3.99-3.82 (m, 4H), 3.79-3.68 (m, 7H), 3.66-3.37 (m, 7H), 3.27-2.81 (m, 4H), 2.68 (s, 3H), 2.58-2.22 (m, 2H), 2.17-2.01 (m, 2H), 1.92-1.58 (m, 6H), 1.48 (m, 2H), 1.36-1.02 (m, 17H); LC-MS (ES⁺): m/z 982.55 [MH⁺]

### Example 33 N-((1r,3r)-3-(3-chloro-4-cyanophenoxy)-2,2,4,4-tetramethylcyclobutyl)-4-(2-(2-(2-(2-(4-(2-(1-((S)-2-cyclohexyl-2-((S)-2-(methylamino)propanamido)acetyl)-octahydropyrrolo[2,3-c]pyridin-6-yl)ethyl)phenoxy)ethoxy)ethoxy)ethoxy)ethylamino)benzamide

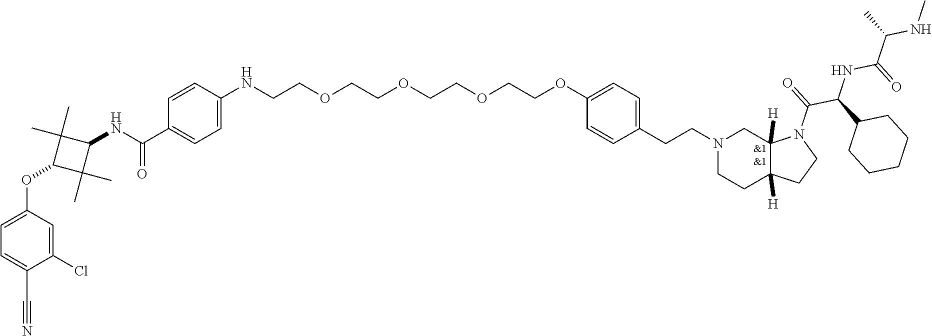 [SMILES: CN[C@@H](C)C(=O)N[C@H](C(=O)N1CC[C@@H]2CCN(CCc3ccc(OCCOCCOCCOCCNc4ccc(C(=O)N[C@H]5C(C)(C)[C@H](Oc6ccc(C#N)c(Cl)c6)C5(C)C)cc4)cc3)C[C@@H]21)C1CCCCC1]

¹H NMR (300 MHz, CDCl₃) δ 7.57 (dd, J=16.0, 8.5 Hz, 3H), 7.07 (t, J=9.1 Hz, 2H), 6.94 (d, J=2.4 Hz, 1H), 6.85-6.70 (m, 2H), 6.58 (d, J=8.3 Hz, 2H), 6.06 (d, J=8.0 Hz, 1H), 4.60 (s, 1H), 4.35 (d, J=55.4 Hz, 2H), 4.19-3.97 (m, 4H), 3.96-3.52 (m, 12H), 3.53-3.39 (m, 1H), 3.35-3.25 (m, 2H), 3.10-3.00 (m, 1H), 2.90-2.45 (m, 4H), 2.40-2.20 (m, 4H), 2.10-1.90 (m, 2H), 1.85-1.50 (m, 13H), 1.45-1.20 (m, 4H), 1.20-1.10 (m, 12H), 1.10-0.96 (m, 3H); LC-MS (ES⁺): m/z 1026.70 [MH⁺]

### Example 34: (S)—N-((1S,2R)-2-(3-(5-(4-(3-(4-Cyano-3-(trifluoromethyl)phenyl)-5,5-dimethyl-4-oxo-2-thioxoimidazolidin-1-yl)phenoxy)pentyloxy)propoxy)-2,3-dihydro-1H-inden-1-yl)-1-((S)-3,3-dimethyl-2-((S)-2-(methylamino)propanamido)butanoyl)-pyrrolidine-2-carboxamide

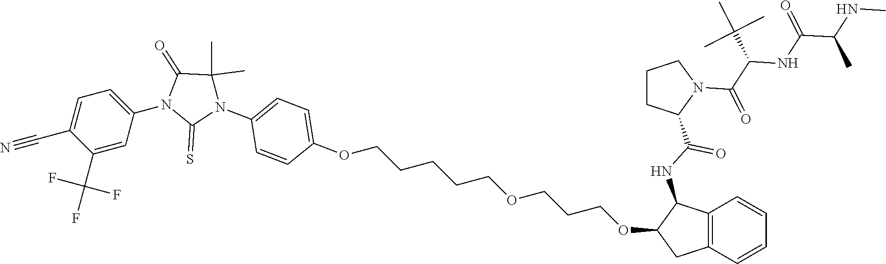 [SMILES: CN[C@@H](C)C(=O)N[C@H](C(=O)N1CCC[C@H]1C(=O)N[C@H]1c2ccccc2C[C@H]1OCCCOCCCCCOc1ccc(N2C(=S)N(c3ccc(C#N)c(C(F)(F)F)c3)C(=O)C2(C)C)cc1)C(C)(C)C]

### Step 1. ({[5-(Prop-2-en-1-yloxy)pentyl]oxy}methyl)benzene

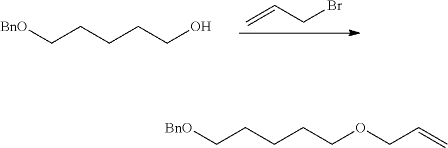

To a stirred solution of 5-(benzyloxy)pentan-1-ol (CAS #4541-15-5, 4.0 g, 20.59 mmol) in N,N-dimethylformamide (50 mL) was added sodium hydride (1.24 g, 51.67 mmol) in portions at 0° C. under an atmosphere of nitrogen. The resulting mixture was then stirred at rt for 1 h. To this mixture was added 3-bromoprop-1-ene (3.71 g, 30.67 mmol), the reaction mixture was stirred overnight at 60° C. in an oil bath. The reaction mixture was cooled to 0° C. and then quenched by water (100 mL), the resulting mixture was extracted with ethyl acetate (200 mL×2). The organic layers were combined, washed with saturated aqueous solution of sodium chloride (60 mL), dried over anhydrous sodium sulfate and then concentrated under reduced pressure to give a crude residue. The residue was purified by a flash silica gel chromatography (eluent: ethyl acetate/petroleum ether (v:v=1:40)) to give 4.57 g of the title product.

¹H NMR (300 MHz, CDCl₃): δ 7.36 (s, 4H), 7.32 (m, 1H), 5.98 (m, 1H), 5.33 (m, 1H), 5.21 (m, 1H), 4.53 (s, 2H), 3.99 (m, 2H), 3.53 (m, 4H), 1.72 (m, 4H), 1.52 (m, 2H). LC-MS (ES⁺): m/z 235.00 [MH⁺]

### Step 2. 3-{[5-(Benzyloxy)pentyl]oxy}propan-1-ol

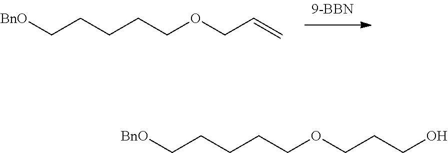 [SMILES: C=CCOCCCCCOCc1ccccc1 ; OCCCOCCCCCOCc1ccccc1]

To a 250-mL round-bottom flask with 9-BBN (0.5 M in THF, 77 mL) was added a solution of ({[5-(prop-2-en-1-yloxy)pentyl]oxy}methyl)benzene (3.0 g, 12.80 mmol) in anhydrous tetrahydrofuran (20 mL) with stirring at 0° C. under an atmosphere of nitrogen. The resulting solution was stirred overnight at rt. Methanol (15 mL, with 30% sodium hydroxide and 30% H₂O₂) was added to the reaction and the resulting mixture was stirred at rt for 2 h. This mixture was then extracted with ethyl acetate (20 mL×3). The organic layers were combined, washed with saturated aqueous solution of sodium chloride (100 mL), dried over anhydrous sodium sulfate and then concentrated under reduced pressure to give a crude residue. The residue was purified by a flash silica gel chromatography (eluent: ethyl acetate/petroleum ether (v:v=1:1)) to provide 1.96 g of the title compound as light yellow oil.

¹H NMR (300 MHz, CDCl₃): δ7.34 (m, 5H), 4.49 (s, 2H), 3.75 (m, 2H), 3.59 (m, 2H), 3.49 (m, 4H), 2.65 (bs, 1H), 1.84 (m, 2H), 1.68 (m, 4H), 1.50 (m, 2H). LC-MS (ES⁺): m/z 253.17 [MH⁺]

### Step 3. 3-{[5-(Benzyloxy)pentyl]oxy}propyl 4-methylbenzene-1-sulfonate

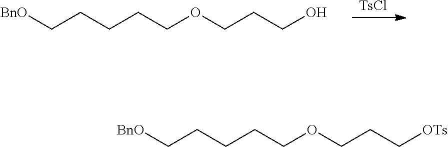 [SMILES: OCCCOCCCCCOCc1ccccc1 ; "TsCl" ; Cc1ccc(S(=O)(=O)OCCCOCCCCCOCc2ccccc2)cc1]

The experiment was run as described for step 2 of Example 1.

### Step 4. (1S,2R)-2-(3-(5-(Benzyloxy)pentyloxy)propoxy)-2,3-dihydro-1H-inden-1-amine

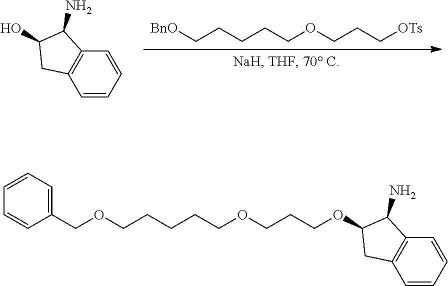

To a solution of (1S,2R)-1-amino-2,3-dihydro-1H-inden-2-ol (500.0 mg, 3.4 mmol, 1.0 eq) in anhydrous tetrahydrofuran (20 mL) were added sodium hydride (160.0 mg, 4.1 mmol, 1.2 eq), and 3-(5-(benzyloxy)pentyloxy)propyl 4-methylbenzenesulfonate (1.3 g, 3.4 mmol, 1.0 eq). The resulting solution was stirred at 70° C. for 16 h. Then the reaction was cooled to rt and quenched by the addition of water (100 mL). The resulting solution was extracted with ethyl acetate (20 mL×3). The combined organic layers were washed with brine (20 mL×2), dried over anhydrous sodium sulfate and concentrated under vacuum. The residue was applied onto a silica gel column with dichloromethane/methanol (10:1) to afford 0.5 g (38%) of (1S, 2R)-2-(3-(5-(benzyloxy)pentyloxy)propoxy)-2,3-dihydro-1H-inden-1-amine as brown oil.

¹HNMR (400 MHz, CDCl₃): δ 7.12 (t, J=3.6 Hz, 1H), 7.31-7.35 (m, 4H), 7.26-7.28 (m, 2H), 7.21-7.23 (m, 2H), 4.49 (s, 2H), 4.33 (d, J=4.4 Hz, 1H), 4.11 (q, J=4.8 Hz, 1H), 3.51-3.75 (m, 2H), 3.45-3.49 (m, 7H), 3.39 (t, J=6.4 Hz, 2H), 2.99 (t, J=4.4 Hz, 1H), 1.83-1.86 (m, 2H), 1.56-1.66 (m, 4H), 1.39-1.44 (m, 2H), 1.24 (t, J=6.8 Hz, 1H).

### Step 5. Tert-Butyl (R)-1-((S)-1-((S)-2-(((1S,2R)-2-(3-(5-(benzyloxy)pentyloxy)propoxy)-2,3-dihydro-1H-inden-1-yl)carbamoyl)pyrrolidin-1-yl)-3,3-dimethyl-1-oxobutan-2-ylamino)-1-oxopropan-2-yl(methyl)carbamate

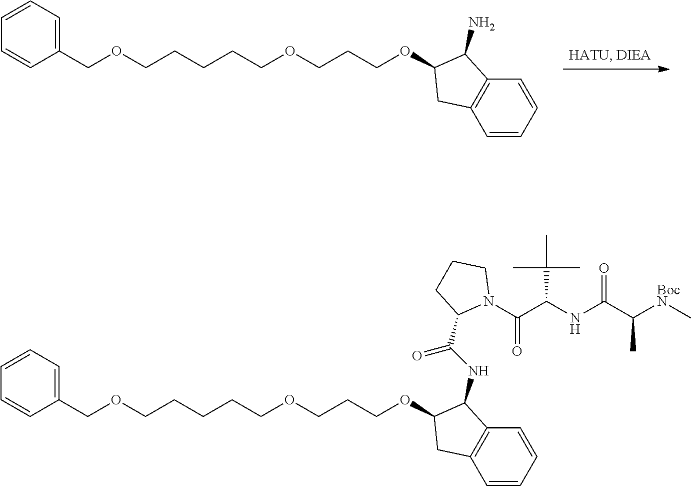 [SMILES: N[C@H]1c2ccccc2C[C@H]1OCCCOCCCCCOCc1ccccc1 ; "HATU, DIEA" ; C[C@@H](C(=O)N[C@H](C(=O)N1CCC[C@H]1C(=O)N[C@H]1c2ccccc2C[C@H]1OCCCOCCCCCOCc1ccccc1)C(C)(C)C)N(C)C(=O)OC(C)(C)C]

To a solution of (1S,2R)-2-(3-(5-(benzyloxy)pentyloxy)propoxy)-2,3-dihydro-1H-inden-1-amine (500.0 mg, 1.0 eq), DIEA (1.0 mL) and (S)-1-((S)-2-((R)-2-(tert-butoxycarbonyl)-propanamido)-3,3-dimethylbutanoyl) pyrrolidine-2-carboxylic acid (640 mg, 1.2 eq) in DMF (5 mL) was added HATU (600 mg, 1.2 eq) at rt. The resulting solution was stirred at rt for 1 h. Then the reaction was quenched by the addition of water (10 mL). The resulting solution was extracted with ethyl acetate (15 mL×3). The combined organic layers were washed with brine (10 mL×2), dried over anhydrous sodium sulfate and concentrated under vacuum. The residue was purified by prep.-TLC with dichloromethane/methanol (30:1) to afford the title compound (500 mg, as a light yellow syrup).

¹H NMR (400 MHz, CDCl₃): δ 7.15-7.34 (m, 10H), 5.42-5.48 (m, 1H), 4.58-4.61 (m, 3H), 4.49 (s, 2H), 4.23 (q, J=3.6 Hz, 1H), 3.81-3.87 (m, 1H), 3.36-3.67 (m, 9H), 3.00 (t, J=4.0 Hz, 2H), 2.77-2.80 (m, 3H), 2.35-2.42 (m, 1H), 2.12-2.18 (m, 1H), 1.93-1.99 (m, 3H), 1.80-1.83 (m, 2H), 1.55-1.65 (m, 4H), 1.41-1.50 (m, 10H), 1.31-1.48 (m, 4H), 1.26 (s, 9H).

### Step 7. tert-Butyl (R)-1-((S)-1-((S)-2-(((1S,2R)-2-(3-(5-hydroxypentyloxy)propoxy)-2,3-dihydro-1H-inden-1-yl)carbamoyl)pyrrolidin-1-yl)-3,3-dimethyl-1-oxobutan-2-ylamino)-1-oxopropan-2-yl(methyl)carbamate

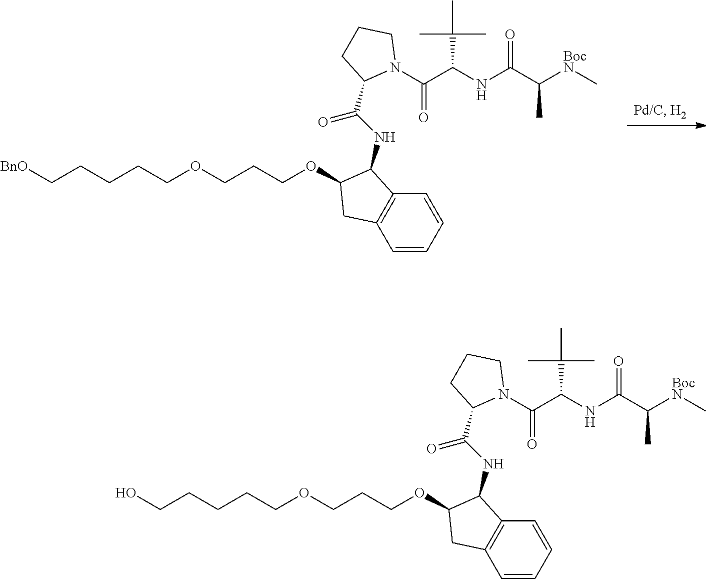 [SMILES: C[C@@H](C(=O)N[C@H](C(=O)N1CCC[C@H]1C(=O)N[C@H]1c2ccccc2C[C@H]1OCCCOCCCCCOCc1ccccc1)C(C)(C)C)N(C)C(=O)OC(C)(C)C ; "Pd/C, H2" ; C[C@@H](C(=O)N[C@H](C(=O)N1CCC[C@H]1C(=O)N[C@H]1c2ccccc2C[C@H]1OCCCOCCCCCO)C(C)(C)C)N(C)C(=O)OC(C)(C)C]

To a solution of benzyl-protected alcohol (500.0 mg, 1.0 equiv) in CH₃OH (10 mL) was added Pd/C (100 mg, 10%) at rt. The resulting solution was stirred at rt for overnight under 1 atm H₂. Then the solid was filtered off and the filtrate was concentrated under vacuum to afford crude title compound (500 mg), which was used in the next reaction without further purification.

¹HNMR (400 MHz, CDCl₃): δ 7.31-7.35 (m, 1H), 7.23 (d, J=6.8 Hz, 1H), 7.08-7.11 (m, 3H), 5.33-5.38 (m, 1H), 4.52-4.71 (m, 3H), 4.15-4.16 (m, 1H), 3.82-3.87 (m, 1H), 3.50-3.58 (m, 5H), 3.39-3.41 (m, 2H), 3.29-3.33 (m, 2H), 2.94 (d, J=4.4 Hz, 2H), 2.71 (s, 3H), 2.31-2.36 (m, 1H), 2.02-2.12 (m, 2H), 1.90-1.93 (m, 3H), 1.74-1.76 (m, 2H), 1.42-1.50 (m, 4H), 1.41 (s, 9H), 1.35-1.38 (m, 2H), 1.18-1.38 (m, 4H), 0.80 (s, 9H)

### Step 8. 5-(3-((1S,2R)-1-((S)-1-((S)-2-((R)-2-(Tert-Butoxycarbonyl)-propanamido)-3,3-dimethylbutanoyl)pyrrolidine-2-carboxamido)-2,3-dihydro-1H-inden-2-yloxy)propoxy)pentyl 4-methylbenzenesulfonate

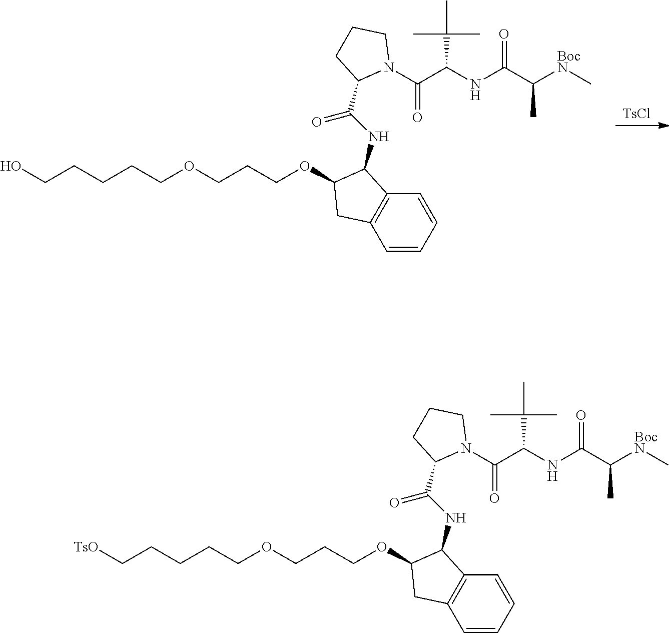

To a solution of the starting alcohol and TEA (500.0 mg, 1.0 equiv) in DCM (20 mL) was added TsCl (250 mg, 2.0 equiv). The resulting solution was stirred at rt for 10 h. Then the reaction was quenched by the addition of water (20 mL). The resulting solution was extracted with DCM (20 mL×3). The combined organic layers were washed with brine (20 mL×2), dried over anhydrous sodium sulfate and concentrated under vacuum to afford crude title compound (500 mg) as light brown syrup, which was used in the next reaction without further purification.

### Step 9. (S)—N-((1S,2R)-2-(3-(5-(4-(3-(4-Cyano-3-(trifluoromethyl)phenyl)-5,5-dimethyl-4-oxo-2-thioxoimidazolidin-1-yl)phenoxy)pentyloxy)propoxy)-2,3-dihydro-1H-inden-1-yl)-1-((S)-3,3-dimethyl-2-((S)-2-(methylamino)propanamido)butanoyl)-pyrrolidine-2-carboxamide

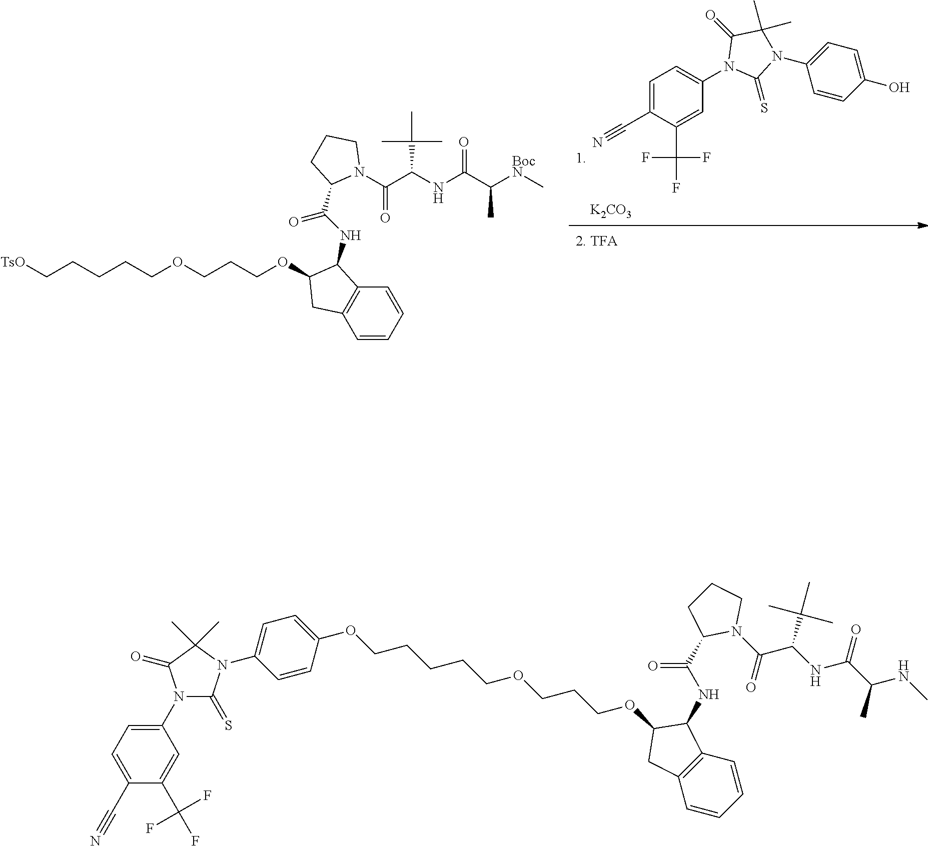

To a solution of starting tosylate (500 mg, 1.5 eq) and phenol [previously prepared as described by Crew, A. P. et al. in US 20150291562] (170 mg, 1.0 equiv) in DMF (6 mL) was added K₂CO₃ (200 mg, 4.0 eq). The resulting solution was stirred at 70° C. for 10 h. Then the reaction was cooled to rt and quenched by the addition of water (20 mL). The resulting solution was extracted with DCM (20 mL×3). The combined organic layers were washed with brine (20 mL×2), dried over anhydrous sodium sulfate and concentrated under vacuum. The crude residue was dissolved 3 mL DCM, and then TFA (3 mL) was added. The reaction mixture was stirred at rt for 1 h. It was diluted with DCM (50 mL), washed with saturated solution of NaHCO₃ (20 mL×2) and brine (20 mL×2). The combined organic layers were dried over anhydrous sodium sulfate and concentrated under vacuum. The residue was purified by prep.-TLC with DCM/CH₃OH (10:1) to afford the title compound as light yellow solid. ¹HNMR (400 MHz, CDCl₃): δ 7.96-7.99 (m, 1H), 7.85 (d, J=1.6 Hz, 1H), 7.75 (d, J=1.6 Hz, 1H), 7.29-7.33 (m, 2H), 7.14-7.20 (m, 5H), 7.02 (d, J=5.2 Hz, 2H), 5.42-5.46 (m, 1H), 4.56-4.62 (m, 2H), 4.22 (q, J=4.0 Hz, 1H), 4.00 (t, J=6.4 Hz, 1H), 3.82-3.85 (m, 1H), 3.41-3.67 (m, 8H), 3.00-3.40 (m, 3H), 2.37 (s, 4H), 2.11-2.20 (m, 1H), 1.92-2.00 (m, 2H), 1.82-1.85 (m, 3H), 1.45-1.70 (m, 14H), 1.21-1.29 (m, 4H), 0.85 (m, 9H).

LC-MS: (ES⁺): m/z 976.4 [M+H]⁺

Using procedures analogous to those described above for Example 1 and Example 34, the following compounds (Examples 35 through 38) were prepared:

### Example 35 (S)—N-((1S,2R)-2-(2-(2-(4-(3-(4-cyano-3-(trifluoromethyl)-phenyl)-5,5-dimethyl-4-oxo-2-thioxoimidazolidin-1-yl)phenoxy)ethoxy)ethoxy)-2,3-dihydro-1H-inden-1-yl)-1-((S)-3,3-dimethyl-2-((S)-2-(methylamino)propanamido)-butanoyl)pyrrolidine-2-carboxamide

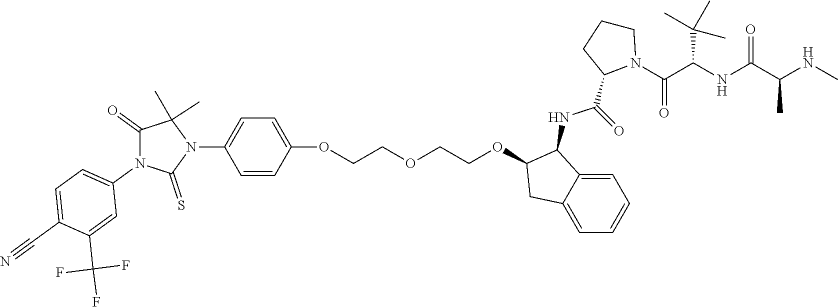 [SMILES: CN[C@@H](C)C(=O)N[C@H](C(=O)N1CCC[C@H]1C(=O)N[C@H]1c2ccccc2C[C@H]1OCCOCCOc1ccc(N2C(=S)N(c3ccc(C#N)c(C(F)(F)F)c3)C(=O)C2(C)C)cc1)C(C)(C)C]

¹H NMR (300 MHz, CD₃OD): δ 8.17-8.14 (m, 2H), 7.99 (d, J=8.1 Hz, 1H), 7.43-7.38 (m, 1H), 7.29-7.23 (m, 2H), 7.22-7.11 (m, 3H), 7.09-6.99 (m, 2H), 5.38 (d, J=5.4 Hz, 1H), 4.68 (s, 1H), 4.56-4.48 (m, 1H), 4.36-4.28 (m, 1H), 4.23-4.08 (m, 2H), 3.88-3.81 (m, 3H), 3.80-3.68 (m, 5H), 3.20-3.11 (m, 1H), 3.10-3.06 (m, 2H), 2.31 (s, 3H), 2.11-1.98 (m, 3H), 1.82-1.73 (m, 1H), 1.55 (s, 6H), 1.29-1.22 (m, 3H), 1.08-1.00 (m, 9H). LC-MS (ES⁺): m/z 920.35 [MH⁺]

### Example 36 (2S)—N-[2-(2-[2-[2-(4-[3-[4-cyano-3-(trifluoromethyl)phenyl]-5,5-dimethyl-4-oxo-2-sulfanylideneimidazolidin-1-yl]phenoxy)ethoxy]ethoxy]ethoxy)-2,3-dihydro-1H-inden-1-yl]-1-[(2S)-3,3-dimethyl-2-[(2S)-2-(methylamino)propanamido]-butanoyl]pyrrolidine-2-carboxamide

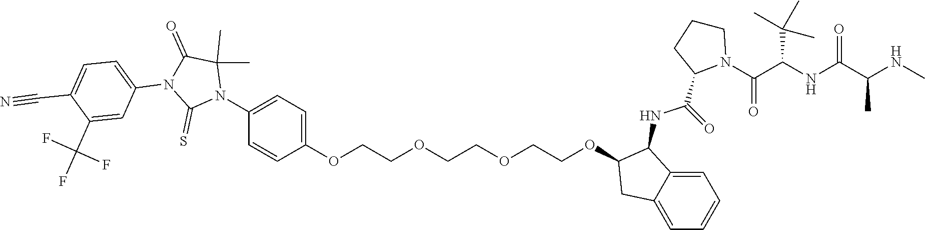 [SMILES: CN[C@@H](C)C(=O)N[C@H](C(=O)N1CCC[C@H]1C(=O)N[C@H]1c2ccccc2C[C@H]1OCCOCCOCCOc1ccc(N2C(=S)N(c3ccc(C#N)c(C(F)(F)F)c3)C(=O)C2(C)C)cc1)C(C)(C)C]

¹H NMR (300 MHz, CD₃OD): δ 8.19-8.11 (m, 2H), 8.03-7.97 (m, 1H), 7.39 (d, J=7.2 Hz, 1H), 7.32-7.25 (m, 2H), 7.24-7.18 (m, 2H), 7.17-7.04 (m, 3H), 5.41-5.38 (m, 1H), 4.66 (s, 1H), 4.56-4.51 (m, 1H), 4.32-4.29 (m, 1H), 4.16-4.13 (m, 2H), 3.97-3.80 (m, 4H), 3.79-3.58 (m, 9H), 3.07-3.05 (m, 2H), 2.66 (s, 3H), 2.26-1.89 (m, 4H), 1.58-1.45 (m, 9H), 1.12-1.04 (m, 9H). LC-MS (ES⁺): m/z 964.45 [MH⁺]

### Example 37 Synthesis of 1-[3,3-dimethyl-2-[(2S)-2-(methylamino)-propanamido]butanoyl]-N-[(1S,2R)-2-[2-[2-(4-[[(1r,3r)-3-(3-chloro-4-cyanophenoxy)-2,2,4,4-tetramethylcyclobutyl]carbamoyl]phenoxy)ethoxy]ethoxy]-2,3-dihydro-1H-inden-1-yl]pyrrolidine-2-carboxamide

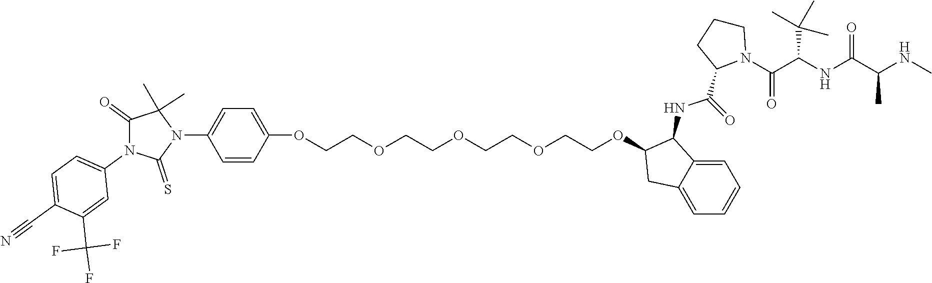 [SMILES: CN[C@@H](C)C(=O)N[C@H](C(=O)N1CCC[C@H]1C(=O)N[C@H]1c2ccccc2C[C@H]1OCCOCCOCCOCCOc1ccc(N2C(=S)N(c3ccc(C#N)c(C(F)(F)F)c3)C(=O)C2(C)C)cc1)C(C)(C)C]

¹H NMR (300 MHz, CD₃OD): δ 8.18-8.10 (m, 2H), 7.98 (d, J=8.4 Hz, 1H), 7.38 (d, J=7.2 Hz, 1H), 7.31-7.23 (m, 2H), 7.22-7.05 (m, 5H), 5.40-5.37 (m, 1H), 4.67 (s, 1H), 4.57-4.51 (m, 1H), 4.35-4.22 (m, 1H), 4.19-4.11 (m, 2H), 3.97-3.82 (m, 4H), 3.80-3.57 (m, 13H), 3.06-3.05 (m, 2H), 2.65 (s, 3H), 2.28-1.89 (m, 4H), 1.55-1.45 (m, 9H), 1.16-1.01 (m, 9H). LC-MS (ES⁺): m/z 1008.45 [MH⁺]

### Example 38 1-[3,3-dimethyl-2-[(2S)-2-(methylamino)propanamido]-butanoyl]-N-[(1S,2R)-2-[2-[2-(4-[[(1r,3r)-3-(3-chloro-4-cyanophenoxy)-2,2,4,4-tetramethylcyclobutyl]carbamoyl]phenoxy)ethoxy]ethoxy]-2,3-dihydro-1H-inden-1-yl]pyrrolidine-2-carboxamide

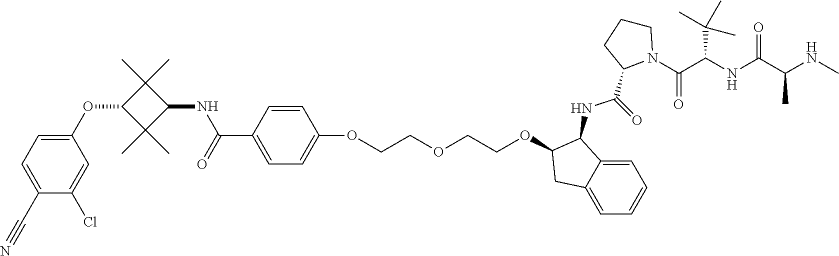 [SMILES: CN[C@@H](C)C(=O)N[C@H](C(=O)N1CCC[C@H]1C(=O)N[C@H]1c2ccccc2C[C@H]1OCCOCCOc1ccc(C(=O)N[C@H]2C(C)(C)[C@H](Oc3ccc(C#N)c(Cl)c3)C2(C)C)cc1)C(C)(C)C]

¹H NMR (300 MHz, CD₃OD):δ7.76-7.72 (m, 2H), 7.71-7.68 (m, 1H), 7.37-7.32 (m, 1H), 7.25-7.06 (m, 4H), 6.98-6.90 (m, 3H), 5.36-5.32 (m, 1H), 4.61 (s, 1H), 4.47-4.40 (m, 1H), 4.30-4.23 (m, 2H), 4.18-4.01 (m, 3H), 3.85-3.54 (m, 8H), 3.22-3.10 (m, 1H), 3.08-2.98 (m, 2H), 2.30 (s, 3H), 2.10-1.90 (m, 3H), 1.80-1.70 (m, 1H), 1.32-1.10 (m, 15H), 1.05 (s, 8H), 0.97 (s, 1H); LC-MS (ES⁺): m/z, 913.35 [MH⁺]

In the particular embodiment of the current invention, examples 1, 2, 3, 29, 30, 31, 32, 33, 34, 35, 36, 37, and 38 are PROTACs targeting degradation of the androgen receptor (AR), in which 501 and 502 are PTMs previously described by Crew, A. P. et al. in US 20150291562.

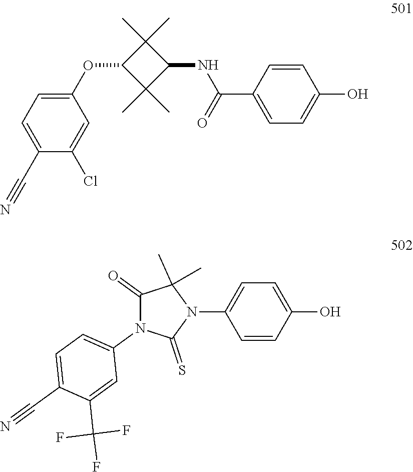 [SMILES: CC1(C)[C@H](NC(=O)c2ccc(O)cc2)C(C)(C)[C@H]1Oc1ccc(C#N)c(Cl)c1 ; CC1(C)C(=O)N(c2ccc(C#N)c(C(F)(F)F)c2)C(=S)N1c1ccc(O)cc1]

### Assays and Degradation Data

Protocol of the Cellular Assay of Androgen Receptor (AR) Degradation (VCaP Cells, ELISA).

For detection Cell Signaling PathScan Sandwich Elisa Catalog #12850 Lot 11 was used. VCaP cells were cultured in ATCC DMEM+ATCC FBS and plated 40,000/well 100 μl/well in RPMI P/S with 5% CSS Omega (bovine) serum into a 96 well plate. The cells were grown for a minimum of 3 days, dosed with compounds in 0.1% DMSO (diluted with 5% CSS) and incubated with aspiration for 4 hours. 100 μl of 1× Cell Signaling lysis buffer #9803 (36 ml dH2O+4 ml Cell Signaling lysis buffer) was added. The incubation was placed on cold room shaker for 10 min at speed 8-9. 5 μl to 100 μl of Diluent was transferred to Elisa plate (0.15 ug/ml-0.075 ug/ml) and stored at 4 C overnight on cold room shaker speed 5 (gentle swirl) and then shaken next morning at 37° C. for 30 min. The preparation was washed 4×200 μl with Elisa wash buffer and aspirated with eight-channel aspirator. 100 μl/well of AR detection Ab was added after which the preparation was covered and shaken at 37° C. for 1 hr. 100 ul TMB was added, and the mixture was shaken for 5 min while under observation. When TMB turned light blue, 100 ul of Stop solution was added, and the mixture was shaken and read at 450 nM. Also read at 562 for background subtraction.

The following PROTACs demonstrated androgen receptor degradation when tested under the conditions described above:

| | PROTAC | Target protein |
|---|---|---|
| Example | concentration, μM | degradation range |
| 1 | 1 | B |
| 2 | 1 | C |
| 3 | 1 | B |
| 29 | 1 | C |
| 30 | 1 | C |
| 31 | 1 | C |
| 32 | 1 | C |
| 33 | 1 | B |
| 35 | 1 | C |
| 36 | 1 | C |
| 37 | 1 | C |
| 38 | 1 | C |

*Protein degradation range at indicated concentration (relative to DMSO control):
A = degradation more than 60%;
B = degradation between 30% and 60%;
C = degradation between 0% and 30%.

In another embodiment of the current invention, examples 4, 5, 6, and 7 are PROTACs targeting degradation of TNIK, in which 503 is PTM previously described by Ho, K. in Bioorganic and Medicinal Chemistry Letters 2013, 23, 569-573.

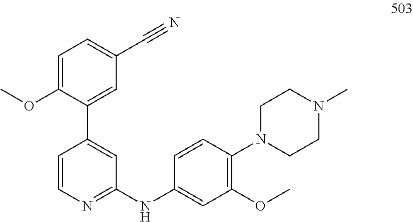

Protocol of the Cellular Assay of TNIK Degradation (HCT-116 Cells, Western Blot).

HCT-116 cells were plated into a 6-well plate at a density of 3×10⁵ cells per well in growth medium (McCoys 5a+10% Gibco FBS) and returned to the incubator for 24 hours to allow for cell attachment. After 24 hours, cells were dosed with PROTACs with a top concentration of 9 uM and 3 additional half-log dilutions in DMSO (9 uM, 3 uM, 1 uM, 0.3 uM, and 0). Cells were dosed such that the DMSO concentration was kept at 0.1%. Plates of cells were returned to the incubator for 24 hours prior to lysate preparation.

TNIK samples were prepared with 1× Cell Signaling Lysis Buffer with PIC, such that the lysate is fairly concentrated (1 well of a 6-well plate is lysed with 100 uL lysis buffer). Media was aspirated from the cells; the cell layer was washed with 3 mL of warm PBS. PBS was aspirated from the cell layer, and 100 uL of the cold lysis buffer was added, ensuring that all of the cells are covered. Plate was incubated on ice for 10 minutes. Plate was placed on the plate shaker and shaken on high speed for 1 to 2 minutes. Cell lysis buffer was pipetted up and down repeatedly until a homogenate was formed. The homogenate was transferred to a well of a 96-well plate.

For protein determination via BCA assay dilutions of the cell lysates (1:2, 1:5, & 1:10) were prepared, and 5 L of the dilutions were transferred to a BCA assay plate containing a BSA standard curve. 100 L of BCA reagent (50:1 Buffer A to Buffer B) was added to each well, and incubation was performed at 37*C for 30 minutes. The plate was read at A562, and protein concentrations were determined for each sample by interpolation.

The lysate samples were prepared in screw cap tubes with 4×SDS-PAGE sample buffer with β-mercaptoethanol (50 L βME/1 mL 4×SDS-PAGE sample buffer; 1:20; 5% βME) in such a way that at least 25 μg lysate could be added to each lane of the PAGE gel. The samples were boiled in a heat block at 100*C for 7 minutes. Samples were then returned to the ice bucket. Samples were then briefly centrifuged to pool the liquid at the bottom of the tube. Immediately prior to loading each sample, they were vortexed on high for 2 seconds.

SDS-PAGE gel (Bis-Tris; 4-12%) was prepared by washing out the wells with water. Gels were placed into the tank and filled with 1×MOPS buffer. Samples were loaded on the PAGE gel, vortexing prior to each addition. Midi gels were run at 100V for 2 hours (or mini gels at 120V for 1.5 hours).

SDS-PAGE gel was transferred onto PVDF (Millipore Immobilon-FL; 0.45 um) by tank transfer for 2.5 hours at 170 mA (˜350-400 mA*hours) in the cold room. After the tank transfer, the PVDF membrane was immediately washed in 1×TBST with 0.1% tween 20 for 2 minutes, followed by 3% BSA in 1×TBST with 0.1% tween 20 for 15 minutes (to block). Primary Santa Cruz TNIK mouse mAB antibody was added (1:1000; Cat #sc-377215) in 3% BSA in 1×TBST with 0.1% tween 20 and the membrane was incubated overnight at 4*C.

Membrane was washed for 30 minutes (3 washes×10 min) with 1×TBST with 0.1% tween 20. The secondary antibody was added (anti-mouse; 1:2,500; Cell Signaling Technology Cat #7076s) and incubation was performed at room temperature for 1 hour.

Membrane was washed for 30 minutes (3 washes×10 min) with 1×TBST with 0.1% tween 20. Membranes were added to the pre-mixed (1:1) Bio-Rad Clarity ECL substrate for 5 minutes. Luminescence was captured with the Bio-Rad Chemidoc MP for 1-30 seconds.

In another embodiment of the current invention, examples 8, 9, 10, and 11 are PROTACs targeting degradation of EZH2, in which 504 is a PTM derived from 505, previously described by Kuntz, K. W. et al. in the Journal of Medicinal Chemistry 2016, 59, 1556-1564.

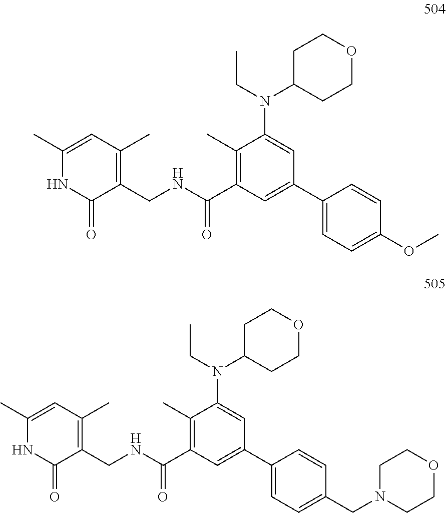

Protocol of the Cellular Assay of EZH2 Degradation (MDA-MB-231 Cells, Western Blot).

MDA-MB-231 cells were plated at 10,000 cells per well in 6 well plates, 2 ml/well in DMEM+10% Fetal Bovine Serum. Cells were allowed to grow for 3 days at 37 degrees C. Cells were dosed with PROTACs. On day 3 and day 7 after dosing, cells were harvested and lysed with RIPA buffer. Lysed cells were transferred to eppendorf tubes, and each lysate was sonicated and then spinned in microfuge for 15 minutes at 20,000×g at 4 degrees C. Supernatant was transferred to a clean tube, and protein concentration was quantitated using the Pierce BCA Protein Assay kit (cat #23225). Lysate concentrations were adjusted to 1 microgram/microliter in lysis buffer. For Western blot samples were loaded in 4×LDS buffer, 10 microliters/lane, onto Noxex NuPage 4-12% Bis-Tris Midi Gel 1.0 millimolar×26 well.

Gels were run on BioRad power source Model 1000/500, at 200V constant voltage for 1 hour. Gels were then transferred onto a nitrocellulose membrane using an iBlot2 transfer apparatus from Life Technologies. Transfers were done on Program 0 and blocked for 1 hour in TBS-T (Tris Buffered Saline with 0.05% Tween 20)+5% BSA (Bovine Serum Albumin). Block solution was decanted, and the primary antibody solutions were added (EZH2-Cell Signaling #5246S; tri-methyl-Histone H3-Cell Signaling #9733S; Histone H3-Cell Signaling #4499; all diluted 1:1000 in 20 ml TBS-T+5% BSA). The preparation was placed in cold room (4 degrees C.) overnight on rocker platform, after which the antibody solution was decanted. The preparation was washed 3 times in TBS-T for 10 minutes each, and anti-rabbit secondary antibody (Cell Signaling #7074S), diluted 1:20,000 in TBS-T+5% BSA, was added. This was followed by incubating for 1 hour at room temperature with gentle rocking and washing 3 times in TBS-T. To develop blots SuperSignal™ West Femto Maximum Sensitivity Substrate (Life Technologies Catalog number: 34095) solution was applied for 5 minutes. Blots were imaged on BioRad ChemiDoc Imager using the “Chemi Hi Sensitivity” protocol. Bands were quantitated using Image Lab software v 5.2.1.

The following PROTACs demonstrated EZH2 degradation when tested under the conditions described above:

| | PROTAC | Target protein |
|---|---|---|
| Example | concentration, μM | degradation range* |
| 8 | 3 | C |
| 9 | 3 | C |
| 10 | 3 | B |
| 11 | 3 | B |

*Protein degradation range at indicated concentration (relative to DMSO control):
A = degradation more than 60%;
B = degradation between 30% and 60%;
C = degradation between 0% and 30%.

In yet another embodiment of the current invention, examples 12, 13, 14, 15, 16, 17, 18, 19 and 20 are PROTACs targeting degradation of JNK1 and JNK2, in which 506 is a PTM previously described by Peng, C. et al. in WO 2007129195.

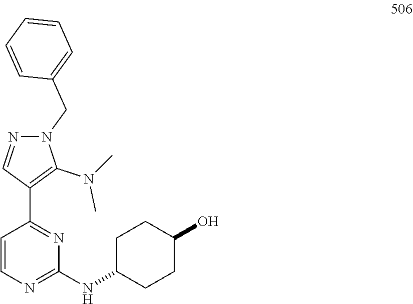 [SMILES: CN(C)c1c(-c2ccnc(N[C@H]3CC[C@H](O)CC3)n2)cnn1Cc1ccccc1]

Protocol of the cellular assay of JNK degradation (A549 cells, Western blot). A549 lung adenocarcinoma cells were purchased from ATCC and cultured in RPMI1640 Medium (Gibco) supplemented with 10% Fetal Bovine Serum (Gibco). DMSO control and PROTAC treatments (30 nM, 300 nM and 3000 nM) were performed in 24-well plates for 24 hours. Cells were stimulated with PMA (Phorbol 12-myristate 13-acetate, Sigma) for 1 hour before harvesting. Cells were lysed in Cell Signaling lysis buffer supplemented with protease inhibitors (Thermo). The lysates were centrifuged at 20,000×g for 15 minutes to clarify, and protein concentration was determined by BCA (Pierce). Equal amounts of protein (5 micrograms) were separated by SDS-PAGE and transferred onto nitrocellulose membranes. The mermbranes were probed with antibodies against SAPK/JNK (Cell Signaling #9252), phospho-c-Jun (Cell Signaling #3270), MDM2 (Sigma M4308) and p53 (Cell Signaling #2527). HRP-conjugated anti-rabbit and anti-mouse secondary antibodies were from Cell Signaling. The bands were visualized with SuperSignal West Femto Substrate (Thermo). Quantitation was done using Image Lab Software v5.2.1.

The following PROTACs demonstrated JNK1 and JNK2 degradation when tested under the conditions described above:

| | PROTAC | JNK1 | JNK1 | JNK2 | JNK2 |
|---|---|---|---|---|---|
| | concentration, | degradation | DC₅₀ | degradation | DC₅₀ |
| Example | μM | range* | range** | range* | range** |
| 12 | 1 | A | D | B | ND |
| 13 | 1 | A | D | A | E |
| 14 | 1 | A | D | B | ND |
| 15 | 1 | A | E | B | ND |
| 17 | 1 | A | E | A | E |
| 18 | 1 | A | D | A | E |
| 19 | 1 | A | D | A | E |

*Protein degradation range at indicated concentration (relative to DMSO control):
A = degradation more than 60%;
B = degradation between 30% and 60%;
C = degradation between 0% and 30%.
**Protein DC₅₀ range:
D = between 1 nM and 100 nM;
E = between 100 nM and 1000 nM;
ND = not determined.

In yet another embodiment of the current invention, examples 20 through 28 are PROTACs targeting degradation of BRD4, in which 507 is a PTM previously described by Filippakopoulos, P. et al. in Nature 2010, 468, 1067-1073 and by Zengerle, M. et al. in ACS Chemical Biology 2015, 10, 1770-1777.

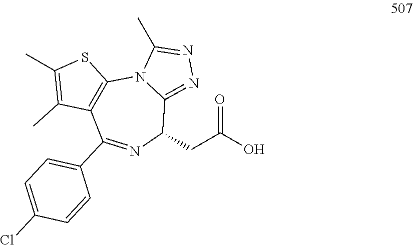 [SMILES: Cc1sc2c(c1C)C(c1ccc(Cl)cc1)=N[C@@H](CC(=O)O)c1nnc(C)n1-2]

The above-mentioned examples 20 through 28 when tested at 1 □M concentration demonstrated modification of the BRD4 protein function as evidenced by the downstream c-Myc suppression.

What is claimed is:

1. A compound having a chemical structure:
PTM-L-ILM,
wherein:
(a) L is a linker group coupling the ILM and the PTM;
(b) PTM is a protein target moiety that binds to a target protein or a target polypeptide;
(c) ILM is a IAP E3 ubiquitin ligase binding moiety represented by a chemical structure selected from: 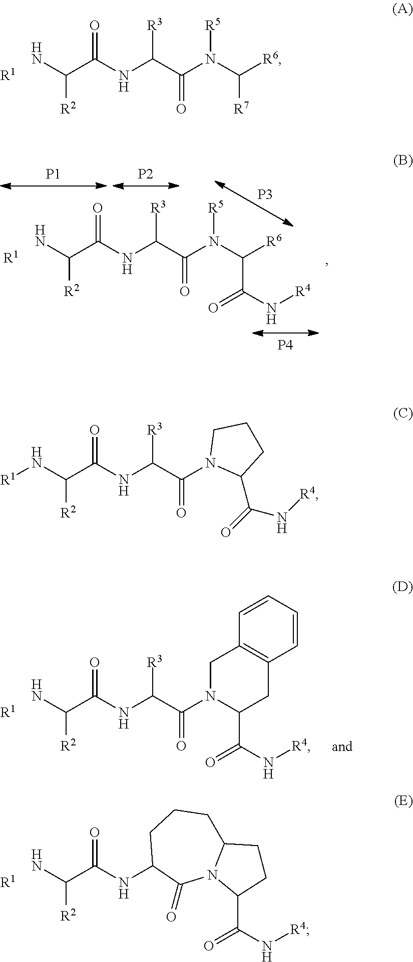
wherein:
R¹ is selected from the group of H and alkyl;
R² is selected from the group of H and alkyl;
R³ is selected from the group of H, alkyl, cycloalkyl and heterocycloalkyl;
R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above;
R⁵ and R⁶ are independently selected from the group of H, alkyl, cycloalkyl, heterocycloalkyl, aryl, heteroaryl or fused rings; and
R⁷ is selected from the group of cycloalkyl, cycloalkylalkyl, heterocycloalkyl, heterocycloalkylalkyl, aryl, arylalkyl, heteroaryl or heteroarylalkyl, each one further optionally substituted with 1-3 substituents selected from halogen, alkyl, haloalkyl, hydroxyl, alkoxy, cyano, (hetero)cycloalkyl or (hetero)aryl, or —C(O)NH—R⁴, where R⁴ is selected from alkyl, cycloalkyl, heterocycloalkyl, cycloalkylalkyl, heterocycloalkylalkyl, aryl, arylalkyl, heteroaryl, heteroarylalkyl, further optionally substituted with 1-3 substituents as described above, or
a pharmaceutically acceptable salt, enantiomer, stereoisomer, solvate or polymorph thereof

2. A method for inducing degradation of a target protein in a subject comprising administering an effective amount of the compound of claim 1 to the subject.

3. A method for treating a disease state or condition in a patient wherein dysregulated protein activity is responsible for said disease state or condition, said method comprising administering an effective amount of a compound according to claim 1.
